(19) United States
(12) Patent Application Publication
(10) Pub. No.: US 2021/0269818 A1
(43) Pub. Date: Sep. 2, 2021

(54) THERMOSTABLE RUBISCO ACTIVASE AND USES THEREOF

(71) Applicant: BASF SE, Ludwigshafen (DE)

(72) Inventors: Andrew Scafaro, Lyneham (AU); Alexander Galle, Gent (BE); Jeroen Van Rie, Gent (BE); Lambertus Den Boer, Gent (BE); Katelijn D'Halluin, Gent (BE)

(21) Appl. No.: 17/254,439

(22) PCT Filed: Jun. 21, 2019
(86) PCT No.: PCT/EP2019/066480

§ 371 Date: Dec. 21, 2020

(30) Foreign Application Priority Data
Jun. 27, 2018 (EP) 18180087.1
Dec. 3, 2018 (EP) 18209806.1

(51) Int. Cl. C12N 15/82

(52) U.S. Cl. CPC C12N 15/8269; C12Y 401/01039

(57) ABSTRACT

The present invention relates to the field of agriculture. In particular, the invention provides a thermostable Rca proteins, a recombinant gene, plants comprising the recombinant genes and a method to improve thermotolerance of a cereal plant under stress conditions.

### FIELD OF THE INVENTION

The present invention relates to methods and means to increase the ratio of thermostable Rubisco Activase (Rca) proteins in cereals and improve the tolerance of cereal plants to heat stress. In particular, the invention provides Rca 2 protein variants the thermostability of which is increased compared to the native Rca 2 proteins.

### BACKGROUND

Ribulose-1,5-bisphosphate carboxylase/oxygenase (Rubisco) is the central enzyme of photosynthesis converting inert CO₂ gas from the atmosphere into sugars. Rubisco is tightly regulated and the active site of Rubisco is prone to inhibition, even by its sugar substrate Ribulose-1,5-bisphosphate (RuBP)¹. The regulation of Rubisco and removal of inhibitors from the Rubisco active site is undertaken by its chaperone enzyme Rubisco activase (Rca)². Rca is a member of the AAA⁺ family of enzymes and utilises ATP to mechanically remove tightly bound inhibitors from the Rubisco active site³. One of the defining characteristics of Rca is that it is a heat-labile protein and in many plant species disassociates, denatures and aggregates out of solution with even moderate heat application, to a much greater extent that Rubisco⁴⁻⁶. As such, Rca is considered one of the leading causes of a lack of photosynthetic function for plants exposed to supra-optimal temperatures⁷⁻¹⁰. Improving the thermostability of Rca is therefore considered one of the most promising ways of improving photosynthesis and thus potentially yield of crops exposed to the detrimental impacts of heat stress^{11,12}.

The thermal stability of Rca is dependent on species and correlates with the climate in which a species has evolved, with temperate species having Rca that is heat-labile relative to tropical species^{13,14} Even closely related species from varying environments, such as rice (Oryza sativa) and a close relative Oryza australiensis, endemic to hot environments in the north of Australia, have divergent thermostable variants of Rca¹⁵. The genetic diversity in thermostability of Rca between species is currently being exploited to improve the stability of more susceptible Rca variants. For example, a recent study attempted to improve rice Rca thermostability based on the more thermostable Rca from Agave tequilana, a CAM desert plant¹⁶.

Cereals, such as wheat (Triticum aestivum) are important food crops. Wheat is a temperate grass and photosynthesis of wheat is already impaired at temperatures well below 40° C.¹⁷. There remains thus a need to improve the thermostability of the cereals Rca proteins, such as wheat Rca proteins.

### SUMMARY

In one aspect, the invention provides a method for increasing the ratio of a thermostable Rca (Rubisco Activase) protein in cereals, such as wheat, comprising (a) providing to cells of a cereal plant a gene, such as a recombinant gene, comprising as operably linked elements a promoter, preferably expressible in plants; a nucleic acid encoding an Rca 1β protein and variants thereof or encoding a thermostable Rca 2 protein variant and, optionally a transcription termination and polyadenylation region, preferably a transcription termination and polyadenylation region functional in plants; and reducing the expression of endogenous non-thermostable Rca 2 protein in said cereal plant cells, wherein said ratio is increased compared to a control cereal plant cell not comprising said recombinant gene; or (b) introducing into cells of a cereal plant at least one thermostable Rca 2 allele wherein said thermostable Rca 2 allele encodes an amino acid comprising the amino acid sequence of SEQ ID NOs: 32 or 35 or an amino acid sequence having 90% identity with SEQ ID NOs: 32 or 35 and comprising at least one amino acid selected from: i) an isoleucine at a position corresponding to position 59 of SEQ ID NO: 32 or 35; ii) an aspartic acid at a position corresponding to position 73 of SEQ ID NO: 32 or 35; iii) an isoleucine at a position corresponding to position 160 of SEQ ID NO: 32 or 35; iv) an arginine at a position corresponding to position 265 of SEQ ID NO: 32 or 35; v) a proline at a position corresponding to position 270 of SEQ ID NO: 32 or 35; vi) a leucine at a position corresponding to position 277 of SEQ ID NO: 32 or 35; vii) a glutamic acid at a position corresponding to position 307 of SEQ ID NO: 32 or 35; viii) an isoleucine at a position corresponding to position 334 of SEQ ID NO: 32 or 35; ix) a lysine at a position corresponding to position 359 of SEQ ID NO: 32 or 35; x) a leucine at a position corresponding to position 361 of SEQ ID NO: 32 or 35; and xi) a glutamic acid at a position corresponding to position 363 of SEQ ID NO: 32 or 35, wherein said ratio is increased compared to a control cereal plant cell not comprising said thermostable Rca 2 allele.

In a further embodiment, the Rca 1β protein and variants thereof comprise an amino acid sequence selected from the amino acid sequence of SEQ ID NO: 8 or an amino acid sequence having at least 90% identity to the amino acid sequence of SEQ ID NO: 8 and comprising at least one amino acid selected from (i) an isoleucine at a position corresponding to position 109 of SEQ ID NO: 8; (ii) an aspartic acid at a position corresponding to position 123 of SEQ ID NO: 8; (iii) an isoleucine at a position corresponding to position 210 of SEQ ID NO: 8; (iv) an arginine at a position corresponding to position 315 of SEQ ID NO: 8; (v) a proline at a position corresponding to position 320 of SEQ ID NO: 8; (vi) a leucine at a position corresponding to position 327 of SEQ ID NO: 8; (vii) a glutamic acid at a position corresponding to position 357 of SEQ ID NO: 8; (viii) an isoleucine at a position corresponding to position 384 of SEQ ID NO: 8; (ix) a lysine at a position corresponding to position 409 of SEQ ID NO: 8; (x) a leucine at a position corresponding to position 411 of SEQ ID NO: 8; and (xi) a glutamic acid at a position corresponding to position 413 of SEQ ID NO: 8.

Furthermore, the nucleic acid encoding an Rca 1β protein and variants thereof may comprise a coding nucleic acid sequence selected from the nucleic acid of SEQ ID NO: 7, or complement thereof, and a nucleic acid having at least 60% identity to the nucleic acid of SEQ ID NO: 7, or complement thereof.

In yet another embodiment, the thermostable Rca 2 protein variants comprise an amino acid sequence selected from the amino acid sequences of SEQ ID NO: 30 or 33 and an amino acid sequence having at least 90% identity to the amino acid sequences of SEQ ID NO: 30 or 33 and comprising at least one amino acid selected from (i) an isoleucine at a position corresponding to position 105 of SEQ ID NO: 30 or 33, (ii) an aspartic acid at a position corresponding to position 119 of SEQ ID NO: 30 or 33, (iii) an isoleucine at a position corresponding to position 206 of SEQ ID NO: 30 or 33, (iv) an arginine at a position corresponding to position 311 of SEQ ID NO: 30 or 33, (v) a proline at a position corresponding to position 316 of SEQ ID NO: 30 or 33, (vi) a leucine at a position corresponding to position 323 of SEQ ID NO: 30 or 33, (vii) a glutamic acid at a position corresponding to position 353 of SEQ ID NO: 30 or 33, (viii) an isoleucine at a position corresponding to position 380 of SEQ ID NO: 30 or 33, (ix) a lysine at a position corresponding to position 405 of SEQ ID NO: 30 or 33, (x) a leucine at a position corresponding to position 407 of SEQ ID NO: 30 or 33 and (xi) a glutamic acid at a position corresponding to position 409 of SEQ ID NO: 30 or 33.

Furthermore, the thermostable Rca 2 protein variant may comprise an amino acid sequence selected from the amino acid sequences of SEQ ID NO: 32 or 35 and further comprising a chloroplast targeting peptide, and an amino acid sequence having at least 90% identity to the amino acid sequences of SEQ ID NO: 32 or 35, further comprising a chloroplast targeting peptide, and comprising at least one amino acid selected from (i) an isoleucine at a position corresponding to position 59 of SEQ ID NO: 32 or 35, (ii) an aspartic acid at a position corresponding to position 73 of SEQ ID NO: 32 or 35, (iii) an isoleucine at a position corresponding to position 160 of SEQ ID NO: 32 or 35, (iv) an arginine at a position corresponding to position 265 of SEQ ID NO: 32 or 35, (v) a proline at a position corresponding to position 270 of SEQ ID NO: 32 or 35, (vi) a leucine at a position corresponding to position 277 of SEQ ID NO: 32 or 35, (vii) a glutamic acid at a position corresponding to position 307 of SEQ ID NO: 32 or 35, (viii) an isoleucine at a position corresponding to position 334 of SEQ ID NO: 32 or 35, (ix) a lysine at a position corresponding to position 359 of SEQ ID NO: 32 or 35, (x) a leucine at a position corresponding to position 361 of SEQ ID NO: 32 or 35 and (xi) a glutamic acid at a position corresponding to position 363 of SEQ ID NO: 32 or 35. In addition, the nucleic acid encoding a thermostable Rca 2 protein variant may comprise a coding nucleotide sequence selected from (a) the nucleotide sequence of SEQ ID Nos: 31, 34, 36 or 37, or the complement thereof, (b) a nucleotide sequence having at least 60% identity with the nucleotide sequence of SEQ ID Nos: 31, 34, 36 or 37, or the complement thereof.

In another embodiment, reducing expression of endogenous non-thermostable Rca 2 protein comprises introducing into said cells of a cereal plant at least one knock out mutant Rca 2 allele or providing said cells of a cereal plant with a second recombinant gene capable of suppressing specifically the expression of the endogenous non-thermostable Rca 2 genes.

In yet another embodiment, the second recombinant gene capable of suppressing specifically the expression of the endogenous non-thermostable Rca 2 genes comprises the following operably linked elements (a) a promoter, preferably expressible in plants, (b) a nucleic acid which when transcribed yields an RNA molecule inhibitory to the endogenous Rca 2 genes encoding a non-thermostable Rca protein but not inhibitory to genes encoding thermostable Rca proteins; and, optionally (c) a transcription termination and polyadenylation region, preferably a transcription termination and polyadenylation region functional in plants.

In still another embodiment, the thermostable mutant Rca 2 allele comprises the coding nucleotide sequence of SEQ ID NOs: 31, 34, 36 or 37 or a coding nucleotide sequence having at least 60% identity with SEQ ID NOs: 31, 34, 36 or 37 and encoding a protein comprising at least one of the amino acids selected from (i) an isoleucine at a position corresponding to position 59 of SEQ ID NO: 32 or 35, (ii) an aspartic acid at a position corresponding to position 73 of SEQ ID NO: 32 or 35, (iii) an isoleucine at a position corresponding to position 160 of SEQ ID NO: 32 or 35, (iv) an arginine at a position corresponding to position 265 of SEQ ID NO: 32 or 35, (v) a proline at a position corresponding to position 270 of SEQ ID NO: 32 or 35, (vi) a leucine at a position corresponding to position 277 of SEQ ID NO: 32 or 35, (vii) a glutamic acid at a position corresponding to position 307 of SEQ ID NO: 32 or 35, (viii) an isoleucine at a position corresponding to position 334 of SEQ ID NO: 32 or 35, (ix) a lysine at a position corresponding to position 359 of SEQ ID NO: 32 or 35, (x) a leucine at a position corresponding to position 361 of SEQ ID NO: 32 or 35 and (xi) a glutamic acid at a position corresponding to position 363 of SEQ ID NO: 32 or 35.

Methods for increasing thermotolerance of a cereal plant, for increasing yield of a cereal plant under heat stress conditions and for producing a cereal plant with increased thermotolerance are also provided. These methods comprise increasing the ratio of a thermostable Rca protein according to the invention and regenerating the cereal plant.

In another aspect, the invention provides a thermostable Rca protein variant and a nucleic acid encoding it, with the thermostable Rca 2 protein variant comprising an amino acid sequence selected from (a) the amino acid sequences of SEQ ID NO: 30 or 33 and (b) an amino acid sequence having at least 90% identity to the amino acid sequences of SEQ ID NO: 30 or 33 and comprising at least one amino acid selected from (i) an isoleucine at a position corresponding to position 105 of SEQ ID NO: 30 or 33, (ii) an aspartic acid at a position corresponding to position 119 of SEQ ID NO: 30 or 33, (iii) an isoleucine at a position corresponding to position 206 of SEQ ID NO: 30 or 33, (iv) an arginine at a position corresponding to position 311 of SEQ ID NO: 30 or 33, (v) a proline at a position corresponding to position 316 of SEQ ID NO: 30 or 33, (vi) a leucine at a position corresponding to position 323 of SEQ ID NO: 30 or 33, (vii) a glutamic acid at a position corresponding to position 353 of SEQ ID NO: 30 or 33, (viii) an isoleucine at a position corresponding to position 380 of SEQ ID NO: 30 or 33, (ix) a lysine at a position corresponding to position 405 of SEQ ID NO: 30 or 33, (x) a leucine at a position corresponding to position 407 of SEQ ID NO: 30 or 33 and (xi) a glutamic acid at a position corresponding to position 409 of SEQ ID NO: 30 or 33, (c) the amino acid sequences of SEQ ID NO: 32 or 35 and optionally further comprising a chloroplast targeting peptide, (d) an amino acid sequence having at least 90% identity to the amino acid sequences of SEQ ID NO: 32 or 35, optionally further comprising a chloroplast targeting peptide, and comprising at least one amino acid selected from (i) an isoleucine at a position corresponding to position 59 of SEQ ID NO: 32 or 35, (ii) an aspartic acid at a position corresponding to position 73 of SEQ ID NO: 32 or 35, (iii) an isoleucine at a position corresponding to position 160 of SEQ ID NO: 32 or 35, (iv) an arginine at a position corresponding to position 265 of SEQ ID NO: 32 or 35, (v) a proline at a position corresponding to position 270 of SEQ ID NO: 32 or 35, (vi) a leucine at a position corresponding to position 277 of SEQ ID NO: 32 or 35, (vii) a glutamic acid at a position corresponding to position 307 of SEQ ID NO: 32 or 35, (viii) an isoleucine at a position corresponding to position 334 of SEQ ID NO: 32 or 35, (ix) a lysine at a position corresponding to position 359 of SEQ ID NO: 32 or 35, (x) a leucine at a position corresponding to position 361 of SEQ ID NO: 32 or 35; and (xi) a glutamic acid at a position corresponding to position 363 of SEQ ID NO: 32 or 35. A cereal plant comprising the thermotolerant Rca 2 protein variant of the invention is also provided.

In yet another aspect, a gene, such as a recombinant gene, comprising the following operably linked elements (a) a promoter, preferably expressible in plants, (b) a nucleic acid encoding a Rca protein selected from (i) an Rca 1β protein and variants thereof, and (ii) a thermostable Rca 2 protein variant and, optionally (c) a transcription termination and polyadenylation region, preferably a transcription termination and polyadenylation region functional in plants. In further embodiments, the Rca 1β protein and variants thereof comprise the amino acid sequence as described above and the thermostable Rca 2 protein variant comprises an amino acid sequence according to the invention.

Further provided are a knock out allele of an Rca 2 gene and a recombinant gene capable of suppressing specifically the expression of the endogenous Rca 2 genes comprising the following operably linked elements (a) a promoter, preferably expressible in plants, (b) a nucleic acid which when transcribed yields an RNA molecule inhibitory to the endogenous Rca 2 genes encoding a non-thermostable Rca protein but not inhibitory to genes encoding thermostable Rca proteins and, optionally (c) a transcription termination and polyadenylation region, preferably a transcription termination and polyadenylation region functional in plants.

In another embodiment, a thermostable allele of a Rca 2 gene is provided. The thermostable allele according to the invention may comprise (a) a coding nucleotide sequence of SEQ ID NOs: 31, 34, 36 or 37, or (b) a coding nucleotide sequence having at least 60% identity to SEQ ID NO: 31, 34, 36 or 37 and encoding a protein comprising at least one amino acid selected from (i) an isoleucine at a position corresponding to position 59 of SEQ ID NO: 32 or 35, (ii) an aspartic acid at a position corresponding to position 73 of SEQ ID NO: 32 or 35, (iii) an isoleucine at a position corresponding to position 160 of SEQ ID NO: 32 or 35, (iv) an arginine at a position corresponding to position 265 of SEQ ID NO: 32 or 35, (v) a proline at a position corresponding to position 270 of SEQ ID NO: 32 or 35, (vi) a leucine at a position corresponding to position 277 of SEQ ID NO: 32 or 35, (vii) a glutamic acid at a position corresponding to position 307 of SEQ ID NO: 32 or 35, (viii) an isoleucine at a position corresponding to position 334 of SEQ ID NO: 32 or 35, (ix) a lysine at a position corresponding to position 359 of SEQ ID NO: 32 or 35, (x) a leucine at a position corresponding to position 361 of SEQ ID NO: 32 or 35 and (xi) a glutamic acid at a position corresponding to position 363 of SEQ ID NO: 32 or 35.

In another aspect, a cell is provided which comprises the (recombinant) gene according to the invention, at least one knock out allele of an Rca 2 gene as described herein, a recombinant gene capable of suppressing specifically the expression of the endogenous Rca 2 genes, at least one thermostable allele of a Rca 2 gene according to the invention and/or the thermostable Rca 2 protein variant described herein. A cereal plant, plant part or seed consisting essentially of the cells according to the invention are also provided. These cereal plant, plant part or seed may be wheat plant, wheat plant part or wheat seed.

Also provided is the use of the thermostable Rca 2 protein variant according to the invention, the nucleic acid encoding a thermostable Rca 2 protein variant according to the invention, the recombinant gene according to the invention, the recombinant gene capable of suppressing specifically the expression of the endogenous Rca 2 genes described herein or the thermostable allele of a Rca 2 gene provided herewith to increase the ratio of a thermostable Rca protein in cereals, to increase thermotolerance of a cereal plant, to increase yield of a cereal plant under heat stress conditions or to produce a cereal plant with increased thermotolerance.

### BRIEF DESCRIPTION OF THE DRAWINGS

FIG. 1. A comparison of gene expression and temperature dependent Rubisco activation velocity of Rca extracted from wheat leaves at either control (22° C. day/night) or after heat treatment (38/22° C. for two diurnal cycles). (A) gene expression by qPCR for primers specific to the wheat Rca1 β (TaRca1-β) gene, and Rca2 gene α (TaRca2-α) and Rca2 β spliced variants (TaRca2-β). (B) absolute initial velocity of Rubisco activation by Rca extracted from control and heat treated leaves of wheat and incubated for 10-min in the presence of 0.2 mM ATP at indicated temperatures prior to assaying at a standard 25° C. (C) Rubisco activation velocity of wheat leaf Rca versus incubation temperature normalised to the fastest velocity achieved. Values are means±SD of three or more biological replicates. Curves are the ordinary least-squares fit of a variable slope model (Eqn. 1).

FIG. 2. Rubisco activation velocity by Rca at 25° C. (A), temperature dependent Rubisco activation by Rca (B) and differential scanning fluorimetry (C) for recombinant wheat Rca2 α (TaRca2-α), β (TaRca2-β), Rca1 β (TaRca1-β) and rice Rca β (OsRca-β) isoforms. Rubisco activation experiments consisted of 1.4±0.2 μM of Rca protomer added to 0.2±0.05 μM of Rubisco active sites inhibited by RuBP (ER). For temperature response curves Rca was incubated for 10-min in the presence of 0.2 mM ATP at indicated temperatures prior to assaying at a standard 25° C. and values are normalised to the fastest velocity achieved. For DSF, samples were heated at 1° C. per minute and fluorescence signal normalised to the maximum value recorded. Values are means±SD of four experimental replicates. Curves are the ordinary least-squares fit of a Boltzmann sigmoidal equation (Eqn. 1 and 2).

FIG. 3. Sequence alignment of wheat Rca2 β (TaRca2-β), Rca1 β (TaRca1-β), rice Rca β (OsRca-β), the consensus sequence of warm and cold adapted species, a mutation of wheat Rca2 β with 11 amino acid changes (TaRca2-β-11AA) and eight amino acid changes (TaRca2-β-8AA). The consensus sequences were generated from alignment of eight and nine species, endemic to warm and cold environments, respectively. The mutations made to TaRca2-β-11AA with positions indicated by open and filled triangles, were selected based on the TaRca1-β sequence with the criteria that TaRca1-β matched the warm species consensus and was different to the cold species consensus. The mutations made to TaRca2-β-8AA with positions indicated by filled triangles, were selected based on the above criteria and the additional criteria that OsRca-β could not match TaRca2-β or the cold species consensus. Residue differences among the sequences are highlighted. The chloroplast signal peptide, which was not included in analysis, is underlined.

FIG. 4. Rubisco activation velocity by Rca at 25° C. (A) temperature dependent Rubisco activation by Rca (B) and differential scanning fluorimetry (C) for recombinant wheat Rca2 β (TaRca2-β), Rca1 β (TaRca1-β) and rice Rca β (OsRca-β) isoforms as presented in FIG. 2, with the addition of TaRca2-β 11 amino acid (TaRca2-β-11AA) and 8 amino acid (TaRca2-β-8AA) mutants. Values are means±SD of four or more experimental replicates. Curves are the ordinary least-squares fit of a Boltzmann sigmoidal equation (Eqn. 1 and 2).

FIG. 5. Schematic representation of the gene replacement strategy followed in Example 6. TS1: target site 1, TS2: target site 2, arrows marked g1, g9, g14 to 18 represent the positions where the guide RNAs cause a double strand break in the genomic DNA. The amino acid substitutions in TaRca2-β-11AA with positions are indicated by open and filled triangles, while the amino acid substitutions in TaRca2-ρ-8AA with positions are indicated by filled triangles. Asterisks represent silent mutations over cleavage site.

### DETAILED DESCRIPTION

The present invention is based on the surprising discovery that the wheat Rca 1β protein is a thermostable Rca isoform and that a limited number of amino acid changes on a non-thermostable Rca 2 protein result in an improved thermostability of the Rca 2 protein by at least 7° C. in in vitro Rubisco activation assays.

### Definitions

As used herein for protein sequences, the term “percent sequence identity” refers to the percentage of conserved amino acids between two segments of a window of optimally aligned polypeptides. Optimal alignment of sequences for aligning a comparison window are well-known to those skilled in the art and the percentage of conservation may be calculated by matrix such as BLOSUM (Blocks Substitution Matrix) and PAM (Point Accepted Mutation) (Henikoff and Henikoff, 1992, PNAS 89(22):10915-10919). An “identity fraction” for aligned segments of a test sequence and a reference sequence is the number of identical or conserved components that are shared by the two aligned sequences divided by the total number of components in the reference sequence segment, i.e., the entire reference sequence or a smaller defined part of the reference sequence. Percent sequence identity is represented as the identity fraction times 100. The comparison of one or more protein sequences may be to a full-length protein sequence or a portion thereof, or to a longer protein sequence.

The term “protein” interchangeably used with the term “polypeptide” as used herein describes a group of molecules consisting of more than 30 amino acids, whereas the term “peptide” describes molecules consisting of up to 30 amino acids. Proteins and peptides may further form dimers, trimers and higher oligomers, i.e. consisting of more than one (poly)peptide molecule. Protein or peptide molecules forming such dimers, trimers etc. may be identical or non-identical. The corresponding higher order structures are, consequently, termed homo- or heterodimers, homo- or heterotrimers etc. The terms “protein” and “peptide” also refer to naturally modified proteins or peptides wherein the modification is effected e.g. by glycosylation, acetylation, phosphorylation and the like. Such modifications are well known in the art.

The term “variant” with respect to the nucleotide sequences of the invention is intended to mean substantially similar sequences. Naturally occurring allelic variants such as these can be identified with the use of well-known molecular biology techniques, as, for example, with polymerase chain reaction (PCR) and hybridization techniques as herein outlined before. Variant nucleotide sequences also include synthetically derived nucleotide sequences, such as those generated, for example, by using site-directed mutagenesis of any one of SEQ ID NOs: 7, 46 or 48. Generally, nucleotide sequence variants of the invention will have at least 40%, at least 50%, at least 60%, to at least 70%, e.g., preferably at least 71%, at least 72%, at least 73%, at least 74%, at least 75%, at least 76%, at least 77%, at least 78%, to at least 79%, generally at least 80%, e.g., at least 81% to at least 84%, at least at least 85%, e.g., at least 86%, at least 87%, at least 88%, at least 89%, at least 90%, at least 91%, at least 92%, at least 93%, at least 94%, at least 95%, at least 96%, at least 97%, to at least 98% and at least 99% nucleotide sequence identity to the native (wild type or endogenous) nucleotide sequence. Derivatives of the DNA molecules disclosed herein may include, but are not limited to, deletions of sequence, single or multiple point mutations, alterations at a particular restriction enzyme site, addition of functional elements, or other means of molecular modification. Techniques for obtaining such derivatives are well-known in the art (see, for example, J. F. Sambrook, D. W. Russell, and N. Irwin (2000) Molecular Cloning: A Laboratory Manual, 3^{rd} edition Volumes 1, 2, and 3. Cold Spring Harbor Laboratory Press). Those of skill in the art are familiar with the standard resource materials that describe specific conditions and procedures for the construction, manipulation, and isolation of macromolecules (e.g., DNA molecules, plasmids, etc.), as well as the generation of recombinant organisms and the screening and isolation of DNA molecules.

As used herein for nucleotide sequences, the term “percent sequence identity” refers to the percentage of identical nucleotides between two segments of a window of optimally aligned DNA. Optimal alignment of sequences for aligning a comparison window are well-known to those skilled in the art and may be conducted by tools such as the local homology algorithm of Smith and Waterman (Waterman, M. S. Introduction to Computational Biology: Maps, sequences and genomes. Chapman & Hall. London (1995), the homology alignment algorithm of Needleman and Wunsch (J. Mol. Biol., 48:443-453 (1970), the search for similarity method of Pearson and Lipman (Proc. Natl. Acad. Sci., 85:2444 (1988), and preferably by computerized implementations of these algorithms such as GAP, BESTFIT, FASTA, and TFASTA available as part of the GCG (Registered Trade Mark), Wisconsin Package (Registered Trade Mark from Accelrys Inc., San Diego, Calif.). An “identity fraction” for aligned segments of a test sequence and a reference sequence is the number of identical components that are shared by the two aligned sequences divided by the total number of components in the reference sequence segment, i.e., the entire reference sequence or a smaller defined part of the reference sequence. Percent sequence identity is represented as the identity fraction times 100. The comparison of one or more DNA sequences may be to a full-length DNA sequence or a portion thereof, or to a longer DNA sequence.

The term “recombinant gene” refers to any artificial gene that contains: a) DNA sequences, including regulatory and coding sequences that are not found together in nature, or b) sequences encoding parts of proteins not naturally adjoined, or c) parts of promoters that are not naturally adjoined. Accordingly, a recombinant gene may comprise regulatory sequences and coding sequences that are derived from different sources, i.e. heterologous sequences, or comprise regulatory sequences, and coding sequences derived from the same source, but arranged in a manner different from that found in nature.

The term “heterologous” refers to the relationship between two or more nucleic acid or protein sequences that are derived from different sources. For example, a promoter is heterologous with respect to an operably linked DNA region, such as a coding sequence if such a combination is not normally found in nature. In addition, a particular sequence may be “heterologous” with respect to a cell or organism into which it is inserted (i.e. does not naturally occur in that particular cell or organism). For example, the recombinant gene disclosed herein is a heterologous nucleic acid.

The term “endogenous” relates to what originate from within the plant or cell. An endogenous gene is thus a gene originally found in a given plant or cell.

“Isolated nucleic acid”, used interchangeably with “isolated DNA” as used herein refers to a nucleic acid not occurring in its natural genomic context, irrespective of its length and sequence. Isolated DNA can, for example, refer to DNA which is physically separated from the genomic context, such as a fragment of genomic DNA. Isolated DNA can also be an artificially produced DNA, such as a chemically synthesized DNA, or such as DNA produced via amplification reactions, such as polymerase chain reaction (PCR) well-known in the art. Isolated DNA can further refer to DNA present in a context of DNA in which it does not occur naturally. For example, isolated DNA can refer to a piece of DNA present in a plasmid. Further, the isolated DNA can refer to a piece of DNA present in another chromosomal context than the context in which it occurs naturally, such as for example at another position in the genome than the natural position, in the genome of another species than the species in which it occurs naturally, or in an artificial chromosome.

Hybridization occurs when the two nucleic acid molecules anneal to one another under appropriate conditions. Nucleic acid hybridization is a technique well known to those of skill in the art of DNA manipulation. The hybridization property of a given pair of nucleic acids is an indication of their similarity or identity. Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence. “Stringent hybridization conditions” and “stringent hybridization wash conditions” in the context of nucleic acid hybridization experiments such as Southern and Northern hybridization are sequence dependent, and are different under different environmental parameters. An example of highly stringent wash conditions is 0.15 M NaCl at 72° C. for about 15 minutes. An example of stringent wash conditions is a 0.2×SSC wash at 65° C. for 15 minutes. Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The phrases “DNA”, “DNA sequence,” “nucleic acid sequence,” “nucleic acid molecule” “nucleotide sequence” and “nucleic acid” refer to a physical structure comprising an orderly arrangement of nucleotides. The terms “sequence” and “molecule” may be used interchangeably. The DNA sequence or nucleotide sequence may be contained within a larger nucleotide molecule, vector, or the like. In addition, the orderly arrangement of nucleic acids in these sequences may be depicted in the form of a sequence listing, figure, table, electronic medium, or the like.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A recombinant gene comprising a nucleic acid which is functionally or structurally defined, may comprise additional DNA regions etc. However, in context with the present disclosure, the term “comprising” also includes “consisting of”.

It is understood that when referring to a word in the singular (e.g. plant or allele), the plural is also included herein (e.g. a plurality of plants, a plurality of alleles). Thus, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, the term “allele(s)” means any of one or more alternative forms of a gene at a particular locus. In a diploid (or amphidiploid) cell of an organism, alleles of a given gene are located at a specific location or locus (loci plural) on a chromosome. One allele is present on each chromosome of the pair of homologous chromosomes.

As used herein, the term “locus” (loci plural) means a specific place or places or a site on a chromosome where for example a gene or genetic marker is found. For example, the “Rca 2 A locus” refers to the position on a chromosome of the A genome where an Rca 2 A gene (and two Rca 2 A alleles) may be found, while the “Rca 2 B locus” refers to the position on a chromosome of the B genome where an Rca 2 B gene (and two Rca 2B alleles) may be found and the “Rca 2 D locus” refers to the position on a chromosome of the D genome where an Rca 2 D gene (and two Rca 2 D alleles) may be found.

“Wild type” (also written “wildtype” or “wild-type”), as used herein, refers to a typical form of a plant or a gene as it most commonly occurs in nature. A “wild type plant” refers to a plant with the most common phenotype of such plant in the natural population. A “wild type allele” refers to an allele of a gene required to produce the wild-type protein and wild type phenotype. By contrast, a “mutant plant” refers to a plant with a different rare phenotype of such plant in the natural population or produced by human intervention, e.g. by mutagenesis or gene editing, and a “mutant allele” refers to an allele of a gene required to produce the mutant protein and/or the mutant phenotype.

“Mutant” as used herein refers to a form of a plant or a gene which is different from such plant or gene in the natural population, and which is produced by human intervention, e.g. by mutagenesis or gene editing, and a “mutant allele” refers to an allele which is not found in plants in the natural population or breeding population, but which is produced by human intervention such as mutagenesis or gene editing.

As used herein, the term “wild type allele” (e.g. wild type Rca 2 B allele, wild type Rca 2 A allele, or wild type Rca 2 D allele), means a naturally occurring allele found within cereal plants, in particular wheat plants, which encodes a functional non-thermotolerant protein (e.g. a functional non-thermotolerant Rca 2A, Rca 2B or Rca 2D protein). In contrast, the term “mutant allele” (e.g. mutant Rca 2A allele, mutant Rca 2B allele or mutant Rca 2D allele), as used herein, refers to an allele, which does not encode a functional non-thermotolerant protein, i.e. an Rca 2 allele encoding a non-functional Rca 2 protein (e.g. a non-functional Rca 2A or Rca 2B or Rca 2D) or an Rca 2 allele encoding a functional thermotolerant protein (e.g. a functional thermotolerant Rca 2A or Rca 2B or Rca 2D). A mutant Rca 2 allele encoding a non-functional Rca 2 protein, as used herein, refers to an Rca 2 protein having no biological activity or a significantly reduced biological activity as compared to the corresponding wild-type functional Rca 2 protein, or encoding no Rca 2 protein at all. An Rca 2 allele encoding a functional thermotolerant protein, as used herein, refers to a thermotolerant Rca 2 protein variant as described below. A knock-out Rca 2 allele is an equivalent term for a mutant Rca 2 allele encoding a non-functional Rca 2 protein. A thermotolerant Rca 2 allele is an equivalent term for an Rca 2 allele encoding a functional thermotolerant protein.

“Mutagenesis”, as used herein, refers to the process in which plant cells (e.g., a plurality of cereal seeds or other parts, such as pollen, etc.) are subjected to a technique which induces mutations in the DNA of the cells, such as contact with a mutagenic agent, such as a chemical substance (such as ethylmethylsulfonate (EMS), ethylnitrosourea (ENU), etc.) or ionizing radiation (neutrons (such as in fast neutron mutagenesis, etc.), alpha rays, gamma rays (such as that supplied by a Cobalt 60 source), X-rays, UV-radiation, etc.), T-DNA insertion mutagenesis (Azpiroz-Leehan et al. (1997) Trends Genet 13:152-156), transposon mutagenesis (McKenzie et al. (2002) Theor Appl Genet 105:23-33), or tissue culture mutagenesis (induction of somaclonal variations), or a combination of two or more of these. Thus, the desired mutagenesis of one or more Rca 2 alleles may be accomplished by use of one of the above methods. While mutations created by irradiation are often large deletions or other gross lesions such as translocations or complex rearrangements, mutations created by chemical mutagens are often more discrete lesions such as point mutations. For example, EMS alkylates guanine bases, which results in base mispairing: an alkylated guanine will pair with a thymine base, resulting primarily in G/C to A/T transitions. Following mutagenesis, cereal plants are regenerated from the treated cells using known techniques. For instance, the resulting cereal seeds may be planted in accordance with conventional growing procedures and following self-pollination seed is formed on the plants. Additional seed that is formed as a result of such self-pollination in the present or a subsequent generation may be harvested and screened for the presence of mutant Rca 2 alleles. Several techniques are known to screen for specific mutant alleles, e.g., Deleteagene™ (Delete-a-gene; Li et al., 2001, Plant J 27: 235-242) uses polymerase chain reaction (PCR) assays to screen for deletion mutants generated by fast neutron mutagenesis, TILLING (targeted induced local lesions in genomes; McCallum et al., 2000, Nat Biotechnol 18:455-457) identifies EMS-induced point mutations, etc.

Gene editing, as used herein, refers to the targeted modification of genomic DNA using sequence-specific enzymes (such as endonuclease, nickases, base conversion enzymes) and/or donor nucleic acids (e.g. dsDNA, oligo's) to introduce desired changes in the DNA. Sequence-specific nucleases that can be programmed to recognize specific DNA sequences include meganucleases (MGNs), zinc-finger nucleases (ZFNs), TAL-effector nucleases (TALENs) and RNA-guided or DNA-guided nucleases such as Cas9, Cpfl, CasX, CasY, C2c1, C2c3, certain Argonaut-based systems (see e.g. Osakabe and Osakabe, Plant Cell Physiol. 2015 March; 56(3):389-400; Ma et al., Mol Plant. 2016 Jul. 6; 9(7):961-74; Bortesie et al., Plant Biotech J, 2016, 14; Murovec et al., Plant Biotechnol J. 2017 Apr. 1; Nakade et al., Bioengineered 8-3, 2017; Burstein et al., Nature 542, 37-241; Komor et al., Nature 533, 420-424, 2016; all incorporated herein by reference). Donor nucleic acids can be used as a template for repair of the DNA break induced by a sequence specific nuclease, but can also be used as such for gene targeting (without DNA break induction) to introduce a desired change into the genomic DNA. Sequence-specific nucleases may also be used without donor nucleic acid, thereby allowing insertion or deletion mutations via non homologous end joining repair mechanism. Gene editing can be used to create mutant Rca 2 alleles.

Mutant nucleic acid molecules or mutant alleles may comprise one or more mutations or modifications, such as:
a. a “missense mutation”, which is a change in the nucleic acid sequence that results in the substitution of an amino acid for another amino acid;
b. a “nonsense mutation” or “STOP codon mutation”, which is a change in the nucleic acid sequence that results in the introduction of a premature STOP codon and thus the termination of translation (resulting in a truncated protein); plant genes contain the translation stop codons “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation;
c. an “insertion mutation” of one or more amino acids, due to one or more codons having been added in the coding sequence of the nucleic acid;
d. a “deletion mutation” of one or more amino acids, due to one or more codons having been deleted in the coding sequence of the nucleic acid;
e. a “frameshift mutation”, resulting in the nucleic acid sequence being translated in a different frame downstream of the mutation. A frameshift mutation can have various causes, such as the insertion, deletion or duplication of one or more nucleotides;
f. a mutated splice site, resulting in altered splicing, which results in an altered mRNA processing and, consequently, in an altered encoded protein which contains either deletions, substitutions or insertions of various lengths, possibly combined with premature translation termination.

### Conserved Residues in Thermostable Variants of Rca Proteins

Eleven amino acid residues conserved in thermostable variants of Rca proteins from various plant species have been here identified (see Example 3). These amino acids are (a) an isoleucine at a position corresponding to position 59 of SEQ ID NO: 4 or position 109 of SEQ ID NO: 8, called herein “AA1”, (b) an aspartic acid at a position corresponding to position 73 of SEQ ID NO: 4 or position 123 of SEQ ID NO: 8, called herein “AA2”, (c) an isoleucine at a position corresponding to position 160 of SEQ ID NO: 4 position 210 of SEQ ID NO: 8, called herein “AA3”, (d) an arginine at a position corresponding to position 265 of SEQ ID NO: 4 or position 315 of SEQ ID NO: 8, called herein “AA4”, (e) a proline at a position corresponding to position 270 of SEQ ID NO: 4 or position 320 of SEQ ID NO: 8, called herein “AA5”, (f) a leucine at a position corresponding to position 277 of SEQ ID NO: 4 or position 327 of SEQ ID NO: 8, called herein “AA6”, (g) a glutamic acid at a position corresponding to position 307 of SEQ ID NO: 4 or position 357 of SEQ ID NO: 8, called herein “AA7”, (h) an isoleucine at a position corresponding to position 334 of SEQ ID NO: 4 or position 384 of SEQ ID NO: 8, called herein “AA8”, (i) a lysine at a position corresponding to position 359 of SEQ ID NO: 4 or position 409 of SEQ ID NO: 8, called herein “AA9”, (j) a leucine at a position corresponding to position 361 of SEQ ID NO: 4 or position 411 of SEQ ID NO: 8, called herein “AA10”, and (k) a glutamic acid at a position corresponding to position 363 of SEQ ID NO: 4 or position 413 of SEQ ID NO: 8, called herein “AA11”.

It is understood that thermostable proteins described herein may comprise an amino acid sequence having at least 90% identity to the amino acid sequences of SEQ ID NOs: 8, 47, 49, 2, 4, 6, 30, 33, 39, 41, 43 or 45 and comprising at least one, at least two, at least three, at least four, at least five, at least six, at least seven, at least eight, at least nine, at least ten or all eleven amino acid residues conserved in thermostable variants of Rca proteins from various plant species.

Such at least two amino acid residues may be (a) AA1 and AA2, (b) AA1 and AA3, (c) AA1 and AA4, (d) AA1 and AA5, (e) AA1 and AA6, (f) AA1 and AA7, (g) AA1 and AA8, (h) AA1 and AA9, (i) AA1 and AA10, or (j) AA1 and AA11. The at least two amino acid residues may also be (a) AA2 and AA3, (b) AA2 and AA4, (c) AA2 and AA5, (d) AA2 and AA6, (e) AA2 and AA7, (f) AA2 and AA8, (g) AA2 and AA9, (h) AA2 and AA10, or (i) AA2 and AA11. The at least two amino acid residues may also be (a) AA3 and AA4, (b) AA3 and AA5, (c) AA3 and AA6, (d) AA3 and AA7, (e) AA3 and AA8, (f) AA3 and AA9, (g) AA3 and AA10, or (h) AA3 and AA11. The at least two amino acid residues may also be (a) AA4 and AA5, (b) AA4 and AA6, (c) AA4 and AA7, (d) AA4 and AA8, (e) AA4 and AA9, (f) AA4 and AA10, or (g) AA4 and AA11. As an alternative the at least two amino acid residues may be (a) AA5 and AA6, (b) AA5 and AA7, (c) AA5 and AA8, (d) AA5 and AA9, (e) AA5 and AA10, or (f) AA5 and AA11. As another alternative the at least two amino acid residues may be (a) AA6 and AA7, (b) AA6 and AA8, (c) AA6 and AA9, (d) AA6 and AA10, or (e) AA6 and AA11. As yet another alternative the at least two amino acid residues may be (a) AA7 and AA8, (b) AA7 and AA9, (c) AA7 and AA10, or (d) AA7 and AA11. The at least two amino acid residues may also be (a) AA8 and AA9, (b) AA8 and AA10, or (c) AA8 and AA11. The at least two amino acid residues may furthermore also be (a) AA9 and AA10, or (b) AA9 and AA11. The at least two amino acid residues may also be AA10 and AA11.

Such at least three amino acid residues may be (a) AA1, AA2 and AA3, (b) AA1, AA2 and AA4, (c) AA1, AA2 and AA5, (d) AA1, AA2 and AA6, (e) AA1, AA2 and AA7, (f) AA1, AA2 and AA8, (g) AA1, AA2 and AA9, (h) AA1, AA2 and AA10, or (i) AA1, AA2 and AA11. The at least three amino acid residues may also be (a) AA1, AA3 and AA4, (b) AA1, AA3 and AA5, (c) AA1, AA3 and AA6, (d) AA1, AA3 and AA7, (e) AA1, AA3 and AA8, (f) AA1, AA3 and AA9, (g) AA1, AA3 and AA10, or (h) AA1, AA3 and AA11. The at least three amino acid residues may also be (a) AA1, AA4 and AA5, (b) AA1, AA4 and AA6, (c) AA1, AA4 and AA7, (d) AA1, AA4 and AA8, (e) AA1, AA4 and AA9, (f) AA1, AA4 and AA10, or (g) AA1, AA4 and AA11. As an alternative the at least three amino acid residues may be (a) AA1, AA5 and AA6, (b) AA1, AA5 and AA7, (c) AA1, AA5 and AA8, (d) AA1, AA5 and AA9, (e) AA1, AA5 and AA10, or (f) AA1, AA5 and AA11. As another alternative the at least three amino acid residues may be (a) AA1, AA6 and AA7, (b) AA1, AA6 and AA8, (c) AA1, AA6 and AA9, (d) AA1, AA6 and AA10, or (e) AA1, AA6 and AA11. As yet another alternative the at least three amino acid residues may be (a) AA1, AA7 and AA8, (b) AA1, AA7 and AA9, (c) AA1, AA7 and AA10, or (d) AA1, AA7 and AA11. The at least three amino acid residues may also be (a) AA1, AA8 and AA9, (b) AA1, AA8 and AA10, or (c) AA1, AA8 and AA11. The at least three amino acid residues may furthermore also be (a) AA1, AA9 and AA10, or (b) AA1, AA9 and AA11. The at least three amino acid residues may also be AA1, AA10 and AA11.

Such at least three amino acid residues may also be (a) AA2, AA3 and AA4, (b) AA2, AA3 and AA5, (c) AA2, AA3 and AA6, (d) AA2, AA3 and AA7, (e) AA2, AA3 and AA8, (f) AA2, AA3 and AA9, (g) AA2, AA3 and AA10, or (h) AA2, AA3 and AA11. The at least three amino acid residues may also be (a) AA2, AA4 and AA5, (b) AA2, AA4 and AA6, (c) AA2, AA4 and AA7, (d) AA2, AA4 and AA8, (e) AA2, AA4 and AA9, (f) AA2, AA4 and AA10, or (g) AA2, AA4 and AA11. As an alternative the at least three amino acid residues may be (a) AA2, AA5 and AA6, (b) AA2, AA5 and AA7, (c) AA2, AA5 and AA8, (d) AA2, AA5 and AA9, (e) AA2, AA5 and AA10, or (f) AA2, AA5 and AA11. As another alternative the at least three amino acid residues may be (a) AA2, AA6 and AA7, (b) AA2, AA6 and AA8, (c) AA2, AA6 and AA9, (d) AA2, AA6 and AA10, or (e) AA2, AA6 and AA11. As yet another alternative the at least three amino acid residues may be (a) AA2, AA7 and AA8, (b) AA2, AA7 and AA9, (c) AA2, AA7 and AA10, or (d) AA2, AA7 and AA11. The at least three amino acid residues may also be (a) AA2, AA8 and AA9, (b) AA2, AA8 and AA10, or (c) AA2, AA8 and AA11. The at least three amino acid residues may furthermore also be (a) AA2, AA9 and AA10, or (b) AA2, AA9 and AA11. The at least three amino acid residues may also be AA2, AA10 and AA11.

Such at least three amino acid residues may be (a) AA3, AA4 and AA5, (b) AA3, AA4 and AA6, (c) AA3, AA4 and AA7, (d) AA3, AA4 and AA8, (e) AA3, AA4 and AA9, (f) AA3, AA4 and AA10, or (g) AA3, AA4 and AA11. As an alternative the at least three amino acid residues may be (a) AA3, AA5 and AA6, (b) AA3, AA5 and AA7, (c) AA3, AA5 and AA8, (d) AA3, AA5 and AA9, (e) AA3, AA5 and AA10, or (f) AA3, AA5 and AA11. As another alternative the at least three amino acid residues may be (a) AA3, AA6 and AA7, (b) AA3, AA6 and AA8, (c) AA3, AA6 and AA9, (d) AA3, AA6 and AA10, or (e) AA3, AA6 and AA11. As yet another alternative the at least three amino acid residues may be (a) AA3, AA7 and AA8, (b) AA3, AA7 and AA9, (c) AA3, AA7 and AA10, or (d) AA3, AA7 and AA11. The at least three amino acid residues may also be (a) AA3, AA8 and AA9, (b) AA3, AA8 and AA10, or (c) AA3, AA8 and AA11. The at least three amino acid residues may furthermore also be (a) AA3, AA9 and AA10, or (b) AA3, AA9 and AA11. The at least three amino acid residues may also be AA3, AA10 and AA11.

Such at least three amino acid residues may be (a) AA4, AA5 and AA6, (b) AA4, AA5 and AA7, (c) AA4, AA5 and AA8, (d) AA4, AA5 and AA9, (e) AA4, AA5 and AA10, or (f) AA4, AA5 and AA11. As another alternative the at least three amino acid residues may be (a) AA4, AA6 and AA7, (b) AA4, AA6 and AA8, (c) AA4, AA6 and AA9, (d) AA4, AA6 and AA10, or (e) AA4, AA6 and AA11. As yet another alternative the at least three amino acid residues may be (a) AA4, AA7 and AA8, (b) AA4, AA7 and AA9, (c) AA4, AA7 and AA10, or (d) AA4, AA7 and AA11. The at least three amino acid residues may also be (a) AA4, AA8 and AA9, (b) AA4, AA8 and AA10, or (c) AA4, AA8 and AA11. The at least three amino acid residues may furthermore also be (a) AA4, AA9 and AA10, or (b) AA4, AA9 and AA11. The at least three amino acid residues may also be AA4, AA10 and AA11.

Such at least three amino acid residues may be (a) AA5, AA6 and AA7, (b) AA5, AA6 and AA8, (c) AA5, AA6 and AA9, (d) AA5, AA6 and AA10, or (e) AA5, AA6 and AA11. As yet another alternative the at least three amino acid residues may be (a) AA5, AA7 and AA8, (b) AA5, AA7 and AA9, (c) AA5, AA7 and AA10, or (d) AA5, AA7 and AA11. The at least three amino acid residues may also be (a) AA5, AA8 and AA9, (b) AA5, AA8 and AA10, or (c) AA5, AA8 and AA11. The at least three amino acid residues may furthermore also be (a) AA5, AA9 and AA10, or (b) AA5, AA9 and AA11. The at least three amino acid residues may also be AA5, AA10 and AA11.

Such at least three amino acid residues may be (a) AA6, AA7 and AA8, (b) AA6, AA7 and AA9, (c) AA6, AA7 and AA10, or (d) AA6, AA7 and AA11. The at least three amino acid residues may also be (a) AA6, AA8 and AA9, (b) AA6, AA8 and AA10, or (c) AA6, AA8 and AA11. The at least three amino acid residues may furthermore also be (a) AA6, AA9 and AA10, or (b) AA6, AA9 and AA11. The at least three amino acid residues may also be AA6, AA10 and AA11. Such at least three amino acid residues may be (a) AA7, AA8 and AA9, (b) AA7, AA8 and AA10, or (c) AA7, AA8 and AA11. The at least three amino acid residues may furthermore also be (a) AA7, AA9 and AA10, or (b) AA7, AA9 and AA11. The at least three amino acid residues may also be AA7, AA10 and AA11. Such at least three amino acid residues may also be (a) AA8, AA9 and AA10, or (b) AA8, AA9 and AA11. The at least three amino acid residues may also be AA8, AA10 and AA11. The at least three amino acid residues may also be AA9, AA10 and AA11.

Such at least four amino acid residues may be (a) AA1, AA2, AA3 and AA4, (b) AA1, AA2, AA3 and AA5, (c) AA1, AA2, AA3 and AA6, (d) AA1, AA2, AA3 and AA7, (e) AA1, AA2, AA3 and AA8, (f) AA1, AA2, AA3 and AA9, (g) AA1, AA2, AA3 and AA10, or (h) AA1, AA2, AA3 and AA11. The at least four amino acid residues may also be (a) AA1, AA2, AA4 and AA5, (b) AA1, AA2, AA4 and AA6, (c) AA1, AA2, AA4 and AA7, (d) AA1, AA2, AA4 and AA8, (e) AA1, AA2, AA4 and AA9, (f) AA1, AA2, AA4 and AA10, or (g) AA1, AA2, AA4 and AA11. As an alternative the at least four amino acid residues may be (a) AA1, AA2, AA5 and AA6, (b) AA1, AA2, AA5 and AA7, (c) AA1, AA2, AA5 and AA8, (d) AA1, AA2, AA5 and AA9, (e) AA1, AA2, AA5 and AA10, or (f) AA1, AA2, AA5 and AA11. As another alternative the at least four amino acid residues may be (a) AA1, AA2, AA6 and AA7, (b) AA1, AA2, AA6 and AA8, (c) AA1, AA2, AA6 and AA9, (d) AA1, AA2, AA6 and AA10, or (e) AA1, AA2, AA6 and AA11. As yet another alternative the at least four amino acid residues may be (a) AA1, AA2, AA7 and AA8, (b) AA1, AA2, AA7 and AA9, (c) AA1, AA2, AA7 and AA10, or (d) AA1, AA2, AA7 and AA11. The at least four amino acid residues may also be (a) AA1, AA2, AA8 and AA9, (b) AA1, AA2, AA8 and AA10, or (c) AA1, AA2, AA8 and AA11. The at least four amino acid residues may furthermore also be (a) AA1, AA2, AA9 and AA10, or (b) AA1, AA2, AA9 and AA11. The at least four amino acid residues may also be AA1, AA2, AA10 and AA11. Such at least four amino acid residues may be (a) AA1, AA3, AA4 and AA5, (b) AA1, AA3, AA4 and AA6, (c) AA1, AA3, AA4 and AA7, (d) AA1, AA3, AA4 and AA8, (e) AA1, AA3, AA4 and AA9, (f) AA1, AA3, AA4 and AA10, or (g) AA1, AA3, AA4 and AA11. As an alternative the at least four amino acid residues may be (a) AA1, AA3, AA5 and AA6, (b) AA1, AA3, AA5 and AA7, (c) AA1, AA3, AA5 and AA8, (d) AA1, AA3, AA5 and AA9, (e) AA1, AA3, AA5 and AA10, or (f) AA1, AA3, AA5 and AA11. As another alternative the at least four amino acid residues may be (a) AA1, AA3, AA6 and AA7, (b) AA1, AA3, AA6 and AA8, (c) AA1, AA3, AA6 and AA9, (d) AA1, AA3, AA6 and AA10, or (e) AA1, AA3, AA6 and AA11. As yet another alternative the at least four amino acid residues may be (a) AA1, AA3, AA7 and AA8, (b) AA1, AA3, AA7 and AA9, (c) AA1, AA3, AA7 and AA10, or (d) AA1, AA3, AA7 and AA11. The at least four amino acid residues may also be (a) AA1, AA3, AA8 and AA9, (b) AA1, AA3, AA8 and AA10, or (c) AA1, AA3, AA8 and AA11. The at least four amino acid residues may furthermore also be (a) AA1, AA3, AA9 and AA10, or (b) AA1, AA3, AA9 and AA11. The at least four amino acid residues may also be AA1, AA3, AA10 and AA11. Such at least four amino acid residues may be (a) AA1, AA4, AA5 and AA6, (b) AA1, AA4, AA5 and AA7, (c) AA1, AA4, AA5 and AA8, (d) AA1, AA4, AA5 and AA9, (e) AA1, AA4, AA5 and AA10, or (f) AA1, AA4, AA5 and AA11. As another alternative the at least four amino acid residues may be (a) AA1, AA4, AA6 and AA7, (b) AA1, AA4, AA6 and AA8, (c) AA1, AA4, AA6 and AA9, (d) AA1, AA4, AA6 and AA10, or (e) AA1, AA4, AA6 and AA11. As yet another alternative the at least four amino acid residues may be (a) AA1, AA4, AA7 and AA8, (b) AA1, AA4, AA7 and AA9, (c) AA1, AA4, AA7 and AA10, or (d) AA1, AA4, AA7 and AA11. The at least four amino acid residues may also be (a) AA1, AA4, AA8 and AA9, (b) AA1, AA4, AA8 and AA10, or (c) AA1, AA4, AA8 and AA11. The at least four amino acid residues may furthermore also be (a) AA1, AA4, AA9 and AA10, or (b) AA1, AA4, AA9 and AA11. The at least four amino acid residues may also be AA1, AA4, AA10 and AA11. Such at least four amino acid residues may be (a) AA1, AA5, AA6 and AA7, (b) AA1, AA5, AA6 and AA8, (c) AA1, AA5, AA6 and AA9, (d) AA1, AA5, AA6 and AA10, or (e) AA1, AA5, AA6 and AA11. As yet another alternative the at least four amino acid residues may be (a) AA1, AA5, AA7 and AA8, (b) AA1, AA5, AA7 and AA9, (c) AA1, AA5, AA7 and AA10, or (d) AA1, AA5, AA7 and AA11. The at least four amino acid residues may also be (a) AA1, AA5, AA8 and AA9, (b) AA1, AA5, AA8 and AA10, or (c) AA1, AA5, AA8 and AA11. The at least four amino acid residues may furthermore also be (a) AA1, AA5, AA9 and AA10, or (b) AA1, AA5, AA9 and AA11. The at least four amino acid residues may also be AA1, AA5, AA10 and AA11. Such at least four amino acid residues may be (a) AA1, AA6, AA7 and AA8, (b) AA1, AA6, AA7 and AA9, (c) AA1, AA6, AA7 and AA10, or (d) AA1, AA6, AA7 and AA11. The at least four amino acid residues may also be (a) AA1, AA6, AA8 and AA9, (b) AA1, AA6, AA8 and AA10, or (c) AA1, AA6, AA8 and AA11. The at least four amino acid residues may furthermore also be (a) AA1, AA6, AA9 and AA10, or (b) AA1, AA6, AA9 and AA11. The at least four amino acid residues may also be AA1, AA6, AA10 and AA11. Such at least four amino acid residues may be (a) AA1, AA7, AA8 and AA9, (b) AA1, AA7, AA8 and AA10, or (c) AA1, AA7, AA8 and AA11. The at least four amino acid residues may furthermore also be (a) AA1, AA7, AA9 and AA10, or (b) AA1, AA7, AA9 and AA11. The at least four amino acid residues may also be AA1, AA7, AA10 and AA11. Such at least four amino acid residues may be (a) AA1, AA8, AA9 and AA10, or (b) AA1, AA8, AA9 and AA11. The at least four amino acid residues may also be AA1, AA8, AA10 and AA11. The at least four amino acid residues may also be AA1, AA9, AA10 and AA11.

Such at least four amino acid residues may be (a) AA2, AA3, AA4 and AA5, (b) AA2, AA3, AA4 and AA6, (c) AA2, AA3, AA4 and AA7, (d) AA2, AA3, AA4 and AA8, (e) AA2, AA3, AA4 and AA9, (f) AA2, AA3, AA4 and AA10, or (g) AA2, AA3, AA4 and AA11. As an alternative the at least four amino acid residues may be (a) AA2, AA3, AA5 and AA6, (b) AA2, AA3, AA5 and AA7, (c) AA2, AA3, AA5 and AA8, (d) AA2, AA3, AA5 and AA9, (e) AA2, AA3, AA5 and AA10, or (f) AA2, AA3, AA5 and AA11. As another alternative the at least four amino acid residues may be (a) AA2, AA3, AA6 and AA7, (b) AA2, AA3, AA6 and AA8, (c) AA2, AA3, AA6 and AA9, (d) AA2, AA3, AA6 and AA10, or (e) AA2, AA3, AA6 and AA11. As yet another alternative the at least four amino acid residues may be (a) AA2, AA3, AA7 and AA8, (b) AA2, AA3, AA7 and AA9, (c) AA2, AA3, AA7 and AA10, or (d) AA2, AA3, AA7 and AA11. The at least four amino acid residues may also be (a) AA2, AA3, AA8 and AA9, (b) AA2, AA3, AA8 and AA10, or (c) AA2, AA3, AA8 and AA11. The at least four amino acid residues may furthermore also be (a) AA2, AA3, AA9 and AA10, or (b) AA2, AA3, AA9 and AA11. The at least four amino acid residues may also be AA2, AA3, AA10 and AA11. Such at least four amino acid residues may be (a) AA2, AA4, AA5 and AA6, (b) AA2, AA4, AA5 and AA7, (c) AA2, AA4, AA5 and AA8, (d) AA2, AA4, AA5 and AA9, (e) AA2, AA4, AA5 and AA10, or (f) AA2, AA4, AA5 and AA11. As another alternative the at least four amino acid residues may be (a) AA2, AA4, AA6 and AA7, (b) AA2, AA4, AA6 and AA8, (c) AA2, AA4, AA6 and AA9, (d) AA2, AA4, AA6 and AA10, or (e) AA2, AA4, AA6 and AA11. As yet another alternative the at least four amino acid residues may be (a) AA2, AA4, AA7 and AA8, (b) AA2, AA4, AA7 and AA9, (c) AA2, AA4, AA7 and AA10, or (d) AA2, AA4, AA7 and AA11. The at least four amino acid residues may also be (a) AA2, AA4, AA8 and AA9, (b) AA2, AA4, AA8 and AA10, or (c) AA2, AA4, AA8 and AA11. The at least four amino acid residues may furthermore also be (a) AA2, AA4, AA9 and AA10, or (b) AA2, AA4, AA9 and AA11. The at least four amino acid residues may also be AA2, AA4, AA10 and AA11. Such at least four amino acid residues may be (a) AA2, AA5, AA6 and AA7, (b) AA2, AA5, AA6 and AA8, (c) AA2, AA5, AA6 and AA9, (d) AA2, AA5, AA6 and AA10, or (e) AA2, AA5, AA6 and AA11. As yet another alternative the at least four amino acid residues may be (a) AA2, AA5, AA7 and AA8, (b) AA2, AA5, AA7 and AA9, (c) AA2, AA5, AA7 and AA10, or (d) AA2, AA5, AA7 and AA11. The at least four amino acid residues may also be (a) AA2, AA5, AA8 and AA9, (b) AA2, AA5, AA8 and AA10, or (c) AA2, AA5, AA8 and AA11. The at least four amino acid residues may furthermore also be (a) AA2, AA5, AA9 and AA10, or (b) AA2, AA5, AA9 and AA11. The at least four amino acid residues may also be AA2, AA5, AA10 and AA11. Such at least four amino acid residues may be (a) AA2, AA6, AA7 and AA8, (b) AA2, AA6, AA7 and AA9, (c) AA2, AA6, AA7 and AA10, or (d) AA2, AA6, AA7 and AA11. The at least four amino acid residues may also be (a) AA2, AA6, AA8 and AA9, (b) AA2, AA6, AA8 and AA10, or (c) AA2, AA6, AA8 and AA11. The at least four amino acid residues may furthermore also be (a) AA2, AA6, AA9 and AA10, or (b) AA2, AA6, AA9 and AA11. The at least four amino acid residues may also be AA2, AA6, AA10 and AA11. Such at least four amino acid residues may be (a) AA2, AA7, AA8 and AA9, (b) AA2, AA7, AA8 and AA10, or (c) AA2, AA7, AA8 and AA11. The at least four amino acid residues may furthermore also be (a) AA2, AA7, AA9 and AA10, or (b) AA2, AA7, AA9 and AA11. The at least four amino acid residues may also be AA2, AA7, AA10 and AA11. Such at least four amino acid residues may be (a) AA2, AA8, AA9 and AA10, or (b) AA2, AA8, AA9 and AA11. The at least four amino acid residues may also be AA2, AA8, AA10 and AA11. The at least four amino acid residues may also be AA2, AA9, AA10 and AA11.

Such at least four amino acid residues may be (a) AA3, AA4, AA5 and AA6, (b) AA3, AA4, AA5 and AA7, (c) AA3, AA4, AA5 and AA8, (d) AA3, AA4, AA5 and AA9, (e) AA3, AA4, AA5 and AA10, or (f) AA3, AA4, AA5 and AA11. As another alternative the at least four amino acid residues may be (a) AA3, AA4, AA6 and AA7, (b) AA3, AA4, AA6 and AA8, (c) AA3, AA4, AA6 and AA9, (d) AA3, AA4, AA6 and AA10, or (e) AA3, AA4, AA6 and AA11. As yet another alternative the at least four amino acid residues may be (a) AA3, AA4, AA7 and AA8, (b) AA3, AA4, AA7 and AA9, (c) AA3, AA4, AA7 and AA10, or (d) AA3, AA4, AA7 and AA11. The at least four amino acid residues may also be (a) AA3, AA4, AA8 and AA9, (b) AA3, AA4, AA8 and AA10, or (c) AA3, AA4, AA8 and AA11. The at least four amino acid residues may furthermore also be (a) AA3, AA4, AA9 and AA10, or (b) AA3, AA4, AA9 and AA11. The at least four amino acid residues may also be AA3, AA4, AA10 and AA11. Such at least four amino acid residues may be (a) AA3, AA5, AA6 and AA7, (b) AA3, AA5, AA6 and AA8, (c) AA3, AA5, AA6 and AA9, (d) AA3, AA5, AA6 and AA10, or (e) AA3, AA5, AA6 and AA11. As yet another alternative the at least four amino acid residues may be (a) AA3, AA5, AA7 and AA8, (b) AA3, AA5, AA7 and AA9, (c) AA3, AA5, AA7 and AA10, or (d) AA3, AA5, AA7 and AA11. The at least four amino acid residues may also be (a) AA3, AA5, AA8 and AA9, (b) AA3, AA5, AA8 and AA10, or (c) AA3, AA5, AA8 and AA11. The at least four amino acid residues may furthermore also be (a) AA3, AA5, AA9 and AA10, or (b) AA3, AA5, AA9 and AA11. The at least four amino acid residues may also be AA3, AA5, AA10 and AA11. Such at least four amino acid residues may be (a) AA3, AA6, AA7 and AA8, (b) AA3, AA6, AA7 and AA9, (c) AA3, AA6, AA7 and AA10, or (d) AA3, AA6, AA7 and AA11. The at least four amino acid residues may also be (a) AA3, AA6, AA8 and AA9, (b) AA3, AA6, AA8 and AA10, or (c) AA3, AA6, AA8 and AA11. The at least four amino acid residues may furthermore also be (a) AA3, AA6, AA9 and AA10, or (b) AA3, AA6, AA9 and AA11. The at least four amino acid residues may also be AA3, AA6, AA10 and AA11. Such at least four amino acid residues may be (a) AA3, AA7, AA8 and AA9, (b) AA3, AA7, AA8 and AA10, or (c) AA3, AA7, AA8 and AA11. The at least four amino acid residues may furthermore also be (a) AA3, AA7, AA9 and AA10, or (b) AA3, AA7, AA9 and AA11. The at least four amino acid residues may also be AA3, AA7, AA10 and AA11. Such at least four amino acid residues may be (a) AA3, AA8, AA9 and AA10, or (b) AA3, AA8, AA9 and AA11. The at least four amino acid residues may also be AA3, AA8, AA10 and AA11. The at least four amino acid residues may also be AA3, AA9, AA10 and AA11.

Such at least four amino acid residues may be (a) AA4, AA5, AA6 and AA7, (b) AA4, AA5, AA6 and AA8, (c) AA4, AA5, AA6 and AA9, (d) AA4, AA5, AA6 and AA10, or (e) AA4, AA5, AA6 and AA11. As yet another alternative the at least four amino acid residues may be (a) AA4, AA5, AA7 and AA8, (b) AA4, AA5, AA7 and AA9, (c) AA4, AA5, AA7 and AA10, or (d) AA4, AA5, AA7 and AA11. The at least four amino acid residues may also be (a) AA4, AA5, AA8 and AA9, (b) AA4, AA5, AA8 and AA10, or (c) AA4, AA5, AA8 and AA11. The at least four amino acid residues may furthermore also be (a) AA4, AA5, AA9 and AA10, or (b) AA4, AA5, AA9 and AA11. The at least four amino acid residues may also be AA4, AA5, AA10 and AA11. Such at least four amino acid residues may be (a) AA4, AA6, AA7 and AA8, (b) AA4, AA6, AA7 and AA9, (c) AA4, AA6, AA7 and AA10, or (d) AA4, AA6, AA7 and AA11. The at least four amino acid residues may also be (a) AA4, AA6, AA8 and AA9, (b) AA4, AA6, AA8 and AA10, or (c) AA4, AA6, AA8 and AA11. The at least four amino acid residues may furthermore also be (a) AA4, AA6, AA9 and AA10, or (b) AA4, AA6, AA9 and AA11. The at least four amino acid residues may also be AA4, AA6, AA10 and AA11. Such at least four amino acid residues may be (a) AA4, AA7, AA8 and AA9, (b) AA4, AA7, AA8 and AA10, or (c) AA4, AA7, AA8 and AA11. The at least four amino acid residues may furthermore also be (a) AA4, AA7, AA9 and AA10, or (b) AA4, AA7, AA9 and AA11. The at least four amino acid residues may also be AA4, AA7, AA10 and AA11. Such at least four amino acid residues may be (a) AA4, AA8, AA9 and AA10, or (b) AA4, AA8, AA9 and AA11. The at least four amino acid residues may also be AA4, AA8, AA10 and AA11. The at least four amino acid residues may also be AA4, AA9, AA10 and AA11.

Such at least four amino acid residues may be (a) AA5, AA6, AA7 and AA8, (b) AA5, AA6, AA7 and AA9, (c) AA5, AA6, AA7 and AA10, or (d) AA5, AA6, AA7 and AA11. The at least four amino acid residues may also be (a) AA5, AA6, AA8 and AA9, (b) AA5, AA6, AA8 and AA10, or (c) AA5, AA6, AA8 and AA11. The at least four amino acid residues may furthermore also be (a) AA5, AA6, AA9 and AA10, or (b) AA5, AA6, AA9 and AA11. The at least four amino acid residues may also be AA5, AA6, AA10 and AA11. Such at least four amino acid residues may be (a) AA5, AA7, AA8 and AA9, (b) AA5, AA7, AA8 and AA10, or (c) AA5, AA7, AA8 and AA11. The at least four amino acid residues may furthermore also be (a) AA5, AA7, AA9 and AA10, or (b) AA5, AA7, AA9 and AA11. The at least four amino acid residues may also be AA5, AA7, AA10 and AA11. Such at least four amino acid residues may be (a) AA5, AA8, AA9 and AA10, or (b) AA5, AA8, AA9 and AA11. The at least four amino acid residues may also be AA5, AA8, AA10 and AA11. The at least four amino acid residues may also be AA5, AA9, AA10 and AA11.

Such at least four amino acid residues may be (a) AA6, AA7, AA8 and AA9, (b) AA6, AA7, AA8 and AA10, or (c) AA6, AA7, AA8 and AA11. The at least four amino acid residues may furthermore also be (a) AA6, AA7, AA9 and AA10, or (b) AA6, AA7, AA9 and AA11. The at least four amino acid residues may also be AA6, AA7, AA10 and AA11. Such at least four amino acid residues may be (a) AA6, AA8, AA9 and AA10, or (b) AA6, AA8, AA9 and AA11. The at least four amino acid residues may also be AA6, AA8, AA10 and AA11. The at least four amino acid residues may also be AA6, AA9, AA10 and AA11. Such at least four amino acid residues may be (a) AA7, AA8, AA9 and AA10, or (b) AA7, AA8, AA9 and AA11. The at least four amino acid residues may also be AA7, AA8, AA10 and AA11. The at least four amino acid residues may also be AA7, AA9, AA10 and AA11. The at least four amino acid residues may also be AA8, AA9, AA10 and AA11.

Such at least five amino acid residues may be (a) AA1, AA2, AA3, AA4 and AA5, (b) AA1, AA2, AA3, AA4 and AA6, (c) AA1, AA2, AA3, AA4 and AA7, (d) AA1, AA2, AA3, AA4 and AA8, (e) AA1, AA2, AA3, AA4 and AA9, (f) AA1, AA2, AA3, AA4 and AA10, or (g) AA1, AA2, AA3, AA4 and AA11. As an alternative the at least five amino acid residues may be (a) AA1, AA2, AA3, AA5 and AA6, (b) AA1, AA2, AA3, AA5 and AA7, (c) AA1, AA2, AA3, AA5 and AA8, (d) AA1, AA2, AA3, AA5 and AA9, (e) AA1, AA2, AA3, AA5 and AA10, or (f) AA1, AA2, AA3, AA5 and AA11. As another alternative the at least five amino acid residues may be (a) AA1, AA2, AA3, AA6 and AA7, (b) AA1, AA2, AA3, AA6 and AA8, (c) AA1, AA2, AA3, AA6 and AA9, (d) AA1, AA2, AA3, AA6 and AA10, or (e) AA1, AA2, AA3, AA6 and AA11. As yet another alternative the at least five amino acid residues may be (a) AA1, AA2, AA3, AA7 and AA8, (b) AA1, AA2, AA3, AA7 and AA9, (c) AA1, AA2, AA3, AA7 and AA10, or (d) AA1, AA2, AA3, AA7 and AA11. The at least five amino acid residues may also be (a) AA1, AA2, AA3, AA8 and AA9, (b) AA1, AA2, AA3, AA8 and AA10, or (c) AA1, AA2, AA3, AA8 and AA11. The at least five amino acid residues may furthermore also be (a) AA1, AA2, AA3, AA9 and AA10, or (b) AA1, AA2, AA3, AA9 and AA11. The at least five amino acid residues may also be AA1, AA2, AA3, AA10 and AA11. Such at least five amino acid residues may be (a) AA1, AA2, AA4, AA5 and AA6, (b) AA1, AA2, AA4, AA5 and AA7, (c) AA1, AA2, AA4, AA5 and AA8, (d) AA1, AA2, AA4, AA5 and AA9, (e) AA1, AA2, AA4, AA5 and AA10, or (f) AA1, AA2, AA4, AA5 and AA11. As another alternative the at least five amino acid residues may be (a) AA1, AA2, AA4, AA6 and AA7, (b) AA1, AA2, AA4, AA6 and AA8, (c) AA1, AA2, AA4, AA6 and AA9, (d) AA1, AA2, AA4, AA6 and AA10, or (e) AA1, AA2, AA4, AA6 and AA11. As yet another alternative the at least five amino acid residues may be (a) AA1, AA2, AA4, AA7 and AA8, (b) AA1, AA2, AA4, AA7 and AA9, (c) AA1, AA2, AA4, AA7 and AA10, or (d) AA1, AA2, AA4, AA7 and AA11. The at least five amino acid residues may also be (a) AA1, AA2, AA4, AA8 and AA9, (b) AA1, AA2, AA4, AA8 and AA10, or (c) AA1, AA2, AA4, AA8 and AA11. The at least five amino acid residues may furthermore also be (a) AA1, AA2, AA4, AA9 and AA10, or (b) AA1, AA2, AA4, AA9 and AA11. The at least five amino acid residues may also be AA1, AA2, AA4, AA10 and AA11. Such at least five amino acid residues may be (a) AA1, AA2, AA5, AA6 and AA7, (b) AA1, AA2, AA5, AA6 and AA8, (c) AA1, AA2, AA5, AA6 and AA9, (d) AA1, AA2, AA5, AA6 and AA10, or (e) AA1, AA2, AA5, AA6 and AA11. As yet another alternative the at least five amino acid residues may be (a) AA1, AA2, AA5, AA7 and AA8, (b) AA1, AA2, AA5, AA7 and AA9, (c) AA1, AA2, AA5, AA7 and AA10, or (d) AA1, AA2, AA5, AA7 and AA11. The at least five amino acid residues may also be (a) AA1, AA2, AA5, AA8 and AA9, (b) AA1, AA2, AA5, AA8 and AA10, or (c) AA1, AA2, AA5, AA8 and AA11. The at least five amino acid residues may furthermore also be (a) AA1, AA2, AA5, AA9 and AA10, or (b) AA1, AA2, AA5, AA9 and AA11. The at least five amino acid residues may also be AA1, AA2, AA5, AA10 and AA11. Such at least five amino acid residues may be (a) AA1, AA2, AA6, AA7 and AA8, (b) AA1, AA2, AA6, AA7 and AA9, (c) AA1, AA2, AA6, AA7 and AA10, or (d) AA1, AA2, AA6, AA7 and AA11. The at least five amino acid residues may also be (a) AA1, AA2, AA6, AA8 and AA9, (b) AA1, AA2, AA6, AA8 and AA10, or (c) AA1, AA2, AA6, AA8 and AA11. The at least five amino acid residues may furthermore also be (a) AA1, AA2, AA6, AA9 and AA10, or (b) AA1, AA2, AA6, AA9 and AA11. The at least five amino acid residues may also be AA1, AA2, AA6, AA10 and AA11. Such at least five amino acid residues may be (a) AA1, AA2, AA7, AA8 and AA9, (b) AA1, AA2, AA7, AA8 and AA10, or (c) AA1, AA2, AA7, AA8 and AA11. The at least five amino acid residues may furthermore also be (a) AA1, AA2, AA7, AA9 and AA10, or (b) AA1, AA2, AA7, AA9 and AA11. The at least five amino acid residues may also be AA1, AA2, AA7, AA10 and AA11. Such at least five amino acid residues may be (a) AA1, AA2, AA8, AA9 and AA10, or (b) AA1, AA2, AA8, AA9 and AA11. The at least five amino acid residues may also be AA1, AA2, AA8, AA10 and AA11. The at least five amino acid residues may also be AA1, AA2, AA9, AA10 and AA11. Such at least five amino acid residues may be (a) AA1, AA3, AA4, AA5 and AA6, (b) AA1, AA3, AA4, AA5 and AA7, (c) AA1, AA3, AA4, AA5 and AA8, (d) AA1, AA3, AA4, AA5 and AA9, (e) AA1, AA3, AA4, AA5 and AA10, or (f) AA1, AA3, AA4, AA5 and AA11. As another alternative the at least five amino acid residues may be (a) AA1, AA3, AA4, AA6 and AA7, (b) AA1, AA3, AA4, AA6 and AA8, (c) AA1, AA3, AA4, AA6 and AA9, (d) AA1, AA3, AA4, AA6 and AA10, or (e) AA1, AA3, AA4, AA6 and AA11. As yet another alternative the at least five amino acid residues may be (a) AA1, AA3, AA4, AA7 and AA8, (b) AA1, AA3, AA4, AA7 and AA9, (c) AA1, AA3, AA4, AA7 and AA10, or (d) AA1, AA3, AA4, AA7 and AA11. The at least five amino acid residues may also be (a) AA1, AA3, AA4, AA8 and AA9, (b) AA1, AA3, AA4, AA8 and AA10, or (c) AA1, AA3, AA4, AA8 and AA11. The at least five amino acid residues may furthermore also be (a) AA1, AA3, AA4, AA9 and AA10, or (b) AA1, AA3, AA4, AA9 and AA11. The at least five amino acid residues may also be AA1, AA3, AA4, AA10 and AA11. Such at least five amino acid residues may be (a) AA1, AA3, AA5, AA6 and AA7, (b) AA1, AA3, AA5, AA6 and AA8, (c) AA1, AA3, AA5, AA6 and AA9, (d) AA1, AA3, AA5, AA6 and AA10, or (e) AA1, AA3, AA5, AA6 and AA11. As yet another alternative the at least five amino acid residues may be (a) AA1, AA3, AA5, AA7 and AA8, (b) AA1, AA3, AA5, AA7 and AA9, (c) AA1, AA3, AA5, AA7 and AA10, or (d) AA1, AA3, AA5, AA7 and AA11. The at least five amino acid residues may also be (a) AA1, AA3, AA5, AA8 and AA9, (b) AA1, AA3, AA5, AA8 and AA10, or (c) AA1, AA3, AA5, AA8 and AA11. The at least five amino acid residues may furthermore also be (a) AA1, AA3, AA5, AA9 and AA10, or (b) AA1, AA3, AA5, AA9 and AA11. The at least five amino acid residues may also be AA1, AA3, AA5, AA10 and AA11. Such at least five amino acid residues may be (a) AA1, AA3, AA6, AA7 and AA8, (b) AA1, AA3, AA6, AA7 and AA9, (c) AA1, AA3, AA6, AA7 and AA10, or (d) AA1, AA3, AA6, AA7 and AA11. The at least five amino acid residues may also be (a) AA1, AA3, AA6, AA8 and AA9, (b) AA1, AA3, AA6, AA8 and AA10, or (c) AA1, AA3, AA6, AA8 and AA11. The at least five amino acid residues may furthermore also be (a) AA1, AA3, AA6, AA9 and AA10, or (b) AA1, AA3, AA6, AA9 and AA11. The at least five amino acid residues may also be AA1, AA3, AA6, AA10 and AA11. Such at least five amino acid residues may be (a) AA1, AA3, AA7, AA8 and AA9, (b) AA1, AA3, AA7, AA8 and AA10, or (c) AA1, AA3, AA7, AA8 and AA11. The at least five amino acid residues may furthermore also be (a) AA1, AA3, AA7, AA9 and AA10, or (b) AA1, AA3, AA7, AA9 and AA11. The at least five amino acid residues may also be AA1, AA3, AA7, AA10 and AA11. Such at least five amino acid residues may be (a) AA1, AA3, AA8, AA9 and AA10, or (b) AA1, AA3, AA8, AA9 and AA11. The at least five amino acid residues may also be AA1, AA3, AA8, AA10 and AA11. The at least five amino acid residues may also be AA1, AA3, AA9, AA10 and AA11. Such at least five amino acid residues may be (a) AA1, AA4, AA5, AA6 and AA7, (b) AA1, AA4, AA5, AA6 and AA8, (c) AA1, AA4, AA5, AA6 and AA9, (d) AA1, AA4, AA5, AA6 and AA10, or (e) AA1, AA4, AA5, AA6 and AA11. As yet another alternative the at least five amino acid residues may be (a) AA1, AA4, AA5, AA7 and AA8, (b) AA1, AA4, AA5, AA7 and AA9, (c) AA1, AA4, AA5, AA7 and AA10, or (d) AA1, AA4, AA5, AA7 and AA11. The at least five amino acid residues may also be (a) AA1, AA4, AA5, AA8 and AA9, (b) AA1, AA4, AA5, AA8 and AA10, or (c) AA1, AA4, AA5, AA8 and AA11. The at least five amino acid residues may furthermore also be (a) AA1, AA4, AA5, AA9 and AA10, or (b) AA1, AA4, AA5, AA9 and AA11. The at least five amino acid residues may also be AA1, AA4, AA5, AA10 and AA11. Such at least five amino acid residues may be (a) AA1, AA4, AA6, AA7 and AA8, (b) AA1, AA4, AA6, AA7 and AA9, (c) AA1, AA4, AA6, AA7 and AA10, or (d) AA1, AA4, AA6, AA7 and AA11. The at least five amino acid residues may also be (a) AA1, AA4, AA6, AA8 and AA9, (b) AA1, AA4, AA6, AA8 and AA10, or (c) AA1, AA4, AA6, AA8 and AA11. The at least five amino acid residues may furthermore also be (a) AA1, AA4, AA6, AA9 and AA10, or (b) AA1, AA4, AA6, AA9 and AA11. The at least five amino acid residues may also be AA1, AA4, AA6, AA10 and AA11. Such at least five amino acid residues may be (a) AA1, AA4, AA7, AA8 and AA9, (b) AA1, AA4, AA7, AA8 and AA10, or (c) AA1, AA4, AA7, AA8 and AA11. The at least five amino acid residues may furthermore also be (a) AA1, AA4, AA7, AA9 and AA10, or (b) AA1, AA4, AA7, AA9 and AA11. The at least five amino acid residues may also be AA1, AA4, AA7, AA10 and AA11. Such at least five amino acid residues may be (a) AA1, AA4, AA8, AA9 and AA10, or (b) AA1, AA4, AA8, AA9 and AA11. The at least five amino acid residues may also be AA1, AA4, AA8, AA10 and AA11. The at least five amino acid residues may also be AA1, AA4, AA9, AA10 and AA11. Such at least five amino acid residues may be (a) AA1, AA5, AA6, AA7 and AA8, (b) AA1, AA5, AA6, AA7 and AA9, (c) AA1, AA5, AA6, AA7 and AA10, or (d) AA1, AA5, AA6, AA7 and AA11. The at least five amino acid residues may also be (a) AA1, AA5, AA6, AA8 and AA9, (b) AA1, AA5, AA6, AA8 and AA10, or (c) AA1, AA5, AA6, AA8 and AA11. The at least five amino acid residues may furthermore also be (a) AA1, AA5, AA6, AA9 and AA10, or (b) AA1, AA5, AA6, AA9 and AA11. The at least five amino acid residues may also be AA1, AA5, AA6, AA10 and AA11. Such at least five amino acid residues may be (a) AA1, AA5, AA7, AA8 and AA9, (b) AA1, AA5, AA7, AA8 and AA10, or (c) AA1, AA5, AA7, AA8 and AA11. The at least five amino acid residues may furthermore also be (a) AA1, AA5, AA7, AA9 and AA10, or (b) AA1, AA5, AA7, AA9 and AA11. The at least five amino acid residues may also be AA1, AA5, AA7, AA10 and AA11. Such at least five amino acid residues may be (a) AA1, AA5, AA8, AA9 and AA10, or (b) AA1, AA5, AA8, AA9 and AA11. The at least five amino acid residues may also be AA1, AA5, AA8, AA10 and AA11. The at least five amino acid residues may also be AA1, AA5, AA9, AA10 and AA11. Such at least five amino acid residues may be (a) AA1, AA6, AA7, AA8 and AA9, (b) AA1, AA6, AA7, AA8 and AA10, or (c) AA1, AA6, AA7, AA8 and AA11. The at least five amino acid residues may furthermore also be (a) AA1, AA6, AA7, AA9 and AA10, or (b) AA1, AA6, AA7, AA9 and AA11. The at least five amino acid residues may also be AA1, AA6, AA7, AA10 and AA11. Such at least five amino acid residues may be (a) AA1, AA6, AA8, AA9 and AA10, or (b) AA1, AA6, AA8, AA9 and AA11. The at least five amino acid residues may also be AA1, AA6, AA8, AA10 and AA11. The at least five amino acid residues may also be AA1, AA6, AA9, AA10 and AA11. Such at least five amino acid residues may be (a) AA1, AA7, AA8, AA9 and AA10, or (b) AA1, AA7, AA8, AA9 and AA11. The at least five amino acid residues may also be AA1, AA7, AA8, AA10 and AA11. The at least five amino acid residues may also be AA1, AA7, AA9, AA10 and AA11. The at least five amino acid residues may also be AA1, AA8, AA9, AA10 and AA11.

Such at least five amino acid residues may be (a) AA2, AA3, AA4, AA5 and AA6, (b) AA2, AA3, AA4, AA5 and AA7, (c) AA2, AA3, AA4, AA5 and AA8, (d) AA2, AA3, AA4, AA5 and AA9, (e) AA2, AA3, AA4, AA5 and AA10, or (f) AA2, AA3, AA4, AA5 and AA11. As another alternative the at least five amino acid residues may be (a) AA2, AA3, AA4, AA6 and AA7, (b) AA2, AA3, AA4, AA6 and AA8, (c) AA2, AA3, AA4, AA6 and AA9, (d) AA2, AA3, AA4, AA6 and AA10, or (e) AA2, AA3, AA4, AA6 and AA11. As yet another alternative the at least five amino acid residues may be (a) AA2, AA3, AA4, AA7 and AA8, (b) AA2, AA3, AA4, AA7 and AA9, (c) AA2, AA3, AA4, AA7 and AA10, or (d) AA2, AA3, AA4, AA7 and AA11. The at least five amino acid residues may also be (a) AA2, AA3, AA4, AA8 and AA9, (b) AA2, AA3, AA4, AA8 and AA10, or (c) AA2, AA3, AA4, AA8 and AA11. The at least five amino acid residues may furthermore also be (a) AA2, AA3, AA4, AA9 and AA10, or (b) AA2, AA3, AA4, AA9 and AA11. The at least five amino acid residues may also be AA2, AA3, AA4, AA10 and AA11. Such at least five amino acid residues may be (a) AA2, AA3, AA5, AA6 and AA7, (b) AA2, AA3, AA5, AA6 and AA8, (c) AA2, AA3, AA5, AA6 and AA9, (d) AA2, AA3, AA5, AA6 and AA10, or (e) AA2, AA3, AA5, AA6 and AA11. As yet another alternative the at least five amino acid residues may be (a) AA2, AA3, AA5, AA7 and AA8, (b) AA2, AA3, AA5, AA7 and AA9, (c) AA2, AA3, AA5, AA7 and AA10, or (d) AA2, AA3, AA5, AA7 and AA11. The at least five amino acid residues may also be (a) AA2, AA3, AA5, AA8 and AA9, (b) AA2, AA3, AA5, AA8 and AA10, or (c) AA2, AA3, AA5, AA8 and AA11. The at least five amino acid residues may furthermore also be (a) AA2, AA3, AA5, AA9 and AA10, or (b) AA2, AA3, AA5, AA9 and AA11. The at least five amino acid residues may also be AA2, AA3, AA5, AA10 and AA11. Such at least five amino acid residues may be (a) AA2, AA3, AA6, AA7 and AA8, (b) AA2, AA3, AA6, AA7 and AA9, (c) AA2, AA3, AA6, AA7 and AA10, or (d) AA2, AA3, AA6, AA7 and AA11. The at least five amino acid residues may also be (a) AA2, AA3, AA6, AA8 and AA9, (b) AA2, AA3, AA6, AA8 and AA10, or (c) AA2, AA3, AA6, AA8 and AA11. The at least five amino acid residues may furthermore also be (a) AA2, AA3, AA6, AA9 and AA10, or (b) AA2, AA3, AA6, AA9 and AA11. The at least five amino acid residues may also be AA2, AA3, AA6, AA10 and AA11. Such at least five amino acid residues may be (a) AA2, AA3, AA7, AA8 and AA9, (b) AA2, AA3, AA7, AA8 and AA10, or (c) AA2, AA3, AA7, AA8 and AA11. The at least five amino acid residues may furthermore also be (a) AA2, AA3, AA7, AA9 and AA10, or (b) AA2, AA3, AA7, AA9 and AA11. The at least five amino acid residues may also be AA2, AA3, AA7, AA10 and AA11. Such at least five amino acid residues may be (a) AA2, AA3, AA8, AA9 and AA10, or (b) AA2, AA3, AA8, AA9 and AA11. The at least five amino acid residues may also be AA2, AA3, AA8, AA10 and AA11. The at least five amino acid residues may also be AA2, AA3, AA9, AA10 and AA11. Such at least five amino acid residues may be (a) AA2, AA4, AA5, AA6 and AA7, (b) AA2, AA4, AA5, AA6 and AA8, (c) AA2, AA4, AA5, AA6 and AA9, (d) AA2, AA4, AA5, AA6 and AA10, or (e) AA2, AA4, AA5, AA6 and AA11. As yet another alternative the at least five amino acid residues may be (a) AA2, AA4, AA5, AA7 and AA8, (b) AA2, AA4, AA5, AA7 and AA9, (c) AA2, AA4, AA5, AA7 and AA10, or (d) AA2, AA4, AA5, AA7 and AA11. The at least five amino acid residues may also be (a) AA2, AA4, AA5, AA8 and AA9, (b) AA2, AA4, AA5, AA8 and AA10, or (c) AA2, AA4, AA5, AA8 and AA11. The at least five amino acid residues may furthermore also be (a) AA2, AA4, AA5, AA9 and AA10, or (b) AA2, AA4, AA5, AA9 and AA11. The at least five amino acid residues may also be AA2, AA4, AA5, AA10 and AA11. Such at least five amino acid residues may be (a) AA2, AA4, AA6, AA7 and AA8, (b) AA2, AA4, AA6, AA7 and AA9, (c) AA2, AA4, AA6, AA7 and AA10, or (d) AA2, AA4, AA6, AA7 and AA11. The at least five amino acid residues may also be (a) AA2, AA4, AA6, AA8 and AA9, (b) AA2, AA4, AA6, AA8 and AA10, or (c) AA2, AA4, AA6, AA8 and AA11. The at least five amino acid residues may furthermore also be (a) AA2, AA4, AA6, AA9 and AA10, or (b) AA2, AA4, AA6, AA9 and AA11. The at least five amino acid residues may also be AA2, AA4, AA6, AA10 and AA11. Such at least five amino acid residues may be (a) AA2, AA4, AA7, AA8 and AA9, (b) AA2, AA4, AA7, AA8 and AA10, or (c) AA2, AA4, AA7, AA8 and AA11. The at least five amino acid residues may furthermore also be (a) AA2, AA4, AA7, AA9 and AA10, or (b) AA2, AA4, AA7, AA9 and AA11. The at least five amino acid residues may also be AA2, AA4, AA7, AA10 and AA11. Such at least five amino acid residues may be (a) AA2, AA4, AA8, AA9 and AA10, or (b) AA2, AA4, AA8, AA9 and AA11. The at least five amino acid residues may also be AA2, AA4, AA8, AA10 and AA11. The at least five amino acid residues may also be AA2, AA4, AA9, AA10 and AA11. Such at least five amino acid residues may be (a) AA2, AA5, AA6, AA7 and AA8, (b) AA2, AA5, AA6, AA7 and AA9, (c) AA2, AA5, AA6, AA7 and AA10, or (d) AA2, AA5, AA6, AA7 and AA11. The at least five amino acid residues may also be (a) AA2, AA5, AA6, AA8 and AA9, (b) AA2, AA5, AA6, AA8 and AA10, or (c) AA2, AA5, AA6, AA8 and AA11. The at least five amino acid residues may furthermore also be (a) AA2, AA5, AA6, AA9 and AA10, or (b) AA2, AA5, AA6, AA9 and AA11. The at least five amino acid residues may also be AA2, AA5, AA6, AA10 and AA11. Such at least five amino acid residues may be (a) AA2, AA5, AA7, AA8 and AA9, (b) AA2, AA5, AA7, AA8 and AA10, or (c) AA2, AA5, AA7, AA8 and AA11. The at least five amino acid residues may furthermore also be (a) AA2, AA5, AA7, AA9 and AA10, or (b) AA2, AA5, AA7, AA9 and AA11. The at least five amino acid residues may also be AA2, AA5, AA7, AA10 and AA11. Such at least five amino acid residues may be (a) AA2, AA5, AA8, AA9 and AA10, or (b) AA2, AA5, AA8, AA9 and AA11. The at least five amino acid residues may also be AA2, AA5, AA8, AA10 and AA11. The at least five amino acid residues may also be AA2, AA5, AA9, AA10 and AA11. Such at least five amino acid residues may be (a) AA2, AA6, AA7, AA8 and AA9, (b) AA2, AA6, AA7, AA8 and AA10, or (c) AA2, AA6, AA7, AA8 and AA11. The at least five amino acid residues may furthermore also be (a) AA2, AA6, AA7, AA9 and AA10, or (b) AA2, AA6, AA7, AA9 and AA11. The at least five amino acid residues may also be AA2, AA6, AA7, AA10 and AA11. Such at least five amino acid residues may be (a) AA2, AA6, AA8, AA9 and AA10, or (b) AA2, AA6, AA8, AA9 and AA11. The at least five amino acid residues may also be AA2, AA6, AA8, AA10 and AA11. The at least five amino acid residues may also be AA2, AA6, AA9, AA10 and AA11. Such at least five amino acid residues may be (a) AA2, AA7, AA8, AA9 and AA10, or (b) AA2, AA7, AA8, AA9 and AA11. The at least five amino acid residues may also be AA2, AA7, AA8, AA10 and AA11. The at least five amino acid residues may also be AA2, AA7, AA9, AA10 and AA11. The at least five amino acid residues may also be AA2, AA8, AA9, AA10 and AA11.

Such at least five amino acid residues may be (a) AA3, AA4, AA5, AA6 and AA7, (b) AA3, AA4, AA5, AA6 and AA8, (c) AA3, AA4, AA5, AA6 and AA9, (d) AA3, AA4, AA5, AA6 and AA10, or (e) AA3, AA4, AA5, AA6 and AA11. As yet another alternative the at least five amino acid residues may be (a) AA3, AA4, AA5, AA7 and AA8, (b) AA3, AA4, AA5, AA7 and AA9, (c) AA3, AA4, AA5, AA7 and AA10, or (d) AA3, AA4, AA5, AA7 and AA11. The at least five amino acid residues may also be (a) AA3, AA4, AA5, AA8 and AA9, (b) AA3, AA4, AA5, AA8 and AA10, or (c) AA3, AA4, AA5, AA8 and AA11. The at least five amino acid residues may furthermore also be (a) AA3, AA4, AA5, AA9 and AA10, or (b) AA3, AA4, AA5, AA9 and AA11. The at least five amino acid residues may also be AA3, AA4, AA5, AA10 and AA11. Such at least five amino acid residues may be (a) AA3, AA4, AA6, AA7 and AA8, (b) AA3, AA4, AA6, AA7 and AA9, (c) AA3, AA4, AA6, AA7 and AA10, or (d) AA3, AA4, AA6, AA7 and AA11. The at least five amino acid residues may also be (a) AA3, AA4, AA6, AA8 and AA9, (b) AA3, AA4, AA6, AA8 and AA10, or (c) AA3, AA4, AA6, AA8 and AA11. The at least five amino acid residues may furthermore also be (a) AA3, AA4, AA6, AA9 and AA10, or (b) AA3, AA4, AA6, AA9 and AA11. The at least five amino acid residues may also be AA3, AA4, AA6, AA10 and AA11. Such at least five amino acid residues may be (a) AA3, AA4, AA7, AA8 and AA9, (b) AA3, AA4, AA7, AA8 and AA10, or (c) AA3, AA4, AA7, AA8 and AA11. The at least five amino acid residues may furthermore also be (a) AA3, AA4, AA7, AA9 and AA10, or (b) AA3, AA4, AA7, AA9 and AA11. The at least five amino acid residues may also be AA3, AA4, AA7, AA10 and AA11. Such at least five amino acid residues may be (a) AA3, AA4, AA8, AA9 and AA10, or (b) AA3, AA4, AA8, AA9 and AA11. The at least five amino acid residues may also be AA3, AA4, AA8, AA10 and AA11. The at least five amino acid residues may also be AA3, AA4, AA9, AA10 and AA11. Such at least five amino acid residues may be (a) AA3, AA5, AA6, AA7 and AA8, (b) AA3, AA5, AA6, AA7 and AA9, (c) AA3, AA5, AA6, AA7 and AA10, or (d) AA3, AA5, AA6, AA7 and AA11. The at least five amino acid residues may also be (a) AA3, AA5, AA6, AA8 and AA9, (b) AA3, AA5, AA6, AA8 and AA10, or (c) AA3, AA5, AA6, AA8 and AA11. The at least five amino acid residues may furthermore also be (a) AA3, AA5, AA6, AA9 and AA10, or (b) AA3, AA5, AA6, AA9 and AA11. The at least five amino acid residues may also be AA3, AA5, AA6, AA10 and AA11. Such at least five amino acid residues may be (a) AA3, AA5, AA7, AA8 and AA9, (b) AA3, AA5, AA7, AA8 and AA10, or (c) AA3, AA5, AA7, AA8 and AA11. The at least five amino acid residues may furthermore also be (a) AA3, AA5, AA7, AA9 and AA10, or (b) AA3, AA5, AA7, AA9 and AA11. The at least five amino acid residues may also be AA3, AA5, AA7, AA10 and AA11. Such at least five amino acid residues may be (a) AA3, AA5, AA8, AA9 and AA10, or (b) AA3, AA5, AA8, AA9 and AA11. The at least five amino acid residues may also be AA3, AA5, AA8, AA10 and AA11. The at least five amino acid residues may also be AA3, AA5, AA9, AA10 and AA11. Such at least five amino acid residues may be (a) AA3, AA6, AA7, AA8 and AA9, (b) AA3, AA6, AA7, AA8 and AA10, or (c) AA3, AA6, AA7, AA8 and AA11. The at least five amino acid residues may furthermore also be (a) AA3, AA6, AA7, AA9 and AA10, or (b) AA3, AA6, AA7, AA9 and AA11. The at least five amino acid residues may also be AA3, AA6, AA7, AA10 and AA11. Such at least five amino acid residues may be (a) AA3, AA6, AA8, AA9 and AA10, or (b) AA3, AA6, AA8, AA9 and AA11. The at least five amino acid residues may also be AA3, AA6, AA8, AA10 and AA11. The at least five amino acid residues may also be AA3, AA6, AA9, AA10 and AA11. Such at least five amino acid residues may be (a) AA3, AA7, AA8, AA9 and AA10, or (b) AA3, AA7, AA8, AA9 and AA11. The at least five amino acid residues may also be AA3, AA7, AA8, AA10 and AA11. The at least five amino acid residues may also be AA3, AA7, AA9, AA10 and AA11. The at least five amino acid residues may also be AA3, AA8, AA9, AA10 and AA11.

Such at least five amino acid residues may be (a) AA4, AA5, AA6, AA7 and AA8, (b) AA4, AA5, AA6, AA7 and AA9, (c) AA4, AA5, AA6, AA7 and AA10, or (d) AA4, AA5, AA6, AA7 and AA11. The at least five amino acid residues may also be (a) AA4, AA5, AA6, AA8 and AA9, (b) AA4, AA5, AA6, AA8 and AA10, or (c) AA4, AA5, AA6, AA8 and AA11. The at least five amino acid residues may furthermore also be (a) AA4, AA5, AA6, AA9 and AA10, or (b) AA4, AA5, AA6, AA9 and AA11. The at least five amino acid residues may also be AA4, AA5, AA6, AA10 and AA11. Such at least five amino acid residues may be (a) AA4, AA5, AA7, AA8 and AA9, (b) AA4, AA5, AA7, AA8 and AA10, or (c) AA4, AA5, AA7, AA8 and AA11. The at least five amino acid residues may furthermore also be (a) AA4, AA5, AA7, AA9 and AA10, or (b) AA4, AA5, AA7, AA9 and AA11. The at least five amino acid residues may also be AA4, AA5, AA7, AA10 and AA11. Such at least five amino acid residues may be (a) AA4, AA5, AA8, AA9 and AA10, or (b) AA4, AA5, AA8, AA9 and AA11. The at least five amino acid residues may also be AA4, AA5, AA8, AA10 and AA11. The at least five amino acid residues may also be AA4, AA5, AA9, AA10 and AA11. Such at least five amino acid residues may be (a) AA4, AA6, AA7, AA8 and AA9, (b) AA4, AA6, AA7, AA8 and AA10, or (c) AA4, AA6, AA7, AA8 and AA11. The at least five amino acid residues may furthermore also be (a) AA4, AA6, AA7, AA9 and AA10, or (b) AA4, AA6, AA7, AA9 and AA11. The at least five amino acid residues may also be AA4, AA6, AA7, AA10 and AA11. Such at least five amino acid residues may be (a) AA4, AA6, AA8, AA9 and AA10, or (b) AA4, AA6, AA8, AA9 and AA11. The at least five amino acid residues may also be AA4, AA6, AA8, AA10 and AA11. The at least five amino acid residues may also be AA4, AA6, AA9, AA10 and AA11. Such at least five amino acid residues may be (a) AA4, AA7, AA8, AA9 and AA10, or (b) AA4, AA7, AA8, AA9 and AA11. The at least five amino acid residues may also be AA4, AA7, AA8, AA10 and AA11. The at least five amino acid residues may also be AA4, AA7, AA9, AA10 and AA11. The at least five amino acid residues may also be AA4, AA8, AA9, AA10 and AA11.

Such at least five amino acid residues may be (a) AA5, AA6, AA7, AA8 and AA9, (b) AA5, AA6, AA7, AA8 and AA10, or (c) AA5, AA6, AA7, AA8 and AA11. The at least five amino acid residues may furthermore also be (a) AA5, AA6, AA7, AA9 and AA10, or (b) AA5, AA6, AA7, AA9 and AA11. The at least five amino acid residues may also be AA5, AA6, AA7, AA10 and AA11. Such at least five amino acid residues may be (a) AA5, AA6, AA8, AA9 and AA10, or (b) AA5, AA6, AA8, AA9 and AA11. The at least five amino acid residues may also be AA5, AA6, AA8, AA10 and AA11. The at least five amino acid residues may also be AA5, AA6, AA9, AA10 and AA11. Such at least five amino acid residues may be (a) AA5, AA7, AA8, AA9 and AA10, or (b) AA5, AA7, AA8, AA9 and AA11. The at least five amino acid residues may also be AA5, AA7, AA8, AA10 and AA11. The at least five amino acid residues may also be AA5, AA7, AA9, AA10 and AA11. The at least five amino acid residues may also be AA5, AA8, AA9, AA10 and AA11. Such at least five amino acid residues may be (a) AA6, AA7, AA8, AA9 and AA10, or (b) AA6, AA7, AA8, AA9 and AA11. The at least five amino acid residues may also be AA6, AA7, AA8, AA10 and AA11. The at least five amino acid residues may also be AA6, AA7, AA9, AA10 and AA11. The at least five amino acid residues may also be AA6, AA8, AA9, AA10 and AA11. The at least five amino acid residues may also be AA7, AA8, AA9, AA10 and AA11.

Such at least six amino acid residues may be (a) AA1, AA2, AA3, AA4, AA5 and AA6, (b) AA1, AA2, AA3, AA4, AA5 and AA7, (c) AA1, AA2, AA3, AA4, AA5 and AA8, (d) AA1, AA2, AA3, AA4, AA5 and AA9, (e) AA1, AA2, AA3, AA4, AA5 and AA10, or (f) AA1, AA2, AA3, AA4, AA5 and AA11. As another alternative the at least six amino acid residues may be (a) AA1, AA2, AA3, AA4, AA6 and AA7, (b) AA1, AA2, AA3, AA4, AA6 and AA8, (c) AA1, AA2, AA3, AA4, AA6 and AA9, (d) AA1, AA2, AA3, AA4, AA6 and AA10, or (e) AA1, AA2, AA3, AA4, AA6 and AA11. As yet another alternative the at least six amino acid residues may be (a) AA1, AA2, AA3, AA4, AA7 and AA8, (b) AA1, AA2, AA3, AA4, AA7 and AA9, (c) AA1, AA2, AA3, AA4, AA7 and AA10, or (d) AA1, AA2, AA3, AA4, AA7 and AA11. The at least six amino acid residues may also be (a) AA1, AA2, AA3, AA4, AA8 and AA9, (b) AA1, AA2, AA3, AA4, AA8 and AA10, or (c) AA1, AA2, AA3, AA4, AA8 and AA11. The at least six amino acid residues may furthermore also be (a) AA1, AA2, AA3, AA4, AA9 and AA10, or (b) AA1, AA2, AA3, AA4, AA9 and AA11. The at least six amino acid residues may also be AA1, AA2, AA3, AA4, AA10 and AA11. Such at least six amino acid residues may be (a) AA1, AA2, AA3, AA5, AA6 and AA7, (b) AA1, AA2, AA3, AA5, AA6 and AA8, (c) AA1, AA2, AA3, AA5, AA6 and AA9, (d) AA1, AA2, AA3, AA5, AA6 and AA10, or (e) AA1, AA2, AA3, AA5, AA6 and AA11. As yet another alternative the at least six amino acid residues may be (a) AA1, AA2, AA3, AA5, AA7 and AA8, (b) AA1, AA2, AA3, AA5, AA7 and AA9, (c) AA1, AA2, AA3, AA5, AA7 and AA10, or (d) AA1, AA2, AA3, AA5, AA7 and AA11. The at least six amino acid residues may also be (a) AA1, AA2, AA3, AA5, AA8 and AA9, (b) AA1, AA2, AA3, AA5, AA8 and AA10, or (c) AA1, AA2, AA3, AA5, AA8 and AA11. The at least six amino acid residues may furthermore also be (a) AA1, AA2, AA3, AA5, AA9 and AA10, or (b) AA1, AA2, AA3, AA5, AA9 and AA11. The at least six amino acid residues may also be AA1, AA2, AA3, AA5, AA10 and AA11. Such at least six amino acid residues may be (a) AA1, AA2, AA3, AA6, AA7 and AA8, (b) AA1, AA2, AA3, AA6, AA7 and AA9, (c) AA1, AA2, AA3, AA6, AA7 and AA10, or (d) AA1, AA2, AA3, AA6, AA7 and AA11. The at least six amino acid residues may also be (a) AA1, AA2, AA3, AA6, AA8 and AA9, (b) AA1, AA2, AA3, AA6, AA8 and AA10, or (c) AA1, AA2, AA3, AA6, AA8 and AA11. The at least six amino acid residues may furthermore also be (a) AA1, AA2, AA3, AA6, AA9 and AA10, or (b) AA1, AA2, AA3, AA6, AA9 and AA11. The at least six amino acid residues may also be AA1, AA2, AA3, AA6, AA10 and AA11. Such at least six amino acid residues may be (a) AA1, AA2, AA3, AA7, AA8 and AA9, (b) AA1, AA2, AA3, AA7, AA8 and AA10, or (c) AA1, AA2, AA3, AA7, AA8 and AA11. The at least six amino acid residues may furthermore also be (a) AA1, AA2, AA3, AA7, AA9 and AA10, or (b) AA1, AA2, AA3, AA7, AA9 and AA11. The at least six amino acid residues may also be AA1, AA2, AA3, AA7, AA10 and AA11. Such at least six amino acid residues may be (a) AA1, AA2, AA3, AA8, AA9 and AA10, or (b) AA1, AA2, AA3, AA8, AA9 and AA11. The at least six amino acid residues may also be AA1, AA2, AA3, AA8, AA10 and AA11. The at least six amino acid residues may also be AA1, AA2, AA3, AA9, AA10 and AA11. Such at least six amino acid residues may be (a) AA1, AA2, AA4, AA5, AA6 and AA7, (b) AA1, AA2, AA4, AA5, AA6 and AA8, (c) AA1, AA2, AA4, AA5, AA6 and AA9, (d) AA1, AA2, AA4, AA5, AA6 and AA10, or (e) AA1, AA2, AA4, AA5, AA6 and AA11. As yet another alternative the at least six amino acid residues may be (a) AA1, AA2, AA4, AA5, AA7 and AA8, (b) AA1, AA2, AA4, AA5, AA7 and AA9, (c) AA1, AA2, AA4, AA5, AA7 and AA10, or (d) AA1, AA2, AA4, AA5, AA7 and AA11. The at least six amino acid residues may also be (a) AA1, AA2, AA4, AA5, AA8 and AA9, (b) AA1, AA2, AA4, AA5, AA8 and AA10, or (c) AA1, AA2, AA4, AA5, AA8 and AA11. The at least six amino acid residues may furthermore also be (a) AA1, AA2, AA4, AA5, AA9 and AA10, or (b) AA1, AA2, AA4, AA5, AA9 and AA11. The at least six amino acid residues may also be AA1, AA2, AA4, AA5, AA10 and AA11. Such at least six amino acid residues may be (a) AA1, AA2, AA4, AA6, AA7 and AA8, (b) AA1, AA2, AA4, AA6, AA7 and AA9, (c) AA1, AA2, AA4, AA6, AA7 and AA10, or (d) AA1, AA2, AA4, AA6, AA7 and AA11. The at least six amino acid residues may also be (a) AA1, AA2, AA4, AA6, AA8 and AA9, (b) AA1, AA2, AA4, AA6, AA8 and AA10, or (c) AA1, AA2, AA4, AA6, AA8 and AA11. The at least six amino acid residues may furthermore also be (a) AA1, AA2, AA4, AA6, AA9 and AA10, or (b) AA1, AA2, AA4, AA6, AA9 and AA11. The at least six amino acid residues may also be AA1, AA2, AA4, AA6, AA10 and AA11. Such at least six amino acid residues may be (a) AA1, AA2, AA4, AA7, AA8 and AA9, (b) AA1, AA2, AA4, AA7, AA8 and AA10, or (c) AA1, AA2, AA4, AA7, AA8 and AA11. The at least six amino acid residues may furthermore also be (a) AA1, AA2, AA4, AA7, AA9 and AA10, or (b) AA1, AA2, AA4, AA7, AA9 and AA11. The at least six amino acid residues may also be AA1, AA2, AA4, AA7, AA10 and AA11. Such at least six amino acid residues may be (a) AA1, AA2, AA4, AA8, AA9 and AA10, or (b) AA1, AA2, AA4, AA8, AA9 and AA11. The at least six amino acid residues may also be AA1, AA2, AA4, AA8, AA10 and AA11. The at least six amino acid residues may also be AA1, AA2, AA4, AA9, AA10 and AA11. Such at least six amino acid residues may be (a) AA1, AA2, AA5, AA6, AA7 and AA8, (b) AA1, AA2, AA5, AA6, AA7 and AA9, (c) AA1, AA2, AA5, AA6, AA7 and AA10, or (d) AA1, AA2, AA5, AA6, AA7 and AA11. The at least six amino acid residues may also be (a) AA1, AA2, AA5, AA6, AA8 and AA9, (b) AA1, AA2, AA5, AA6, AA8 and AA10, or (c) AA1, AA2, AA5, AA6, AA8 and AA11. The at least six amino acid residues may furthermore also be (a) AA1, AA2, AA5, AA6, AA9 and AA10, or (b) AA1, AA2, AA5, AA6, AA9 and AA11. The at least six amino acid residues may also be AA1, AA2, AA5, AA6, AA10 and AA11. Such at least six amino acid residues may be (a) AA1, AA2, AA5, AA7, AA8 and AA9, (b) AA1, AA2, AA5, AA7, AA8 and AA10, or (c) AA1, AA2, AA5, AA7, AA8 and AA11. The at least six amino acid residues may furthermore also be (a) AA1, AA2, AA5, AA7, AA9 and AA10, or (b) AA1, AA2, AA5, AA7, AA9 and AA11. The at least six amino acid residues may also be AA1, AA2, AA5, AA7, AA10 and AA11. Such at least six amino acid residues may be (a) AA1, AA2, AA5, AA8, AA9 and AA10, or (b) AA1, AA2, AA5, AA8, AA9 and AA11. The at least six amino acid residues may also be AA1, AA2, AA5, AA8, AA10 and AA11. The at least six amino acid residues may also be AA1, AA2, AA5, AA9, AA10 and AA11. Such at least six amino acid residues may be (a) AA1, AA2, AA6, AA7, AA8 and AA9, (b) AA1, AA2, AA6, AA7, AA8 and AA10, or (c) AA1, AA2, AA6, AA7, AA8 and AA11. The at least six amino acid residues may furthermore also be (a) AA1, AA2, AA6, AA7, AA9 and AA10, or (b) AA1, AA2, AA6, AA7, AA9 and AA11. The at least six amino acid residues may also be AA1, AA2, AA6, AA7, AA10 and AA11. Such at least six amino acid residues may be (a) AA1, AA2, AA6, AA8, AA9 and AA10, or (b) AA1, AA2, AA6, AA8, AA9 and AA11. The at least six amino acid residues may also be AA1, AA2, AA6, AA8, AA10 and AA11. The at least six amino acid residues may also be AA1, AA2, AA6, AA9, AA10 and AA11. Such at least six amino acid residues may be (a) AA1, AA2, AA7, AA8, AA9 and AA10, or (b) AA1, AA2, AA7, AA8, AA9 and AA11. The at least six amino acid residues may also be AA1, AA2, AA7, AA8, AA10 and AA11. The at least six amino acid residues may also be AA1, AA2, AA7, AA9, AA10 and AA11. The at least six amino acid residues may also be AA1, AA2, AA8, AA9, AA10 and AA11. Such at least six amino acid residues may be (a) AA1, AA3, AA4, AA5, AA6 and AA7, (b) AA1, AA3, AA4, AA5, AA6 and AA8, (c) AA1, AA3, AA4, AA5, AA6 and AA9, (d) AA1, AA3, AA4, AA5, AA6 and AA10, or (e) AA1, AA3, AA4, AA5, AA6 and AA11. As yet another alternative the at least six amino acid residues may be (a) AA1, AA3, AA4, AA5, AA7 and AA8, (b) AA1, AA3, AA4, AA5, AA7 and AA9, (c) AA1, AA3, AA4, AA5, AA7 and AA10, or (d) AA1, AA3, AA4, AA5, AA7 and AA11. The at least six amino acid residues may also be (a) AA1, AA3, AA4, AA5, AA8 and AA9, (b) AA1, AA3, AA4, AA5, AA8 and AA10, or (c) AA1, AA3, AA4, AA5, AA8 and AA11. The at least six amino acid residues may furthermore also be (a) AA1, AA3, AA4, AA5, AA9 and AA10, or (b) AA1, AA3, AA4, AA5, AA9 and AA11. The at least six amino acid residues may also be AA1, AA3, AA4, AA5, AA10 and AA11. Such at least six amino acid residues may be (a) AA1, AA3, AA4, AA6, AA7 and AA8, (b) AA1, AA3, AA4, AA6, AA7 and AA9, (c) AA1, AA3, AA4, AA6, AA7 and AA10, or (d) AA1, AA3, AA4, AA6, AA7 and AA11. The at least six amino acid residues may also be (a) AA1, AA3, AA4, AA6, AA8 and AA9, (b) AA1, AA3, AA4, AA6, AA8 and AA10, or (c) AA1, AA3, AA4, AA6, AA8 and AA11. The at least six amino acid residues may furthermore also be (a) AA1, AA3, AA4, AA6, AA9 and AA10, or (b) AA1, AA3, AA4, AA6, AA9 and AA11. The at least six amino acid residues may also be AA1, AA3, AA4, AA6, AA10 and AA11. Such at least six amino acid residues may be (a) AA1, AA3, AA4, AA7, AA8 and AA9, (b) AA1, AA3, AA4, AA7, AA8 and AA10, or (c) AA1, AA3, AA4, AA7, AA8 and AA11. The at least six amino acid residues may furthermore also be (a) AA1, AA3, AA4, AA7, AA9 and AA10, or (b) AA1, AA3, AA4, AA7, AA9 and AA11. The at least six amino acid residues may also be AA1, AA3, AA4, AA7, AA10 and AA11. Such at least six amino acid residues may be (a) AA1, AA3, AA4, AA8, AA9 and AA10, or (b) AA1, AA3, AA4, AA8, AA9 and AA11. The at least six amino acid residues may also be AA1, AA3, AA4, AA8, AA10 and AA11. The at least six amino acid residues may also be AA1, AA3, AA4, AA9, AA10 and AA11. Such at least six amino acid residues may be (a) AA1, AA3, AA5, AA6, AA7 and AA8, (b) AA1, AA3, AA5, AA6, AA7 and AA9, (c) AA1, AA3, AA5, AA6, AA7 and AA10, or (d) AA1, AA3, AA5, AA6, AA7 and AA11. The at least six amino acid residues may also be (a) AA1, AA3, AA5, AA6, AA8 and AA9, (b) AA1, AA3, AA5, AA6, AA8 and AA10, or (c) AA1, AA3, AA5, AA6, AA8 and AA11. The at least six amino acid residues may furthermore also be (a) AA1, AA3, AA5, AA6, AA9 and AA10, or (b) AA1, AA3, AA5, AA6, AA9 and AA11. The at least six amino acid residues may also be AA1, AA3, AA5, AA6, AA10 and AA11. Such at least six amino acid residues may be (a) AA1, AA3, AA5, AA7, AA8 and AA9, (b) AA1, AA3, AA5, AA7, AA8 and AA10, or (c) AA1, AA3, AA5, AA7, AA8 and AA11. The at least six amino acid residues may furthermore also be (a) AA1, AA3, AA5, AA7, AA9 and AA10, or (b) AA1, AA3, AA5, AA7, AA9 and AA11. The at least six amino acid residues may also be AA1, AA3, AA5, AA7, AA10 and AA11. Such at least six amino acid residues may be (a) AA1, AA3, AA5, AA8, AA9 and AA10, or (b) AA1, AA3, AA5, AA8, AA9 and AA11. The at least six amino acid residues may also be AA1, AA3, AA5, AA8, AA10 and AA11. The at least six amino acid residues may also be AA1, AA3, AA5, AA9, AA10 and AA11. Such at least six amino acid residues may be (a) AA1, AA3, AA6, AA7, AA8 and AA9, (b) AA1, AA3, AA6, AA7, AA8 and AA10, or (c) AA1, AA3, AA6, AA7, AA8 and AA11. The at least six amino acid residues may furthermore also be (a) AA1, AA3, AA6, AA7, AA9 and AA10, or (b) AA1, AA3, AA6, AA7, AA9 and AA11. The at least six amino acid residues may also be AA1, AA3, AA6, AA7, AA10 and AA11. Such at least six amino acid residues may be (a) AA1, AA3, AA6, AA8, AA9 and AA10, or (b) AA1, AA3, AA6, AA8, AA9 and AA11. The at least six amino acid residues may also be AA1, AA3, AA6, AA8, AA10 and AA11. The at least six amino acid residues may also be AA1, AA3, AA6, AA9, AA10 and AA11. Such at least six amino acid residues may be (a) AA1, AA3, AA7, AA8, AA9 and AA10, or (b) AA1, AA3, AA7, AA8, AA9 and AA11. The at least six amino acid residues may also be AA1, AA3, AA7, AA8, AA10 and AA11. The at least six amino acid residues may also be AA1, AA3, AA7, AA9, AA10 and AA11. The at least six amino acid residues may also be AA1, AA3, AA8, AA9, AA10 and AA11. Such at least six amino acid residues may be (a) AA1, AA4, AA5, AA6, AA7 and AA8, (b) AA1, AA4, AA5, AA6, AA7 and AA9, (c) AA1, AA4, AA5, AA6, AA7 and AA10, or (d) AA1, AA4, AA5, AA6, AA7 and AA11. The at least six amino acid residues may also be (a) AA1, AA4, AA5, AA6, AA8 and AA9, (b) AA1, AA4, AA5, AA6, AA8 and AA10, or (c) AA1, AA4, AA5, AA6, AA8 and AA11. The at least six amino acid residues may furthermore also be (a) AA1, AA4, AA5, AA6, AA9 and AA10, or (b) AA1, AA4, AA5, AA6, AA9 and AA11. The at least six amino acid residues may also be AA1, AA4, AA5, AA6, AA10 and AA11. Such at least six amino acid residues may be (a) AA1, AA4, AA5, AA7, AA8 and AA9, (b) AA1, AA4, AA5, AA7, AA8 and AA10, or (c) AA1, AA4, AA5, AA7, AA8 and AA11. The at least six amino acid residues may furthermore also be (a) AA1, AA4, AA5, AA7, AA9 and AA10, or (b) AA1, AA4, AA5, AA7, AA9 and AA11. The at least six amino acid residues may also be AA1, AA4, AA5, AA7, AA10 and AA11. Such at least six amino acid residues may be (a) AA1, AA4, AA5, AA8, AA9 and AA10, or (b) AA1, AA4, AA5, AA8, AA9 and AA11. The at least six amino acid residues may also be AA1, AA4, AA5, AA8, AA10 and AA11. The at least six amino acid residues may also be AA1, AA4, AA5, AA9, AA10 and AA11. Such at least six amino acid residues may be (a) AA1, AA4, AA6, AA7, AA8 and AA9, (b) AA1, AA4, AA6, AA7, AA8 and AA10, or (c) AA1, AA4, AA6, AA7, AA8 and AA11. The at least six amino acid residues may furthermore also be (a) AA1, AA4, AA6, AA7, AA9 and AA10, or (b) AA1, AA4, AA6, AA7, AA9 and AA11. The at least six amino acid residues may also be AA1, AA4, AA6, AA7, AA10 and AA11. Such at least six amino acid residues may be (a) AA1, AA4, AA6, AA8, AA9 and AA10, or (b) AA1, AA4, AA6, AA8, AA9 and AA11. The at least six amino acid residues may also be AA1, AA4, AA6, AA8, AA10 and AA11. The at least six amino acid residues may also be AA1, AA4, AA6, AA9, AA10 and AA11. Such at least six amino acid residues may be (a) AA1, AA4, AA7, AA8, AA9 and AA10, or (b) AA1, AA4, AA7, AA8, AA9 and AA11. The at least six amino acid residues may also be AA1, AA4, AA7, AA8, AA10 and AA11. The at least six amino acid residues may also be AA1, AA4, AA7, AA9, AA10 and AA11. The at least six amino acid residues may also be AA1, AA4, AA8, AA9, AA10 and AA11. Such at least six amino acid residues may be (a) AA1, AA5, AA6, AA7, AA8 and AA9, (b) AA1, AA5, AA6, AA7, AA8 and AA10, or (c) AA1, AA5, AA6, AA7, AA8 and AA11. The at least six amino acid residues may furthermore also be (a) AA1, AA5, AA6, AA7, AA9 and AA10, or (b) AA1, AA5, AA6, AA7, AA9 and AA11. The at least six amino acid residues may also be AA1, AA5, AA6, AA7, AA10 and AA11. Such at least six amino acid residues may be (a) AA1, AA5, AA6, AA8, AA9 and AA10, or (b) AA1, AA5, AA6, AA8, AA9 and AA11. The at least six amino acid residues may also be AA1, AA5, AA6, AA8, AA10 and AA11. The at least six amino acid residues may also be AA1, AA5, AA6, AA9, AA10 and AA11. Such at least six amino acid residues may be (a) AA1, AA5, AA7, AA8, AA9 and AA10, or (b) AA1, AA5, AA7, AA8, AA9 and AA11. The at least six amino acid residues may also be AA1, AA5, AA7, AA8, AA10 and AA11. The at least six amino acid residues may also be AA1, AA5, AA7, AA9, AA10 and AA11. The at least six amino acid residues may also be AA1, AA5, AA8, AA9, AA10 and AA11. Such at least six amino acid residues may be (a) AA1, AA6, AA7, AA8, AA9 and AA10, or (b) AA1, AA6, AA7, AA8, AA9 and AA11. The at least six amino acid residues may also be AA1, AA6, AA7, AA8, AA10 and AA11. The at least six amino acid residues may also be AA1, AA6, AA7, AA9, AA10 and AA11. The at least six amino acid residues may also be AA1, AA6, AA8, AA9, AA10 and AA11. The at least six amino acid residues may also be AA1, AA7, AA8, AA9, AA10 and AA11.

Such at least six amino acid residues may be (a) AA2, AA3, AA4, AA5, AA6 and AA7, (b) AA2, AA3, AA4, AA5, AA6 and AA8, (c) AA2, AA3, AA4, AA5, AA6 and AA9, (d) AA2, AA3, AA4, AA5, AA6 and AA10, or (e) AA2, AA3, AA4, AA5, AA6 and AA11. As yet another alternative the at least six amino acid residues may be (a) AA2, AA3, AA4, AA5, AA7 and AA8, (b) AA2, AA3, AA4, AA5, AA7 and AA9, (c) AA2, AA3, AA4, AA5, AA7 and AA10, or (d) AA2, AA3, AA4, AA5, AA7 and AA11. The at least six amino acid residues may also be (a) AA2, AA3, AA4, AA5, AA8 and AA9, (b) AA2, AA3, AA4, AA5, AA8 and AA10, or (c) AA2, AA3, AA4, AA5, AA8 and AA11. The at least six amino acid residues may furthermore also be (a) AA2, AA3, AA4, AA5, AA9 and AA10, or (b) AA2, AA3, AA4, AA5, AA9 and AA11. The at least six amino acid residues may also be AA2, AA3, AA4, AA5, AA10 and AA11. Such at least six amino acid residues may be (a) AA2, AA3, AA4, AA6, AA7 and AA8, (b) AA2, AA3, AA4, AA6, AA7 and AA9, (c) AA2, AA3, AA4, AA6, AA7 and AA10, or (d) AA2, AA3, AA4, AA6, AA7 and AA11. The at least six amino acid residues may also be (a) AA2, AA3, AA4, AA6, AA8 and AA9, (b) AA2, AA3, AA4, AA6, AA8 and AA10, or (c) AA2, AA3, AA4, AA6, AA8 and AA11. The at least six amino acid residues may furthermore also be (a) AA2, AA3, AA4, AA6, AA9 and AA10, or (b) AA2, AA3, AA4, AA6, AA9 and AA11. The at least six amino acid residues may also be AA2, AA3, AA4, AA6, AA10 and AA11. Such at least six amino acid residues may be (a) AA2, AA3, AA4, AA7, AA8 and AA9, (b) AA2, AA3, AA4, AA7, AA8 and AA10, or (c) AA2, AA3, AA4, AA7, AA8 and AA11. The at least six amino acid residues may furthermore also be (a) AA2, AA3, AA4, AA7, AA9 and AA10, or (b) AA2, AA3, AA4, AA7, AA9 and AA11. The at least six amino acid residues may also be AA2, AA3, AA4, AA7, AA10 and AA11. Such at least six amino acid residues may be (a) AA2, AA3, AA4, AA8, AA9 and AA10, or (b) AA2, AA3, AA4, AA8, AA9 and AA11. The at least six amino acid residues may also be AA2, AA3, AA4, AA8, AA10 and AA11. The at least six amino acid residues may also be AA2, AA3, AA4, AA9, AA10 and AA11. Such at least six amino acid residues may be (a) AA2, AA3, AA5, AA6, AA7 and AA8, (b) AA2, AA3, AA5, AA6, AA7 and AA9, (c) AA2, AA3, AA5, AA6, AA7 and AA10, or (d) AA2, AA3, AA5, AA6, AA7 and AA11. The at least six amino acid residues may also be (a) AA2, AA3, AA5, AA6, AA8 and AA9, (b) AA2, AA3, AA5, AA6, AA8 and AA10, or (c) AA2, AA3, AA5, AA6, AA8 and AA11. The at least six amino acid residues may furthermore also be (a) AA2, AA3, AA5, AA6, AA9 and AA10, or (b) AA2, AA3, AA5, AA6, AA9 and AA11. The at least six amino acid residues may also be AA2, AA3, AA5, AA6, AA10 and AA11. Such at least six amino acid residues may be (a) AA2, AA3, AA5, AA7, AA8 and AA9, (b) AA2, AA3, AA5, AA7, AA8 and AA10, or (c) AA2, AA3, AA5, AA7, AA8 and AA11. The at least six amino acid residues may furthermore also be (a) AA2, AA3, AA5, AA7, AA9 and AA10, or (b) AA2, AA3, AA5, AA7, AA9 and AA11. The at least six amino acid residues may also be AA2, AA3, AA5, AA7, AA10 and AA11. Such at least six amino acid residues may be (a) AA2, AA3, AA5, AA8, AA9 and AA10, or (b) AA2, AA3, AA5, AA8, AA9 and AA11. The at least six amino acid residues may also be AA2, AA3, AA5, AA8, AA10 and AA11. The at least six amino acid residues may also be AA2, AA3, AA5, AA9, AA10 and AA11. Such at least six amino acid residues may be (a) AA2, AA3, AA6, AA7, AA8 and AA9, (b) AA2, AA3, AA6, AA7, AA8 and AA10, or (c) AA2, AA3, AA6, AA7, AA8 and AA11. The at least six amino acid residues may furthermore also be (a) AA2, AA3, AA6, AA7, AA9 and AA10, or (b) AA2, AA3, AA6, AA7, AA9 and AA11. The at least six amino acid residues may also be AA2, AA3, AA6, AA7, AA10 and AA11. Such at least six amino acid residues may be (a) AA2, AA3, AA6, AA8, AA9 and AA10, or (b) AA2, AA3, AA6, AA8, AA9 and AA11. The at least six amino acid residues may also be AA2, AA3, AA6, AA8, AA10 and AA11. The at least six amino acid residues may also be AA2, AA3, AA6, AA9, AA10 and AA11. Such at least six amino acid residues may be (a) AA2, AA3, AA7, AA8, AA9 and AA10, or (b) AA2, AA3, AA7, AA8, AA9 and AA11. The at least six amino acid residues may also be AA2, AA3, AA7, AA8, AA10 and AA11. The at least six amino acid residues may also be AA2, AA3, AA7, AA9, AA10 and AA11. The at least six amino acid residues may also be AA2, AA3, AA8, AA9, AA10 and AA11. Such at least six amino acid residues may be (a) AA2, AA4, AA5, AA6, AA7 and AA8, (b) AA2, AA4, AA5, AA6, AA7 and AA9, (c) AA2, AA4, AA5, AA6, AA7 and AA10, or (d) AA2, AA4, AA5, AA6, AA7 and AA11. The at least six amino acid residues may also be (a) AA2, AA4, AA5, AA6, AA8 and AA9, (b) AA2, AA4, AA5, AA6, AA8 and AA10, or (c) AA2, AA4, AA5, AA6, AA8 and AA11. The at least six amino acid residues may furthermore also be (a) AA2, AA4, AA5, AA6, AA9 and AA10, or (b) AA2, AA4, AA5, AA6, AA9 and AA11. The at least six amino acid residues may also be AA2, AA4, AA5, AA6, AA10 and AA11. Such at least six amino acid residues may be (a) AA2, AA4, AA5, AA7, AA8 and AA9, (b) AA2, AA4, AA5, AA7, AA8 and AA10, or (c) AA2, AA4, AA5, AA7, AA8 and AA11. The at least six amino acid residues may furthermore also be (a) AA2, AA4, AA5, AA7, AA9 and AA10, or (b) AA2, AA4, AA5, AA7, AA9 and AA11. The at least six amino acid residues may also be AA2, AA4, AA5, AA7, AA10 and AA11. Such at least six amino acid residues may be (a) AA2, AA4, AA5, AA8, AA9 and AA10, or (b) AA2, AA4, AA5, AA8, AA9 and AA11. The at least six amino acid residues may also be AA2, AA4, AA5, AA8, AA10 and AA11. The at least six amino acid residues may also be AA2, AA4, AA5, AA9, AA10 and AA11. Such at least six amino acid residues may be (a) AA2, AA4, AA6, AA7, AA8 and AA9, (b) AA2, AA4, AA6, AA7, AA8 and AA10, or (c) AA2, AA4, AA6, AA7, AA8 and AA11. The at least six amino acid residues may furthermore also be (a) AA2, AA4, AA6, AA7, AA9 and AA10, or (b) AA2, AA4, AA6, AA7, AA9 and AA11. The at least six amino acid residues may also be AA2, AA4, AA6, AA7, AA10 and AA11. Such at least six amino acid residues may be (a) AA2, AA4, AA6, AA8, AA9 and AA10, or (b) AA2, AA4, AA6, AA8, AA9 and AA11. The at least six amino acid residues may also be AA2, AA4, AA6, AA8, AA10 and AA11. The at least six amino acid residues may also be AA2, AA4, AA6, AA9, AA10 and AA11. Such at least six amino acid residues may be (a) AA2, AA4, AA7, AA8, AA9 and AA10, or (b) AA2, AA4, AA7, AA8, AA9 and AA11. The at least six amino acid residues may also be AA2, AA4, AA7, AA8, AA10 and AA11. The at least six amino acid residues may also be AA2, AA4, AA7, AA9, AA10 and AA11. The at least six amino acid residues may also be AA2, AA4, AA8, AA9, AA10 and AA11. Such at least six amino acid residues may be (a) AA2, AA5, AA6, AA7, AA8 and AA9, (b) AA2, AA5, AA6, AA7, AA8 and AA10, or (c) AA2, AA5, AA6, AA7, AA8 and AA11. The at least six amino acid residues may furthermore also be (a) AA2, AA5, AA6, AA7, AA9 and AA10, or (b) AA2, AA5, AA6, AA7, AA9 and AA11. The at least six amino acid residues may also be AA2, AA5, AA6, AA7, AA10 and AA11. Such at least six amino acid residues may be (a) AA2, AA5, AA6, AA8, AA9 and AA10, or (b) AA2, AA5, AA6, AA8, AA9 and AA11. The at least six amino acid residues may also be AA2, AA5, AA6, AA8, AA10 and AA11. The at least six amino acid residues may also be AA2, AA5, AA6, AA9, AA10 and AA11. Such at least six amino acid residues may be (a) AA2, AA5, AA7, AA8, AA9 and AA10, or (b) AA2, AA5, AA7, AA8, AA9 and AA11. The at least six amino acid residues may also be AA2, AA5, AA7, AA8, AA10 and AA11. The at least six amino acid residues may also be AA2, AA5, AA7, AA9, AA10 and AA11. The at least six amino acid residues may also be AA2, AA5, AA8, AA9, AA10 and AA11. Such at least six amino acid residues may be (a) AA2, AA6, AA7, AA8, AA9 and AA10, or (b) AA2, AA6, AA7, AA8, AA9 and AA11. The at least six amino acid residues may also be AA2, AA6, AA7, AA8, AA10 and AA11. The at least six amino acid residues may also be AA2, AA6, AA7, AA9, AA10 and AA11. The at least six amino acid residues may also be AA2, AA6, AA8, AA9, AA10 and AA11. The at least six amino acid residues may also be AA2, AA7, AA8, AA9, AA10 and AA11.

Such at least six amino acid residues may be (a) AA3, AA4, AA5, AA6, AA7 and AA8, (b) AA3, AA4, AA5, AA6, AA7 and AA9, (c) AA3, AA4, AA5, AA6, AA7 and AA10, or (d) AA3, AA4, AA5, AA6, AA7 and AA11. The at least six amino acid residues may also be (a) AA3, AA4, AA5, AA6, AA8 and AA9, (b) AA3, AA4, AA5, AA6, AA8 and AA10, or (c) AA3, AA4, AA5, AA6, AA8 and AA11. The at least six amino acid residues may furthermore also be (a) AA3, AA4, AA5, AA6, AA9 and AA10, or (b) AA3, AA4, AA5, AA6, AA9 and AA11. The at least six amino acid residues may also be AA3, AA4, AA5, AA6, AA10 and AA11. Such at least six amino acid residues may be (a) AA3, AA4, AA5, AA7, AA8 and AA9, (b) AA3, AA4, AA5, AA7, AA8 and AA10, or (c) AA3, AA4, AA5, AA7, AA8 and AA11. The at least six amino acid residues may furthermore also be (a) AA3, AA4, AA5, AA7, AA9 and AA10, or (b) AA3, AA4, AA5, AA7, AA9 and AA11. The at least six amino acid residues may also be AA3, AA4, AA5, AA7, AA10 and AA11. Such at least six amino acid residues may be (a) AA3, AA4, AA5, AA8, AA9 and AA10, or (b) AA3, AA4, AA5, AA8, AA9 and AA11. The at least six amino acid residues may also be AA3, AA4, AA5, AA8, AA10 and AA11. The at least six amino acid residues may also be AA3, AA4, AA5, AA9, AA10 and AA11. Such at least six amino acid residues may be (a) AA3, AA4, AA6, AA7, AA8 and AA9, (b) AA3, AA4, AA6, AA7, AA8 and AA10, or (c) AA3, AA4, AA6, AA7, AA8 and AA11. The at least six amino acid residues may furthermore also be (a) AA3, AA4, AA6, AA7, AA9 and AA10, or (b) AA3, AA4, AA6, AA7, AA9 and AA11. The at least six amino acid residues may also be AA3, AA4, AA6, AA7, AA10 and AA11. Such at least six amino acid residues may be (a) AA3, AA4, AA6, AA8, AA9 and AA10, or (b) AA3, AA4, AA6, AA8, AA9 and AA11. The at least six amino acid residues may also be AA3, AA4, AA6, AA8, AA10 and AA11. The at least six amino acid residues may also be AA3, AA4, AA6, AA9, AA10 and AA11. Such at least six amino acid residues may be (a) AA3, AA4, AA7, AA8, AA9 and AA10, or (b) AA3, AA4, AA7, AA8, AA9 and AA11. The at least six amino acid residues may also be AA3, AA4, AA7, AA8, AA10 and AA11. The at least six amino acid residues may also be AA3, AA4, AA7, AA9, AA10 and AA11. The at least six amino acid residues may also be AA3, AA4, AA8, AA9, AA10 and AA11. Such at least six amino acid residues may be (a) AA3, AA5, AA6, AA7, AA8 and AA9, (b) AA3, AA5, AA6, AA7, AA8 and AA10, or (c) AA3, AA5, AA6, AA7, AA8 and AA11. The at least six amino acid residues may furthermore also be (a) AA3, AA5, AA6, AA7, AA9 and AA10, or (b) AA3, AA5, AA6, AA7, AA9 and AA11. The at least six amino acid residues may also be AA3, AA5, AA6, AA7, AA10 and AA11. Such at least six amino acid residues may be (a) AA3, AA5, AA6, AA8, AA9 and AA10, or (b) AA3, AA5, AA6, AA8, AA9 and AA11. The at least six amino acid residues may also be AA3, AA5, AA6, AA8, AA10 and AA11. The at least six amino acid residues may also be AA3, AA5, AA6, AA9, AA10 and AA11. Such at least six amino acid residues may be (a) AA3, AA5, AA7, AA8, AA9 and AA10, or (b) AA3, AA5, AA7, AA8, AA9 and AA11. The at least six amino acid residues may also be AA3, AA5, AA7, AA8, AA10 and AA11. The at least six amino acid residues may also be AA3, AA5, AA7, AA9, AA10 and AA11. The at least six amino acid residues may also be AA3, AA5, AA8, AA9, AA10 and AA11. Such at least six amino acid residues may be (a) AA3, AA6, AA7, AA8, AA9 and AA10, or (b) AA3, AA6, AA7, AA8, AA9 and AA11. The at least six amino acid residues may also be AA3, AA6, AA7, AA8, AA10 and AA11. The at least six amino acid residues may also be AA3, AA6, AA7, AA9, AA10 and AA11. The at least six amino acid residues may also be AA3, AA6, AA8, AA9, AA10 and AA11. The at least six amino acid residues may also be AA3, AA7, AA8, AA9, AA10 and AA11.

Such at least six amino acid residues may be (a) AA4, AA5, AA6, AA7, AA8 and AA9, (b) AA4, AA5, AA6, AA7, AA8 and AA10, or (c) AA4, AA5, AA6, AA7, AA8 and AA11. The at least six amino acid residues may furthermore also be (a) AA4, AA5, AA6, AA7, AA9 and AA10, or (b) AA4, AA5, AA6, AA7, AA9 and AA11. The at least six amino acid residues may also be AA4, AA5, AA6, AA7, AA10 and AA11. Such at least six amino acid residues may be (a) AA4, AA5, AA6, AA8, AA9 and AA10, or (b) AA4, AA5, AA6, AA8, AA9 and AA11. The at least six amino acid residues may also be AA4, AA5, AA6, AA8, AA10 and AA11. The at least six amino acid residues may also be AA4, AA5, AA6, AA9, AA10 and AA11. Such at least six amino acid residues may be (a) AA4, AA5, AA7, AA8, AA9 and AA10, or (b) AA4, AA5, AA7, AA8, AA9 and AA11. The at least six amino acid residues may also be AA4, AA5, AA7, AA8, AA10 and AA11. The at least six amino acid residues may also be AA4, AA5, AA7, AA9, AA10 and AA11. The at least six amino acid residues may also be AA4, AA5, AA8, AA9, AA10 and AA11. Such at least six amino acid residues may be (a) AA4, AA6, AA7, AA8, AA9 and AA10, or (b) AA4, AA6, AA7, AA8, AA9 and AA11. The at least six amino acid residues may also be AA4, AA6, AA7, AA8, AA10 and AA11. The at least six amino acid residues may also be AA4, AA6, AA7, AA9, AA10 and AA11. The at least six amino acid residues may also be AA4, AA6, AA8, AA9, AA10 and AA11. The at least six amino acid residues may also be AA4, AA7, AA8, AA9, AA10 and AA11. Such at least six amino acid residues may be (a) AA5, AA6, AA7, AA8, AA9 and AA10, or (b) AA5, AA6, AA7, AA8, AA9 and AA11. The at least six amino acid residues may also be AA5, AA6, AA7, AA8, AA10 and AA11. The at least six amino acid residues may also be AA5, AA6, AA7, AA9, AA10 and AA11. The at least six amino acid residues may also be AA5, AA6, AA8, AA9, AA10 and AA11. The at least six amino acid residues may also be AA5, AA7, AA8, AA9, AA10 and AA11. The at least six amino acid residues may also be AA6, AA7, AA8, AA9, AA10 and AA11.

Such at least seven amino acid residues may be (a) AA1, AA2, AA3, AA4, AA5, AA6 and AA7, (b) AA1, AA2, AA3, AA4, AA5, AA6 and AA8, (c) AA1, AA2, AA3, AA4, AA5, AA6 and AA9, (d) AA1, AA2, AA3, AA4, AA5, AA6 and AA10, or (e) AA1, AA2, AA3, AA4, AA5, AA6 and AA11. As yet another alternative the at least seven amino acid residues may be (a) AA1, AA2, AA3, AA4, AA5, AA7 and AA8, (b) AA1, AA2, AA3, AA4, AA5, AA7 and AA9, (c) AA1, AA2, AA3, AA4, AA5, AA7 and AA10, or (d) AA1, AA2, AA3, AA4, AA5, AA7 and AA11. The at least seven amino acid residues may also be (a) AA1, AA2, AA3, AA4, AA5, AA8 and AA9, (b) AA1, AA2, AA3, AA4, AA5, AA8 and AA10, or (c) AA1, AA2, AA3, AA4, AA5, AA8 and AA11. The at least seven amino acid residues may furthermore also be (a) AA1, AA2, AA3, AA4, AA5, AA9 and AA10, or (b) AA1, AA2, AA3, AA4, AA5, AA9 and AA11. The at least seven amino acid residues may also be AA1, AA2, AA3, AA4, AA5, AA10 and AA11. Such at least seven amino acid residues may be (a) AA1, AA2, AA3, AA4, AA6, AA7 and AA8, (b) AA1, AA2, AA3, AA4, AA6, AA7 and AA9, (c) AA1, AA2, AA3, AA4, AA6, AA7 and AA10, or (d) AA1, AA2, AA3, AA4, AA6, AA7 and AA11. The at least seven amino acid residues may also be (a) AA1, AA2, AA3, AA4, AA6, AA8 and AA9, (b) AA1, AA2, AA3, AA4, AA6, AA8 and AA10, or (c) AA1, AA2, AA3, AA4, AA6, AA8 and AA11. The at least seven amino acid residues may furthermore also be (a) AA1, AA2, AA3, AA4, AA6, AA9 and AA10, or (b) AA1, AA2, AA3, AA4, AA6, AA9 and AA11. The at least seven amino acid residues may also be AA1, AA2, AA3, AA4, AA6, AA10 and AA11. Such at least seven amino acid residues may be (a) AA1, AA2, AA3, AA4, AA7, AA8 and AA9, (b) AA1, AA2, AA3, AA4, AA7, AA8 and AA10, or (c) AA1, AA2, AA3, AA4, AA7, AA8 and AA11. The at least seven amino acid residues may furthermore also be (a) AA1, AA2, AA3, AA4, AA7, AA9 and AA10, or (b) AA1, AA2, AA3, AA4, AA7, AA9 and AA11. The at least seven amino acid residues may also be AA1, AA2, AA3, AA4, AA7, AA10 and AA11. Such at least seven amino acid residues may be (a) AA1, AA2, AA3, AA4, AA8, AA9 and AA10, or (b) AA1, AA2, AA3, AA4, AA8, AA9 and AA11. The at least seven amino acid residues may also be AA1, AA2, AA3, AA4, AA8, AA10 and AA11. The at least seven amino acid residues may also be AA1, AA2, AA3, AA4, AA9, AA10 and AA11. Such at least seven amino acid residues may be (a) AA1, AA2, AA3, AA5, AA6, AA7 and AA8, (b) AA1, AA2, AA3, AA5, AA6, AA7 and AA9, (c) AA1, AA2, AA3, AA5, AA6, AA7 and AA10, or (d) AA1, AA2, AA3, AA5, AA6, AA7 and AA11. The at least seven amino acid residues may also be (a) AA1, AA2, AA3, AA5, AA6, AA8 and AA9, (b) AA1, AA2, AA3, AA5, AA6, AA8 and AA10, or (c) AA1, AA2, AA3, AA5, AA6, AA8 and AA11. The at least seven amino acid residues may furthermore also be (a) AA1, AA2, AA3, AA5, AA6, AA9 and AA10, or (b) AA1, AA2, AA3, AA5, AA6, AA9 and AA11. The at least seven amino acid residues may also be AA1, AA2, AA3, AA5, AA6, AA10 and AA11. Such at least seven amino acid residues may be (a) AA1, AA2, AA3, AA5, AA7, AA8 and AA9, (b) AA1, AA2, AA3, AA5, AA7, AA8 and AA10, or (c) AA1, AA2, AA3, AA5, AA7, AA8 and AA11. The at least seven amino acid residues may furthermore also be (a) AA1, AA2, AA3, AA5, AA7, AA9 and AA10, or (b) AA1, AA2, AA3, AA5, AA7, AA9 and AA11. The at least seven amino acid residues may also be AA1, AA2, AA3, AA5, AA7, AA10 and AA11. Such at least seven amino acid residues may be (a) AA1, AA2, AA3, AA5, AA8, AA9 and AA10, or (b) AA1, AA2, AA3, AA5, AA8, AA9 and AA11. The at least seven amino acid residues may also be AA1, AA2, AA3, AA5, AA8, AA10 and AA11. The at least seven amino acid residues may also be AA1, AA2, AA3, AA5, AA9, AA10 and AA11. Such at least seven amino acid residues may be (a) AA1, AA2, AA3, AA6, AA7, AA8 and AA9, (b) AA1, AA2, AA3, AA6, AA7, AA8 and AA10, or (c) AA1, AA2, AA3, AA6, AA7, AA8 and AA11. The at least seven amino acid residues may furthermore also be (a) AA1, AA2, AA3, AA6, AA7, AA9 and AA10, or (b) AA1, AA2, AA3, AA6, AA7, AA9 and AA11. The at least seven amino acid residues may also be AA1, AA2, AA3, AA6, AA7, AA10 and AA11. Such at least seven amino acid residues may be (a) AA1, AA2, AA3, AA6, AA8, AA9 and AA10, or (b) AA1, AA2, AA3, AA6, AA8, AA9 and AA11. The at least seven amino acid residues may also be AA1, AA2, AA3, AA6, AA8, AA10 and AA11. The at least seven amino acid residues may also be AA1, AA2, AA3, AA6, AA9, AA10 and AA11. Such at least seven amino acid residues may be (a) AA1, AA2, AA3, AA7, AA8, AA9 and AA10, or (b) AA1, AA2, AA3, AA7, AA8, AA9 and AA11. The at least seven amino acid residues may also be AA1, AA2, AA3, AA7, AA8, AA10 and AA11. The at least seven amino acid residues may also be AA1, AA2, AA3, AA7, AA9, AA10 and AA11. The at least seven amino acid residues may also be AA1, AA2, AA3, AA8, AA9, AA10 and AA11. Such at least seven amino acid residues may be (a) AA1, AA2, AA4, AA5, AA6, AA7 and AA8, (b) AA1, AA2, AA4, AA5, AA6, AA7 and AA9, (c) AA1, AA2, AA4, AA5, AA6, AA7 and AA10, or (d) AA1, AA2, AA4, AA5, AA6, AA7 and AA11. The at least seven amino acid residues may also be (a) AA1, AA2, AA4, AA5, AA6, AA8 and AA9, (b) AA1, AA2, AA4, AA5, AA6, AA8 and AA10, or (c) AA1, AA2, AA4, AA5, AA6, AA8 and AA11. The at least seven amino acid residues may furthermore also be (a) AA1, AA2, AA4, AA5, AA6, AA9 and AA10, or (b) AA1, AA2, AA4, AA5, AA6, AA9 and AA11. The at least seven amino acid residues may also be AA1, AA2, AA4, AA5, AA6, AA10 and AA11. Such at least seven amino acid residues may be (a) AA1, AA2, AA4, AA5, AA7, AA8 and AA9, (b) AA1, AA2, AA4, AA5, AA7, AA8 and AA10, or (c) AA1, AA2, AA4, AA5, AA7, AA8 and AA11. The at least seven amino acid residues may furthermore also be (a) AA1, AA2, AA4, AA5, AA7, AA9 and AA10, or (b) AA1, AA2, AA4, AA5, AA7, AA9 and AA11. The at least seven amino acid residues may also be AA1, AA2, AA4, AA5, AA7, AA10 and AA11. Such at least seven amino acid residues may be (a) AA1, AA2, AA4, AA5, AA8, AA9 and AA10, or (b) AA1, AA2, AA4, AA5, AA8, AA9 and AA11. The at least seven amino acid residues may also be AA1, AA2, AA4, AA5, AA8, AA10 and AA11. The at least seven amino acid residues may also be AA1, AA2, AA4, AA5, AA9, AA10 and AA11. Such at least seven amino acid residues may be (a) AA1, AA2, AA4, AA6, AA7, AA8 and AA9, (b) AA1, AA2, AA4, AA6, AA7, AA8 and AA10, or (c) AA1, AA2, AA4, AA6, AA7, AA8 and AA11. The at least seven amino acid residues may furthermore also be (a) AA1, AA2, AA4, AA6, AA7, AA9 and AA10, or (b) AA1, AA2, AA4, AA6, AA7, AA9 and AA11. The at least seven amino acid residues may also be AA1, AA2, AA4, AA6, AA7, AA10 and AA11. Such at least seven amino acid residues may be (a) AA1, AA2, AA4, AA6, AA8, AA9 and AA10, or (b) AA1, AA2, AA4, AA6, AA8, AA9 and AA11. The at least seven amino acid residues may also be AA1, AA2, AA4, AA6, AA8, AA10 and AA11. The at least seven amino acid residues may also be AA1, AA2, AA4, AA6, AA9, AA10 and AA11. Such at least seven amino acid residues may be (a) AA1, AA2, AA4, AA7, AA8, AA9 and AA10, or (b) AA1, AA2, AA4, AA7, AA8, AA9 and AA11. The at least seven amino acid residues may also be AA1, AA2, AA4, AA7, AA8, AA10 and AA11. The at least seven amino acid residues may also be AA1, AA2, AA4, AA7, AA9, AA10 and AA11. The at least seven amino acid residues may also be AA1, AA2, AA4, AA8, AA9, AA10 and AA11. Such at least seven amino acid residues may be (a) AA1, AA2, AA5, AA6, AA7, AA8 and AA9, (b) AA1, AA2, AA5, AA6, AA7, AA8 and AA10, or (c) AA1, AA2, AA5, AA6, AA7, AA8 and AA11. The at least seven amino acid residues may furthermore also be (a) AA1, AA2, AA5, AA6, AA7, AA9 and AA10, or (b) AA1, AA2, AA5, AA6, AA7, AA9 and AA11. The at least seven amino acid residues may also be AA1, AA2, AA5, AA6, AA7, AA10 and AA11. Such at least seven amino acid residues may be (a) AA1, AA2, AA5, AA6, AA8, AA9 and AA10, or (b) AA1, AA2, AA5, AA6, AA8, AA9 and AA11. The at least seven amino acid residues may also be AA1, AA2, AA5, AA6, AA8, AA10 and AA11. The at least seven amino acid residues may also be AA1, AA2, AA5, AA6, AA9, AA10 and AA11. Such at least seven amino acid residues may be (a) AA1, AA2, AA5, AA7, AA8, AA9 and AA10, or (b) AA1, AA2, AA5, AA7, AA8, AA9 and AA11. The at least seven amino acid residues may also be AA1, AA2, AA5, AA7, AA8, AA10 and AA11. The at least seven amino acid residues may also be AA1, AA2, AA5, AA7, AA9, AA10 and AA11. The at least seven amino acid residues may also be AA1, AA2, AA5, AA8, AA9, AA10 and AA11. Such at least seven amino acid residues may be (a) AA1, AA2, AA6, AA7, AA8, AA9 and AA10, or (b) AA1, AA2, AA6, AA7, AA8, AA9 and AA11. The at least seven amino acid residues may also be AA1, AA2, AA6, AA7, AA8, AA10 and AA11. The at least seven amino acid residues may also be AA1, AA2, AA6, AA7, AA9, AA10 and AA11. The at least seven amino acid residues may also be AA1, AA2, AA6, AA8, AA9, AA10 and AA11. The at least seven amino acid residues may also be AA1, AA2, AA7, AA8, AA9, AA10 and AA11. Such at least seven amino acid residues may be (a) AA1, AA3, AA4, AA5, AA6, AA7 and AA8, (b) AA1, AA3, AA4, AA5, AA6, AA7 and AA9, (c) AA1, AA3, AA4, AA5, AA6, AA7 and AA10, or (d) AA1, AA3, AA4, AA5, AA6, AA7 and AA11. The at least seven amino acid residues may also be (a) AA1, AA3, AA4, AA5, AA6, AA8 and AA9, (b) AA1, AA3, AA4, AA5, AA6, AA8 and AA10, or (c) AA1, AA3, AA4, AA5, AA6, AA8 and AA11. The at least seven amino acid residues may furthermore also be (a) AA1, AA3, AA4, AA5, AA6, AA9 and AA10, or (b) AA1, AA3, AA4, AA5, AA6, AA9 and AA11. The at least seven amino acid residues may also be AA1, AA3, AA4, AA5, AA6, AA10 and AA11. Such at least seven amino acid residues may be (a) AA1, AA3, AA4, AA5, AA7, AA8 and AA9, (b) AA1, AA3, AA4, AA5, AA7, AA8 and AA10, or (c) AA1, AA3, AA4, AA5, AA7, AA8 and AA11. The at least seven amino acid residues may furthermore also be (a) AA1, AA3, AA4, AA5, AA7, AA9 and AA10, or (b) AA1, AA3, AA4, AA5, AA7, AA9 and AA11. The at least seven amino acid residues may also be AA1, AA3, AA4, AA5, AA7, AA10 and AA11. Such at least seven amino acid residues may be (a) AA1, AA3, AA4, AA5, AA8, AA9 and AA10, or (b) AA1, AA3, AA4, AA5, AA8, AA9 and AA11. The at least seven amino acid residues may also be AA1, AA3, AA4, AA5, AA8, AA10 and AA11. The at least seven amino acid residues may also be AA1, AA3, AA4, AA5, AA9, AA10 and AA11. Such at least seven amino acid residues may be (a) AA1, AA3, AA4, AA6, AA7, AA8 and AA9, (b) AA1, AA3, AA4, AA6, AA7, AA8 and AA10, or (c) AA1, AA3, AA4, AA6, AA7, AA8 and AA11. The at least seven amino acid residues may furthermore also be (a) AA1, AA3, AA4, AA6, AA7, AA9 and AA10, or (b) AA1, AA3, AA4, AA6, AA7, AA9 and AA11. The at least seven amino acid residues may also be AA1, AA3, AA4, AA6, AA7, AA10 and AA11. Such at least seven amino acid residues may be (a) AA1, AA3, AA4, AA6, AA8, AA9 and AA10, or (b) AA1, AA3, AA4, AA6, AA8, AA9 and AA11. The at least seven amino acid residues may also be AA1, AA3, AA4, AA6, AA8, AA10 and AA11. The at least seven amino acid residues may also be AA1, AA3, AA4, AA6, AA9, AA10 and AA11. Such at least seven amino acid residues may be (a) AA1, AA3, AA4, AA7, AA8, AA9 and AA10, or (b) AA1, AA3, AA4, AA7, AA8, AA9 and AA11. The at least seven amino acid residues may also be AA1, AA3, AA4, AA7, AA8, AA10 and AA11. The at least seven amino acid residues may also be AA1, AA3, AA4, AA7, AA9, AA10 and AA11. The at least seven amino acid residues may also be AA1, AA3, AA4, AA8, AA9, AA10 and AA11. Such at least seven amino acid residues may be (a) AA1, AA3, AA5, AA6, AA7, AA8 and AA9, (b) AA1, AA3, AA5, AA6, AA7, AA8 and AA10, or (c) AA1, AA3, AA5, AA6, AA7, AA8 and AA11. The at least seven amino acid residues may furthermore also be (a) AA1, AA3, AA5, AA6, AA7, AA9 and AA10, or (b) AA1, AA3, AA5, AA6, AA7, AA9 and AA11. The at least seven amino acid residues may also be AA1, AA3, AA5, AA6, AA7, AA10 and AA11. Such at least seven amino acid residues may be (a) AA1, AA3, AA5, AA6, AA8, AA9 and AA10, or (b) AA1, AA3, AA5, AA6, AA8, AA9 and AA11. The at least seven amino acid residues may also be AA1, AA3, AA5, AA6, AA8, AA10 and AA11. The at least seven amino acid residues may also be AA1, AA3, AA5, AA6, AA9, AA10 and AA11. Such at least seven amino acid residues may be (a) AA1, AA3, AA5, AA7, AA8, AA9 and AA10, or (b) AA1, AA3, AA5, AA7, AA8, AA9 and AA11. The at least seven amino acid residues may also be AA1, AA3, AA5, AA7, AA8, AA10 and AA11. The at least seven amino acid residues may also be AA1, AA3, AA5, AA7, AA9, AA10 and AA11. The at least seven amino acid residues may also be AA1, AA3, AA5, AA8, AA9, AA10 and AA11. Such at least seven amino acid residues may be (a) AA1, AA3, AA6, AA7, AA8, AA9 and AA10, or (b) AA1, AA3, AA6, AA7, AA8, AA9 and AA11. The at least seven amino acid residues may also be AA1, AA3, AA6, AA7, AA8, AA10 and AA11. The at least seven amino acid residues may also be AA1, AA3, AA6, AA7, AA9, AA10 and AA11. The at least seven amino acid residues may also be AA1, AA3, AA6, AA8, AA9, AA10 and AA11. The at least seven amino acid residues may also be AA1, AA3, AA7, AA8, AA9, AA10 and AA11. Such at least seven amino acid residues may be (a) AA1, AA4, AA5, AA6, AA7, AA8 and AA9, (b) AA1, AA4, AA5, AA6, AA7, AA8 and AA10, or (c) AA1, AA4, AA5, AA6, AA7, AA8 and AA11. The at least seven amino acid residues may furthermore also be (a) AA1, AA4, AA5, AA6, AA7, AA9 and AA10, or (b) AA1, AA4, AA5, AA6, AA7, AA9 and AA11. The at least seven amino acid residues may also be AA1, AA4, AA5, AA6, AA7, AA10 and AA11. Such at least seven amino acid residues may be (a) AA1, AA4, AA5, AA6, AA8, AA9 and AA10, or (b) AA1, AA4, AA5, AA6, AA8, AA9 and AA11. The at least seven amino acid residues may also be AA1, AA4, AA5, AA6, AA8, AA10 and AA11. The at least seven amino acid residues may also be AA1, AA4, AA5, AA6, AA9, AA10 and AA11. Such at least seven amino acid residues may be (a) AA1, AA4, AA5, AA7, AA8, AA9 and AA10, or (b) AA1, AA4, AA5, AA7, AA8, AA9 and AA11. The at least seven amino acid residues may also be AA1, AA4, AA5, AA7, AA8, AA10 and AA11. The at least seven amino acid residues may also be AA1, AA4, AA5, AA7, AA9, AA10 and AA11. The at least seven amino acid residues may also be AA1, AA4, AA5, AA8, AA9, AA10 and AA11. Such at least seven amino acid residues may be (a) AA1, AA4, AA6, AA7, AA8, AA9 and AA10, or (b) AA1, AA4, AA6, AA7, AA8, AA9 and AA11. The at least seven amino acid residues may also be AA1, AA4, AA6, AA7, AA8, AA10 and AA11. The at least seven amino acid residues may also be AA1, AA4, AA6, AA7, AA9, AA10 and AA11. The at least seven amino acid residues may also be AA1, AA4, AA6, AA8, AA9, AA10 and AA11. The at least seven amino acid residues may also be AA1, AA4, AA7, AA8, AA9, AA10 and AA11. Such at least seven amino acid residues may be (a) AA1, AA5, AA6, AA7, AA8, AA9 and AA10, or (b) AA1, AA5, AA6, AA7, AA8, AA9 and AA11. The at least seven amino acid residues may also be AA1, AA5, AA6, AA7, AA8, AA10 and AA11. The at least seven amino acid residues may also be AA1, AA5, AA6, AA7, AA9, AA10 and AA11. The at least seven amino acid residues may also be AA1, AA5, AA6, AA8, AA9, AA10 and AA11. The at least seven amino acid residues may also be AA1, AA5, AA7, AA8, AA9, AA10 and AA11. The at least seven amino acid residues may also be AA1, AA6, AA7, AA8, AA9, AA10 and AA11.

Such at least seven amino acid residues may be (a) AA2, AA3, AA4, AA5, AA6, AA7 and AA8, (b) AA2, AA3, AA4, AA5, AA6, AA7 and AA9, (c) AA2, AA3, AA4, AA5, AA6, AA7 and AA10, or (d) AA2, AA3, AA4, AA5, AA6, AA7 and AA11. The at least seven amino acid residues may also be (a) AA2, AA3, AA4, AA5, AA6, AA8 and AA9, (b) AA2, AA3, AA4, AA5, AA6, AA8 and AA10, or (c) AA2, AA3, AA4, AA5, AA6, AA8 and AA11. The at least seven amino acid residues may furthermore also be (a) AA2, AA3, AA4, AA5, AA6, AA9 and AA10, or (b) AA2, AA3, AA4, AA5, AA6, AA9 and AA11. The at least seven amino acid residues may also be AA2, AA3, AA4, AA5, AA6, AA10 and AA11. Such at least seven amino acid residues may be (a) AA2, AA3, AA4, AA5, AA7, AA8 and AA9, (b) AA2, AA3, AA4, AA5, AA7, AA8 and AA10, or (c) AA2, AA3, AA4, AA5, AA7, AA8 and AA11. The at least seven amino acid residues may furthermore also be (a) AA2, AA3, AA4, AA5, AA7, AA9 and AA10, or (b) AA2, AA3, AA4, AA5, AA7, AA9 and AA11. The at least seven amino acid residues may also be AA2, AA3, AA4, AA5, AA7, AA10 and AA11. Such at least seven amino acid residues may be (a) AA2, AA3, AA4, AA5, AA8, AA9 and AA10, or (b) AA2, AA3, AA4, AA5, AA8, AA9 and AA11. The at least seven amino acid residues may also be AA2, AA3, AA4, AA5, AA8, AA10 and AA11. The at least seven amino acid residues may also be AA2, AA3, AA4, AA5, AA9, AA10 and AA11. Such at least seven amino acid residues may be (a) AA2, AA3, AA4, AA6, AA7, AA8 and AA9, (b) AA2, AA3, AA4, AA6, AA7, AA8 and AA10, or (c) AA2, AA3, AA4, AA6, AA7, AA8 and AA11. The at least seven amino acid residues may furthermore also be (a) AA2, AA3, AA4, AA6, AA7, AA9 and AA10, or (b) AA2, AA3, AA4, AA6, AA7, AA9 and AA11. The at least seven amino acid residues may also be AA2, AA3, AA4, AA6, AA7, AA10 and AA11. Such at least seven amino acid residues may be (a) AA2, AA3, AA4, AA6, AA8, AA9 and AA10, or (b) AA2, AA3, AA4, AA6, AA8, AA9 and AA11. The at least seven amino acid residues may also be AA2, AA3, AA4, AA6, AA8, AA10 and AA11. The at least seven amino acid residues may also be AA2, AA3, AA4, AA6, AA9, AA10 and AA11. Such at least seven amino acid residues may be (a) AA2, AA3, AA4, AA7, AA8, AA9 and AA10, or (b) AA2, AA3, AA4, AA7, AA8, AA9 and AA11. The at least seven amino acid residues may also be AA2, AA3, AA4, AA7, AA8, AA10 and AA11. The at least seven amino acid residues may also be AA2, AA3, AA4, AA7, AA9, AA10 and AA11. The at least seven amino acid residues may also be AA2, AA3, AA4, AA8, AA9, AA10 and AA11. Such at least seven amino acid residues may be (a) AA2, AA3, AA5, AA6, AA7, AA8 and AA9, (b) AA2, AA3, AA5, AA6, AA7, AA8 and AA10, or (c) AA2, AA3, AA5, AA6, AA7, AA8 and AA11. The at least seven amino acid residues may furthermore also be (a) AA2, AA3, AA5, AA6, AA7, AA9 and AA10, or (b) AA2, AA3, AA5, AA6, AA7, AA9 and AA11. The at least seven amino acid residues may also be AA2, AA3, AA5, AA6, AA7, AA10 and AA11. Such at least seven amino acid residues may be (a) AA2, AA3, AA5, AA6, AA8, AA9 and AA10, or (b) AA2, AA3, AA5, AA6, AA8, AA9 and AA11. The at least seven amino acid residues may also be AA2, AA3, AA5, AA6, AA8, AA10 and AA11. The at least seven amino acid residues may also be AA2, AA3, AA5, AA6, AA9, AA10 and AA11. Such at least seven amino acid residues may be (a) AA2, AA3, AA5, AA7, AA8, AA9 and AA10, or (b) AA2, AA3, AA5, AA7, AA8, AA9 and AA11. The at least seven amino acid residues may also be AA2, AA3, AA5, AA7, AA8, AA10 and AA11. The at least seven amino acid residues may also be AA2, AA3, AA5, AA7, AA9, AA10 and AA11. The at least seven amino acid residues may also be AA2, AA3, AA5, AA8, AA9, AA10 and AA11. Such at least seven amino acid residues may be (a) AA2, AA3, AA6, AA7, AA8, AA9 and AA10, or (b) AA2, AA3, AA6, AA7, AA8, AA9 and AA11. The at least seven amino acid residues may also be AA2, AA3, AA6, AA7, AA8, AA10 and AA11. The at least seven amino acid residues may also be AA2, AA3, AA6, AA7, AA9, AA10 and AA11. The at least seven amino acid residues may also be AA2, AA3, AA6, AA8, AA9, AA10 and AA11. The at least seven amino acid residues may also be AA2, AA3, AA7, AA8, AA9, AA10 and AA11. Such at least seven amino acid residues may be (a) AA2, AA4, AA5, AA6, AA7, AA8 and AA9, (b) AA2, AA4, AA5, AA6, AA7, AA8 and AA10, or (c) AA2, AA4, AA5, AA6, AA7, AA8 and AA11. The at least seven amino acid residues may furthermore also be (a) AA2, AA4, AA5, AA6, AA7, AA9 and AA10, or (b) AA2, AA4, AA5, AA6, AA7, AA9 and AA11. The at least seven amino acid residues may also be AA2, AA4, AA5, AA6, AA7, AA10 and AA11. Such at least seven amino acid residues may be (a) AA2, AA4, AA5, AA6, AA8, AA9 and AA10, or (b) AA2, AA4, AA5, AA6, AA8, AA9 and AA11. The at least seven amino acid residues may also be AA2, AA4, AA5, AA6, AA8, AA10 and AA11. The at least seven amino acid residues may also be AA2, AA4, AA5, AA6, AA9, AA10 and AA11. Such at least seven amino acid residues may be (a) AA2, AA4, AA5, AA7, AA8, AA9 and AA10, or (b) AA2, AA4, AA5, AA7, AA8, AA9 and AA11. The at least seven amino acid residues may also be AA2, AA4, AA5, AA7, AA8, AA10 and AA11. The at least seven amino acid residues may also be AA2, AA4, AA5, AA7, AA9, AA10 and AA11. The at least seven amino acid residues may also be AA2, AA4, AA5, AA8, AA9, AA10 and AA11. Such at least seven amino acid residues may be (a) AA2, AA4, AA6, AA7, AA8, AA9 and AA10, or (b) AA2, AA4, AA6, AA7, AA8, AA9 and AA11. The at least seven amino acid residues may also be AA2, AA4, AA6, AA7, AA8, AA10 and AA11. The at least seven amino acid residues may also be AA2, AA4, AA6, AA7, AA9, AA10 and AA11. The at least seven amino acid residues may also be AA2, AA4, AA6, AA8, AA9, AA10 and AA11. The at least seven amino acid residues may also be AA2, AA4, AA7, AA8, AA9, AA10 and AA11. Such at least seven amino acid residues may be (a) AA2, AA5, AA6, AA7, AA8, AA9 and AA10, or (b) AA2, AA5, AA6, AA7, AA8, AA9 and AA11. The at least seven amino acid residues may also be AA2, AA5, AA6, AA7, AA8, AA10 and AA11. The at least seven amino acid residues may also be AA2, AA5, AA6, AA7, AA9, AA10 and AA11. The at least seven amino acid residues may also be AA2, AA5, AA6, AA8, AA9, AA10 and AA11. The at least seven amino acid residues may also be AA2, AA5, AA7, AA8, AA9, AA10 and AA11. The at least seven amino acid residues may also be AA2, AA6, AA7, AA8, AA9, AA10 and AA11.

Such at least seven amino acid residues may be (a) AA3, AA4, AA5, AA6, AA7, AA8 and AA9, (b) AA3, AA4, AA5, AA6, AA7, AA8 and AA10, or (c) AA3, AA4, AA5, AA6, AA7, AA8 and AA11. The at least seven amino acid residues may furthermore also be (a) AA3, AA4, AA5, AA6, AA7, AA9 and AA10, or (b) AA3, AA4, AA5, AA6, AA7, AA9 and AA11. The at least seven amino acid residues may also be AA3, AA4, AA5, AA6, AA7, AA10 and AA11. Such at least seven amino acid residues may be (a) AA3, AA4, AA5, AA6, AA8, AA9 and AA10, or (b) AA3, AA4, AA5, AA6, AA8, AA9 and AA11. The at least seven amino acid residues may also be AA3, AA4, AA5, AA6, AA8, AA10 and AA11. The at least seven amino acid residues may also be AA3, AA4, AA5, AA6, AA9, AA10 and AA11. Such at least seven amino acid residues may be (a) AA3, AA4, AA5, AA7, AA8, AA9 and AA10, or (b) AA3, AA4, AA5, AA7, AA8, AA9 and AA11. The at least seven amino acid residues may also be AA3, AA4, AA5, AA7, AA8, AA10 and AA11. The at least seven amino acid residues may also be AA3, AA4, AA5, AA7, AA9, AA10 and AA11. The at least seven amino acid residues may also be AA3, AA4, AA5, AA8, AA9, AA10 and AA11. Such at least seven amino acid residues may be (a) AA3, AA4, AA6, AA7, AA8, AA9 and AA10, or (b) AA3, AA4, AA6, AA7, AA8, AA9 and AA11. The at least seven amino acid residues may also be AA3, AA4, AA6, AA7, AA8, AA10 and AA11. The at least seven amino acid residues may also be AA3, AA4, AA6, AA7, AA9, AA10 and AA11. The at least seven amino acid residues may also be AA3, AA4, AA6, AA8, AA9, AA10 and AA11. The at least seven amino acid residues may also be AA3, AA4, AA7, AA8, AA9, AA10 and AA11. Such at least seven amino acid residues may be (a) AA3, AA5, AA6, AA7, AA8, AA9 and AA10, or (b) AA3, AA5, AA6, AA7, AA8, AA9 and AA11. The at least seven amino acid residues may also be AA3, AA5, AA6, AA7, AA8, AA10 and AA11. The at least seven amino acid residues may also be AA3, AA5, AA6, AA7, AA9, AA10 and AA11. The at least seven amino acid residues may also be AA3, AA5, AA6, AA8, AA9, AA10 and AA11. The at least seven amino acid residues may also be AA3, AA5, AA7, AA8, AA9, AA10 and AA11. The at least seven amino acid residues may also be AA3, AA6, AA7, AA8, AA9, AA10 and AA11. Such at least seven amino acid residues may be (a) AA4, AA5, AA6, AA7, AA8, AA9 and AA10, or (b) AA4, AA5, AA6, AA7, AA8, AA9 and AA11. The at least seven amino acid residues may also be AA4, AA5, AA6, AA7, AA8, AA10 and AA11. The at least seven amino acid residues may also be AA4, AA5, AA6, AA7, AA9, AA10 and AA11. The at least seven amino acid residues may also be AA4, AA5, AA6, AA8, AA9, AA10 and AA11. The at least seven amino acid residues may also be AA4, AA5, AA7, AA8, AA9, AA10 and AA11. The at least seven amino acid residues may also be AA4, AA6, AA7, AA8, AA9, AA10 and AA11. The at least seven amino acid residues may also be AA5, AA6, AA7, AA8, AA9, AA10 and AA11.

Such at least eight amino acid residues may be (a) AA1, AA2, AA3, AA4, AA5, AA6, AA7 and AA8, (b) AA1, AA2, AA3, AA4, AA5, AA6, AA7 and AA9, (c) AA1, AA2, AA3, AA4, AA5, AA6, AA7 and AA10, or (d) AA1, AA2, AA3, AA4, AA5, AA6, AA7 and AA11. The at least eight amino acid residues may also be (a) AA1, AA2, AA3, AA4, AA5, AA6, AA8 and AA9, (b) AA1, AA2, AA3, AA4, AA5, AA6, AA8 and AA10, or (c) AA1, AA2, AA3, AA4, AA5, AA6, AA8 and AA11. The at least eight amino acid residues may furthermore also be (a) AA1, AA2, AA3, AA4, AA5, AA6, AA9 and AA10, or (b) AA1, AA2, AA3, AA4, AA5, AA6, AA9 and AA11. The at least eight amino acid residues may also be AA1, AA2, AA3, AA4, AA5, AA6, AA10 and AA11. Such at least eight amino acid residues may be (a) AA1, AA2, AA3, AA4, AA5, AA7, AA8 and AA9, (b) AA1, AA2, AA3, AA4, AA5, AA7, AA8 and AA10, or (c) AA1, AA2, AA3, AA4, AA5, AA7, AA8 and AA11. The at least eight amino acid residues may furthermore also be (a) AA1, AA2, AA3, AA4, AA5, AA7, AA9 and AA10, or (b) AA1, AA2, AA3, AA4, AA5, AA7, AA9 and AA11. The at least eight amino acid residues may also be AA1, AA2, AA3, AA4, AA5, AA7, AA10 and AA11. Such at least eight amino acid residues may be (a) AA1, AA2, AA3, AA4, AA5, AA8, AA9 and AA10, or (b) AA1, AA2, AA3, AA4, AA5, AA8, AA9 and AA11. The at least eight amino acid residues may also be AA1, AA2, AA3, AA4, AA5, AA8, AA10 and AA11. The at least eight amino acid residues may also be AA1, AA2, AA3, AA4, AA5, AA9, AA10 and AA11. Such at least eight amino acid residues may be (a) AA1, AA2, AA3, AA4, AA6, AA7, AA8 and AA9, (b) AA1, AA2, AA3, AA4, AA6, AA7, AA8 and AA10, or (c) AA1, AA2, AA3, AA4, AA6, AA7, AA8 and AA11. The at least eight amino acid residues may furthermore also be (a) AA1, AA2, AA3, AA4, AA6, AA7, AA9 and AA10, or (b) AA1, AA2, AA3, AA4, AA6, AA7, AA9 and AA11. The at least eight amino acid residues may also be AA1, AA2, AA3, AA4, AA6, AA7, AA10 and AA11. Such at least eight amino acid residues may be (a) AA1, AA2, AA3, AA4, AA6, AA8, AA9 and AA10, or (b) AA1, AA2, AA3, AA4, AA6, AA8, AA9 and AA11. The at least eight amino acid residues may also be AA1, AA2, AA3, AA4, AA6, AA8, AA10 and AA11. The at least eight amino acid residues may also be AA1, AA2, AA3, AA4, AA6, AA9, AA10 and AA11. Such at least eight amino acid residues may be (a) AA1, AA2, AA3, AA4, AA7, AA8, AA9 and AA10, or (b) AA1, AA2, AA3, AA4, AA7, AA8, AA9 and AA11. The at least eight amino acid residues may also be AA1, AA2, AA3, AA4, AA7, AA8, AA10 and AA11. The at least eight amino acid residues may also be AA1, AA2, AA3, AA4, AA7, AA9, AA10 and AA11. The at least eight amino acid residues may also be AA1, AA2, AA3, AA4, AA8, AA9, AA10 and AA11. Such at least eight amino acid residues may be (a) AA1, AA2, AA3, AA5, AA6, AA7, AA8 and AA9, (b) AA1, AA2, AA3, AA5, AA6, AA7, AA8 and AA10, or (c) AA1, AA2, AA3, AA5, AA6, AA7, AA8 and AA11. The at least eight amino acid residues may furthermore also be (a) AA1, AA2, AA3, AA5, AA6, AA7, AA9 and AA10, or (b) AA1, AA2, AA3, AA5, AA6, AA7, AA9 and AA11. The at least eight amino acid residues may also be AA1, AA2, AA3, AA5, AA6, AA7, AA10 and AA11. Such at least eight amino acid residues may be (a) AA1, AA2, AA3, AA5, AA6, AA8, AA9 and AA10, or (b) AA1, AA2, AA3, AA5, AA6, AA8, AA9 and AA11. The at least eight amino acid residues may also be AA1, AA2, AA3, AA5, AA6, AA8, AA10 and AA11. The at least eight amino acid residues may also be AA1, AA2, AA3, AA5, AA6, AA9, AA10 and AA11. Such at least eight amino acid residues may be (a) AA1, AA2, AA3, AA5, AA7, AA8, AA9 and AA10, or (b) AA1, AA2, AA3, AA5, AA7, AA8, AA9 and AA11. The at least eight amino acid residues may also be AA1, AA2, AA3, AA5, AA7, AA8, AA10 and AA11. The at least eight amino acid residues may also be AA1, AA2, AA3, AA5, AA7, AA9, AA10 and AA11. The at least eight amino acid residues may also be AA1, AA2, AA3, AA5, AA8, AA9, AA10 and AA11. Such at least eight amino acid residues may be (a) AA1, AA2, AA3, AA6, AA7, AA8, AA9 and AA10, or (b) AA1, AA2, AA3, AA6, AA7, AA8, AA9 and AA11. The at least eight amino acid residues may also be AA1, AA2, AA3, AA6, AA7, AA8, AA10 and AA11. The at least eight amino acid residues may also be AA1, AA2, AA3, AA6, AA7, AA9, AA10 and AA11. The at least eight amino acid residues may also be AA1, AA2, AA3, AA6, AA8, AA9, AA10 and AA11. The at least eight amino acid residues may also be AA1, AA2, AA3, AA7, AA8, AA9, AA10 and AA11. Such at least eight amino acid residues may be (a) AA1, AA2, AA4, AA5, AA6, AA7, AA8 and AA9, (b) AA1, AA2, AA4, AA5, AA6, AA7, AA8 and AA10, or (c) AA1, AA2, AA4, AA5, AA6, AA7, AA8 and AA11. The at least eight amino acid residues may furthermore also be (a) AA1, AA2, AA4, AA5, AA6, AA7, AA9 and AA10, or (b) AA1, AA2, AA4, AA5, AA6, AA7, AA9 and AA11. The at least eight amino acid residues may also be AA1, AA2, AA4, AA5, AA6, AA7, AA10 and AA11. Such at least eight amino acid residues may be (a) AA1, AA2, AA4, AA5, AA6, AA8, AA9 and AA10, or (b) AA1, AA2, AA4, AA5, AA6, AA8, AA9 and AA11. The at least eight amino acid residues may also be AA1, AA2, AA4, AA5, AA6, AA8, AA10 and AA11. The at least eight amino acid residues may also be AA1, AA2, AA4, AA5, AA6, AA9, AA10 and AA11. Such at least eight amino acid residues may be (a) AA1, AA2, AA4, AA5, AA7, AA8, AA9 and AA10, or (b) AA1, AA2, AA4, AA5, AA7, AA8, AA9 and AA11. The at least eight amino acid residues may also be AA1, AA2, AA4, AA5, AA7, AA8, AA10 and AA11. The at least eight amino acid residues may also be AA1, AA2, AA4, AA5, AA7, AA9, AA10 and AA11. The at least eight amino acid residues may also be AA1, AA2, AA4, AA5, AA8, AA9, AA10 and AA11. Such at least eight amino acid residues may be (a) AA1, AA2, AA4, AA6, AA7, AA8, AA9 and AA10, or (b) AA1, AA2, AA4, AA6, AA7, AA8, AA9 and AA11. The at least eight amino acid residues may also be AA1, AA2, AA4, AA6, AA7, AA8, AA10 and AA11. The at least eight amino acid residues may also be AA1, AA2, AA4, AA6, AA7, AA9, AA10 and AA11. The at least eight amino acid residues may also be AA1, AA2, AA4, AA6, AA8, AA9, AA10 and AA11. The at least eight amino acid residues may also be AA1, AA2, AA4, AA7, AA8, AA9, AA10 and AA11. Such at least eight amino acid residues may be (a) AA1, AA2, AA5, AA6, AA7, AA8, AA9 and AA10, or (b) AA1, AA2, AA5, AA6, AA7, AA8, AA9 and AA11. The at least eight amino acid residues may also be AA1, AA2, AA5, AA6, AA7, AA8, AA10 and AA11. The at least eight amino acid residues may also be AA1, AA2, AA5, AA6, AA7, AA9, AA10 and AA11. The at least eight amino acid residues may also be AA1, AA2, AA5, AA6, AA8, AA9, AA10 and AA11. The at least eight amino acid residues may also be AA1, AA2, AA5, AA7, AA8, AA9, AA10 and AA11. The at least eight amino acid residues may also be AA1, AA2, AA6, AA7, AA8, AA9, AA10 and AA11. Such at least eight amino acid residues may be (a) AA1, AA3, AA4, AA5, AA6, AA7, AA8 and AA9, (b) AA1, AA3, AA4, AA5, AA6, AA7, AA8 and AA10, or (c) AA1, AA3, AA4, AA5, AA6, AA7, AA8 and AA11. The at least eight amino acid residues may furthermore also be (a) AA1, AA3, AA4, AA5, AA6, AA7, AA9 and AA10, or (b) AA1, AA3, AA4, AA5, AA6, AA7, AA9 and AA11. The at least eight amino acid residues may also be AA1, AA3, AA4, AA5, AA6, AA7, AA10 and AA11. Such at least eight amino acid residues may be (a) AA1, AA3, AA4, AA5, AA6, AA8, AA9 and AA10, or (b) AA1, AA3, AA4, AA5, AA6, AA8, AA9 and AA11. The at least eight amino acid residues may also be AA1, AA3, AA4, AA5, AA6, AA8, AA10 and AA11. The at least eight amino acid residues may also be AA1, AA3, AA4, AA5, AA6, AA9, AA10 and AA11. Such at least eight amino acid residues may be (a) AA1, AA3, AA4, AA5, AA7, AA8, AA9 and AA10, or (b) AA1, AA3, AA4, AA5, AA7, AA8, AA9 and AA11. The at least eight amino acid residues may also be AA1, AA3, AA4, AA5, AA7, AA8, AA10 and AA11. The at least eight amino acid residues may also be AA1, AA3, AA4, AA5, AA7, AA9, AA10 and AA11. The at least eight amino acid residues may also be AA1, AA3, AA4, AA5, AA8, AA9, AA10 and AA11. Such at least eight amino acid residues may be (a) AA1, AA3, AA4, AA6, AA7, AA8, AA9 and AA10, or (b) AA1, AA3, AA4, AA6, AA7, AA8, AA9 and AA11. The at least eight amino acid residues may also be AA1, AA3, AA4, AA6, AA7, AA8, AA10 and AA11. The at least eight amino acid residues may also be AA1, AA3, AA4, AA6, AA7, AA9, AA10 and AA11. The at least eight amino acid residues may also be AA1, AA3, AA4, AA6, AA8, AA9, AA10 and AA11. The at least eight amino acid residues may also be AA1, AA3, AA4, AA7, AA8, AA9, AA10 and AA11. Such at least eight amino acid residues may be (a) AA1, AA3, AA5, AA6, AA7, AA8, AA9 and AA10, or (b) AA1, AA3, AA5, AA6, AA7, AA8, AA9 and AA11. The at least eight amino acid residues may also be AA1, AA3, AA5, AA6, AA7, AA8, AA10 and AA11. The at least eight amino acid residues may also be AA1, AA3, AA5, AA6, AA7, AA9, AA10 and AA11. The at least eight amino acid residues may also be AA1, AA3, AA5, AA6, AA8, AA9, AA10 and AA11. The at least eight amino acid residues may also be AA1, AA3, AA5, AA7, AA8, AA9, AA10 and AA11. The at least eight amino acid residues may also be AA1, AA3, AA6, AA7, AA8, AA9, AA10 and AA11. Such at least eight amino acid residues may be (a) AA1, AA4, AA5, AA6, AA7, AA8, AA9 and AA10, or (b) AA1, AA4, AA5, AA6, AA7, AA8, AA9 and AA11. The at least eight amino acid residues may also be AA1, AA4, AA5, AA6, AA7, AA8, AA10 and AA11. The at least eight amino acid residues may also be AA1, AA4, AA5, AA6, AA7, AA9, AA10 and AA11. The at least eight amino acid residues may also be AA1, AA4, AA5, AA6, AA8, AA9, AA10 and AA11. The at least eight amino acid residues may also be AA1, AA4, AA5, AA7, AA8, AA9, AA10 and AA11. The at least eight amino acid residues may also be AA1, AA4, AA6, AA7, AA8, AA9, AA10 and AA11. The at least eight amino acid residues may also be AA1, AA5, AA6, AA7, AA8, AA9, AA10 and AA11.

Such at least eight amino acid residues may be (a) AA2, AA3, AA4, AA5, AA6, AA7, AA8 and AA9, (b) AA2, AA3, AA4, AA5, AA6, AA7, AA8 and AA10, or (c) AA2, AA3, AA4, AA5, AA6, AA7, AA8 and AA11. The at least eight amino acid residues may furthermore also be (a) AA2, AA3, AA4, AA5, AA6, AA7, AA9 and AA10, or (b) AA2, AA3, AA4, AA5, AA6, AA7, AA9 and AA11. The at least eight amino acid residues may also be AA2, AA3, AA4, AA5, AA6, AA7, AA10 and AA11. Such at least eight amino acid residues may be (a) AA2, AA3, AA4, AA5, AA6, AA8, AA9 and AA10, or (b) AA2, AA3, AA4, AA5, AA6, AA8, AA9 and AA11. The at least eight amino acid residues may also be AA2, AA3, AA4, AA5, AA6, AA8, AA10 and AA11. The at least eight amino acid residues may also be AA2, AA3, AA4, AA5, AA6, AA9, AA10 and AA11. Such at least eight amino acid residues may be (a) AA2, AA3, AA4, AA5, AA7, AA8, AA9 and AA10, or (b) AA2, AA3, AA4, AA5, AA7, AA8, AA9 and AA11. The at least eight amino acid residues may also be AA2, AA3, AA4, AA5, AA7, AA8, AA10 and AA11. The at least eight amino acid residues may also be AA2, AA3, AA4, AA5, AA7, AA9, AA10 and AA11. The at least eight amino acid residues may also be AA2, AA3, AA4, AA5, AA8, AA9, AA10 and AA11. Such at least eight amino acid residues may be (a) AA2, AA3, AA4, AA6, AA7, AA8, AA9 and AA10, or (b) AA2, AA3, AA4, AA6, AA7, AA8, AA9 and AA11. The at least eight amino acid residues may also be AA2, AA3, AA4, AA6, AA7, AA8, AA10 and AA11. The at least eight amino acid residues may also be AA2, AA3, AA4, AA6, AA7, AA9, AA10 and AA11. The at least eight amino acid residues may also be AA2, AA3, AA4, AA6, AA8, AA9, AA10 and AA11. The at least eight amino acid residues may also be AA2, AA3, AA4, AA7, AA8, AA9, AA10 and AA11. Such at least eight amino acid residues may be (a) AA2, AA3, AA5, AA6, AA7, AA8, AA9 and AA10, or (b) AA2, AA3, AA5, AA6, AA7, AA8, AA9 and AA11. The at least eight amino acid residues may also be AA2, AA3, AA5, AA6, AA7, AA8, AA10 and AA11. The at least eight amino acid residues may also be AA2, AA3, AA5, AA6, AA7, AA9, AA10 and AA11. The at least eight amino acid residues may also be AA2, AA3, AA5, AA6, AA8, AA9, AA10 and AA11. The at least eight amino acid residues may also be AA2, AA3, AA5, AA7, AA8, AA9, AA10 and AA11. The at least eight amino acid residues may also be AA2, AA3, AA6, AA7, AA8, AA9, AA10 and AA11. Such at least eight amino acid residues may be (a) AA2, AA4, AA5, AA6, AA7, AA8, AA9 and AA10, or (b) AA2, AA4, AA5, AA6, AA7, AA8, AA9 and AA11. The at least eight amino acid residues may also be AA2, AA4, AA5, AA6, AA7, AA8, AA10 and AA11. The at least eight amino acid residues may also be AA2, AA4, AA5, AA6, AA7, AA9, AA10 and AA11. The at least eight amino acid residues may also be AA2, AA4, AA5, AA6, AA8, AA9, AA10 and AA11. The at least eight amino acid residues may also be AA2, AA4, AA5, AA7, AA8, AA9, AA10 and AA11. The at least eight amino acid residues may also be AA2, AA4, AA6, AA7, AA8, AA9, AA10 and AA11. The at least eight amino acid residues may also be AA2, AA5, AA6, AA7, AA8, AA9, AA10 and AA11.

Such at least eight amino acid residues may be (a) AA3, AA4, AA5, AA6, AA7, AA8, AA9 and AA10, or (b) AA3, AA4, AA5, AA6, AA7, AA8, AA9 and AA11. The at least eight amino acid residues may also be AA3, AA4, AA5, AA6, AA7, AA8, AA10 and AA11. The at least eight amino acid residues may also be AA3, AA4, AA5, AA6, AA7, AA9, AA10 and AA11. The at least eight amino acid residues may also be AA3, AA4, AA5, AA6, AA8, AA9, AA10 and AA11. The at least eight amino acid residues may also be AA3, AA4, AA5, AA7, AA8, AA9, AA10 and AA11. The at least eight amino acid residues may also be AA3, AA4, AA6, AA7, AA8, AA9, AA10 and AA11. The at least eight amino acid residues may also be AA3, AA5, AA6, AA7, AA8, AA9, AA10 and AA11. The at least eight amino acid residues may also be AA4, AA5, AA6, AA7, AA8, AA9, AA10 and AA11.

Such at least nine amino acid residues may be (a) AA1, AA2, AA3, AA4, AA5, AA6, AA7, AA8 and AA9, (b) AA1, AA2, AA3, AA4, AA5, AA6, AA7, AA8 and AA10, or (c) AA1, AA2, AA3, AA4, AA5, AA6, AA7, AA8 and AA11. The at least nine amino acid residues may furthermore also be (a) AA1, AA2, AA3, AA4, AA5, AA6, AA7, AA9 and AA10, or (b) AA1, AA2, AA3, AA4, AA5, AA6, AA7, AA9 and AA11. The at least nine amino acid residues may also be AA1, AA2, AA3, AA4, AA5, AA6, AA7, AA10 and AA11. Such at least nine amino acid residues may be (a) AA1, AA2, AA3, AA4, AA5, AA6, AA8, AA9 and AA10, or (b) AA1, AA2, AA3, AA4, AA5, AA6, AA8, AA9 and AA11. The at least nine amino acid residues may also be AA1, AA2, AA3, AA4, AA5, AA6, AA8, AA10 and AA11. The at least nine amino acid residues may also be AA1, AA2, AA3, AA4, AA5, AA6, AA9, AA10 and AA11. Such at least nine amino acid residues may be (a) AA1, AA2, AA3, AA4, AA5, AA7, AA8, AA9 and AA10, or (b) AA1, AA2, AA3, AA4, AA5, AA7, AA8, AA9 and AA11. The at least nine amino acid residues may also be AA1, AA2, AA3, AA4, AA5, AA7, AA8, AA10 and AA11. The at least nine amino acid residues may also be AA1, AA2, AA3, AA4, AA5, AA7, AA9, AA10 and AA11. The at least nine amino acid residues may also be AA1, AA2, AA3, AA4, AA5, AA8, AA9, AA10 and AA11. Such at least nine amino acid residues may be (a) AA1, AA2, AA3, AA4, AA6, AA7, AA8, AA9 and AA10, or (b) AA1, AA2, AA3, AA4, AA6, AA7, AA8, AA9 and AA11. The at least nine amino acid residues may also be AA1, AA2, AA3, AA4, AA6, AA7, AA8, AA10 and AA11. The at least nine amino acid residues may also be AA1, AA2, AA3, AA4, AA6, AA7, AA9, AA10 and AA11. The at least nine amino acid residues may also be AA1, AA2, AA3, AA4, AA6, AA8, AA9, AA10 and AA11. The at least nine amino acid residues may also be AA1, AA2, AA3, AA4, AA7, AA8, AA9, AA10 and AA11. Such at least nine amino acid residues may be (a) AA1, AA2, AA3, AA5, AA6, AA7, AA8, AA9 and AA10, or (b) AA1, AA2, AA3, AA5, AA6, AA7, AA8, AA9 and AA11. The at least nine amino acid residues may also be AA1, AA2, AA3, AA5, AA6, AA7, AA8, AA10 and AA11. The at least nine amino acid residues may also be AA1, AA2, AA3, AA5, AA6, AA7, AA9, AA10 and AA11. The at least nine amino acid residues may also be AA1, AA2, AA3, AA5, AA6, AA8, AA9, AA10 and AA11. The at least nine amino acid residues may also be AA1, AA2, AA3, AA5, AA7, AA8, AA9, AA10 and AA11. The at least nine amino acid residues may also be AA1, AA2, AA3, AA6, AA7, AA8, AA9, AA10 and AA11. Such at least nine amino acid residues may be (a) AA1, AA2, AA4, AA5, AA6, AA7, AA8, AA9 and AA10, or (b) AA1, AA2, AA4, AA5, AA6, AA7, AA8, AA9 and AA11. The at least nine amino acid residues may also be AA1, AA2, AA4, AA5, AA6, AA7, AA8, AA10 and AA11. The at least nine amino acid residues may also be AA1, AA2, AA4, AA5, AA6, AA7, AA9, AA10 and AA11. The at least nine amino acid residues may also be AA1, AA2, AA4, AA5, AA6, AA8, AA9, AA10 and AA11. The at least nine amino acid residues may also be AA1, AA2, AA4, AA5, AA7, AA8, AA9, AA10 and AA11. The at least nine amino acid residues may also be AA1, AA2, AA4, AA6, AA7, AA8, AA9, AA10 and AA11. The at least nine amino acid residues may also be AA1, AA2, AA5, AA6, AA7, AA8, AA9, AA10 and AA11. Such at least nine amino acid residues may be (a) AA1, AA3, AA4, AA5, AA6, AA7, AA8, AA9 and AA10, or (b) AA1, AA3, AA4, AA5, AA6, AA7, AA8, AA9 and AA11. The at least nine amino acid residues may also be AA1, AA3, AA4, AA5, AA6, AA7, AA8, AA10 and AA11. The at least nine amino acid residues may also be AA1, AA3, AA4, AA5, AA6, AA7, AA9, AA10 and AA11. The at least nine amino acid residues may also be AA1, AA3, AA4, AA5, AA6, AA8, AA9, AA10 and AA11. The at least nine amino acid residues may also be AA1, AA3, AA4, AA5, AA7, AA8, AA9, AA10 and AA11. The at least nine amino acid residues may also be AA1, AA3, AA4, AA6, AA7, AA8, AA9, AA10 and AA11. The at least nine amino acid residues may also be AA1, AA3, AA5, AA6, AA7, AA8, AA9, AA10 and AA11. The at least nine amino acid residues may also be AA1, AA4, AA5, AA6, AA7, AA8, AA9, AA10 and AA11.

Such at least nine amino acid residues may be (a) AA2, AA3, AA4, AA5, AA6, AA7, AA8, AA9 and AA10, or (b) AA2, AA3, AA4, AA5, AA6, AA7, AA8, AA9 and AA11. The at least nine amino acid residues may also be AA2, AA3, AA4, AA5, AA6, AA7, AA8, AA10 and AA11. The at least nine amino acid residues may also be AA2, AA3, AA4, AA5, AA6, AA7, AA9, AA10 and AA11. The at least nine amino acid residues may also be AA2, AA3, AA4, AA5, AA6, AA8, AA9, AA10 and AA11. The at least nine amino acid residues may also be AA2, AA3, AA4, AA5, AA7, AA8, AA9, AA10 and AA11. The at least nine amino acid residues may also be AA2, AA3, AA4, AA6, AA7, AA8, AA9, AA10 and AA11. The at least nine amino acid residues may also be AA2, AA3, AA5, AA6, AA7, AA8, AA9, AA10 and AA11. The at least nine amino acid residues may also be AA2, AA4, AA5, AA6, AA7, AA8, AA9, AA10 and AA11. The at least nine amino acid residues may also be AA3, AA4, AA5, AA6, AA7, AA8, AA9, AA10 and AA11.

Such at least ten amino acid residues may furthermore be (a) AA1, AA2, AA3, AA4, AA5, AA6, AA7, AA8, AA9 and AA10, (b) AA1, AA2, AA3, AA4, AA5, AA6, AA7, AA8, AA9 and AA11 or (c) AA1, AA2, AA3, AA4, AA5, AA6, AA7, AA8, AA10 and AA11. Such at least ten amino acid residues may even be AA1, AA2, AA3, AA4, AA5, AA6, AA7, AA9, AA10 and AA11. Such at least ten amino acid residues may also be (a) AA1, AA2, AA3, AA4, AA5, AA6, AA8, AA9, AA10 and AA11, (b) AA1, AA2, AA3, AA4, AA5, AA7, AA8, AA9, AA10 and AA11, (c) AA1, AA2, AA3, AA4, AA6, AA7, AA8, AA9, AA10 and AA11 or (d) AA1, AA2, AA3, AA5, AA6, AA7, AA8, AA9, AA10 and AA11. Such at least ten amino acid residues may also be (a) AA1, AA2, AA4, AA5, AA6, AA7, AA8, AA9, AA10 and AA11, (b) AA1, AA3, AA4, AA5, AA6, AA7, AA8, AA9, AA10 and AA11 or (c) AA2, AA3, AA4, AA5, AA6, AA7, AA8, AA9, AA10 and AA11.

### Rca Proteins and Nucleic Acids

In many species including rice there is a single Rca gene that encodes two protein isoforms based on alternative splicing of pre mRNA into a larger α isoform and a shorter β isoform^{18,19}. Similarly, for wheat there is an α and ρ isoform. In addition, wheat has a separate Rca gene in close proximity to the first, on wheat chromosome four that encodes a second variant of the β isoform. The latter is referred to as form 1 and the alternatively spliced form is referred to as form 2 Rca¹¹. Thus in wheat—excluding the near identical copies on each of the three wheat chromosome sub genomes—there are three distinct Rca gene products that can be expressed, a form 1 β (TaRca1-β), form 2 α (TaRca2-α) and form 2 β (TaRca2-β).

Rca proteins are AAA+ chaperones which can form hexameric protein complexes and interact with Rubisco. Functional Rca proteins comprise a central ATPase domain (the AAA+ module) and a C-terminal domain involved in Rubisco-Rca and Rca-Rca interactions³³⁻³⁵. The AAA+ module is located from amino acid at a position equivalent to position 57 to a position equivalent to position 345 on SEQ ID NO: 2, the ATPase core is located from amino acid at a position equivalent to position 182 to a position equivalent to position 282 on SEQ ID NO: 2 and the C-terminal domain is located from amino acid at a position equivalent to position 346 to a position equivalent to position 427 on SEQ ID NO: 2. More specifically, functional Rca proteins furthermore comprise an N-linker (IA) at the amino acid positions equivalent to position 123 and 124 on SEQ ID NO: 2, a Walker A motif (GxxxxGK) at the amino acid positions equivalent to positions 155 to 161 on SEQ ID NO: 2, a Walker B motif (LxxxD) at the amino acid positions equivalent to positions 215 to 219 on SEQ ID NO: 2, a Rubisco interaction loop (Shivhare et al 2017) at the amino acid positions equivalent to positions 253 to 265 on SEQ ID NO: 2, an Rca-Rca interface (Stotz et al. 2011, Nature Structural and Molecular Biology 18: 1366-1370) at the amino acid positions equivalent to positions 339 to 347 on SEQ ID NO: 2, and a tyrosine (Y) at the amino acid positions equivalent to position 406 on SEQ ID NO: 2.

Conferring thermostability to a protein complex comprising the Rubisco Activase and the Rubisco protein means to increase the midpoint temperature at which Rubisco activation velocity by Rca is reduced by half (Tm, see Example 2). The conferred thermostability of such protein complex may be an increase of the midpoint temperature by about 1° C., by about 2° C., by about 3° C., by about 4° C., by about 5° C., by about 6° C., by about 7° C., by about 8° C., by about 9° C., or about 10° C. It may also be an increase of the midpoint temperature of at least about 1° C., by at least about 2° C., by at least about 3° C., by at least about 4° C., by at least about 5° C., by at least about 6° C., by at least about 7° C., by at least about 8° C., by at least about 9° C., or by at least about 10° C. It may also be an increase of the midpoint temperature of between about 1° C. and 4° C., between about 1° C. and 5° C., between about 1° C. and 6° C., between about 1° C. and 7° C., between about 1° C. and 8° C., between about 1° C. and 9° C., between about 1° C. and 10° C., between about 2° C. and 5° C., between about 2° C. and 6° C., between about 2° C. and 7° C., between about 2° C. and 8° C., between about 2° C. and 9° C., between about 2° C. and 10° C., between about 3° C. and 6° C., between about 3° C. and 7° C., between about 3° C. and 8° C., between about 3° C. and 9° C., between about 3° C. and 10° C., between about 4° C. and 7° C., between about 4° C. and 8° C., between about 4° C. and 9° C., between about 4° C. and 10° C., between about 5° C. and 8° C., between about 5° C. and 9° C., between about 5° C. and 10° C., between about 6° C. and 9° C., between about 6° C. and 10° C., or between about 7° C. and 10° C.

The midpoint temperature at which Rubisco activation velocity by Rca is reduced by half (Tm) for such thermostable complex may be at least about or about 37° C., at least about or about 38° C., at least about or about 39° C., at least about or about 40° C., at least about or about 41° C., at least about or about 42° C., at least about or about 43° C., at least about or about 44° C., or at least about or about 45° C. It may also be between about 37° C. and about 40° C., between about 37° C. and about 42° C., between about 37° C. and about 45° C., between about 39° C. and about 41° C., between about 39° C. and about 43° C., or between about 39° C. and about 45° C.

Such thermostable complex may have an enzymatic activity under heat stress conditions.

Complexes comprising the Rubisco Activase and the Rubisco protein may be formed in vitro or in vivo. For example the complexes may be formed in vitro by contacting a thermostable Rca 2 according to the invention or a wheat Rca1-β as described herein with Rubisco from wheat present in a leaf extract or Rubisco that has been purified from wheat. Alternatively, the complexes may be formed in vivo by expression of an Rca polypeptide of the invention in a plant such that it forms a complex with the endogenous Rubisco of the plant.

Any method known in the art can be used to assay the activity of complexes comprising Rca polypeptides (including, but not limited to, Rubisco activation and ATP hydrolysis, see for example Chakrabarti et al. 2002 J. Biochem. Biophys. Methods 52:179-187 and McC Lilley and Portis 1997 Plant Physiol. 114:605-613).

A “thermostable Rca protein” is an Rca protein capable of conferring thermostability to a protein complex comprising the Rubisco Activase and the Rubisco protein. A thermostable Rca protein may also be an Rca protein with a higher midpoint temperature at which the Rca unfolds.

The midpoint temperature at which a thermostable Rca protein unfolds may be of at least about or about 1° C., by at least about or about 2° C., by at least about or about 3° C., by at least about or about 4° C., by at least about or about 5° C., by at least about or about 6° C., by at least about or about 7° C., by at least about or about 8° C., by at least about or about 9° C., or by at least about or about 10° C. higher than the midpoint temperature at which a non-thermostable Rca protein unfolds. It may also be between about 1° C. and 4° C., between about 1° C. and 5° C., between about 1° C. and 6° C., between about 1° C. and 7° C., between about 1° C. and 8° C., between about 1° C. and 9° C., between about 1° C. and 10° C., between about 2° C. and 5° C., between about 2° C. and 6° C., between about 2° C. and 7° C., between about 2° C. and 8° C., between about 2° C. and 9° C., between about 2° C. and 10° C., between about 3° C. and 6° C., between about 3° C. and 7° C., between about 3° C. and 8° C., between about 3° C. and 9° C., between about 3° C. and 10° C., between about 4° C. and 7° C., between about 4° C. and 8° C., between about 4° C. and 9° C., between about 4° C. and 10° C., between about 5° C. and 8° C., between about 5° C. and 9° C., between about 5° C. and 10° C., between about 6° C. and 9° C., between about 6° C. and 10° C., or between about 7° C. and 10° C. higher than the midpoint temperature at which a non-thermostable Rca protein unfolds.

The midpoint temperature at which the thermostable protein unfolds may be at least about or about 35° C., at least about or about 36° C., at least about or about 37° C., at least about or about 38° C., at least about or about 39° C., at least about or about 40° C., at least about or about 41° C., at least about or about 42° C., or at least about or about 43° C. It may also be between about 35° C. and about 38° C., between about 35° C. and about 40° C., between about 35° C. and about 43° C., between about 37° C. and about 39° C., between about 37° C. and about 41° C., or between about 37° C. and about 43° C.

### Rca1-β

The Rca1-β protein is shown here to confer thermostability to a protein complex comprising this Rubisco activase and the Rubisco protein.

The Rca 1β protein and variants thereof described and used herein comprise an amino acid sequence selected from (a) the amino acid sequence of SEQ ID NOs: 8, 47 or 49 and (b) an amino acid sequence having at least 90% identity to the amino acid sequence of SEQ ID NOs: 8, 47 or 49 and comprising at least one amino acid selected from (i) an isoleucine at a position corresponding to position 109 of SEQ ID NO: 8, (ii) an aspartic acid at a position corresponding to position 123 of SEQ ID NO: 8, (iii) an isoleucine at a position corresponding to position 210 of SEQ ID NO: 8, (iv) an arginine at a position corresponding to position 315 of SEQ ID NO: 8, (v) a proline at a position corresponding to position 320 of SEQ ID NO: 8, (vi) a leucine at a position corresponding to position 327 of SEQ ID NO: 8, (vii) a glutamic acid at a position corresponding to position 357 of SEQ ID NO: 8, (viii) an isoleucine at a position corresponding to position 384 of SEQ ID NO: 8, (ix) a lysine at a position corresponding to position 409 of SEQ ID NO: 8, (x) a leucine at a position corresponding to position 411 of SEQ ID NO: 8 and (xi) a glutamic acid at a position corresponding to position 413 of SEQ ID NO: 8.

SEQ ID NO: 8 represents the amino acid sequence of the Rca 1β protein from the wheat subgenome B, SEQ ID NO: 47 represents the amino acid sequence of the Rca 1β protein from the wheat subgenome A and SEQ ID NO: 49 represents the amino acid sequence of the Rca 1β protein from the wheat subgenome D.

Suitable for the invention are amino acid sequences of Rca 1β protein, which comprise an amino acid sequence having at least 80%, or at least 85%, or at least 90%, or at least 91%, or at least 92%, or at least 93%, or at least 94%, or at least 95%, or at least 96%, or at least 97%, or at least 98%, or at least 99%, or at least 100% sequence identity to the herein described protein and are also referred to as variants. The term “variant” with respect to the amino acid sequence SEQ ID NO: 8 of the invention is intended to mean substantially similar sequences. The amino acid sequences of SEQ ID NO: 47 and SEQ ID NO: 49 are variants of the amino acid sequence SEQ ID NO: 8.

Furthermore, it is clear that variants of Rca 1β protein, wherein one or more amino acid residues have been deleted, substituted or inserted, can also be used to the same effect in the methods according to the invention, provided that the central ATPase domain (the AAA+ module), the C-terminal domain, the N-linker, Walker A, Walker B motives, the Rubisco interaction loop, the Rca-Rca interface and the tyrosine (Y) at the amino acid positions equivalent to position 406 of SEQ ID NO: 2, are not affected by the deletion, substitution or insertion of amino-acid.

Such Rca 1β protein variant may also comprise at least one, or at least two, or at least three, or at least four, or at least five, or at least six, or at least seven, or at least eight, or at least nine, or at least ten or all eleven of the amino acid residues identified as conserved in thermostable variants of Rca proteins from various plant species and listed above. The possible combinations of such at least two, or at least three, or at least four, or at least five, or at least six, or at least seven, or at least eight, or at least nine, or at least ten of the amino acid residues are listed above.

Furthermore, nucleic acid encoding such Rca 1p protein and variants thereof may comprise a coding nucleic acid sequence selected from (a) the nucleic acid of SEQ ID NOs: 7, 46 or 48, or complement thereof and (b) a nucleic acid having at least 60% identity to the nucleic acid of SEQ ID NOs: 7, 46 or 48, or complement thereof.

SEQ ID NO: 7 represents the coding nucleotide sequence of the wheat Rca 1β gene from the subgenome B, SEQ ID NO: 46 represents the coding nucleotide sequence of the wheat Rca 1β gene from the subgenome A and SEQ ID NO: 48 represents the coding nucleotide sequence of the wheat Rca 1β gene from the subgenome D.

Suitable for the invention are nucleic acids encoding an Rca 1β protein, which comprise a nucleotide sequence having at least 60%, or at least 70%, or at least 80%, or at least 85%, or at least 90%, or at least 95%, or at least 98% sequence identity to the herein described gene and are also referred to as variants.

A nucleic acid comprising a nucleotide sequence having at least 60% sequence identity to SEQ ID NOs: 7, 46 or 48 can thus be a nucleic acid comprising a nucleotide sequence having at least 60%, or at least 70%, or at least 80%, or at least 85%, or at least 90%, or at least 95%, or at least 98%, or 100% sequence identity to SEQ ID NOs: 7, 46 or 48 respectively.

### Endogenous Non-Thermostable Rca 2

In contrast with the Rca 1β, the Rca 2 endogenous proteins are known to be non-thermostable. As described herein, the endogenous non-thermostable Rca 2 protein may comprise the amino acid sequences of SEQ ID NOs: 2, 6, 39, 41, 43 or 45. The endogenous non-thermostable Rca 2 protein may also comprise an amino acid sequence having at least 90% sequence identity with the amino acid sequences of SEQ ID NOs: 2, 6, 39, 41, 43 or 45 and not encoding the amino acids selected from (a) an isoleucine at a position corresponding to position 59 of SEQ ID NO: 4, (b) an aspartic acid at a position corresponding to position 73 of SEQ ID NO: 4, (c) an isoleucine at a position corresponding to position 160 of SEQ ID NO: 4, (d) an arginine at a position corresponding to position 265 of SEQ ID NO: 4, (e) a proline at a position corresponding to position 270 of SEQ ID NO: 4, (f) a leucine at a position corresponding to position 277 of SEQ ID NO: 4, (g) a glutamic acid at a position corresponding to position 307 of SEQ ID NO: 4, (h) an isoleucine at a position corresponding to position 334 of SEQ ID NO: 4, (i) a lysine at a position corresponding to position 359 of SEQ ID NO: 4, (j) a leucine at a position corresponding to position 361 of SEQ ID NO: 4 and (k) a glutamic acid at a position corresponding to position 363 of SEQ ID NO: 4.

A non-thermostable endogenous Rca 2 protein may thus also comprise an amino acid sequence having at least 90% sequence identity with the amino acid sequences of SEQ ID NOs: 2, 6, 39, 41, 43 or 45 and not comprising the amino acids AA1, AA2, AA3, AA4, AA5, AA6, AA7, AA8, AA9, AA10 and AA11.

A non-thermostable endogenous Rca 2 protein may comprise an amino acid sequence having at least 80%, or at least 85%, or at least 90%, or at least 91%, or at least 92%, or at least 93%, or at least 94%, or at least 95%, or at least 96%, or at least 97%, or at least 98%, or at least 99%, or at least 100% sequence identity to the herein described protein which does not comprise the amino acids AA1, AA2, AA3, AA4, AA5, AA6, AA7, AA8, AA9, AA10 and AA11.

SEQ ID NOs: 2 and 6 represent respectively the amino acid sequences of the wheat Rca 2P and 2a proteins from the subgenome B. SEQ ID NOs: 39 and 43 represent respectively the amino acid sequences of the wheat Rca 2P and 2a proteins from the subgenome A. SEQ ID NOs: 41 and 45 represent respectively the amino acid sequences of the wheat Rca 2P and 2a proteins from the subgenome D. SEQ ID NO: 4 represent the amino acid acid sequence, excluding the chloroplast targeting peptide, of the wheat Rca 2P from the subgenome B.

Furthermore, the endogenous non-thermostable Rca 2 genes encoding said non-thermostable Rca 2 proteins may comprise the coding nucleotide sequence of SEQ ID NOs: 1, 5, 38, 40, 42 or 44.

SEQ ID NOs: 1 and 5 represent respectively the coding nucleotide sequences of the wheat Rca 2P and 2a gene from the subgenome B. SEQ ID NOs: 38 and 42 represent respectively the coding nucleotide sequences of the wheat Rca 2P and 2a gene from the subgenome A. SEQ ID NOs: 40 and 44 represent respectively the coding nucleotide sequences of the wheat Rca 2P and 2a gene from the subgenome D.

The endogenous non-thermostable Rca 2 gene encoding said non-thermostable Rca 2 proteins may comprise the coding nucleotide sequence of SEQ ID NOs: 1, 5, 38, 40, 42 or 44 or a coding nucleotide sequence having at least 60% identity with the nucleotide sequence of SEQ ID NO: 1, 5, 38, 40, 42 or 44 and not encoding the amino acids selected from (a) an isoleucine at a position corresponding to position 59 of SEQ ID NO: 4, (b) an aspartic acid at a position corresponding to position 73 of SEQ ID NO: 4, (c) an isoleucine at a position corresponding to position 160 of SEQ ID NO: 4, (d) an arginine at a position corresponding to position 265 of SEQ ID NO: 4, (e) a proline at a position corresponding to position 270 of SEQ ID NO: 4, (f) a leucine at a position corresponding to position 277 of SEQ ID NO: 4, (g) a glutamic acid at a position corresponding to position 307 of SEQ ID NO: 4, (h) an isoleucine at a position corresponding to position 334 of SEQ ID NO: 4, (i) a lysine at a position corresponding to position 359 of SEQ ID NO: 4, (j) a leucine at a position corresponding to position 361 of SEQ ID NO: 4 and (k) a glutamic acid at a position corresponding to position 363 of SEQ ID NO: 4.

An endogenous non-thermostable Rca 2 gene encoding said non-thermostable Rca 2 proteins may thus also comprise a nucleotide sequence having at least 60% sequence identity with the nucleotide sequences of SEQ ID NOs: 1, 5, 38, 40, 42 or 44 and not encoding the amino acids AA1, AA2, AA3, AA4, AA5, AA6, AA7, AA8, AA9, AA10 and AA11.

A nucleic acid comprising a nucleotide sequence having at least 60% sequence identity to SEQ ID NOs: 1, 5, 38, 40, 42 or 44 and not encoding the amino acids AA1, AA2, AA3, AA4, AA5, AA6, AA7, AA8, AA9, AA10 and AA11 may be a nucleic acid comprising a nucleotide sequence having at least 60%, or at least 70%, or at least 80%, or at least 85%, or at least 90%, or at least 95%, or at least 98%, or 100% sequence identity to SEQ ID NOs: 1, 5, 38, 40, 42 or 44 respectively and not encoding the amino acids AA1, AA2, AA3, AA4, AA5, AA6, AA7, AA8, AA9, AA10 and AA11.

### Rca 2 Knock Out Alleles

In another embodiment, a knock out allele of an Rca 2 gene is provided. In a further embodiment the Rca 2 gene is the Rca 2P gene from the wheat subgenome B, A or D or the Rca 2a gene from the wheat subgenome B, A or D.

A knock out allele of an Rca 2 gene may be a full knock out allele or a partial knock out allele.

A “full knock-out” or “null” allele, as used herein, refers to a mutant allele, which encodes an protein having no biological activity as compared to the corresponding wild-type functional protein or which encodes no protein at all. Such a “full knock-out mutant allele” is, for example, a wild-type allele, which comprises one or more mutations in its nucleic acid sequence, for example, one or more non-sense, mis-sense, insertion, deletion, frameshift or mutated splice site mutations. In particular, such a full knock-out mutant Rca 2 allele is a wild-type Rca 2 allele, which comprises a mutation that preferably result in the production of an Rca 2 protein lacking at least one functional domain or motif, such as the central ATPase domain (the AAA+ module), the C-terminal domain, the N-linker, Walker A, Walker B motives, the Rubisco interaction loop, the Rca-Rca interface, or lacking at least one amino acid critical for its function such as the tyrosine (Y) at the amino acid positions equivalent to position 406 of SEQ ID NO: 2, such that the biological activity of the Rca 2 protein is completely abolished, or whereby the modification(s) preferably result in no production of an Rca 2 protein.

A “partial knock-out” mutant allele, as used herein, refers to a mutant allele, which encodes an protein having a significantly reduced biological activity as compared to the corresponding wild-type functional protein. Such a “partial knock-out mutant allele” is, for example, a wild-type allele, which comprises one or more mutations in its nucleic acid sequence, for example, one or more missense mutations. In particular, such a partial knockout mutant allele is a wild-type allele, which comprises a mutation that preferably results in the production of a protein wherein at least one conserved and/or functional amino acid is substituted for another amino acid, such that the biological activity is significantly reduced but not completely abolished.

A missense mutation in an Rca 2 allele, as used herein, is any mutation (deletion, insertion or substitution) in an Rca 2 allele whereby one or more codons are changed into the coding DNA and the corresponding mRNA sequence of the corresponding wild type Rca 2 allele, resulting in the substitution of one or more amino acids in the wild type Rca 2 protein for one or more other amino acids in the mutant Rca 2 protein. A mutant Rca 2 allele comprising a missense mutation is an Rca 2 allele wherein one amino acid is substituted.

A nonsense mutation in an Rca 2 allele, as used herein, is a mutation in an Rca 2 allele whereby one or more translation stop codons are introduced into the coding DNA and the corresponding mRNA sequence of the corresponding wild type Rca 2 allele. Translation stop codons are TGA (UGA in the mRNA), TAA (UAA) and TAG (UAG). Thus, any mutation (deletion, insertion or substitution) that leads to the generation of an in-frame stop codon in the coding sequence will result in termination of translation and truncation of the amino acid chain. The truncated protein lacks the amino acids encoded by the coding DNA downstream of the mutation (i.e. the C-terminal part of the Rca 2 protein) and maintains the amino acids encoded by the coding DNA upstream of the mutation (i.e. the N-terminal part of the Rca 2 protein). The more truncated the mutant Rca 2 protein is in comparison to the wild type Rca 2 protein, the more the truncation may result in a significantly reduced activity of the Rca 2 protein. It is believed that, in order for the mutant Rca 2 protein to lose some biological activity, it should at least no longer comprise the tyrosine (Y) at the amino acid positions equivalent to position 406 of SEQ ID NO: 2.

TABLE 1
Examples of substitution mutation resulting
in the generation of an in-frame stop codon.

| Substitution position | Codon before | Resulting |
|---|---|---|
| on SEQ ID NO: 2 | substitution | stop codon |
| 79 | CAG | TAG |
| 182 | TGG | TAG |
| 183 | TGG | TGA |
| 214 | CAG | TAG |
| 217 | CAG | TAG |
| 259 | CAG | TAG |
| 319 | CAG | TAG |
| 461 | TGG | TAG |
| 462 | TGG | TGA |
| 475 | CAA | TAA |
| 490 | CAG | TAG |
| 592 | CAG | TAG |
| 694 | CAG | TAG |
| 712 | CAG | TAG |
| 763 | CAG | TAG |
| 884 | TGG | TAG |
| 885 | TGG | TGA |
| 931 | CAG | TAG |
| 988 | CAA | TAA |
| 1049 | TGG | TAG |
| 1050 | TGG | TGA |
| 1123 | CAG | TAG |
| 1174 | CAG | TAG |
| 1180 | CAG | TAG |
| 1201 | CAG | TAG |

A frameshift mutation in an Rca 2 allele, as used herein, is a mutation (deletion, insertion, duplication, and the like) in an Rca 2 allele that results in the nucleic acid sequence being translated in a different frame downstream of the mutation.

A splice site mutation in an Rca 2 allele, as used herein, is a mutation (deletion, insertion, substitution, duplication, and the like) in an Rca 2 allele whereby a splice donor site or a splice acceptor site is mutated, resulting in altered processing of the mRNA and, consequently, an altered encoded protein, which can have insertions, deletions, substitutions of various lengths, or which can be truncated.

A deletion mutation in an Rca 2 allele, as used herein, is a mutation in an Rca 2 allele that results in the production of an Rca 2 protein which lacks the amino acids encoded by the deleted coding DNA and maintains the amino acids encoded by the coding DNA upstream of the deletion (i.e. the N-terminal part of the Rca 2 protein) and encoding by the coding DNA downstream of the deletion (i.e. the C-terminal part of the Rca 2 protein).

A “significantly reduced amount of functional Rca 2 protein” (e.g. functional Rca 2A, Rca 2B or Rca 2C protein) refers to a reduction in the amount of a functional protein produced by the cell comprising a mutant Rca 2 allele by at least 30%, 40%, 50%, 60%, 70%, or 80% as compared to the amount of the functional Rca 2 protein produced by the cell not comprising the mutant Rca 2 allele. The production of functional Rca 2 protein is however not abolished. This definition encompasses the production of a “non-functional” Rca 2 protein (e.g. truncated Rca 2 protein) having reduced biological activity in vivo, the reduction in the absolute amount of the functional Rca 2 protein (e.g. no functional Rca 2 protein being made due to the mutation in the Rca 2 gene), the production of an Rca 2 protein with significantly reduced biological activity compared to the activity of a functional wild type Rca 2 protein (such as an Rca 2 protein in which one or more amino acid residues that are crucial for the biological activity of the encoded Rca 2 protein are substituted for another amino acid residue).

### Thermostable Rca 2 Variants

It is furthermore an object of the invention to provide a thermostable Rca 2 protein variant. Such thermostable Rca 2 protein variant may comprise an amino acid sequence selected from the amino acid sequences of SEQ ID NO: 30 or 33 and an amino acid sequence having at least 90% identity to the amino acid sequences of SEQ ID NO: 30 or 33 and comprising at least one of the amino acid residues identified as conserved in thermostable variants of Rca proteins from various plant species and listed above.

SEQ ID NO: 30 represent an amino acid sequence based on the one of the wheat Rca 2P from the subgenome B (i.e. SEQ ID NO: 2) but comprising all eleven amino acid residues identified as conserved in thermostable variants of Rca proteins from various plant species. SEQ ID NO: 33 represents the amino acid sequence based on the one of the wheat Rca 2P from the subgenome B (SEQ ID NO: 2) but comprising eight out of the eleven amino acid residues identified as conserved in thermostable variants of Rca proteins from various plant species. These eight amino acid residues are (a) AA1, (b) AA3, (d) AA4, (e) AA5, (f) AA8, (g) AA9, (h) AA10 and (i) AA11.

The thermostable Rca 2 protein variant may also comprise an amino acid sequence selected from the amino acid sequences of SEQ ID NO: 32 or 35 and further comprising a chloroplast targeting peptide, and an amino acid sequence having at least 90% identity to the amino acid sequences of SEQ ID NO: 32 or 35, further comprising a chloroplast targeting peptide, and comprising at least one amino acid selected from (i) an isoleucine at a position corresponding to position 59 of SEQ ID NO: 32 or 35, (ii) an aspartic acid at a position corresponding to position 73 of SEQ ID NO: 32 or 35, (iii) an isoleucine at a position corresponding to position 160 of SEQ ID NO: 32 or 35, (iv) an arginine at a position corresponding to position 265 of SEQ ID NO: 32 or 35, (v) a proline at a position corresponding to position 270 of SEQ ID NO: 32 or 35, (vi) a leucine at a position corresponding to position 277 of SEQ ID NO: 32 or 35, (vii) a glutamic acid at a position corresponding to position 307 of SEQ ID NO: 32 or 35, (viii) an isoleucine at a position corresponding to position 334 of SEQ ID NO: 32 or 35, (ix) a lysine at a position corresponding to position 359 of SEQ ID NO: 32 or 35, (x) a leucine at a position corresponding to position 361 of SEQ ID NO: 32 or 35 and (xi) a glutamic acid at a position corresponding to position 363 of SEQ ID NO: 32 or 35.

It is understood that thermostable Rca 2 protein variant according to the invention may comprise an amino acid sequence having at least 90% identity to the amino acid sequences of SEQ ID NOs: 30, 32, 33 or 35 and comprising at least one, or at least two, or at least three, or at least four, or at least five, or at least six, or at least seven, or at least eight, or at least nine, or at least ten, or all of the amino acid residues identified as conserved in thermostable variants of Rca proteins from various plant species and listed herein as AA1, AA2, AA3, AA4, AA5, AA6, AA7, AA8, AA9, AA10 and AA11.

A thermostable Rca 2 protein variant may comprise an amino acid sequence having at least 80%, or at least 85%, or at least 90%, or at least 91%, or at least 92%, or at least 93%, or at least 94%, or at least 95%, or at least 96%, or at least 97%, or at least 98%, or at least 99%, or at least 100% sequence identity to the amino acid sequences of SEQ ID NOs: 30, 32, 33 or 35 and comprise at least one of the amino acid residues identified as conserved in thermostable variants of Rca proteins from various plant species and listed herein as AA1, AA2, AA3, AA4, AA5, AA6, AA7, AA8, AA9, AA10 and AA11.

A thermostable Rca 2 protein variant may comprise an amino acid sequence having at least 80%, or at least 85%, or at least 90%, or at least 91%, or at least 92%, or at least 93%, or at least 94%, or at least 95%, or at least 96%, or at least 97%, or at least 98%, or at least 99%, or at least 100% sequence identity to the amino acid sequences of SEQ ID NOs: 30, 32, 33 or 35 and comprise at least two of the amino acid residues identified as conserved in thermostable variants of Rca proteins from various plant species and listed herein as AA1, AA2, AA3, AA4, AA5, AA6, AA7, AA8, AA9, AA10 and AA11.

A thermostable Rca 2 protein variant may comprise an amino acid sequence having at least 80%, or at least 85%, or at least 90%, or at least 91%, or at least 92%, or at least 93%, or at least 94%, or at least 95%, or at least 96%, or at least 97%, or at least 98%, or at least 99%, or at least 100% sequence identity to the amino acid sequences of SEQ ID NOs: 30, 32, 33 or 35 and comprise at least three of the amino acid residues identified as conserved in thermostable variants of Rca proteins from various plant species and listed herein as AA1, AA2, AA3, AA4, AA5, AA6, AA7, AA8, AA9, AA10 and AA11.

A thermostable Rca 2 protein variant may comprise an amino acid sequence having at least 80%, or at least 85%, or at least 90%, or at least 91%, or at least 92%, or at least 93%, or at least 94%, or at least 95%, or at least 96%, or at least 97%, or at least 98%, or at least 99%, or at least 100% sequence identity to the amino acid sequences of SEQ ID NOs: 30, 32, 33 or 35 and comprise at least four of the amino acid residues identified as conserved in thermostable variants of Rca proteins from various plant species and listed herein as AA1, AA2, AA3, AA4, AA5, AA6, AA7, AA8, AA9, AA10 and AA11.

A thermostable Rca 2 protein variant may comprise an amino acid sequence having at least 80%, or at least 85%, or at least 90%, or at least 91%, or at least 92%, or at least 93%, or at least 94%, or at least 95%, or at least 96%, or at least 97%, or at least 98%, or at least 99%, or at least 100% sequence identity to the amino acid sequences of SEQ ID NOs: 30, 32, 33 or 35 and comprise at least five of the amino acid residues identified as conserved in thermostable variants of Rca proteins from various plant species and listed herein as AA1, AA2, AA3, AA4, AA5, AA6, AA7, AA8, AA9, AA10 and AA11.

A thermostable Rca 2 protein variant may comprise an amino acid sequence having at least 80%, or at least 85%, or at least 90%, or at least 91%, or at least 92%, or at least 93%, or at least 94%, or at least 95%, or at least 96%, or at least 97%, or at least 98%, or at least 99%, or at least 100% sequence identity to the amino acid sequences of SEQ ID NOs: 30, 32, 33 or 35 and comprise at least six of the amino acid residues identified as conserved in thermostable variants of Rca proteins from various plant species and listed herein as AA1, AA2, AA3, AA4, AA5, AA6, AA7, AA8, AA9, AA10 and AA11.

A thermostable Rca 2 protein variant may comprise an amino acid sequence having at least 80%, or at least 85%, or at least 90%, or at least 91%, or at least 92%, or at least 93%, or at least 94%, or at least 95%, or at least 96%, or at least 97%, or at least 98%, or at least 99%, or at least 100% sequence identity to the amino acid sequences of SEQ ID NOs: 30, 32, 33 or 35 and comprise at least seven of the amino acid residues identified as conserved in thermostable variants of Rca proteins from various plant species and listed herein as AA1, AA2, AA3, AA4, AA5, AA6, AA7, AA8, AA9, AA10 and AA11.

A thermostable Rca 2 protein variant may comprise an amino acid sequence having at least 80%, or at least 85%, or at least 90%, or at least 91%, or at least 92%, or at least 93%, or at least 94%, or at least 95%, or at least 96%, or at least 97%, or at least 98%, or at least 99%, or at least 100% sequence identity to the amino acid sequences of SEQ ID NOs: 30, 32, 33 or 35 and comprise at least eight of the amino acid residues identified as conserved in thermostable variants of Rca proteins from various plant species and listed herein as AA1, AA2, AA3, AA4, AA5, AA6, AA7, AA8, AA9, AA10 and AA11.

A thermostable Rca 2 protein variant may comprise an amino acid sequence having at least 80%, or at least 85%, or at least 90%, or at least 91%, or at least 92%, or at least 93%, or at least 94%, or at least 95%, or at least 96%, or at least 97%, or at least 98%, or at least 99%, or at least 100% sequence identity to the amino acid sequences of SEQ ID NOs: 30, 32, 33 or 35 and comprise at least nine of the amino acid residues identified as conserved in thermostable variants of Rca proteins from various plant species and listed herein as AA1, AA2, AA3, AA4, AA5, AA6, AA7, AA8, AA9, AA10 and AA11.

A thermostable Rca 2 protein variant may comprise an amino acid sequence having at least 80%, or at least 85%, or at least 90%, or at least 91%, or at least 92%, or at least 93%, or at least 94%, or at least 95%, or at least 96%, or at least 97%, or at least 98%, or at least 99%, or at least 100% sequence identity to the amino acid sequences of SEQ ID NOs: 30, 32, 33 or 35 and comprise at least ten of the amino acid residues identified as conserved in thermostable variants of Rca proteins from various plant species and listed herein as AA1, AA2, AA3, AA4, AA5, AA6, AA7, AA8, AA9, AA10 and AA11.

In addition, it is clear that thermostable variants of Rca 2 proteins, wherein one or more amino acid residues have been deleted, substituted or inserted, can also be used to the same effect in the methods according to the invention, provided that the central ATPase domain (the AAA+ module), the C-terminal domain, the N-linker, Walker A, Walker B motives, the Rubisco interaction loop, the Rca-Rca interface and the tyrosine (Y) at the amino acid positions equivalent to position 406 of SEQ ID NO: 2, are not affected by the deletion, substitution or insertion of amino-acid.

Suitable for the invention are chloroplast targeting peptides which enable the subcellular targeting of the Rca proteins according to the invention to the chloroplast. Chloroplast transit peptide, chloroplast targeting sequence and stromal-targeting transit peptide are equivalent terms. Chloroplast targeting peptides are recognizable based on the presence of three domains: an uncharged N-terminal domain of about 10 residues beginning with a methionine followed by an alanine and terminating with a glycine or a proline, a central domain lacking acidic residues but enriched in serines and threonines and a C-terminal domain enriched in arginines and forming an amphiphilic p strand (Bruce, 2000, trends in cell biology, Vol 10, 440-447).

Such chloroplast targeting peptides are identified herein as the amino acid sequence from position 1 to position 47 of SEQ ID NOs: 8, 47 and 49, the amino acid sequence from position 1 to position 46 of SEQ ID NOs: 2, 6, 30, 33, 39, 41, 43, and 45, the amino acid sequence from position 1 to position 46 of SEQ ID NO: 11, the amino acid sequence from position 1 to position 46 of SEQ ID NO: 12, the amino acid sequence from position 1 to position 55 of SEQ ID NO: 13, the amino acid sequence from position 1 to position 56 of SEQ ID NO: 14, the amino acid sequence from position 1 to position 57 of SEQ ID NO: 15, the amino acid sequence from position 1 to position 50 of SEQ ID NO: 16, the amino acid sequence from position 1 to position 57 of SEQ ID NO: 17, the amino acid sequence from position 1 to position 57 of SEQ ID NO: 20, the amino acid sequence from position 1 to position 58 of SEQ ID NO: 21, the amino acid sequence from position 1 to position 59 of SEQ ID NO: 22, the amino acid sequence from position 1 to position 56 of SEQ ID NO: 23, the amino acid sequence from position 1 to position 51 of SEQ ID NO: 24, the amino acid sequence from position 1 to position 66 of SEQ ID NO: 25, the amino acid sequence from position 1 to position 57 of SEQ ID NO: 26, the amino acid sequence from position 1 to position 55 of SEQ ID NO: 27, the amino acid sequence from position 1 to position 74 of SEQ ID NO: 28.

Also suitable for the invention are chloroplast targeting peptides having an amino acid sequence having at least 70%, at least 80%, at least 85%, at least 90%, at least 92%, at least 95%, at least 98%, at least 99% identity to the amino acid sequences of SEQ ID NO: 30 from position 1 to position 46 or the amino acid sequence of SEQ ID NO: 8 from position 1 to position 47. A chloroplast targeting peptide having an amino acid sequence having at least 80% sequence identity to the amino acid sequences of SEQ ID NO: 30 from position 1 to position 46 or the amino acid sequence of SEQ ID NO: 8 from position 1 to position 47 can thus be a chloroplast targeting peptide having an amino acid sequence having at least at least 80%, or at least 85%, or at least 90%, at least 92%, at least 95%, at least 98%, at least 99% or even 100% sequence identity to the amino acid sequences of SEQ ID NO: 30 from position 1 to position 46 or the amino acid sequence of SEQ ID NO: 8 from position 1 to position 47.

In addition, the nucleic acid encoding a thermostable Rca 2 protein variant may comprise a coding nucleotide sequence selected from (a) the nucleotide sequence of SEQ ID Nos: 31, 34, 36 or 37, or the complement thereof, (b) a nucleotide sequence having at least 60% identity with the nucleotide sequence of SEQ ID NOs: 31, 34, 36 or 37, or the complement thereof.

SEQ ID NOs: 31 and 36 represents the nucleotide sequence encoding the amino acid sequence of SEQ ID NO: 30. SEQ ID NOs: 31 and 36 therefore represent the coding nucleotide sequence of the thermostable Rca 2P gene variant encoding all eleven amino acid residues identified as conserved in thermostable variants of Rca proteins from various plant species. SEQ ID NOs: 34 and 37 represents the nucleotide sequence encoding the amino acid sequence of SEQ ID NO: 33. SEQ ID NOs: 34 and 37 therefore represent the coding nucleotide sequence of the thermostable Rca 2P gene variant encoding eight of the eleven amino acid residues identified as conserved in thermostable variants of Rca proteins from various plant species. These eight amino acid residues are (a) AA1, (b) AA3, (d) AA4, (e) AA5, (f) AA8, (g) AA9, (h) A A10 and (i) AA11.

Suitable for the invention are nucleic acids encoding an Rca 2 protein, which comprise a nucleotide sequence having at least 40%, at least 50%, or at least 60%, or at least 70%, or at least 80%, or at least 85%, or at least 90%, or at least 95%, or at least 98% sequence identity to the herein described gene and are also referred to as variants.

A nucleic acid comprising a nucleotide sequence having at least 60% sequence identity to SEQ ID NOs: 31, 34, 36 or 37 can thus be a nucleic acid comprising a nucleotide sequence having at least 60%, or at least 70%, or at least 80%, or at least 85%, or at least 90%, or at least 95%, or at least 98%, or 100% sequence identity to SEQ ID NOs: 31, 34, 36 or 37 respectively.

### Thermostable Rca 2 Allele

The thermostable Rca 2 protein variant may be encoded by a thermostable allele of an Rca 2 gene.

In an embodiment, said thermostable allele may comprise (a) a coding nucleotide sequence of SEQ ID NOs: 31, 34, 36 or 37, or (b) a coding nucleotide sequence having at least 60% identity to SEQ ID NO: 31, 34, 36 or 37 and encoding a protein comprising at least one amino acid selected from (i) an isoleucine at a position corresponding to position 59 of SEQ ID NO: 32 or 35, (ii) an aspartic acid at a position corresponding to position 73 of SEQ ID NO: 32 or 35, (iii) an isoleucine at a position corresponding to position 160 of SEQ ID NO: 32 or 35, (iv) an arginine at a position corresponding to position 265 of SEQ ID NO: 32 or 35, (v) a proline at a position corresponding to position 270 of SEQ ID NO: 32 or 35, (vi) a leucine at a position corresponding to position 277 of SEQ ID NO: 32 or 35, (vii) a glutamic acid at a position corresponding to position 307 of SEQ ID NO: 32 or 35, (viii) an isoleucine at a position corresponding to position 334 of SEQ ID NO: 32 or 35, (ix) a lysine at a position corresponding to position 359 of SEQ ID NO: 32 or 35, (x) a leucine at a position corresponding to position 361 of SEQ ID NO: 32 or 35 and (xi) a glutamic acid at a position corresponding to position 363 of SEQ ID NO: 32 or 35.

The thermostable allele of a wheat Rca 2 gene is also provided, wherein the wheat Rca 2 gene is the Rca 2β gene from the subgenome B, A or D or the Rca 2α gene from the subgenome B, A or D.

Using the technologies of gene editing, endogenous alleles in a plant encoding a non-thermostable Rca 2 protein can be converted to thermostable Rca 2 alleles by making the desired changes (missense mutations) to existing Rca 2 genes, or by replacing one or more endogenous sequences encoding non-thermostable Rca 2 proteins with sequences encoding thermostable Rca 2 proteins, e.g. as described herein (deletion and insertion mutations).

An endogenous allele in a cereal plant, such as wheat, encoding a non-thermostable Rca 2 protein can also be converted to a thermostable Rca 2 allele by making the desired changes (missense mutations) to existing Rca 2 genes using mutagenesis.

### Recombinant Genes and Vectors

In yet another aspect, a recombinant gene comprising the following operably linked elements (a) a promoter, preferably expressible in plants, (b) a nucleic acid encoding an Rca protein selected from (i) a Rca 1β protein and variants thereof, and (ii) a thermostable Rca 2 protein variant and, optionally (c) a transcription termination and polyadenylation region, preferably a transcription termination and polyadenylation region functional in plants. In further embodiments, the Rca 1β protein and nucleic acids, and variants thereof, comprise the amino acid sequences and nucleotide sequences as described above and the thermostable Rca 2 protein and nucleic acid variants comprise an amino acid sequences and nucleotide sequences according to the invention. In a further embodiment, said promoter is a constitutive promoter, tissue-specific promoter or an inducible promoter. In yet a further embodiment, the promoter may be a green tissue specific promoter, a mesophyll specific promoter, a light-induced promoter or a temperature induced promoter.

Furthermore, a recombinant gene capable of suppressing specifically the expression of the endogenous Rca 2 genes is also provided which comprises the following operably linked elements (a) a promoter, preferably expressible in plants, (b) a nucleic acid which when transcribed yields an RNA molecule inhibitory to the endogenous Rca 2 genes encoding non-thermostable Rca 2 proteins but not inhibitory to genes encoding thermostable Rca protein variants; and, optionally (c) a transcription termination and polyadenylation region, preferably a transcription termination and polyadenylation region functional in plants. In a subsequent embodiment, said endogenous Rca 2 genes comprise the coding nucleotide sequence of SEQ ID NO: 1 or a coding nucleotide sequence having at least 60% identity with the nucleotide sequence of SEQ ID NO: 1.

Such inhibitory RNA molecule can reduce the expression of a gene for example through the mechanism of RNA-mediated gene silencing. It can be a silencing RNA downregulating expression of a target gene. As used herein, “silencing RNA” or “silencing RNA molecule” refers to any RNA molecule, which upon introduction into a plant cell, reduces the expression of a target gene. Such silencing RNA may e.g. be so-called “antisense RNA”, whereby the RNA molecule comprises a sequence of at least 20 consecutive nucleotides having 95% sequence identity to the complement of the sequence of the target nucleic acid, preferably the coding sequence of the target gene. However, antisense RNA may also be directed to regulatory sequences of target genes, including the promoter sequences and transcription termination and polyadenylation signals. Silencing RNA further includes so-called “sense RNA” whereby the RNA molecule comprises a sequence of at least 20 consecutive nucleotides having 95% sequence identity to the sequence of the target nucleic acid. Other silencing RNA may be “unpolyadenylated RNA” comprising at least 20 consecutive nucleotides having 95% sequence identity to the complement of the sequence of the target nucleic acid, such as described in WO01/12824 or U.S. Pat. No. 6,423,885 (both documents herein incorporated by reference). Yet another type of silencing RNA is an RNA molecule as described in WO03/076619 (herein incorporated by reference) comprising at least 20 consecutive nucleotides having 95% sequence identity to the sequence of the target nucleic acid or the complement thereof, and further comprising a largely-double stranded region as described in WO03/076619 (including largely double stranded regions comprising a nuclear localization signal from a viroid of the Potato spindle tuber viroid-type or comprising CUG trinucleotide repeats). Silencing RNA may also be double stranded RNA comprising a sense and antisense strand as herein defined, wherein the sense and antisense strand are capable of base-pairing with each other to form a double stranded RNA region (preferably the said at least 20 consecutive nucleotides of the sense and antisense RNA are complementary to each other). The sense and antisense region may also be present within one RNA molecule such that a hairpin RNA (hpRNA) can be formed when the sense and antisense region form a double stranded RNA region. hpRNA is well-known within the art (see e.g WO99/53050, herein incorporated by reference). The hpRNA may be classified as long hpRNA, having long, sense and antisense regions which can be largely complementary, but need not be entirely complementary (typically larger than about 200 bp, ranging between 200-1000 bp). hpRNA can also be rather small ranging in size from about 30 to about 42 bp, but not much longer than 94 bp (see WO04/073390, herein incorporated by reference). Silencing RNA may also be artificial micro-RNA molecules as described e.g. in WO2005/052170, WO2005/047505 or US 2005/0144667, or ta-siRNAs as described in WO2006/074400 (all documents incorporated herein by reference). Said RNA capable of modulating the expression of a gene can also be an RNA ribozyme.

The phrase “operably linked” refers to the functional spatial arrangement of two or more nucleic acid regions or nucleic acid sequences. For example, a promoter region may be positioned relative to a nucleic acid sequence such that transcription of a nucleic acid sequence is directed by the promoter region. Thus, a promoter region is “operably linked” to the nucleic acid sequence. “Functionally linked” is an equivalent term.

A “transcription termination and polyadenylation region” as used herein is a sequence that controls the cleavage of the nascent RNA, whereafter a poly(A) tail is added at the resulting RNA 3′ end, functional in plant cells. Transcription termination and polyadenylation signals functional in plant cells include, but are not limited to, 3′nos, 3′35S, 3′his and 3′g7.

As used herein, the term “plant-expressible promoter” means a DNA sequence that is capable of controlling (initiating) transcription in a plant cell. This includes any promoter of plant origin, but also any promoter of non-plant origin which is capable of directing transcription in a plant cell, i.e., certain promoters of viral or bacterial origin such as the CaMV35S (Harpster et al. (1988) Mol Gen Genet. 212(1):182-90, the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al. (1996) Plant Cell 8(1):15-30), stem-specific promoters (Keller et al., (1988) EMBO J. 7(12): 3625-3633), leaf specific promoters (Hudspeth et al. (1989) Plant Mol Biol. 12: 579-589), mesophyl-specific promoters (such as the light-inducible Rubisco promoters), root-specific promoters (Keller et al. (1989) Genes Dev. 3: 1639-1646), tuber-specific promoters (Keil et al. (1989) EMBO J. 8(5): 1323-1330), vascular tissue specific promoters (Peleman et al. (1989) Gene 84: 359-369), stamen-selective promoters (WO 89/10396, WO 92/13956), dehiscence zone specific promoters (WO 97/13865) and the like.

Suitable promoters for the invention are constitutive plant-expressible promoters. Constitutive plant-expressible promoters are well known in the art, and include the CaMV35S promoter (Harpster et al. (1988) Mol Gen Genet. 212(1):182-90), Actin promoters, such as, for example, the promoter from the Rice Actin gene (McElroy et al., 1990, Plant Cell 2:163), the promoter of the Cassava Vein Mosaic Virus (Verdaguer et al., 1996 Plant Mol. Biol. 31: 1129), the GOS promoter (de Pater et al., 1992, Plant J. 2:837), the Histone H3 promoter (Chaubet et al., 1986, Plant Mol Biol 6:253), the Agrobacterium tumefaciens Nopaline Synthase (Nos) promoter (Depicker et al., 1982, J. Mol. Appl. Genet. 1: 561), or Ubiquitin promoters, such as, for example, the promoter of the maize Ubiquitin-1 gene (Christensen et al., 1992, Plant Mol. Biol. 18:675).

A further promoter suitable for the invention is the endogenous promoter driving expression of the gene encoding an Rca protein.

Any of the nucleic acid sequences described above may be provided in a recombinant vector. A recombinant vector typically comprises, in a 5′ to 3′ orientation: a promoter to direct the transcription of a nucleic acid sequence and a nucleic acid sequence. The recombinant vector may further comprise a 3′ transcriptional terminator, a 3′ polyadenylation signal, other untranslated nucleic acid sequences, transit and targeting nucleic acid sequences, selectable markers, enhancers, and operators, as desired. The wording “5′ UTR” refers to the untranslated region of DNA upstream, or 5′ of the coding region of a gene and “3′ UTR” refers to the untranslated region of DNA downstream, or 3′ of the coding region of a gene. Means for preparing recombinant vectors are well known in the art. Methods for making recombinant vectors particularly suited to plant transformation are described in U.S. Pat. Nos. 4,971,908, 4,940,835, 4,769,061 and 4,757,011. Typical vectors useful for expression of nucleic acids in higher plants are well known in the art and include vectors derived from the tumor-inducing (Ti) plasmid of Agrobacterium tumefaciens. One or more additional promoters may also be provided in the recombinant vector. These promoters may be operably linked, for example, without limitation, to any of the nucleic acid sequences described above. Alternatively, the promoters may be operably linked to other nucleic acid sequences, such as those encoding transit peptides, selectable marker proteins, or antisense sequences. These additional promoters may be selected on the basis of the cell type into which the vector will be inserted. Also, promoters which function in bacteria, yeast, and plants are all well taught in the art. The additional promoters may also be selected on the basis of their regulatory features. Examples of such features include enhancement of transcriptional activity, inducibility, tissue specificity, and developmental stage-specificity.

The recombinant vector may also contain one or more additional nucleic acid sequences. These additional nucleic acid sequences may generally be any sequences suitable for use in a recombinant vector. Such nucleic acid sequences include, without limitation, any of the nucleic acid sequences, and modified forms thereof, described above. The additional structural nucleic acid sequences may also be operably linked to any of the above described promoters. The one or more structural nucleic acid sequences may each be operably linked to separate promoters. Alternatively, the structural nucleic acid sequences may be operably linked to a single promoter (i.e. a single operon).

### Methods and Uses

In one aspect, the invention provides a method for increasing the ratio of a thermostable Rca (Rubisco Activase) protein in cereals comprising (a) providing to cells of a cereal plant a recombinant gene comprising as operably linked elements a promoter, preferably expressible in plants; a nucleic acid encoding an Rca 13 protein and variants thereof or encoding a thermostable Rca 2 protein variant and, optionally a transcription termination and polyadenylation region, preferably a transcription termination and polyadenylation region functional in plants; and reducing the expression of endogenous non-thermostable Rca 2 protein in said cereal plant cells, wherein said ratio is increased compared to a control cereal plant cell not comprising said recombinant gene; or (b) introducing into cells of a cereal plant at least one thermostable Rca 2 allele according to the invention, wherein said ratio is increased compared to a control cereal plant cell not comprising said thermostable Rca 2 allele. In a further embodiment, the cereal plant is a wheat plant.

In a further aspect, the Rca 1β protein and variants thereof comprise an amino acid sequence as described above and are encoded by nucleic acids comprising the coding nucleic acid sequences described above.

In another embodiment, said thermostable Rca 2 protein variants comprise an amino acid sequence as described above and is encoded by nucleic acids comprising the coding nucleic acid sequences described above.

“Increasing the ratio of thermostable Rca” as used herein mean increasing the relative abundance of thermostable Rca proteins over the overall abundance of Rca proteins (thermostable and non-thermostable). This can be achieved by increasing the abundance of thermostable Rca proteins, by decreasing the abundance of non-thermostable Rca proteins and/or by both increasing the abundance of thermostable Rca proteins and decreasing the abundance of non-thermostable Rca proteins. The increased ratio of thermostable Rca may be of at least about or about 15%, at least about or about 30%, at least about or about 45%, at least about or about 60%, at least about or about 75%, at least about or about 90%, or at least about or about 100%. The increased ratio of thermostable Rca may be between about 15% and about 30%, between about 15% and about 45%, between about 15% and about 60%, between about 15% and about 75%, between about 15% and about 90%, between about 15% and about 100%, between about 30% and about 45%, between about 30% and about 60%, between about 30% and about 75%, between about 30% and about 90%, between about 30% and about 100%, between about 45% and about 60%, between about 45% and about 75%, between about 45% and about 90%, between about 45% and about 100%, between about 60% and about 75%, between about 60% and about 90%, between about 60% and about 100%, between about 75% and about 90%, between about 75% and about 100%, between about 90% and about 100%.

Furthermore by increasing the ratio of thermostable Rca proteins, the thermostability of the complexes comprising Rca proteins and Rubisco is increased proportionally (Shivhare and Mueller-Cajar, 2017, Plant Physiol DOI 10.1104/pp17.00554).

“Introducing” in connection with the present application relates to the placing of genetic information in a plant cell or plant by artificial means. This can be effected by any method known in the art for introducing RNA or DNA into plant cells, protoplasts, calli, roots, tubers, seeds, stems, leaves, seedlings, embryos, pollen and microspores, other plant tissues, or whole plants. “Introducing” also comprises stably integrating into the plant's genome. Introducing the recombinant gene can be performed by transformation or by crossing with a plant obtained by transformation or its descendant (also referred to as “introgression”). Introducing an allele also may be performed by mutagenesis of by gene editing.

The term “providing” may refer to introduction of an exogenous DNA molecule to a plant cell by transformation, optionally followed by regeneration of a plant from the transformed plant cell. The term may also refer to introduction of the recombinant DNA molecule by crossing of a transgenic plant comprising the recombinant DNA molecule with another plant and selecting progeny plants which have inherited the recombinant DNA molecule or transgene. Yet another alternative meaning of providing refers to introduction of the recombinant DNA molecule by techniques such as protoplast fusion, optionally followed by regeneration of a plant from the fused protoplasts.

The recombinant gene may be provided to a plant cell by methods well-known in the art.

The term “transformation” herein refers to the introduction (or transfer) of nucleic acid into a recipient host such as a plant or any plant parts or tissues including plant cells, protoplasts, calli, roots, tubers, seeds, stems, leaves, fibers, seedlings, embryos and pollen. Plants containing the transformed nucleic acid sequence are referred to as “transgenic plants”. Transformed, transgenic and recombinant refer to a host organism such as a plant into which a heterologous nucleic acid molecule (e.g. an expression cassette or a recombinant vector) has been introduced. The nucleic acid can be stably integrated into the genome of the plant.

As used herein, the phrase “transgenic plant” refers to a plant having a nucleic acid stably integrated into a genome of the plant, for example, the nuclear or plastid genomes. In other words, plants containing transformed nucleic acid sequence are referred to as “transgenic plants” and includes plants directly obtained from transformation and their descendants (Tx generations). Transgenic and recombinant refer to a host organism such as a plant into which a heterologous nucleic acid molecule (e.g. the promoter, the recombinant gene or the vector as described herein) has been introduced. The nucleic acid can be stably integrated into the genome of the plant.

It will be clear that the methods of transformation used are of minor relevance to the current invention. Transformation of plants is now a routine technique. Advantageously, any of several transformation methods may be used to introduce the nucleic acid/gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens et al. (1982) Nature 296: 72-74; Negrutiu et al. (1987) Plant. Mol. Biol. 8: 363-373); electroporation of protoplasts (Shillito et al. (1985) Bio/Technol. 3: 1099-1102); microinjection into plant material (Crossway et al. (1986) Mol. Gen. Genet. 202: 179-185); DNA or RNA-coated particle bombardment (Klein et al. (1987) Nature 327: 70) infection with (non-integrative) viruses and the like.

Different transformation systems could be established for various cereals: the electroporation of tissue, the transformation of protoplasts and the DNA transfer by particle bombardment in regenerable tissue and cells (for an overview see Jane, Euphytica 85 (1995), 35-44). The transformation of wheat has been described several times in literature (for an overview see Maheshwari, Critical Reviews in Plant Science 14 (2) (1995), 149-178, Nehra et al., Plant J. 5 (1994), 285-297).

The recombinant DNA molecules according to the invention may be provided to plants in a stable manner or in a transient manner using methods well known in the art. The recombinant genes may be introduced into plants, or may be generated inside the plant cell as described e.g. in EP 1339859.

“Control plant” as used herein refers to a plant genetically resembling the tested plant but not carrying the recombinant gene, such as wild type plants or null segregant plants, or not carrying the mutant allele, such as wild type plants or wild type segregant plants.

The transformed plant cells and plants obtained by the methods described herein may be further used in breeding procedures well known in the art, such as crossing, selfing, and backcrossing. Breeding programs may involve crossing to generate an F1 (first filial) generation, followed by several generations of selfing (generating F2, F3, etc). The breeding program may also involve backcrossing (BC) steps, whereby the offspring is backcrossed to one of the parental lines, termed the recurrent parent.

The transformed plant cells and plants obtained by the methods disclosed herein may also be further used in subsequent transformation procedures, e. g. to introduce a further recombinant gene.

In a further embodiment, reducing the expression of endogenous non-thermostable Rca 2 proteins comprises introducing into cells of the cereal plant at least one knock out mutant Rca 2 allele according to the invention, or providing said cells of a cereal plant with a second recombinant gene capable of suppressing specifically the expression of the endogenous non-thermostable Rca 2 gene, as described above.

Suitable for the invention are methods for increasing the ratio of a thermostable Rca protein in cereals comprising introducing into cells of the cereal plant at least two, at least three, at least four, at least five or even all six knock out mutant Rca 2 alleles according to the invention. Such at least two knock out mutant Rca 2 alleles may be two knock out mutant Rca 2 alleles from the subgenome B, two knock out mutant Rca 2 alleles from the subgenome D, two knock out mutant Rca 2 alleles from the subgenome A, one knock out mutant Rca 2 allele from the subgenome B and one knock out mutant Rca 2 allele from the subgenome D, one knock out mutant Rca 2 allele from the subgenome B and one knock out mutant Rca 2 allele from the subgenome A or one knock out mutant Rca 2 allele from the subgenome D and one knock out mutant Rca 2 allele from the subgenome A. Such at least three knock out mutant Rca 2 alleles may be two knock out mutant Rca 2 alleles from the subgenome B and one knock out mutant Rca 2 allele from the subgenome A, two knock out mutant Rca 2 alleles from the subgenome B and one knock out mutant Rca 2 allele from the subgenome D, two knock out mutant Rca 2 alleles from the subgenome D and one knock out mutant Rca 2 allele from the subgenome B, two knock out mutant Rca 2 alleles from the subgenome D and one knock out mutant Rca 2 allele from the subgenome A, two knock out mutant Rca 2 alleles from the subgenome A and one knock out mutant Rca 2 allele from the subgenome B, two knock out mutant Rca 2 alleles from the subgenome A and one knock out mutant Rca 2 allele from the subgenome D or one knock out mutant Rca 2 alleles from the subgenome B, one knock out mutant Rca 2 allele from the subgenome A and one knock out mutant Rca 2 allele from the subgenome D.

Such at least four knock out mutant Rca 2 alleles may be two knock out mutant Rca 2 alleles from the subgenome B and two knock out mutant Rca 2 alleles from the subgenome A, two knock out mutant Rca 2 alleles from the subgenome B and two knock out mutant Rca 2 allele from the subgenome D, or two knock out mutant Rca 2 alleles from the subgenome D and two knock out mutant Rca 2 allele from the subgenome A. Such at least four knock out mutant Rca 2 alleles may also be two knock out mutant Rca 2 alleles from the subgenome B, one knock out mutant Rca 2 alleles from the subgenome A and one knock out mutant Rca 2 alleles from the subgenome D, or two knock out mutant Rca 2 alleles from the subgenome D, one knock out mutant Rca 2 alleles from the subgenome A and one knock out mutant Rca 2 alleles from the subgenome B, or two knock out mutant Rca 2 alleles from the subgenome A, one knock out mutant Rca 2 alleles from the subgenome B and one knock out mutant Rca 2 alleles from the subgenome D. Such at least five knock out mutant Rca 2 alleles may be two knock out mutant Rca 2 alleles from the subgenome B, two knock out mutant Rca 2 alleles from the subgenome A and one knock out mutant Rca 2 allele from the subgenome D, or two knock out mutant Rca 2 alleles from the subgenome B, two knock out mutant Rca 2 alleles from the subgenome D and one knock out mutant Rca 2 allele from the subgenome A, or two knock out mutant Rca 2 alleles from the subgenome D, two knock out mutant Rca 2 alleles from the subgenome A and one knock out mutant Rca 2 allele from the subgenome B.

“reducing the expression of endogenous non-thermostable Rca 2 protein in cereal plant cells” refers to a reduction in the amount of a functional non-thermostable Rca 2 protein produced by the cell comprising the at least one knock out mutant Rca 2 allele according to the invention or the second recombinant gene capable of suppressing specifically the expression of the endogenous non-thermostable Rca 2 gene as described above, by at least 30%, 40%, 50%, 60%, 70%, 80%, 90% or even 100% as compared to the amount of the function non-thermostable Rca 2 protein produced by the cells not comprising the at least one knock out mutant Rca 2 allele according to the invention or the second recombinant gene capable of suppressing specifically the expression of the endogenous non-thermostable Rca 2 gene as described above.

Also suitable for the invention are methods for increasing the ratio of a thermostable Rca protein in cereals, such as wheat, comprising introducing into cells of the cereal plant at least two, at least three, at least four, at least five or even all six thermostable Rca 2 alleles according to the invention. Such at least two thermostable mutant Rca 2 alleles may be two thermostable Rca 2 alleles from the subgenome B, two thermostable Rca 2 alleles from the subgenome D, two thermostable Rca 2 alleles from the subgenome A, one thermostable Rca 2 allele from the subgenome B and one thermostable Rca 2 allele from the subgenome D, one thermostable Rca 2 allele from the subgenome B and one thermostable Rca 2 allele from the subgenome A or one thermostable Rca 2 allele from the subgenome D and one thermostable Rca 2 allele from the subgenome A. Such at least three thermostable Rca 2 alleles may be two thermostable Rca 2 alleles from the subgenome B and one thermostable Rca 2 allele from the subgenome A, two thermostable Rca 2 alleles from the subgenome B and one thermostable Rca 2 allele from the subgenome D, two thermostable Rca 2 alleles from the subgenome D and one thermostable Rca 2 allele from the subgenome B, two thermostable Rca 2 alleles from the subgenome D and one thermostable Rca 2 allele from the subgenome A, two thermostable Rca 2 alleles from the subgenome A and one thermostable Rca 2 allele from the subgenome B, two thermostable Rca 2 alleles from the subgenome A and one thermostable Rca 2 allele from the subgenome D or one thermostable Rca 2 alleles from the subgenome B, one thermostable Rca 2 allele from the subgenome A and one thermostable Rca 2 allele from the subgenome D.

Such at least four thermostable Rca 2 alleles may be two thermostable Rca 2 alleles from the subgenome B and two thermostable Rca 2 alleles from the subgenome A, two thermostable Rca 2 alleles from the subgenome B and two thermostable Rca 2 allele from the subgenome D, or two thermostable Rca 2 alleles from the subgenome D and two thermostable Rca 2 allele from the subgenome A. Such at least four thermostable Rca 2 alleles may also be two thermostable Rca 2 alleles from the subgenome B, one thermostable Rca 2 alleles from the subgenome A and one thermostable Rca 2 alleles from the subgenome D, or two thermostable Rca 2 alleles from the subgenome D, one thermostable Rca 2 alleles from the subgenome A and one thermostable Rca 2 alleles from the subgenome B, or two thermostable Rca 2 alleles from the subgenome A, one thermostable Rca 2 alleles from the subgenome B and one thermostable Rca 2 alleles from the subgenome D. Such at least five thermostable Rca 2 alleles may be two thermostable Rca 2 alleles from the subgenome B, two thermostable Rca 2 alleles from the subgenome A and one thermostable Rca 2 allele from the subgenome D, or two thermostable Rca 2 alleles from the subgenome B, two thermostable Rca 2 alleles from the subgenome D and one thermostable Rca 2 allele from the subgenome A, or two thermostable Rca 2 alleles from the subgenome D, two thermostable Rca 2 alleles from the subgenome A and one thermostable Rca 2 allele from the subgenome B.

In another aspect, a method for increasing thermotolerance of a cereal plant is provided which comprises increasing the ratio of a thermostable Rca protein and regenerating said plant, wherein the thermotolerance is increased compared to a cereal plant not comprising said increased ratio of a thermostable Rca protein. In a further embodiment, the ratio of a thermostable Rca protein is increased according to the method for increasing the ratio of a thermostable Rca (Rubisco Activase) protein in cereals described herein. In further embodiments, said thermostable Rca protein is an Rca 1β protein or variants thereof as described herein or said thermostable Rca protein is the thermostable Rca 2 protein variant according to the invention.

Under heat stress the non-thermotolerant Rca proteins dissociate from Rubisco. The Rubisco enzyme is therefore less active or even inactive and photosynthesis is reduced or stopped. The thermostable Rca proteins disclosed herein confer thermostability to the complex comprising Rca proteins and Rubisco. Such thermostable complex allows the Rubisco to remain active under heat stress and consequently to maintain photosynthetic activity. A plant thermotolerance can thus be measured by measuring photosynthetic activity of the plant. Methods for measuring photosynthetic activity in a plant are well known in the art (see for example Kalaji et al 2012 Photosynth Res 114:69-96).

In yet another aspect of the invention, a method for increasing yield of a cereal plant, such as a wheat plant, under heat stress conditions is provided, comprising increasing the ratio of a thermostable Rca protein and regenerating said plant, wherein the yield is increased compared to a cereal plant not comprising said increased ratio of a thermostable Rca protein. In a further embodiment, the ratio of a thermostable Rca protein is increased according to the method for increasing the ratio of a thermostable Rca (Rubisco Activase) protein in cereals described herein. In further embodiments, said thermostable Rca protein is a Rca 1β protein or variants thereof as described herein or said thermostable Rca protein is the thermostable Rca 2 protein variant according to the invention. The yield increased may be seed yield or thousand seed weight.

A method for producing a cereal plant, such as a wheat plant, with increased thermotolerance is furthermore provided, comprising increasing the ratio of a thermostable Rca protein as disclosed herein and regenerating said plant.

Also provided is the use of the thermostable Rca 2 protein variant according to the invention, the nucleic acid encoding a thermostable Rca 2 protein variant according to the invention, the recombinant gene according to the invention, the recombinant gene capable of suppressing specifically the expression of the endogenous Rca 2 genes described herein or the thermostable allele of a Rca 2 gene provided herewith to increase the ratio of a thermostable Rca protein in cereals, to increase thermotolerance of a cereal plant, to increase yield of a cereal plant under heat stress conditions or to produce a cereal plant with increased thermotolerance. Such cereal plant may be a wheat plant.

“Yield” as used herein can comprise yield of the plant or plant part which is harvested, such as biomass, or seed, including seed protein content, seed weight (measured as thousand seed weight), seed number. Increased yield can be increased yield per spike, increased yield per tiller, increased yield per plant, and increased yield per surface unit of cultivated land, such as yield per hectare. Yield can be increased by increasing, for example, the tolerance to abiotic stress conditions.

When the yield is the seed yield, the yield increase achieved with the method described herein compared to plants wherein the ratio of thermostable Rca protein is not increased may be of at least about 5%, at least about 6%, at least about 7%, at least about 8%, at least about 9% or at least about 10%. When the yield is the seed weight, the yield increase achieved with the method described herein compared to plants wherein the ratio of thermostable Rca protein is not increased may be of at least about 5%, at least about 6%, at least about 7% or at least about 8%, at least about 9% or at least about 10%.

“Stress” refers to non-optimal environmental conditions such as abiotic stress. Abiotic stress can comprise environmental stress factors such as drought, flood, extreme (high or low) temperatures, soil salinity or heavy metals, hypoxia, anoxia, osmotic stress, oxidative stress, low nutrient levels such as nitrogen or phosphorus.

“Heat stress” as used herein relates to the exposure of a plant to high temperatures for a specified time. Such high temperature may last only a few hours per day and may occur at least or up to 2, at least or up to 3, at least or up to 4, at least or up to 5, at least or up to 6, at least or up to 7, at least or up to 8, at least or up to 9, at least or up to 10, at least or up to 15 or at least or up to 20 days. It may as well be for a longer period such as at least or up to 3 weeks, at least or up to 4 weeks, at least or up to 5 weeks, at least or up to 6 weeks, at least or up to 2 months, or at least or up to 3 months. High temperatures for cereals, such as wheat, mean a temperature exceeding the optimum range for a cereal, such as wheat, and may be of at least about 28 degree Celsius (° C.), at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C. High temperatures for a cereal such as wheat may also be temperature between about 28° C. and about 30° C., between about 28° C. and about 32° C., between about 28° C. and about 34° C., between about 28° C. and about 36° C., between about 28° C. and about 38° C., between about 28° C. and about 40° C., between about 28° C. and about 42° C., between about 28° C. and about 45° C., between about 30° C. and about 32° C., between about 30° C. and about 34° C., between about 30° C. and about 36° C., between about 30° C. and about 38° C., between about 30° C. and about 40° C., between about 30° C. and about 42° C., between about 30° C. and about 45° C., between about 32° C. and about 34° C., between about 32° C. and about 36° C., between about 32° C. and about 38° C., between about 32° C. and about 40° C., between about 32° C. and about 42° C., between about 32° C. and about 45° C., between about 34° C. and about 36° C., between about 34° C. and about 38° C., between about 34° C. and about 40° C., between about 34° C. and about 42° C., between about 34° C. and about 45° C., between about 36° C. and about 38° C., between about 36° C. and about 40° C., between about 36° C. and about 42° C., between about 36° C. and about 45° C., between about 38° C. and about 40° C., between about 38° C. and about 42° C., between about 38° C. and about 45° C., between about 40° C. and about 42° C., between about 40° C. and about 45° C., or between about 42° C. and 45° C.

Another aspect of the invention provides a method of producing food, feed, such as meal, grain, starch, flour or protein, or an industrial product, such as biofuel, fiber, industrial chemicals, a pharmaceutical or a nutraceutical, said method comprising obtaining the plant according to the invention or a part thereof, and preparing the food, feed or industrial product from the plant or part thereof.

In case of a wheat plant or other cereal plant, examples of food products include flour, starch, leavened or unleavened breads, pasta, noodles, animal fodder, breakfast cereals, snack foods, cakes, malt, pastries, seitan and foods containing flour-based sauces.

Method of producing such food, feed or industrial product from wheat are well known in the art. For example, the flour is produced by grinding finely grains in a mill (see for example www.madehow.com/Volume-3/Flour.html) and the biofuel is produced from wheat straw or mixtures of wheat straw and wheat meal (see for example Erdei et al., Biotechnology for Biofuels, 2010, 3:16).

In yet another embodiment, a method of increasing the thermostability of a Rca 2 protein is provided, comprising introducing at least one amino acid substitution to the amino acid sequence of said Rca 2 protein, wherein the amino acid substitution is selected from (a) substituting or replacing a valine with an isoleucine at a position corresponding to position 59 of SEQ ID NO: 4, (b) substituting or replacing a glycine with an aspartic acid at a position corresponding to position 73 of SEQ ID NO: 4, (c) substituting or replacing a methionine with an isoleucine at a position corresponding to position 160 of SEQ ID NO: 4, (d) substituting or replacing a glutamine with an arginine at a position corresponding to position 265 of SEQ ID NO: 4, (e) substituting or replacing a serine with a proline at a position corresponding to position 270 of SEQ ID NO: 4, (f) substituting or replacing an isoleucine with a leucine at a position corresponding to position 277 of SEQ ID NO: 4, (g) substituting or replacing a serine with a glutamic acid at a position corresponding to position 307 of SEQ ID NO: 4, (h) substituting or replacing a valine with an isoleucine at a position corresponding to position 334 of SEQ ID NO: 4, (i) substituting or replacing threonine with a lysine at a position corresponding to position 359 of SEQ ID NO: 4, (j) substituting or replacing methionine with a leucine at a position corresponding to position 361 of SEQ ID NO: 4 and (k) substituting or replacing a glutamine with a glutamic acid at a position corresponding to position 363 of SEQ ID NO: 4, wherein the thermostability of the Rca 2 protein is increased compared to the Rca 2 protein not comprising any of the listed amino acid substitutions. In a further embodiment the thermostability of the Rca 2 protein is increased by about 7° C.

Suitable for the invention are increases in thermostability of the Rca 2 protein comprising said amino acid substitutions by at least about or about 3° C., at least about or about 4° C., at least about or about 5° C., at least about or about 6° C., at least about or about 7° C., at least about or about 8° C., at least about or about 9° C., at least about or about 10° C.

It is understood that the method described herein may comprise introducing at least two, at least three, at least four, at least five, at least six, at least seven, at least eight, at least nine, at least ten or even all eleven amino acid substitutions. As the amino acid substitutions listed above result in the introduction of one or more amino acid identified herein as relevant for the thermostability of an Rca protein (AA1, AA2, AA3, AA4, AA5, AA6, AA7, AA8, AA9, AA10 and AA11), it is clear that the different combinations of at least two, at least three, at least four, at least five, at least six, at least seven, at least eight, at least nine or at least ten amino acid substitutions correspond to the combinations of at least two, at least three, at least four, at least five, at least six, at least seven, at least eight, at least nine or at least ten amino acid residues identified as relevant for the thermostability of an Rca protein and listed herein.

In addition, a method for producing a thermostable Rca protein or thermostable Rca protein variant is herein provided, comprising culturing the host cell comprising the recombinant gene comprising the nucleic acid encoding a thermostable Rca protein or thermostable Rca protein variant as described above and isolating the protein produced.

Said host cell expresses or over-expresses the thermostable Rca protein or thermostable Rca protein variant of the invention. Accordingly, said protein of the invention is produced in and isolated from the host cell. In case that the host cell produces the protein of the invention and secretes it to the surrounding media, e. g. due to a suitable signal peptide attached to the protein, isolation denotes separation of the media comprising the protein from the host cell. Said media may then be the subject of further purification steps (see below).

Suitable conditions for culturing a prokaryotic or eukaryotic host are well known to the person skilled in the art. For example, suitable conditions for culturing bacteria are growing them under aeration in Luria Bertani (LB) medium. To increase the yield and the solubility of the expression product, the medium can be buffered or supplemented with suitable additives known to enhance or facilitate both. E. coli can be cultured from 4 to about 37° C., the exact temperature or sequence of temperatures depends on the molecule to be over-expressed. In general, the skilled person is also aware that these conditions may have to be adapted to the needs of the host and the requirements of the polypeptide expressed. In case an inducible promoter controls the nucleic acid of the invention in the vector present in the host cell, expression of the polypeptide can be induced by addition of an appropriate inducing agent Suitable expression protocols and strategies are known to the skilled person.

Suitable expression protocols for eukaryotic cells are well known to the skilled person and can be retrieved e.g. from Sambrook, 2001.

Suitable media for insect cell culture are e.g. TNM+10% FCS or SF900 medium. Insect cells are usually grown at 27° C. as adhesion or suspension culture.

Methods of isolation of the polypeptide produced are well-known in the art and comprise without limitation method steps such as ammonium sulphate precipitation, ion exchange chromatography, gel filtration chromatography (size exclusion chromatography), affinity chromatography, high pressure liquid chromatography (HPLC), reversed phase HPLC, disc gel electrophoresis or immunoprecipitation, see, for example, in Sambrook, 2001.

### Cells and Plants

Other embodiments provide a host cell, such as an E. coli cell, an Agrobacterium cell, a yeast cell, or a plant cell, comprising (a) the recombinant gene comprising a nucleic acid encoding a thermostable Rca protein according to the invention or the vector comprising this recombinant gene, (b) the recombinant gene capable of suppressing specifically the expression of the endogenous non-thermostable Rca 2 genes as described herein or the vector comprising this recombinant gene, or (c) the thermostable Rca 2 protein variant according to the invention.

Further embodiments provide a plant cell comprising (a) at least one knock out Rca 2 allele as described herein and/or (b) at least one thermostable Rca 2 allele according to the invention. In yet another embodiment the plant cell comprising the recombinant gene comprising a nucleic acid encoding a thermostable Rca protein according to the invention or the vector comprising this recombinant gene may further comprise the recombinant gene capable of suppressing specifically the expression of the endogenous non-thermostable Rca 2 genes as described herein or the vector comprising that recombinant gene or at least one knock out Rca 2 allele as described herein. The plant cell may be a cereal plant cell or a wheat plant cell.

In yet another embodiment a plant is provided that expresses the Rca 2 thermotolerant protein variant according to the invention. Said plant may be a cereal plant or a wheat plant.

Other nucleic acid sequences may also be introduced into the host cell along with the described recombinant genes described herein, e. g. also in connection with the vector of the invention. These other sequences may include 3′ transcriptional terminators, 3′ polyadenylation signals, other untranslated nucleic acid sequences, transit or targeting sequences, selectable markers, enhancers, and operators. Preferred nucleic acid sequences of the present invention, including recombinant vectors, structural nucleic acid sequences, promoters, and other regulatory elements, are described above.

In further embodiments, a plant is provided comprising any of the recombinant genes and alleles according to the invention. A further embodiment provides plant parts and seeds obtainable from the plant according to the invention. These plant parts and seeds comprise the recombinant genes or alleles described above. In another embodiment, the plants, plant parts or seeds according to the invention are wheat plants, plant parts or seeds.

The plant cell or plant comprising any of the recombinant gene according to the invention can be a plant cell or a plant comprising a recombinant gene of which either the promoter, or the heterologous nucleic acid sequence operably linked to said promoter, are heterologous with respect to the plant cell. Such plant cells or plants may be transgenic plant in which the recombinant gene is introduced via transformation. Alternatively, the plant cell of plant may comprise the promoter according to the invention derived from the same species operably linked to a nucleic acid which is also derived from the same species, i.e. neither the promoter nor the operably linked nucleic acid is heterologous with respect to the plant cell, but the promoter is operably linked to a nucleic acid to which it is not linked in nature. A recombinant gene can be introduced in the plant or plant cell via transformation, such that both the promoter and the operably linked nucleotide are at a position in the genome in which they do not occur naturally. Alternatively, the promoter according to the invention can be integrated in a targeted manner in the genome of the plant or plant cell upstream of an endogenous nucleic acid encoding an expression product of interest, i.e. to modulate the expression pattern of an endogenous gene. The promoter that is integrated in a targeted manner upstream of an endogenous nucleic acid can be integrated in cells of a plant species from which it is originally derived, or in cells of a heterologous plant species. Alternatively, a heterologous nucleic acid can be integrated in a targeted manner in the genome of the plant or plant cell downstream of the promoter according to the invention, such that said heterologous nucleic acid is expressed root-preferentially and is stress-inducible. Said heterologous nucleic acid is a nucleic acid which is heterologous with respect to the promoter, i.e. the combination of the promoter with said heterologous nucleic acid is not normally found in nature. Said heterologous nucleic acid may be a nucleic acid which is heterologous to said plant species in which it is inserted, but it may also naturally occur in said plant species at a different location in the plant genome. Said promoter or said heterologous nucleic acid can be integrated in a targeted manner in the plant genome via targeted sequence insertion, using, for example, the methods as described in WO2005/049842.

“Plants” encompasses “monocotyledonous plants”. “Monocotyledonous plants”, also known as “monocot plants” or “monocots” are well known in the art and are plants of which the seed typically has one cotyledon. Examples of monocotyledons plants are grasses, such as meadow grass (blue grass, Poa), forage grass such as festuca, lolium, temperate grass, such as Agrostis, and cereals, e.g., wheat, oats, rye, barley, rice, triticale, spelt, einkorn, emmer, durum wheat, kamut, sorghum, and maize (corn).

The plants according to the invention may be cereal plants. The cereal plants according to the invention may be wheat plants.

“Wheat” or “wheat plant” as used herein can be any variety useful for growing wheat. Examples of wheat are, but are not limited to, Triticum aestivum, Triticum aethiopicum, Triticum Compactum, Triticum dicoccoides, Triticum dicoccon, Triticum durum, Triticum monococcum, Triticum spelta, Triticum turgidum. “Wheat” furthermore encompasses spring and winter wheat varieties, with the winter wheat varieties being defined by a vernalization requirement to flower while the spring wheat varieties do not require such vernalization to flower.

“Plant parts” as used herein are parts of the plant, which can be cells, tissues or organs, such as seeds, severed parts such as roots, leaves, flowers, pollen, etc.

The plants according to the invention may additionally contain an endogenous or a transgene, which confers herbicide resistance, such as the bar or pat gene, which confer resistance to glufosinate ammonium (Liberty®, Basta® or Ignite®) [EP 0 242 236 and EP 0 242 246 incorporated by reference]; or any modified EPSPS gene, such as the 2mEPSPS gene from maize [EPO 508 909 and EP 0 507 698 incorporated by reference], or glyphosate acetyltransferase, or glyphosate oxidoreductase, which confer resistance to glyphosate (RoundupReady®), or bromoxynitril nitrilase to confer bromoxynitril tolerance, or any modified AHAS gene, which confers tolerance to sulfonylureas, imidazolinones, sulfonylaminocarbonyltriazolinones, triazolopyrimidines or pyrimidyl(oxy/thio)benzoates.

The plants or seeds of the plants according to the invention may be further treated with a chemical compound, such as a chemical compound selected from the following lists: Herbicides: Clethodim, Clopyralid, Diclofop, Ethametsulfuron, Fluazifop, Glufosinate, Glyphosate, Metazachlor, Quinmerac, Quizalofop, Tepraloxydim, Trifluralin. Fungicides/PGRs: Azoxystrobin, N-[9-(dichloromethylene)-1,2,3,4-tetrahydro-1,4-methanonaphthalen-5-yl]-3-(difluoromethyl)-1-methyl-1H-pyrazole-4-carboxamide (Benzovindiflupyr, Benzodiflupyr), Bixafen, Boscalid, Carbendazim, Carboxin, Chlormequat-chloride, Coniothryrium minitans, Cyproconazole, Cyprodinil, Difenoconazole, Dimethomorph, Dimoxystrobin, Epoxiconazole, Famoxadone, Fluazinam, Fludioxonil, Fluopicolide, Fluopyram, Fluoxastrobin, Fluquinconazole, Flusilazole, Fluthianil, Flutriafol, Fluxapyroxad, Iprodione, Isopyrazam, Mefenoxam, Mepiquat-chloride, Metalaxyl, Metconazole, Metominostrobin, Paclobutrazole, Penflufen, Penthiopyrad, Picoxystrobin, Prochloraz, Prothioconazole, Pyraclostrobin, Sedaxane, Tebuconazole, Tetraconazole, Thiophanate-methyl, Thiram, Triadimenol, Trifloxystrobin, Bacillus firmus, Bacillus firmus strain I-1582, Bacillus subtilis, Bacillus subtilis strain GB03, Bacillus subtilis strain QST 713, Bacillus pumulis, Bacillus. pumulis strain GB34. Insecticides: Acetamiprid, Aldicarb, Azadirachtin, Carbofuran, Chlorantraniliprole (Rynaxypyr), Clothianidin, Cyantraniliprole (Cyazypyr), (beta-)Cyfluthrin, gamma-Cyhalothrin, lambda-Cyhalothrin, Cypermethrin, Deltamethrin, Dimethoate, Dinetofuran, Ethiprole, Flonicamid, Flubendiamide, Fluensulfone, Fluopyram, Flupyradifurone, tau-Fluvalinate, Imicyafos, Imidacloprid, Metaflumizone, Methiocarb, Pymetrozine, Pyrifluquinazon, Spinetoram, Spinosad, Spirotetramate, Sulfoxaflor, Thiacloprid, Thiamethoxam, 1-(3-chloropyridin-2-yl)-N-[4-cyano-2-methyl-6-(methylcarbamoyl)phenyl]-3-{[5-(trifluoromethyl)-2H-tetrazol-2-yl]methyl}-1H-pyrazole-5-carboxamide, 1-(3-chloropyridin-2-yl)-N-[4-cyano-2-methyl-6-(methylcarbamoyl)phenyl]-3-{[5-(trifluoromethyl)-1H-tetrazol-1-yl]methyl}-1H-pyrazole-5-carboxamide, 1-{2-fluoro-4-methyl-5-[(2,2,2-trifluorethyl)sulfinyl]phenyl}-3-(trifluoromethyl)-1H-1,2,4-triazol-5-amine, (1E)-N-[(6-chloropyridin-3-yl)methyl]-N′-cyano-N-(2,2-difluoroethyl)ethanimidamide, Bacillus firmus, Bacillus firmus strain I-1582, Bacillus subtilis, Bacillus subtilis strain GB03, Bacillus subtilis strain QST 713, Metarhizium anisopliae F52.

Whenever reference to a “plant” or “plants” according to the invention is made, it is understood that also plant parts (cells, tissues or organs, seed pods, seeds, severed parts such as roots, leaves, flowers, pollen, etc.), progeny of the plants which retain the distinguishing characteristics of the parents, such as seed obtained by selfing or crossing, e.g. hybrid seed (obtained by crossing two inbred parental lines), hybrid plants and plant parts derived there from are encompassed herein, unless otherwise indicated.

In some embodiments, the plant cells of the invention as well as plant cells generated according to the methods of the invention, may be non-propagating cells.

The obtained plants according to the invention can be used in a conventional breeding scheme to produce more plants with the same characteristics or to introduce the same characteristic in other varieties of the same or related plant species, or in hybrid plants. The obtained plants can further be used for creating propagating material. Plants according to the invention can further be used to produce gametes, seeds, embryos, either zygotic or somatic, progeny or hybrids of plants obtained by methods of the invention. Seeds obtained from the plants according to the invention are also encompassed by the invention.

“Creating propagating material”, as used herein, relates to any means know in the art to produce further plants, plant parts or seeds and includes inter alia vegetative reproduction methods (e.g. air or ground layering, division, (bud) grafting, micropropagation, stolons or runners, storage organs such as bulbs, corms, tubers and rhizomes, striking or cutting, twin-scaling), sexual reproduction (crossing with another plant) and asexual reproduction (e.g. apomixis, somatic hybridization).

In certain jurisdictions, plants according to the invention, which however have been obtained exclusively by essentially biological processes, wherein a process for the production of plants is considered essentially biological if it consists entirely of natural phenomena such as crossing or selection, may be excluded from patentability. Plants according to the invention thus also encompass those plants not exclusively obtained by essentially biological processes.

The sequence listing contained in the file named “BCS18-2016_ST25.txt”, which is 178 kilobytes (size as measured in Microsoft Windows®), contains 69 sequences SEQ ID NO: 1 through SEQ ID NO: 69 is filed herewith by electronic submission and is incorporated by reference herein.

In the description and examples, reference is made to the following sequences:

### SEQUENCES

SEQ ID NO: 1: nucleotide sequence of the TaRca 2b from the subgenome B
SEQ ID NO: 2: amino acid sequence of the TaRca 2b from the subgenome B
SEQ ID NO: 3: nucleotide sequence of the TaRca 2b from the subgenome B minus the signal peptide
SEQ ID NO: 4: amino acid sequence of the TaRca 2b from the subgenome B minus the signal peptide
SEQ ID NO: 5: nucleotide sequence of the TaRca 2α from the subgenome B
SEQ ID NO: 6: amino acid sequence of the TaRca 2α from the subgenome B
SEQ ID NO: 7: nucleotide sequence of the TaRca 1b from the subgenome B
SEQ ID NO: 8: amino acid sequence of the TaRca 1b from the subgenome B
SEQ ID NO: 9: nucleotide sequence of the TaRca 1b from the subgenome B minus the signal peptide
SEQ ID NO: 10: amino acid sequence of the TaRca 1b from the subgenome B minus the signal peptide
SEQ ID NO: 11: amino acid sequence of the Rca from Oryza sativa BAA97584.1
SEQ ID NO: 12: amino acid sequence of the Rca from Oryza australiensis ANHI 1447.1
SEQ ID NO: 13: amino acid sequence of the Rca from Larrea tridentate Q7X999.1
SEQ ID NO: 14: amino acid sequence of the Rca from Musa acuminate XP_009419709.1
SEQ ID NO: 15: amino acid sequence of the Rca from Datisca glomerata AAC62207.1
SEQ ID NO: 16: amino acid sequence of the Rca from Theobroma cacao EOY07450.1
SEQ ID NO: 17: amino acid sequence of the Rca from Nicotiana tabacum AAA78277.1
SEQ ID NO: 18: amino acid sequence of the Rca from Gossypium hirsutum XP_016753736.1
SEQ ID NO: 19: amino acid consensus sequence of the Rca from the “warm” species
SEQ ID NO: 20: amino acid sequence of the Rca from Arabidopsis thaliana NP_850321.1
SEQ ID NO: 21: amino acid sequence of the Rca from Brassica oleracea AFH35543.1
SEQ ID NO: 22: amino acid sequence of the Rca from Picea sitchensis ABK25255.1
SEQ ID NO: 23: amino acid sequence of the Rca from Spinacia Oleracea AAA34038.1
SEQ ID NO: 24: amino acid sequence of the Rca from Fragaria vesca XP_004305457.1
SEQ ID NO: 25: amino acid sequence of the Rca from Arachis duranensis XP_015938754.1
SEQ ID NO: 26: amino acid sequence of the Rca from Brachypodium distachyon XP_003580722.1
SEQ ID NO: 27: amino acid sequence of the Rca from Arabis alpine KFK41750.1
SEQ ID NO: 28: amino acid sequence of the Rca from Mesembryanthemum crystallinum AAZ41846.1
SEQ ID NO: 29: amino acid consensus sequence of the Rca from the “cold” species
SEQ ID NO: 30: amino acid sequence of the TaRca 2b from the subgenome B with 11 amino acid permutations
SEQ ID NO: 31: nucleotide sequence of the TaRca 2b from the subgenome B with 11 amino acid permutations minus the signal peptide, codon optimized for expression in E. coli
SEQ ID NO: 32: amino acid sequence of the TaRca 2b from the subgenome B with 11 amino acid permutations minus the signal peptide
SEQ ID NO: 33: amino acid sequence of the TaRca 2b from the subgenome B with 8 amino acid permutations
SEQ ID NO: 34: nucleotide sequence of the TaRca 2b from the subgenome B with 8 amino acid permutations minus the signal peptide, codon optimized for expression in E. coli
SEQ ID NO: 35: amino acid sequence of the TaRca 2b from the subgenome B with 8 amino acid permutations minus the signal peptide
SEQ ID NO: 36: nucleotide sequence of the T-DNA PrcaOm::Rca 1β
SEQ ID NO: 37: nucleotide sequence of the T-DNA Prbcs::Rca 1β
SEQ ID NO: 38: nucleotide sequence of the TaRca 2b from the subgenome A
SEQ ID NO: 39: amino acid sequence of the TaRca 2b from the subgenome A
SEQ ID NO: 40: nucleotide sequence of the TaRca 2b from the subgenome D
SEQ ID NO: 41: amino acid sequence of the TaRca 2b from the subgenome D
SEQ ID NO: 42: nucleotide sequence of the TaRca 2a from the subgenome A
SEQ ID NO: 43: amino acid sequence of the TaRca 2a from the subgenome A
SEQ ID NO: 44: nucleotide sequence of the TaRca 2a from the subgenome D
SEQ ID NO: 45: amino acid sequence of the TaRca 2a from the subgenome D
SEQ ID NO: 46: nucleotide sequence of the TaRca 1b from the subgenome A
SEQ ID NO: 47: amino acid sequence of the TaRca 1b from the subgenome A
SEQ ID NO: 48: nucleotide sequence of the TaRca 1b from the subgenome D
SEQ ID NO: 49: amino acid sequence of the TaRca 1b from the subgenome D
SEQ ID NO: 50: TaRca-1b forward primer
SEQ ID NO: 51: TaRca-1b reverse primer
SEQ ID NO: 52: TaRca-2a forward primer
SEQ ID NO: 53: TaRca-2a reverse primer
SEQ ID NO: 54: TaRca-2b forward primer
SEQ ID NO: 55: TaRca-2b reverse primer
SEQ ID NO: 56: Ta54227 forward primer
SEQ ID NO: 57: Ta54227 reverse primer
SEQ ID NO: 58: Ta54238 forward primer
SEQ ID NO: 59: Ta54238 reverse primer
SEQ ID NO: 60: nucleotide sequence of the T-DNA PubiZm::hpRca2
SEQ ID NO: 61: nucleotide sequence of the guide RNA g1
SEQ ID NO: 62: nucleotide sequence of the guide RNA g2
SEQ ID NO: 63: nucleotide sequence of the guide RNA g13
SEQ ID NO: 64: nucleotide sequence of the guide RNA g9
SEQ ID NO: 65: nucleotide sequence of the guide RNA g14
SEQ ID NO: 66: nucleotide sequence of the guide RNA g15
SEQ ID NO: 67: nucleotide sequence of the guide RNA g16
SEQ ID NO: 68: nucleotide sequence of the guide RNA g17
SEQ ID NO: 69: nucleotide sequence of the guide RNA g18

### EXAMPLES

Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1—Determination of the Expression Profile of the 3 Wheat Rea Isoforms

### In Situ Heating Experiment

Triticum aestivum cv. Fielder (wheat) seeds were wet and placed on germination paper with stratification for 1 week at 4° C., followed by sowing in 17 cm diameter pots with potting soil. Pots were placed in a growth chamber with a 12-h photoperiod at a constant 22° C. and 300 μmol m⁻² s⁻¹ photosynthetic active radiation. 35 days after sowing half of the pots were relocated to an adjacent growth chamber with a 38° C. light period temperature. At 38 days after sowing all healthy leaf material was harvested in the middle of the light period and snap frozen in liquid N₂ before being stored at −80° C. until use. To determine Rca gene expression qRT-PCR was performed on leaf material using primers listed as SEQ ID NOs: 50 to 59 and a PCR cycle of 10 min at 95° C. followed by 40 cycles of 15 sec at 95° C., 60 sec at 60° C. and a melt curve of 15 sec at 95° C., 60 sec at 60° C., 60° C. to 95° C. in 0.3° C. increments and 15 sec at 95° C., and normalizing with the reference genes Ta54227 and Ta54238.

### Gene Expression Results

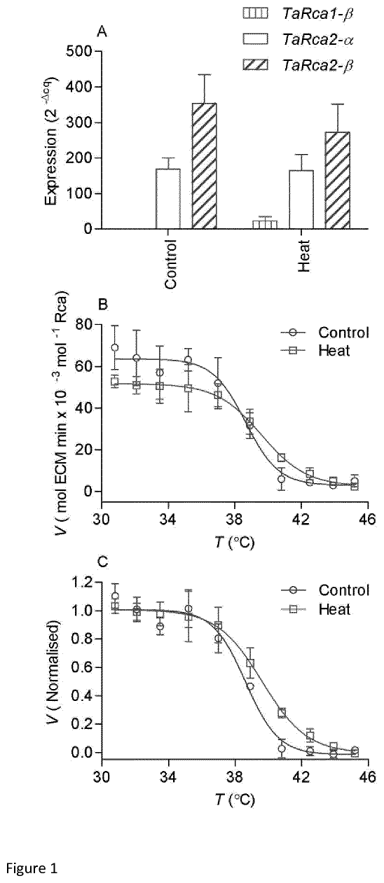

When wheat was exposed to a 38° C. day temperature over two diurnal cycles there was a shift in Rca isoform expression from control at 22° C. While expression of the TaRca2-α isoform remained constant, there was a decline in expression of the TaRca2-β isoform when the heat treatment was applied (FIG. 1A). More interestingly, expression of TaRca1-β which is encoded by a separate allele and has a high sequence divergence from the other spliced isoforms, went from undetectable expression levels at control temperature to a substantial detection under the heat treatment. Even so, TaRca1-β expression was well below expression of the TaRca2 spliced variants. Nevertheless, it seems heat induces the expression of the TaRca1-β gene implying the TaRca1-β protein is involved in the heat response of wheat.

### Rca and Rubisco Leaf Extraction and Purification

Protein was extracted from the leaves grown under standard physiological conditions described above. Frozen leaf tissue was ground into a fine powder using liquid N₂ and a mortar and pestle. While on ice, leaf powder was added to and repeatedly vortexed in an extraction buffer consisting of 100 mM Tris pH 8.0, 1 mM EDTA, 7.5 mM MgCl2, 2 mM DTT, 1 mM ATP, 2% W/V PVPP and protease inhibitor cocktail, before being passed through a single layer of Miricloth and Lingette Gaze to remove solid matter. The sample was spun at 24,000 g for 20-min at 4° C. and supernatant kept. 35% V/V of saturated ammonium sulfate was added and the sample kept on ice for 30-min before re-spinning. For Rca purification, the pellet was resuspended in leaf extraction buffer minus PVPP, desalted in the same buffer using Sephadex PD-10 desalting columns and loaded onto a 1 ml HiTrap Q FF column with a flow rate of 1 ml/min and equilibrated with desalting buffer. A gradient of desalting buffer containing 0.5 mM KCl from 0 to 100% over 20 ml at a 1 ml/min flow rate was used to elute Rca and fractions determine to contain protein by Bradford assay were pooled and concentrated using 10 kDa Cutoff Amicon concentrators (Merck) to a concentration of 2.2±0.5 mg/ml and stored at −80° C. until use. For Rubisco purification, to the supernatant after the 35% V/V saturated ammonium sulfate precipitation step given above, 60% V/V of saturated ammonium sulfate was added dropwise and slowly stirred at 4° C. for 30 min before being re-spun. The resulting pellet was suspended in a sample buffer of 100 mM Tricine pH 8.0, 0.5 mM EDTA and desalted into the same buffer using PD-10 desalting columns. 20% glycerol was added and the sample aliquoted and snap frozen and stored at −80° C. until use.

### Increased Thermostability of Heat Treated Wheat Rcas

To establish if the observed changes in the expression profile of Rca due to heat had an effect on the activity and heat stability of the Rca holoenzyme, the protein was extracted and isolated from leaves and measured at 25° C. after incubation for 10 minutes at a range of temperatures to determine the thermal midpoint (Tm), the temperature at which half of Rca velocity was impaired. Absolute rates of Rubisco activation velocity by Rca measured at 25° C. (V₂₅) were lower for Rca extracted from heat treated versus control leaves (FIG. 1B), suggesting that changes in the Rca isoform makeup caused by the heat treatment reduced the potential maximum velocity of Rca. However, there was a small but significant 1° C. increase in the Rca Tm for the heat treated plants (Table 2), with the shift in temperature response clearly observable when Rca velocity was normalised (FIG. 1C).

### Example 2—Characterization of the Thermostability of Each of the Three Wheat Rea Isoforms

### Recombinant Protein Generation

All Rca genes of interest were synthesised de novo (GENEWIZ, South Plainfield, N.J., USA) with 46 amino acids at the N-terminus corresponding to the signal peptide deleted and a 6 amino acid His-tag attached to the C-terminus. Genes were ligated into Novagen pET-23d+ vectors (Merck KGaA, Darmstadt, Germany) before being transformed into BL21(DE3) Star Escherichia coli strain following standard procedures. His-tagged Rca expression and purification was performed as outlined by Scafaro et al. (2016)¹⁵. Final Rca protein was desalted into a buffer containing 20 mM Tris pH 8, 0.2 mM EDTA, 7.5 mM MgCl₂, 1 mM DTT and 50 mM KCl, at a concentration of 2.2±0.5 mg/ml, snap frozen and stored at −80° C. until use. Rca protein concentration was determined using Protein Assay Dye Reagent Concentrate (Bio-Rad) with a bovine serum albumin (BSA) standard and molar concentration calculated using the molecular masses of 50,954 and 47,110 Da for the α and β isoforms of wheat, respectively and 47,930 for the rice β isoform. Impurities in isolated Rca samples were also accounted when calculating Rca concentration by gel image analysis using IMAGEJ software (National Institutes of Health, Bethesda, Mass., USA).

### Rca Pre-Incubation and Rubisco Activation Assays

Prior to measuring the Rubisco activation velocity, 5 μl of 2.2±0.5 mg/ml Rca sample with or without 0.2 mM ATP added was placed in 4Titude PCR tubes (BIOKE, Leiden, The Netherlands) and heated on a Labcycler (SensoQuest GmBH, Goettingen, Germay) set at 38° C. or equivalent temperature dependent on Rca variant, with a 8° C. temperature range over 12 positions. All tubes were initially heated for 2 min at 25° C. followed by heating of individual tubes across the 8° C. temperature range for 10 minutes. Tubes were then centrifuged at 4000 g for 5 minutes prior to determination of enzymatic velocity. The ability of Rca to activate Rubisco was measured following the ADP insensitive coupled-enzyme spectrophotometric method of Scales et al.²³ with the following modified details. All reagents were purchased from Merck KGaA except for d-2,3-phosphoglycerate mutase which was expressed and purified as previously outlined²⁴. The assay was scaled down to 100 μl reactions and measured in Coster 96-well flat-bottom polystyrene plates (Corning, N.Y., USA), heated to 25° C. using an Eppendorf Thermomixer (Eppendorf, Hamburg, Germany). In one set of wells a reaction solution with final volume of 80 μl was added consisting of N₂ sparged MiliQ H₂O, 5% W/V PEG-4000, 100 mM Tricine pH 8, 10 mM MgCl₂, 10 mM NaHCO₃, 5 mM DTT, 2.4 U ml Enolase, 3.75 U ml Phosphoenolpyruvate carboxylase, 6 U ml Malate dehydrogenase, 0.2 mM 2,3-bis-Phosphoglycerate, 4 U ml d-2,3-phosphoglycerate mutase, 10 U ml carbonic anhydrase, 4 mM phosphocreatine, 20 U ml creatine phosphokinase, 2 mM ATP and 0.8 mM NADH. In another set of wells a final volume of 20 μl consisted of 0.25±0.05 μM of Rubisco active sites added to either; 1. an activation solution (N₂ sparged MiliQ H₂O, 20 mM Tricine pH 8, 20 mM NaHCO₃ and 10 mM MgCL₂) to determine Rubisco total carbamylated activity (ECM), or 2.4 mM of Ribulose-1,5-bisphosphate (RuBP; 99% pure) for Rubisco substrate inhibition (ER). Two minutes prior to measurements 4 μl of the preincubated Rca was added to ER wells as a separate droplet from the Rubisco solution. Rca was not added to ER samples when measuring spontaneous baseline activity. 10-min after addition of Rubisco, the contents of the reaction solution wells were added to the Rubisco containing wells by multi-pipette and measurements of absorbance at a wavelength of 340 nm immediately made on an Infinate M200 Pro plate reader (TECAN, Minnedorf, Switzerland) every 15-sec over an 8-min period. Up to 10 samples were assayed simultaneously. The quantification of ECM regenerated reactions by Rca per minute (mol ECM min×10⁻³ mol⁻¹ Rca) was calculated by the method outlined by Loganathan et al. 2016²³. The amount of Rubisco active sites added to the assay was determined from the slope of a linear regression through the data points corresponding to the first 60-sec of 3-Phosphoglycetic acid (3PG) product generated from ECM samples and factoring in a wheat Rubisco reaction rate constant (K_{cat}) of 2.1²⁵ at 25° C.

### Temperature Curve Analysis

To determine the midpoint temperature at which Rubisco activation velocity (V) by Rca was reduced by half (Tₘ), an ordinary least-squares fit of V versus pre-incubation temperature (T) plots were made using the variable slope model:

$$v = {V}_{min} + \frac{{V}_{max}}{{V}_{min}} / 1 + 1 {0}^{( {T}_{m - T} ) * Hillslope} \qquad Equation 1$$

where Vₘᵢₙ is the slowest Rubisco activation velocity recorded, Vₘₐₓ the fastest activation velocity, T is the pre-incubation temperature in ° C. and Hillslope is the steepness of the decline in V. Tₘ was taken as the Tat which V was at the midpoint between Vₘᵢₙ and Vₘₐₓ, and the Hillslope was taken as Sign(V at Tₘₐₓ−V at Tₘᵢₙ). All data and statistical analysis was carried out using Graphpad Prism 5.0 software (GraphPad Prism Software Inc., San Diego, Calif., USA) or R programming language (R Core Team 2017; www.R-project.org) All experiments were repeated between 3-6 times and all values and error bars presented are means and standard deviation (SD).

The Wheat Rca 1β Isoform is More Thermostable than the Rca 2 Isoforms

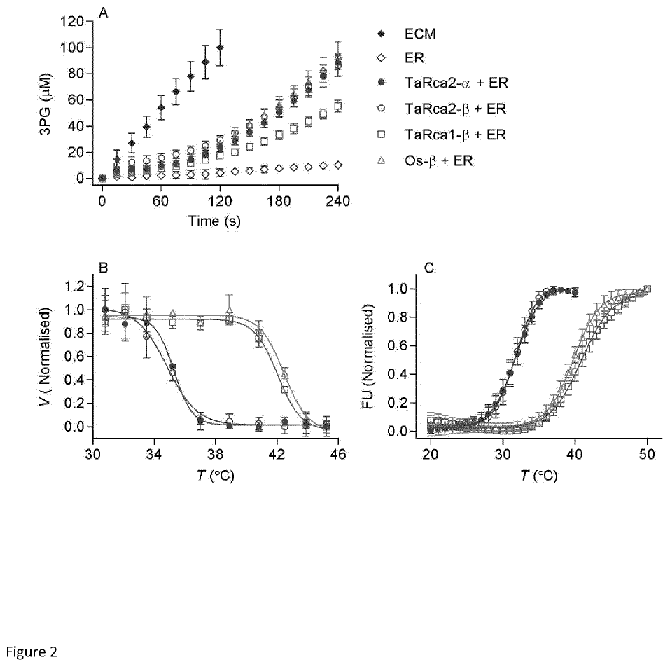

Recombinant expressed and purified Rca isoforms were used to test if induction of TaRca1-β expression and the associated increase in Rca thermal stability observed in heat treated leaves was in fact due to TaRca1-β being a more thermostable variant of Rca than TaRca2-β or TaRca2-α. The β isoform of Rca from rice (OsRca-β) was used as a reference for heat stability as rice is a tropical species unlike temperate wheat. The Tₘ of the two TaRca2 α and β isoforms was not significantly different and within 1° C. of each other and somewhat expectedly, OsRca-β had a 7.2° C. and 7.7° C. higher Tₘ than the TaRca2-α and TaRca2-β spliced variants, respectively (FIG. 2, Table 2). Of interest, the TaRca1-β isoform was indeed a heat stable variant of Rca for wheat, to the extent that its Tₘ was 42° C., also 7° C. above both TaRca2 spliced variants and within 1° C. of the rice OsRca-β isoform. The V₂₅ of TaRca1-β was however significantly less than the velocity of the other wheat and rice Rca isoforms, which had similar V₂₅.

### Example 3—Design of Thermostable Wheat Rea 2 Variants

### Sequences Alignments

All protein sequence utilized the analysis software CLC Main Workbench V. 7.8.1 (QIAGEN Aarhus A/S). The warm species consensus sequence was generated through alignment of the Rca protein sequence of Oryza sativa (BAA97584.1), Oryza australiensis (ANH11447.1), Larrea tridentate (Q7X999.1), Musa acuminate (XP_009419709.1), Datisca glomerata (AAC62207.1), Theobroma cacao (EOY07450.1), Nicotiana tabacum (AAA78277.1) and Gossypium hirsutum (XP_016753736.1). The cold species consensus sequence was generated from Arabidopsis thaliana (NP_850321.1), Brassica oleracea (AFH35543.1), Picea sitchensis (ABK25255.1), Spinacia Oleracea (AAA34038.1), Fragaria vesca (XP_004305457.1), Arachis duranensis (XP_015938754.1), Brachypodium distachyon (XP_003580722.1), Arabis alpine (KFK41750.1) and Mesembryanthemum crystallinum (AAZ41846.1).

### Identification of Amino Acids Relevant to the Thermostability of Rcas

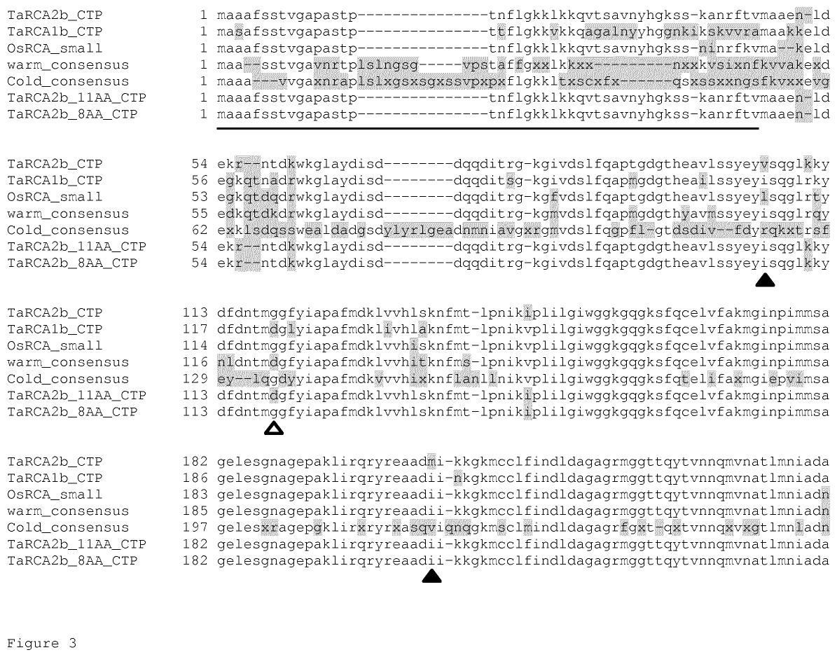

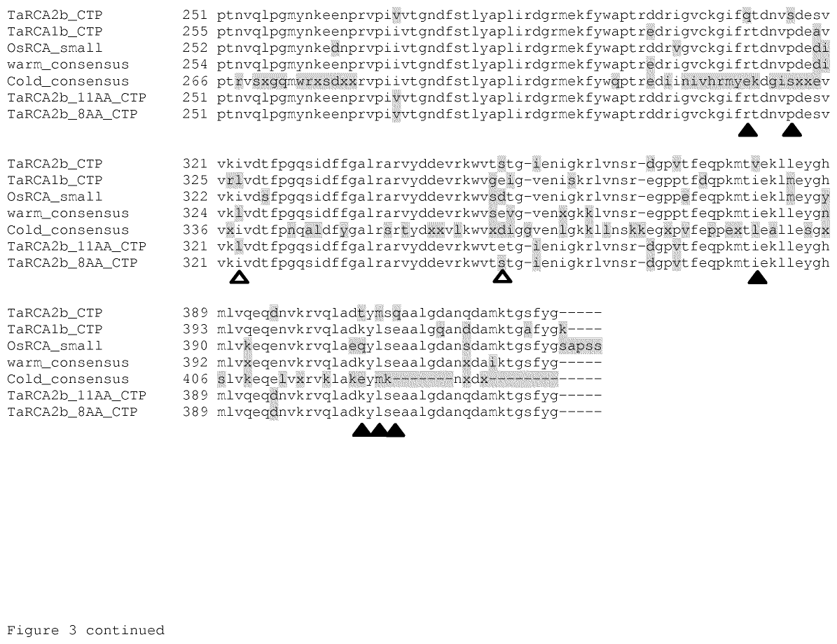

Sequence alignments were used as a tool to determine the residues that are conserved in thermostable variants of Rca so that mutations could be made to the heat sensitive TaRca2-β to artificially raise its thermal stability. Mutations were generated through protein alignment of TaRca2-β with thermostable TaRca1-β and OsRca-β as well as a cold and warm species consensus sequences (FIG. 3). The cold and warm consensus sequences were generated by aligning the Rca sequence of species we classified as endemic to cold or warm environments. The first TaRca2-β mutant generated had 11 amino acid substitutions (TaRca2-β-11AA) based on differences between the TaRca2-β and TaRca1-β sequence with the criteria that TaRca1-β matched the warm species consensus sequence and was different to the cold species consensus sequence. To potentially reduce the number of mutations needed to impart thermal stability a second mutant was generated based on TaRca2-β-11AA but with the additional criteria that at these 11 amino acid mutation sites OsRca-β could not match TaRca2-β or the cold species consensus sequence. This reduced the number of mutations by three, leading to an eight amino acid mutant (TaRca2-β-8AA).

### Evaluation of the Thermostability of the Complex Comprising the Created Rca 2 Protein Variants and the Rubisco Protein

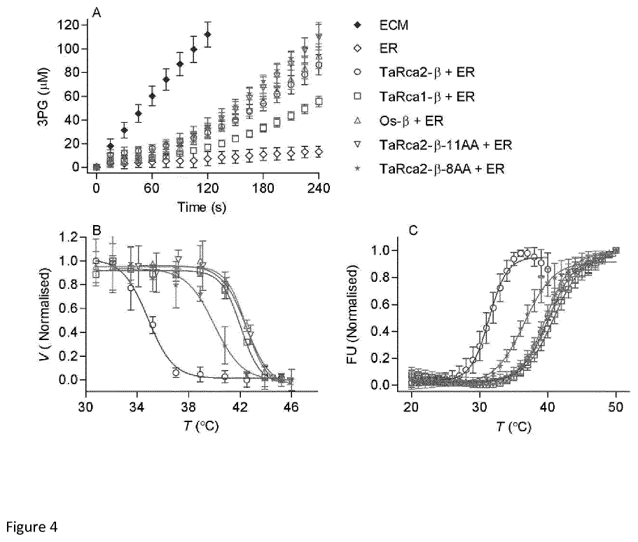

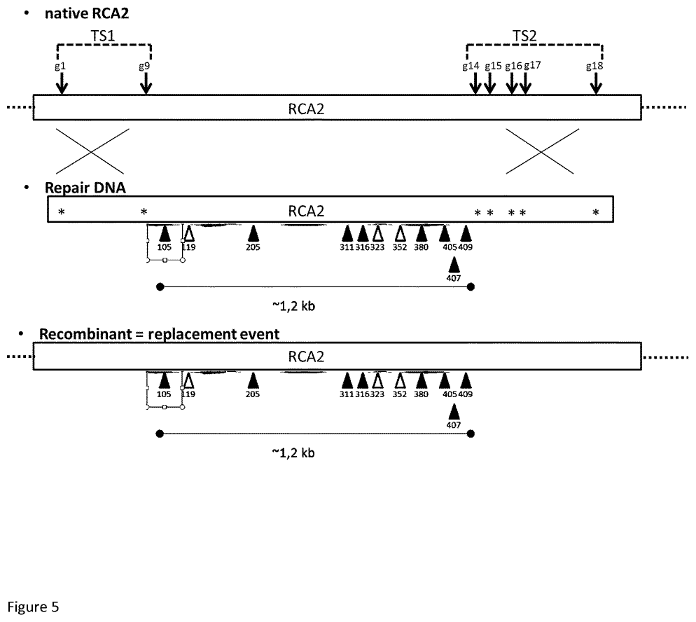

Temperature response curves of Rubisco activation by Rca showed that the TaRca2-β-11AA and TaRca2-β-8AA mutants did have greater thermal stability than the heat sensitive TaRca2-β (FIG. 4, Table 2). In fact, TaRca2-β-11AA had a temperature response and Tₘ of 42.4° C. similar to the TaRca1-β and OsRca-β thermostable isoforms, and again 7° C. greater than TaRca2-β. While TaRca2-β-8AA had improved thermal stability with a Tₘ of 40° C., it was intermediate between TaRca2-β and the other thermostable isoforms, increasing TaRca2-β Tₘ by 5.2° C. This indicates that the extra three residues substitutions of the TaRca2-β-11AA mutant provides a further significant role in promoting heat stability of Rca in wheat. Both the TaRca2-β-11AA and TaRca2-β-8AA mutant had similar V₂₅ values to TaRca2-β indicating that improvements to thermal stability did not come at a cost to kinetic activity for the mutant enzymes.

### Evaluation of the Thermostability of the Created Rca 2 Protein Variants

The point at which Rca unfolded was determined by differential scanning fluorimetry (DSF) as described in Niesen et al. 2007, Nat Protoc. 2, 2212-2221. 10±0.2 μM of Rca protomer in a final volume of 20 μl made of 1:5000 diluted Sypro Orange dye (Invitrogen), Rca desalting buffer and 0.2 mM ATP. Samples were heated on a CFX384 qPCR instrument (Bio-Rad) at 1° C. per minute from 20 to 50° C. and fluorescence excited at 490 nm and emission (FU) recorded at 610 nm at each temperature. An ordinary least-squares model was iteratively fit using the Boltzmann sigmoidal equation:

$$v = {V [ FU ]}_{min} + \frac{{V [ FU ]}_{max}}{{V}_{[ F U ] mi n}} / 1 + {exp}^{( {T}_{m - T} ) * slope} \qquad Equation 2$$

Where V[FU]ₘᵢₙ is the minimum fluorescence units recorded, V[FU]ₘₐₓ the maximum fluorescence units, T is the temperature in ° C. and slope is the steepness of the incline/decline. Tₘ was taken as the T at which V[FU] was at the midpoint between V[FU]ₘᵢₙ and V[FU]ₘₐₓ, and the slope set at an initial value of 2. All data and statistical analysis was carried out using Graphpad Prism 5.0 software (GraphPad Prism Software Inc., San Diego, Calif., USA) or R programming language (R Core Team 2017; https://www.R-project.org/.) All DSF experiments were repeated 20 or more times. All values and error bars presented are means and standard deviation (SD).

Determination of the temperature at which half of Rea structural integrity was impaired (i.e. unfolds) (Tₘ) using differential scanning fluoromitry (DSF) gave comparable results to the ones obtained when determining the temperature at which Rca velocity is reduced by half (Table 2), although Tₘ from DSF was always 2 to 4° C. below values obtained from velocity assays. Tₘ did not significantly differ for Fielder control samples. Using DSF, heat treated Fielder had a significantly higher Tₘ than the control treatments.

These results also confirmed that the TaRca2-β-11AA and TaRca2-β-8AA mutants did have greater thermal stability than the heat sensitive TaRca2-β wild type (FIG. 4, Table 2). In fact, TaRca2-β-11AA had a temperature response and Tₘ of 40.2° C., similar to the TaRca1-β and OsRca-β thermostable isoforms, and again >7° C. above TaRca2-β. While TaRca2-β-8AA had improved thermal stability with a Tₘ of 36.1° C., it was intermediate between TaRca2-β and the other thermostable isoforms, increasing TaRca2-β Tₘ by 5.2° C. This indicates that the extra three residues substitutions of the TaRca2-β-11AA mutant provides a further significant role in promoting heat stability of Rca in wheat.

TABLE 2
The thermal midpoint at which half of Rca velocity or structural
stability is lost (Tm) calculated from Rca activation assays
(V) or differential scanning fluorimetry (DSF). Values are the
means ± SD of four or more experimental replicates. The
pre-incubation heating of Rca was undertaken in the presence
of 0.2 mM ATP for the selected variants. Superscript letters
refer to significant differences between variants at p ≤
0.05 using a One-way ANOVA and Tukey's multiple comparison
test with leaf extracted and recombinant protein analyzed separately.

| Variant | Tₘ (° C.) (V) | Tm (° C.) (DSF) |
|---|---|---|
| TaRca-leaf-25° C. | 38.6 ± 0.2^{a} | 31.4 ± 0.4^{a} |
| TaRca-leaf-38° C. | 39.5 ± 0.5^{b} | 32.6 ± 0.4^{b} |
| TaRca2-α | 35.3 ± 0.2^{c} | 31.8 ± 0.5^{a} |
| TaRca2-β | 34.8 ± 0.5^{c} | 31.8 ± 0.6^{a} |
| TaRca1-β | 42.0 ± 0.2^{d} | 40.9 ± 0.7^{c} |
| OsRca-β | 42.5 ± 0.4^{d} | 39.8 ± 0.5^{d} |
| TaRca2-β-11AA | 42.4 ± 0.5^{d} | 40.2 ± 0.5^{d} |
| TaRca2-β-8AA | 40 ± 0.4^{b} | 36.1 ± 0.6^{e} |

### Example 4—Generation of Wheat Plants with Reduced Level of Endogenous Rca2 Proteins

### Generation of Constructs Silencing Specifically the Endogenous Rca 2 Genes

Using standard recombinant DNA techniques, the constitutive promoter region of the Ubiquitin gene of Zea mays according to the sequence from nucleotide position 157 to 2153 of SEQ ID NO: 60, the hairpin DNA fragment targeting the Rca 2 genes from the subgenomes A, B and D of wheat according to the sequence from nucleotide position 2162 to 3543 of SEQ ID NO: 60, and the 3′ untranslated sequence of the 35S transcript gene of Cauliflower mosaic virus according to the sequence from nucleotide position 3547 to 3771 of SEQ ID NO: 60 were assembled in a vector which contains the bar selectable marker cassette (position 3856 to 5520 of SEQ ID NO: 60) to result in the T-DNA PubiZm::hpRca2 (SEQ ID NO: 60).

### Generation of Transgenic Wheatplants Comprising the Above Mentioned Silencing Construct

The recombinant vectors comprising the expression cassettes PubiZm::hpRca2 is used to stably transform wheat using the method described in Yuji Ishida et al. 2015, Methods in Molecular Biology, 1223: 189-198. Homozygous and null segregant plants are selected.

### Generation of Knock Out Rca 2 Mutant Wheat Plants

### By Mutagenesis

A mutagenized wheat population was constructed by EMS mutagenesis. Based on sequencing of the region around the Rca 2 genes, mutant plants with a knock out mutation in the Rca 2 gene from either the B subgenome, from the A subgenome or from the D subgenome are identified. The homozygous mutant plants and their wildtype segregant are retrieved.

Such mutant plants are crossed to produce double mutant plants with a knock out mutation in the Rca 2 gene from both the subgenome A and B, or from both the subgenome A and D or from both the subgenome B and D. Such resulting double mutant plants are further crossed to produce mutant plants with a knock out mutation in the Rca 2 gene from all three subgenomes (namely A, B and D).

### By Targeted Knock-Out

Guide RNAs for CRISPR-mediated gene editing targeting the mRNA coding sequence, preferably the protein coding sequence of the Rca 2 gene from the D subgenome, targeting the mRNA coding sequence, preferably the protein coding sequence of the Rca 2 genes from both the D and the A subgenomes, targeting the mRNA coding sequence, preferably the protein coding sequence of the Rca 2 genes from both the A and the B subgenomes, or targeting the mRNA coding sequence, preferably the protein coding sequence of the Rca 2 genes from both the A, the B and the D subgenomes were designed by using e.g. the CAS-finder tool. The guide RNAs were tested for targeting efficiency by PEG-mediated transient co-delivery of the gRNA expression vector with an expression vector for the respective nuclease, e.g. Cas9 or Cpfl, under control of appropriate promoters, to protoplasts of a wheat line containing the Rca 2 genes. Genomic DNA was extracted from the protoplasts after delivery of the guide RNA and nuclease vectors. After PCR amplification, integrity of the targeted Rca 2 gene sequence was assessed by sequencing.

The most efficient guide RNAs were used for stable gene editing in wheat. The selected guide RNAs are g1 (SEQ ID NO: 61) targeting the subgenomes A, B and D; g2 (SEQ ID NO: 62) targeting the subgenomes A and D; g13 (SEQ ID NO: 63) targeting the subgenomes A and B; and g9 (SEQ ID NO: 64) targeting the subgenome D. For this purpose, the selected guide RNA expression vector, together with a nuclease expression module and a selectable marker gene, were introduced into wheat embryos using e.g. particle gun bombardment. Transgenic plants showing resistance to the selection agent were regenerated using methods known to those skilled in the art. At least 13 transgenic TO plants containing gene targeting events, preferably small deletions or insertions resulting in a non-functional Rca 2 gene were identified by PCR amplification and sequencing. Examples of knock-out mutant obtained are shown is table 3.

Transgenic T0 plants containing a knock out mutation of at least one of the Rca 2 genes, preferably in homozygous state, but alternatively in heterozygous state, are crossed to produce plants with a knock out mutation in the Rca 2 gene from both the subgenome A and B, or from both the subgenome A and D or from both the subgenome B and D. Such resulting plants are further crossed to produce mutant plants with a knock out mutation in the Rca 2 gene from all three subgenomes (namely A, B and D).

TABLE 3

| | | | mutant |
|---|---|---|---|
| mutant | | AA mutation | protein |
| line | Mutation created | position | length |
| 1 | insertion of a C after | sequence modified | 165 |
| | nucleotide at position | compared to SEQ ID | |
| | 268 of SEQ ID NO: 38 | NO: 39 as of amino | |
| | | acid position 90 | |
| 2 | insertion of an A after | sequence modified | 274 |
| | nucleotide at position | compared to SEQ ID | |
| | 268 of SEQ ID NO: 38 | NO: 39 as of amino | |
| | | acid position 268 | |
| 3 | deletion of the nucleotides | sequence modified | 199 |
| | 97 to 101 of | compared to SEQ ID | |
| | SEQ ID NO: 1 | NO: 2 as of amino | |
| | | acid position 33 | |
| 4 | deletion of the nucleotides | sequence modified | 200 |
| | 100 and 101 | compared to SEQ ID | |
| | of SEQ ID NO: 1 | NO: 2 as of amino | |
| | | acid position 34 | |
| 5 | deletion of the nucleotides | sequence modified | 33 |
| | 102 to 110 of | compared to SEQ ID | |
| | SEQ ID NO: 1 | NO: 2 as of amino | |
| | | acid position 34 | |
| 6 | insertion of an A after | sequence modified | 33 |
| | nucleotide at position | compared to SEQ ID | |
| | 101 of SEQ ID NO: 1 | NO: 2 as of amino | |
| | | acid position 34 | |
| 7 | insertion of an A after | sequence modified | 274 |
| | nucleotide at position | compared to SEQ ID | |
| | 800 of SEQ ID NO: 1 | NO: 2 as of amino | |
| | | acid position 268 | |
| 8 | deletion of the nucleotides | sequence modified | 273 |
| | 803 and 804 of | compared to SEQ ID | |
| | SEQ ID NO: 1 | NO: 2 as of amino | |
| | | acid position 268 | |
| 9 | deletion of the nucleotides | sequence modified | 164 |
| | 100 and 101 of | compared to SEQ ID | |
| | SEQ ID NO: 40 | NO: 41 as of amino | |
| | | acid position 34 | |
| 10 | deletion of the nucleotides | sequence modified | 134 |
| | 270 to 285 of | compared to SEQ ID | |
| | SEQ ID NO: 40 | NO: 41 as of amino | |
| | | acid position 91 | |
| 11 | deletion of the nucleotide | sequence modified | 139 |
| | 267 of SEQ ID | compared to SEQ ID | |
| | NO: 40 | NO: 41 as of amino | |
| | | acid position 90 | |
| 12 | insertion of an A after | sequence modified | 165 |
| | nucleotide at position | compared to SEQ ID | |
| | 299 of SEQ ID NO: 40 | NO: 41 as of amino | |
| | | acid position 100 | |
| 13 | deletion of the nucleotide | sequence modified | 139 |
| | 299 of SEQ ID | compared to SEQ ID | |
| | NO: 40 | NO: 41 as of amino | |
| | | acid position 100 | |

### Example 5—Generation of Wheat Plants with Increased Thermotolerance

Generation of Expression Constructs with the Wheat Thermostable Rcas

Using standard recombinant DNA techniques, the promoter region and 5′UTR of the Rubisco activase gene from Oryza meridionalis according to the sequence from nucleotide position 81 to 880 of SEQ ID NO: 36, the DNA fragment coding for the wheat Rca 1β P according to the sequence from nucleotide position 890 to 2188 of SEQ ID NO: 36, and the 3′ untranslated sequence of the Rubisco activase gene from Oryza meridionalis according to the sequence from nucleotide position 2203 to 2462 of SEQ ID NO: 36 were assembled in a vector which contains the bar selectable marker cassette (position 2537 to 4201 of SEQ ID NO: 36) to result in the T-DNA PrcaOm::Rca 1β (SEQ ID NO: 36).

Using standard recombinant DNA techniques, the promoter region of the Rubisco small subunit gene from Oryza sativa according to the sequence from nucleotide position 75 to 2821 of SEQ ID NO: 37, the first intron of the Actin 1 gene from rice according to the sequence from nucleotide position 2825 to 3286 of SEQ ID NO: 37, DNA fragment coding for the wheat Rca 1β according to the sequence from nucleotide position 3294 to 4592 of SEQ ID NO: 37, and the 3′ untranslated sequence of the Nopalin synthase gene from Agrobacterium tumefaciens according to the sequence from nucleotide position 4630 to 4890 of SEQ ID NO: 37 were assembled in a vector which contains the bar selectable marker cassette (position 4971 to 6635 of SEQ ID NO: 37) to result in the T-DNA Prbcs::Rca 1P (SEQ ID NO: 37).

A DNA molecule is synthesized de novo and designed i) to encode a polypeptide according to SEQ ID 30 or 33, ii) to optimize the nucleotide sequence for expression in wheat plant cells and iii) to avoid that it be targeted for silencing by the hairpin construct described above. For this purpose, factors such as codon usage, mRNA secondary structure, the AT content, cryptic splice sites or restriction sites were taken into account. Using standard recombinant DNA techniques, each of these DNA molecules is assembled with a plant-expressible promoter and a transcription terminator in a vector.

### Generation of Transgenic Wheat Plants Comprising an Expression Construct Comprising a Wheat Thermostable Rca

The recombinant vectors comprising the expression cassettes PrcaOm::Rca 1β and Prbcs::Rca 1β, were used to stably transform wheat using the method described in Yuji Ishida et al. 2015, Methods in Molecular Biology, 1223: 189-198. Homozygous and null segregant plants have been selected.

The recombinant vectors comprising the expression cassettes with the Rca 2 β variants are used to stably transform wheat using the method described in Yuji Ishida et al. 2015, Methods in Molecular Biology, 1223: 189-198. Homozygous and null segregant plants are selected.

The obtained transgenic wheat plants are then crossed with the above described wheat lines wherein the endogenous Rca 2 genes are silenced or knocked out (see Example 4) and lines expressing homozygous and null segregant lines are selected.

### Example 6—Generation of Wheat Plants Comprising the Thermostable Rea 2 Allele

An approach to introduce 11 AA substitutions in Rca2 over an ˜1.2 kb region is based on the simultaneous induction of a D SB at Target Site 1 (TS1) in close proximity of the first amino acid substitution (AA1) and a DBS at Target Site 2 (TS2) in close proximity of the last amino acid substitution (AA2), and the replacement of the ˜1.2 kb native Rca2 sequence by the ˜1.2 kb Rca2 mutated sequence containing the 11 AA by homologous recombination. Hereto, a repair DNA has been developed comprising the ˜1.2 kb Rca2 mutated sequence flanked by regions of homology to the sequence immediately upstream of TS1 and immediately downstream of TS2 with silent mutations over the gRNA target sites to prevent cleavage of the repair DNA (FIG. 6).

For the identification of two Target Sites (TS1 and TS2) that can be cut simultaneously, the Cas9 nuclease and pairs of sgRNAs for targeting TS1 and TS2 were co-delivered into wheat protoplasts by PEG-mediated transfection. A simultaneous cleavage of TS1 and TS2 can result in the deletion of the ˜1.2 kb region between the 2 target sites. By PCR amplification using primers upstream and downstream of TS1 and TS2 respectively, a smaller PCR fragment corresponding with the expected size as when the deletion of the ˜1.2 kb region has occurred, was observed. Table3 shows pairs of gRNAs that upon co-delivery with the Cas9 nuclease cleaved their respective Target Site TS1 and TS2 simultaneously, resulting in the deletion of the ˜1.2 kb Rca2 region.

TABLE 4

| pair of | | | |
|---|---|---|---|
| gRNAs | subgenomes | TS1 sequence | TS2 sequence |
| g1-g14 | A, B, D | SEQ ID NO: 61 | SEQ ID NO: 65 |
| g1-g15 | A, B, D | SEQ ID NO: 61 | SEQ ID NO: 66 |
| g1-g16 | A, B, D | SEQ ID NO: 61 | SEQ ID NO: 67 |
| g1-g17 | A, B, D | SEQ ID NO: 61 | SEQ ID NO: 68 |
| g9-g18 | D | SEQ ID NO: 64 | SEQ ID NO: 69 |

The Cas9 gene, the 2 sgRNAs for cleavage at Target Sites TS1 and TS2, and the repair DNA have been introduced through particle bombardment into the scutellum cells of immature embryos. Regenerated plants have been obtained from the bombarded embryos using either selection-free tissue culture methods or by selection-based tissue culture methods by doing a co-delivery of the Cas9, the 2sgRNAs and the repair DNA with a selectable marker gene (e.g. epsps, bar). Removal of the selectable marker gene from replacement events containing the Rca2 11 AA substitutions can be done by progeny segregation.

1. A method for increasing the ratio of a thermostable Rca (Rubisco Activase) protein in cereals comprising:
a.
i. providing to cells of a cereal plant a recombinant gene comprising the following operably linked elements:
1. a promoter, preferably expressible in plants;
2. a nucleic acid encoding a thermostable Rca protein selected from:
a. an Rca 1β protein and variants thereof; and
b. a thermostable Rca 2 protein variant and, optionally
3. a transcription termination and polyadenylation region, preferably a transcription termination and polyadenylation region functional in plants; and
ii. reducing the expression of endogenous non-thermostable Rca 2 protein in said cereal plant cells wherein said ratio is increased compared to a control cereal plant cell not comprising said recombinant gene; or
b. introducing into cells of a cereal plant at least one thermostable Rca 2 allele wherein said thermostable Rca 2 allele encodes the amino acid comprising:
i. the amino acid sequence of SEQ ID NOs: 32 or 35 or
ii. an amino acid sequence having 90% identity with SEQ ID NOs: 32 or 35 and comprising at least one amino acid selected from:
1. an isoleucine at a position corresponding to position 59 of SEQ ID NO: 32 or 35;
2. an aspartic acid at a position corresponding to position 73 of SEQ ID NO: 32 or 35;
3. an isoleucine at a position corresponding to position 160 of SEQ ID NO: 32 or 35;
4. an arginine at a position corresponding to position 265 of SEQ ID NO: 32 or 35;
5. a proline at a position corresponding to position 270 of SEQ ID NO: 32 or 35;
6. a leucine at a position corresponding to position 277 of SEQ ID NO: 32 or 35;
7. a glutamic acid at a position corresponding to position 307 of SEQ ID NO: 32 or 35;
8. an isoleucine at a position corresponding to position 334 of SEQ ID NO: 32 or 35;
9. a lysine at a position corresponding to position 359 of SEQ ID NO: 32 or 35;
10. a leucine at a position corresponding to position 361 of SEQ ID NO: 32 or 35; and
11. a glutamic acid at a position corresponding to position 363 of SEQ ID NO: 32 or 35
wherein said ratio is increased compared to a control cereal plant cell not comprising said thermostable Rca 2 allele.

2. The method according to claim 1 wherein said Rca 1β protein and variants thereof comprise an amino acid sequence selected from:
a. the amino acid sequence of SEQ ID NO: 8;
b. an amino acid sequence having at least 90% identity to the amino acid sequence of SEQ ID NO: 8 and comprising at least one amino acid selected from:
i. an isoleucine at a position corresponding to position 109 of SEQ ID NO: 8;
ii. an aspartic acid at a position corresponding to position 123 of SEQ ID NO: 8;
iii. an isoleucine at a position corresponding to position 210 of SEQ ID NO: 8;
iv. an arginine at a position corresponding to position 315 of SEQ ID NO: 8;
v. a proline at a position corresponding to position 320 of SEQ ID NO: 8;
vi. a leucine at a position corresponding to position 327 of SEQ ID NO: 8;
vii. a glutamic acid at a position corresponding to position 357 of SEQ ID NO: 8;
viii. an isoleucine at a position corresponding to position 384 of SEQ ID NO: 8;
ix. a lysine at a position corresponding to position 409 of SEQ ID NO: 8;
x. a leucine at a position corresponding to position 411 of SEQ ID NO: 8; and
xi. a glutamic acid at a position corresponding to position 413 of SEQ ID NO: 8.

3. The method according to claim 1 wherein said nucleic acid encoding a Rca 1β protein and variants thereof comprise a coding nucleic acid sequence selected from:
a. the nucleic acid of SEQ ID NO: 7, or complement thereof;
b. a nucleic acid having at least 60% identity to the nucleic acid of SEQ ID NO: 7, or complement thereof.

4. The method according to claim 1, wherein said thermostable Rca 2 protein variant comprise an amino acid sequence selected from:
a. the amino acid sequences of SEQ ID NO: 30 or 33;
b. an amino acid sequence having at least 90% identity to the amino acid sequences of SEQ ID NO: 30 or 33 and comprising at least one amino acid selected from:
i. an isoleucine at a position corresponding to position 105 of SEQ ID NO: 30 or 33;
ii. an aspartic acid at a position corresponding to position 119 of SEQ ID NO: 30 or 33;
iii. an isoleucine at a position corresponding to position 206 of SEQ ID NO: 30 or 33;
iv. an arginine at a position corresponding to position 311 of SEQ ID NO: 30 or 33;
v. a proline at a position corresponding to position 316 of SEQ ID NO: 30 or 33;
vi. a leucine at a position corresponding to position 323 of SEQ ID NO: 30 or 33;
vii. a glutamic acid at a position corresponding to position 353 of SEQ ID NO: 30 or 33;
viii. an isoleucine at a position corresponding to position 380 of SEQ ID NO: 30 or 33;
ix. a lysine at a position corresponding to position 405 of SEQ ID NO: 30 or 33;
x. a leucine at a position corresponding to position 407 of SEQ ID NO: 30 or 33; and
xi. a glutamic acid at a position corresponding to position 409 of SEQ ID NO: 30 or 33.

5. The method according to claim 1, wherein said thermostable Rca 2 protein variant comprise an amino acid sequence selected from:
a. the amino acid sequences of SEQ ID NO: 32 or 35 and further comprising a chloroplast targeting peptide;
b. an amino acid sequence having at least 90% identity to the amino acid sequences of SEQ ID NO: 32 or 35, further comprising a chloroplast targeting peptide, and comprising at least one amino acid selected from:
i. an isoleucine at a position corresponding to position 59 of SEQ ID NO: 32 or 35;
ii. an aspartic acid at a position corresponding to position 73 of SEQ ID NO: 32 or 35;
iii. an isoleucine at a position corresponding to position 160 of SEQ ID NO: 32 or 35;
iv. an arginine at a position corresponding to position 265 of SEQ ID NO: 32 or 35;
v. a proline at a position corresponding to position 270 of SEQ ID NO: 32 or 35;
vi. a leucine at a position corresponding to position 277 of SEQ ID NO: 32 or 35;
vii. a glutamic acid at a position corresponding to position 307 of SEQ ID NO: 32 or 35;
viii. an isoleucine at a position corresponding to position 334 of SEQ ID NO: 32 or 35;
ix. a lysine at a position corresponding to position 359 of SEQ ID NO: 32 or 35;
x. a leucine at a position corresponding to position 361 of SEQ ID NO: 32 or 35; and
xi. a glutamic acid at a position corresponding to position 363 of SEQ ID NO: 32 or 35.

6. The method according to claim 5, wherein said chloroplast targeting peptide comprises an amino acid sequence selected from:
a. the amino acid sequence of SEQ ID NO: 30 from position 1 to position 46 or the amino acid sequence of SEQ ID NO: 8 from position 1 to position 47; and
b. an amino acid sequence having at least 80% identity to the amino acid sequences of SEQ ID NO: 30 from position 1 to position 46 or the amino acid sequence of SEQ ID NO: 8 from position 1 to position 47.

7. The method according to any-ene-f claim 1, wherein said nucleic acid encoding a thermostable Rca 2 protein variant comprises a coding nucleotide sequence selected from:
a. the nucleotide sequence of SEQ ID Nos: 31, 34, 36 or 37, or the complement thereof;
b. a nucleotide sequence having at least 60% identity with the nucleotide sequence of SEQ ID Nos: 31, 34, 36 or 37, or the complement thereof

8. The method according to claim 1, wherein reducing expression of endogenous non-thermostable Rca 2 protein comprises:
a. introducing into said cells of a cereal plant at least one knock out mutant Rca 2 allele; or
b. providing said cells of a cereal plant with a second recombinant gene capable of suppressing specifically the expression of the endogenous non-thermostable Rca 2 genes.

9. The method according to claim 8, wherein said knock out mutant Rca 2 allele is a knock out mutant allele of the Rca 2β gene from the wheat subgenome B, A or D, or the Rca 2α gene from the wheat subgenome B, A or D.

10. The method according to claim 8, wherein said second recombinant gene capable of suppressing specifically the expression of the endogenous non-thermostable Rca 2 genes comprises the following operably linked elements:
a. a promoter, preferably expressible in plants;
b. a nucleic acid which when transcribed yields an RNA molecule inhibitory to the endogenous Rca 2 genes encoding a non thermostable Rca protein but not inhibitory to genes encoding thermostable Rca proteins; and, optionally
c. a transcription termination and polyadenylation region, preferably a transcription termination and polyadenylation region functional in plants.

11. The method according to claim 8, wherein the endogenous non-thermostable Rca 2 genes comprise the coding nucleotide sequence of SEQ ID NO: 1 or a coding nucleotide sequence having at least 60% identity with the nucleotide sequence of SEQ ID NO: 1 and not encoding the amino acids selected from:
a. an isoleucine at a position corresponding to position 59 of SEQ ID NO: 4;
b. an aspartic acid at a position corresponding to position 73 of SEQ ID NO: 4;
c. an isoleucine at a position corresponding to position 160 of SEQ ID NO: 4;
d. an arginine at a position corresponding to position 265 of SEQ ID NO: 4;
e. a proline at a position corresponding to position 270 of SEQ ID NO: 4;
f. a leucine at a position corresponding to position 277 of SEQ ID NO: 4;
g. a glutamic acid at a position corresponding to position 307 of SEQ ID NO: 4;
h. an isoleucine at a position corresponding to position 334 of SEQ ID NO: 4;
i. a lysine at a position corresponding to position 359 of SEQ ID NO: 4;
j. a leucine at a position corresponding to position 361 of SEQ ID NO: 4; and
k. a glutamic acid at a position corresponding to position 363 of SEQ ID NO: 4.

12. The method according to claim 1, wherein said promoter is a constitutive promoter, a tissue-specific promoter or an inducible promoter.

13. The method according to claim 1, wherein said thermostable mutant Rca 2 allele comprises the coding nucleotide sequence of SEQ ID NOs: 31, 34, 36 or 37 or a coding nucleotide sequence having at least 60% identity with SEQ ID NOs: 31, 34, 36 or 37 and encoding a protein comprising at least one of the amino acids selected from:
a. an isoleucine at a position corresponding to position 59 of SEQ ID NO: 32 or 35;
b. an aspartic acid at a position corresponding to position 73 of SEQ ID NO: 32 or 35;
c. an isoleucine at a position corresponding to position 160 of SEQ ID NO: 32 or 35;
d. an arginine at a position corresponding to position 265 of SEQ ID NO: 32 or 35;
e. a proline at a position corresponding to position 270 of SEQ ID NO: 32 or 35;
f. a leucine at a position corresponding to position 277 of SEQ ID NO: 32 or 35;
g. a glutamic acid at a position corresponding to position 307 of SEQ ID NO: 32 or 35;
h. an isoleucine at a position corresponding to position 334 of SEQ ID NO: 32 or 35;
i. a lysine at a position corresponding to position 359 of SEQ ID NO: 32 or 35;
j. a leucine at a position corresponding to position 361 of SEQ ID NO: 32 or 35; and
k. a glutamic acid at a position corresponding to position 363 of SEQ ID NO: 32 or 35.

14. A method for increasing thermotolerance of a cereal plant comprising:
a. Increasing the ratio of a thermostable Rca protein according to claim 1; and
b. Regenerating said cereal plant
wherein said thermotolerance is increased compared to a cereal plant not comprising said increased ratio of a thermostable Rca protein.

15. A method for increasing yield of a cereal plant under heat stress conditions comprising:
a. Increasing the ratio of a thermostable Rca according to claim 1; and
b. Regenerating said cereal plant
wherein said yield increase is achieved compared to the yield of a cereal plant herein the ratio of a thermostable Rca is not increased.

16. The method according to claim 15, wherein said yield is seed yield.

17. The method according to claim 15, wherein said yield is thousand seed weight.

18. Method for producing a cereal plant with increased thermotolerance comprising
a. Increasing the ratio of a thermostable Rca according to claim 1; and
b. Regenerating said cereal plant.

19. A thermostable Rca 2 protein variant comprising an amino acid sequence selected from:
a. the amino acid sequences of SEQ ID NO: 30 or 33;
b. an amino acid sequence having at least 90% identity to the amino acid sequences of SEQ ID NO: 30 or 33 and comprising at least one amino acid selected from:
i. an isoleucine at a position corresponding to position 105 of SEQ ID NO: 30 or 33;
ii. an aspartic acid at a position corresponding to position 119 of SEQ ID NO: 30 or 33;
iii. an isoleucine at a position corresponding to position 206 of SEQ ID NO: 30 or 33;
iv. an arginine at a position corresponding to position 311 of SEQ ID NO: 30 or 33;
v. a proline at a position corresponding to position 316 of SEQ ID NO: 30 or 33;
vi. a leucine at a position corresponding to position 323 of SEQ ID NO: 30 or 33;
vii. a glutamic acid at a position corresponding to position 353 of SEQ ID NO: 30 or 33;
viii. an isoleucine at a position corresponding to position 380 of SEQ ID NO: 30 or 33;
ix. a lysine at a position corresponding to position 405 of SEQ ID NO: 30 or 33;
x. a leucine at a position corresponding to position 407 of SEQ ID NO: 30 or 33; and
xi. a glutamic acid at a position corresponding to position 409 of SEQ ID NO: 30 or 33;
c. the amino acid sequences of SEQ ID NO: 32 or 35 and optionally further comprising a chloroplast targeting peptide;
d. an amino acid sequence having at least 90% identity to the amino acid sequences of SEQ ID NO: 32 or 35, optionally further comprising a chloroplast targeting peptide, and comprising at least one amino acid selected from:
i. an isoleucine at a position corresponding to position 59 of SEQ ID NO: 32 or 35;
ii. an aspartic acid at a position corresponding to position 73 of SEQ ID NO: 32 or 35;
iii. an isoleucine at a position corresponding to position 160 of SEQ ID NO: 32 or 35;
iv. an arginine at a position corresponding to position 265 of SEQ ID NO: 32 or 35;
v. a proline at a position corresponding to position 270 of SEQ ID NO: 32 or 35;
vi. a leucine at a position corresponding to position 277 of SEQ ID NO: 32 or 35;
vii. a glutamic acid at a position corresponding to position 307 of SEQ ID NO: 32 or 35;
viii. an isoleucine at a position corresponding to position 334 of SEQ ID NO: 32 or 35;
ix. a lysine at a position corresponding to position 359 of SEQ ID NO: 32 or 35;
x. a leucine at a position corresponding to position 361 of SEQ ID NO: 32 or 35; and
xi. a glutamic acid at a position corresponding to position 363 of SEQ ID NO: 32 or 35.

20. The thermostable Rca 2 protein variant according to claim 19, wherein said chloroplast targeting peptide comprises an amino acid sequence selected from:
a. the amino acid sequence of SEQ ID NO: 30 from position 1 to position 46 or the amino acid sequence of SEQ ID NO: 8 from position 1 to position 47; and
b. an amino acid sequence having at least 80% identity to the amino acid sequences of SEQ ID NO: 30 from position 1 to position 46 or the amino acid sequence of SEQ ID NO: 8 from position 1 to position 47.

21. A nucleic acid encoding the thermostable Rca 2 protein variant according to claim 19 comprising a coding nucleotide sequence selected from:
a. the nucleotide sequence of SEQ ID Nos: 31, 34, 36 or 37, or the complement thereof;
b. a nucleotide sequence having at least 60% identity with the nucleotide sequence of SEQ ID Nos: 31, 34, 36 or 37, or the complement thereof

22. A recombinant gene comprising the following operably linked elements:
a. a promoter, preferably expressible in plants;
b. a nucleic acid encoding a Rca protein selected from:
a. a Rca 1β protein and variants thereof, and
b. a thermostable Rca 2 protein variant; and, optionally
c. a transcription termination and polyadenylation region, preferably a transcription termination and polyadenylation region functional in plants.

23. The recombinant gene according to claim 22 wherein said promoter is a constitutive promoter, tissue-specific promoter or an inducible promoter.

24. The recombinant gene according to claim 22, wherein said Rca 1β protein and variants thereof comprise an amino acid sequence selected from:
a. the amino acid sequence of SEQ ID NO: 8;
b. an amino acid sequence having at least 90% identity to the amino acid sequence of SEQ ID NO: 8 and comprising at least one amino acid selected from:
i. an isoleucine at a position corresponding to position 109 of SEQ ID NO: 8;
ii. an aspartic acid at a position corresponding to position 123 of SEQ ID NO: 8;
iii. an isoleucine at a position corresponding to position 210 of SEQ ID NO: 8;
iv. an arginine at a position corresponding to position 315 of SEQ ID NO: 8;
v. a proline at a position corresponding to position 320 of SEQ ID NO: 8;
vi. a leucine at a position corresponding to position 327 of SEQ ID NO: 8;
vii. a glutamic acid at a position corresponding to position 357 of SEQ ID NO: 8;
viii. an isoleucine at a position corresponding to position 384 of SEQ ID NO: 8;
ix. a lysine at a position corresponding to position 409 of SEQ ID NO: 8;
x. a leucine at a position corresponding to position 411 of SEQ ID NO: 8; and
xi. a glutamic acid at a position corresponding to position 413 of SEQ ID NO: 8.

25. The recombinant gene according to claim 22, wherein said nucleic acid encoding an Rca 1β protein and variants thereof comprise a coding nucleic acid sequence selected from:
a. the nucleic acid of SEQ ID NO: 7, or complement thereof;
b. a nucleic acid having at least 60% identity to the nucleic acid of SEQ ID NO: 7, or complement thereof.

26. The recombinant gene according to claim 22, wherein said thermostable Rca 2 protein variant comprises an amino acid sequence selected from:
a. the amino acid sequences of SEQ ID NO: 30 or 33;
b. an amino acid sequence having at least 90% identity to the amino acid sequences of SEQ ID NO: 30 or 33 and comprising at least one amino acid selected from:
i. an isoleucine at a position corresponding to position 105 of SEQ ID NO: 30 or 33;
ii. an aspartic acid at a position corresponding to position 119 of SEQ ID NO: 30 or 33;
iii. an isoleucine at a position corresponding to position 206 of SEQ ID NO: 30 or 33;
iv. an arginine at a position corresponding to position 311 of SEQ ID NO: 30 or 33;
v. a proline at a position corresponding to position 316 of SEQ ID NO: 30 or 33;
vi. a leucine at a position corresponding to position 323 of SEQ ID NO: 30 or 33;
vii. a glutamic acid at a position corresponding to position 353 of SEQ ID NO: 30 or 33;
viii. an isoleucine at a position corresponding to position 380 of SEQ ID NO: 30 or 33;
ix. a lysine at a position corresponding to position 405 of SEQ ID NO: 30 or 33;
x. a leucine at a position corresponding to position 407 of SEQ ID NO: 30 or 33; and
xi. a glutamic acid at a position corresponding to position 409 of SEQ ID NO: 30 or 33;

27. The recombinant gene according to claim 22, wherein said thermostable Rca 2 protein variant comprises an amino acid sequence selected from
a. the amino acid sequences of SEQ ID NO: 32 or 35 and optionally further comprising a chloroplast targeting peptide;
b. an amino acid sequence having at least 90% identity to the amino acid sequences of SEQ ID NO: 32 or 35, optionally further comprising a chloroplast targeting peptide, and comprising at least one amino acid selected from:
i. an isoleucine at a position corresponding to position 59 of SEQ ID NO: 32 or 35;
ii. an aspartic acid at a position corresponding to position 73 of SEQ ID NO: 32 or 35;
iii. an isoleucine at a position corresponding to position 160 of SEQ ID NO: 32 or 35;
iv. an arginine at a position corresponding to position 265 of SEQ ID NO: 32 or 35;
v. a proline at a position corresponding to position 270 of SEQ ID NO: 32 or 35;
vi. a leucine at a position corresponding to position 277 of SEQ ID NO: 32 or 35;
vii. a glutamic acid at a position corresponding to position 307 of SEQ ID NO: 32 or 35;
viii. an isoleucine at a position corresponding to position 334 of SEQ ID NO: 32 or 35;
ix. a lysine at a position corresponding to position 359 of SEQ ID NO: 32 or 35;
x. a leucine at a position corresponding to position 361 of SEQ ID NO: 32 or 35; and
xi. a glutamic acid at a position corresponding to position 363 of SEQ ID NO: 32 or 35.

28. The recombinant gene according to claim 27, wherein said chloroplast targeting peptide comprises an amino acid sequence selected from:
a. the amino acid sequence of SEQ ID NO: 30 from position 1 to position 46 or the amino acid sequence of SEQ ID NO: 8 from position 1 to position 47; and
b. an amino acid sequence having at least 80% identity to the amino acid sequences of SEQ ID NO: 30 from position 1 to position 46 or the amino acid sequence of SEQ ID NO: 8 from position 1 to position 47.

29. The recombinant gene according to claim 22, wherein said nucleic acid encoding the thermostable Rca 2 protein variant comprises a coding nucleotide sequence selected from:
a. the nucleotide sequence of SEQ ID Nos: 31, 34, 36 or 37, or the complement thereof;
b. a nucleotide sequence having at least 60% identity with the nucleotide sequence of SEQ ID Nos: 31, 34, 36 or 37, or the complement thereof.

30. A vector comprising the recombinant gene according to claim 22.

31. A host cell comprising the recombinant gene of claim 22.

32. The cell of claim 31 which is a plant cell.

33. A knock out allele of an Rca 2 gene.

34. The knock out allele according to claim 33, wherein the Rca 2 gene is the Rca 2β gene from the wheat subgenome B, A or D or the Rca 2α gene from the wheat subgenome B, A or D.

35. A recombinant gene capable of suppressing specifically the expression of the endogenous Rca 2 genes comprising the following operably linked elements:
a. a promoter, preferably expressible in plants;
b. a nucleic acid which when transcribed yields an RNA molecule inhibitory to the endogenous Rca 2 genes encoding a non-thermostable Rca protein but not inhibitory to genes encoding thermostable Rca proteins; and, optionally
c. a transcription termination and polyadenylation region, preferably a transcription termination and polyadenylation region functional in plants.

36. The recombinant gene according to claim 35, wherein the endogenous Rca 2 genes comprise the coding nucleotide sequence of SEQ ID NO: 1 or a coding nucleotide sequence having at least 60% identity with the nucleotide sequence of SEQ ID NO: 1.

37. The recombinant gene according to claim 35, wherein said promoter is a constitutive promoter, a tissue-specific promoter or an inducible promoter.

38. A vector comprising the recombinant gene according to claim 35.

39. A host cell comprising the recombinant gene of claim 35.

40. The cell of claim 39 which is a plant cell.

41. A thermostable allele of a Rca 2 gene.

42. The thermostable allele according to claim 41 comprising
a. a coding nucleotide sequence of SEQ ID NOs: 31, 34, 36 or 37, or
b. a coding nucleotide sequence having at least 60% identity to SEQ ID NO: 31, 34, 36 or 37 and encoding a protein comprising at least one amino acid selected from:
i. an isoleucine at a position corresponding to position 59 of SEQ ID NO: 32 or 35;
ii. an aspartic acid at a position corresponding to position 73 of SEQ ID NO: 32 or 35;
iii. an isoleucine at a position corresponding to position 160 of SEQ ID NO: 32 or 35;
iv. an arginine at a position corresponding to position 265 of SEQ ID NO: 32 or 35;
v. a proline at a position corresponding to position 270 of SEQ ID NO: 32 or 35;
vi. a leucine at a position corresponding to position 277 of SEQ ID NO: 32 or 35;
vii. a glutamic acid at a position corresponding to position 307 of SEQ ID NO: 32 or 35;
viii. an isoleucine at a position corresponding to position 334 of SEQ ID NO: 32 or 35;
ix. a lysine at a position corresponding to position 359 of SEQ ID NO: 32 or 35;
x. a leucine at a position corresponding to position 361 of SEQ ID NO: 32 or 35; and
xi. a glutamic acid at a position corresponding to position 363 of SEQ ID NO: 32 or 35.

43. The thermostable allele of a Rca 2 according to claim 41, wherein the Rca 2 gene is the wheat Rca 2β gene from the subgenome B, A or D or the wheat Rca 2α gene from the subgenome B, A or D.

44. The plant cell according to claim 32 further comprising:
a. at least one knock out allele of a Rca 2 gene; or
b. a recombinant gene capable of suppressing specifically the expression of the endogenous Rca 2 genes according to claim 35, or the vector according to claim 38.

45. A cereal plant cell comprising at least one thermostable mutant allele of an Rca 2 gene according to claim 41.

46. A cell comprising the thermostable Rca 2 protein variant according to claim 19.

47. A plant, plant part or seed consisting essentially of the plant cells of claim 32.

48. The plant, plant part or seed according to claim 47 which is a cereal plant, cereal plant part or a cereal seed.

49. A method for increasing thermotolerance of a cereal plant comprising:
a. Increasing the ratio of a thermostable Rca protein; and
b. Regenerating said cereal plant.

50. The method according to claim 49 wherein said thermostable Rca protein is a Rca 1β protein or variants thereof.

51. The method according to claim 49 wherein said thermostable Rca protein is the thermostable Rca 2 protein variant.

52. (canceled)

53. A method of producing food, feed or an industrial product comprising
a. obtaining the plant, part thereof or seed according to claim 47; and
b. preparing the food, feed or industrial product from said plant, part thereof or seed.

54. The method according to claim 53, wherein
a. the food or feed is meal, grain, starch, flour or protein; or
b. the industrial product is biofuel, fiber, industrial chemicals, a pharmaceutical or a nutraceutical.

55. A method of increasing thermostability of a Rca 2 protein comprising introducing at least one amino acid substitution to the amino acid sequence of said Rca 2 protein, wherein the amino acid substitution is selected from:
a. Substituting a valine with an isoleucine at a position corresponding to position 59 of SEQ ID NO: 4;
b. Substituting a glycine with an aspartic acid at a position corresponding to position 73 of SEQ ID NO: 4;
c. Substituting a methionine with an isoleucine at a position corresponding to position 160 of SEQ ID NO: 4;
d. Substituting a glutamine with an arginine at a position corresponding to position 265 of SEQ ID NO: 4;
e. Substituting a serine with a proline at a position corresponding to position 270 of SEQ ID NO: 4;
f. Substituting an isoleucine with a leucine at a position corresponding to position 277 of SEQ ID NO: 4;
g. Substituting a serine with a glutamic acid at a position corresponding to position 307 of SEQ ID NO: 4;
h. Substituting a valine with an isoleucine at a position corresponding to position 334 of SEQ ID NO: 4;
i. Substituting threonine with a lysine at a position corresponding to position 359 of SEQ ID NO: 4;
j. Substituting methionine with a leucine at a position corresponding to position 361 of SEQ ID NO: 4; and
k. Substituting a glutamine with a glutamic acid at a position corresponding to position 363 of SEQ ID NO: 4.

56. The method according to claim 55, wherein the thermostability is increased by about 7° C.

57. A method for producing a thermostable Rca 2 protein variant comprising culturing the host cell according to claim 31 and isolating the protein produced.

58. A cereal plant comprising the thermostable Rca 2 protein variant according to claim 19.


SEQUENCE LISTING (69 sequences)

SEQ ID NO: 1 - DNA, 1284 bp, Triticum aestivum
atggctgctgccttctcctccaccgtcggtgccccggcttctacgccgaccaacttccttgggaagaagctcaagaagcaggtgacctcggccgtgaactaccatggcaagagctccaaggccaacaggttcacagtcatggcagcggaaaacctcgacgagaagaggaacacggacaagtggaagggtcttgcgtacgatatctccgacgaccagcaggacatcaccagagggaagggcatcgtggactcgctcttccaggcgcccacgggcgacggcacccacgaggccgtcctcagctcctacgagtacgtcagccagggactcaagaagtacgacttcgacaacaccatgggaggcttctacatcgctcctgctttcatggacaagcttgttgtccatctctccaagaacttcatgaccctgcccaacatcaagatcccactcatcttgggtatctggggaggcaagggtcaaggaaaatcattccagtgcgagcttgtcttcgccaagatgggcatcaacccaatcatgatgagtgccggagagctggagagtggcaacgccggagagccagccaagctcatcaggcagcggtaccgtgaggctgcagacatgatcaagaagggtaagatgtgctgcctcttcatcaacgatcttgacgctggtgcgggtcggatgggcgggaccacacagtacaccgtcaacaaccagatggtgaacgccaccctcatgaacatcgccgatgcccccaccaacgtgcagctcccaggcatgtacaacaaggaggagaaccctcgtgtgcccatcgtcgtcactggtaacgatttctcgacgttgtacgcccctctgatccgtgatggccgtatggagaagttctactgggctcccacccgcgacgaccgtatcggtgtctgcaagggtatcttccagaccgacaatgtcagcgacgagtccgtcgtcaagatcgtcgacaccttcccaggacaatccatcgactttttcggtgctctgcgtgctcgggtgtacgacgacgaggtgcgcaagtgggtgacctctaccggtatcgagaacattggcaagaggctggtgaactcgcgggacggacccgtgacctttgagcagccaaagatgacagtggagaagctgctagagtacgggcacatgcttgtccaggagcaggacaatgtcaagcgtgtgcagcttgctgacacctacatgagccaggcagctctgggtgatgctaaccaggatgcgatgaagactggttccttctacggttag

SEQ ID NO: 2 - protein, 427 aa, Triticum aestivum
MAAAFSSTVGAPASTPTNFLGKKLKKQVTSAVNYHGKSSKANRFTVMAAENLDEKRNTDKWKGLAYDISDDQQDITRGKGIVDSLFQAPTGDGTHEAVLSSYEYVSQGLKKYDFDNTMGGFYIAPAFMDKLVVHLSKNFMTLPNIKIPLILGIWGGKGQGKSFQCELVFAKMGINPIMMSAGELESGNAGEPAKLIRQRYREAADMIKKGKMCCLFINDLDAGAGRMGGTTQYTVNNQMVNATLMNIADAPTNVQLPGMYNKEENPRVPIVVTGNDFSTLYAPLIRDGRMEKFYWAPTRDDRIGVCKGIFQTDNVSDESVVKIVDTFPGQSIDFFGALRARVYDDEVRKWVTSTGIENIGKRLVNSRDGPVTFEQPKMTVEKLLEYGHMLVQEQDNVKRVQLADTYMSQAALGDANQDAMKTGSFYG

SEQ ID NO: 3 - DNA, 1284 bp, Triticum aestivum; identical to SEQ ID NO: 1

SEQ ID NO: 4 - protein, 381 aa, Triticum aestivum
MAAENLDEKRNTDKWKGLAYDISDDQQDITRGKGIVDSLFQAPTGDGTHEAVLSSYEYVSQGLKKYDFDNTMGGFYIAPAFMDKLVVHLSKNFMTLPNIKIPLILGIWGGKGQGKSFQCELVFAKMGINPIMMSAGELESGNAGEPAKLIRQRYREAADMIKKGKMCCLFINDLDAGAGRMGGTTQYTVNNQMVNATLMNIADAPTNVQLPGMYNKEENPRVPIVVTGNDFSTLYAPLIRDGRMEKFYWAPTRDDRIGVCKGIFQTDNVSDESVVKIVDTFPGQSIDFFGALRARVYDDEVRKWVTSTGIENIGKRLVNSRDGPVTFEQPKMTVEKLLEYGHMLVQEQDNVKRVQLADTYMSQAALGDANQDAMKTGSFYG

SEQ ID NO: 5 - DNA, 1395 bp, Triticum aestivum
atggctgctgccttctcctccaccgtcggtgccccggcttctacgccgaccaacttccttgggaagaagctcaagaagcaggtgacctcggccgtgaactaccatggcaagagctccaaggccaacaggttcacagtcatggcagcggaaaacctcgacgagaagaggaacacggacaagtggaagggtcttgcgtacgatatctccgacgaccagcaggacatcaccagagggaagggcatcgtggactcgctcttccaggcgcccacgggcgacggcacccacgaggccgtcctcagctcctacgagtacgtcagccagggactcaagaagtacgacttcgacaacaccatgggaggcttctacatcgctcctgctttcatggacaagcttgttgtccatctctccaagaacttcatgaccctgcccaacatcaagatcccactcatcttgggtatctggggaggcaagggtcaaggaaaatcattccagtgcgagcttgtcttcgccaagatgggcatcaacccaatcatgatgagtgccggagagctggagagtggcaacgccggagagccagccaagctcatcaggcagcggtaccgtgaggctgcagacatgatcaagaagggtaagatgtgctgcctcttcatcaacgatcttgacgctggtgcgggtcggatgggcgggaccacacagtacaccgtcaacaaccagatggtgaacgccaccctcatgaacatcgccgatgcccccaccaacgtgcagctcccaggcatgtacaacaaggaggagaaccctcgtgtgcccatcgtcgtcactggtaacgatttctcgacgttgtacgcccctctgatccgtgatggccgtatggagaagttctactgggctcccacccgcgacgaccgtatcggtgtctgcaagggtatcttccagaccgacaatgtcagcgacgagtccgtcgtcaagatcgtcgacaccttcccaggacaatccatcgactttttcggtgctctgcgtgctcgggtgtacgacgacgaggtgcgcaagtgggtgacctctaccggtatcgagaacattggcaagaggctggtgaactcgcgggacggacccgtgacctttgagcagccaaagatgacagtggagaagctgctagagtacgggcacatgcttgtccaggagcaggacaatgtcaagcgtgtgcagcttgctgacacctacatgagccaggcagctctgggtgatgctaaccaggatgcgatgaagactggttccttctacggtaaaggggcacagcaaggtactttgcctgtaccggcaggatgcaccgaccagactgccaagaacttcgacccaacggcgaggagtgacgacgggagctgcctttacaccttttaa

SEQ ID NO: 6 - protein, 464 aa, Triticum aestivum
MAAAFSSTVGAPASTPTNFLGKKLKKQVTSAVNYHGKSSKANRFTVMAAENLDEKRNTDKWKGLAYDISDDQQDITRGKGIVDSLFQAPTGDGTHEAVLSSYEYVSQGLKKYDFDNTMGGFYIAPAFMDKLVVHLSKNFMTLPNIKIPLILGIWGGKGQGKSFQCELVFAKMGINPIMMSAGELESGNAGEPAKLIRQRYREAADMIKKGKMCCLFINDLDAGAGRMGGTTQYTVNNQMVNATLMNIADAPTNVQLPGMYNKEENPRVPIVVTGNDFSTLYAPLIRDGRMEKFYWAPTRDDRIGVCKGIFQTDNVSDESVVKIVDTFPGQSIDFFGALRARVYDDEVRKWVTSTGIENIGKRLVNSRDGPVTFEQPKMTVEKLLEYGHMLVQEQDNVKRVQLADTYMSQAALGDANQDAMKTGSFYGKGAQQGTLPVPAGCTDQTAKNFDPTARSDDGSCLYTF

SEQ ID NO: 7 - DNA, 1299 bp, Triticum aestivum
atggcttctgctttctcgtccaccgttggagctccggcgtcaaccccgaccaccttcctcgggaagaaggtgaagaagcaggccggtgcgttgaactactaccatggtggcaacaagatcaagagtaaggtggtcagggccatggcggccaaaaaggaacttgacgagggcaagcagaccaatgccgataggtggaagggtctcgcttacgacatctccgacgaccagcaggacatcacgagtgggaaaggtatcgtagactccctgttccaggcccccatgggcgacggcacccacgaggccatcctgagctcctacgagtacatcagccagggcctgcggaagtacgacttcgacaacaccatggacggcctctacatcgccccggcattcatggacaagctcatcgtccacctcgccaagaacttcatgacactccccaacatcaaggttcctctcatcctgggcatctggggaggcaagggacagggcaagtcgttccagtgcgagctggtgttcgccaagatgggcatcaaccccatcatgatgagcgccggtgagctggagagcggcaacgccggcgagccggcgaagctgatccggcagaggtaccgcgaggctgccgacattatcaacaagggcaagatgtgctgcctcttcatcaacgacctggacgccggcgcggggcggatgggcgggacgacgcagtacacggtgaacaaccagatggtgaacgccaccctgatgaacatcgcggacgcgcccaccaacgtgcagctccccgggatgtacaacaaggaggagaacccccgcgtgcccatcatcgtcacgggcaacgacttctcaacgctgtacgcgcccctgatccgcgatggccgcatggagaagttctactgggcgcccacccgcgaggaccgcatcggcgtgtgcaagggcatcttccgcaccgacaacgtccccgacgaggccgtggtgaggctggtggacaccttcccggggcagtccatcgacttcttcggcgcgctgcgggcgcgggtgtacgacgacgaggtgcgcaagtgggtcggcgagatcggcgtggagaacatctccaagcggctcgtcaactccagggaggggccgccgacgttcgaccagcccaagatgaccatcgagaagctcatggagtacggccacatgctggtccaggagcaggagaacgtgaagcgcgtgcagctcgccgacaagtacctcagcgaggcggcgctcggccaagccaacgacgacgccatgaagaccggcgccttctacggcaagtag

SEQ ID NO: 8 - protein, 432 aa, Triticum aestivum
MASAFSSTVGAPASTPTTFLGKKVKKQAGALNYYHGGNKIKSKVVRAMAAKKELDEGKQTNADRWKGLAYDISDDQQDITSGKGIVDSLFQAPMGDGTHEAILSSYEYISQGLRKYDFDNTMDGLYIAPAFMDKLIVHLAKNFMTLPNIKVPLILGIWGGKGQGKSFQCELVFAKMGINPIMMSAGELESGNAGEPAKLIRQRYREAADIINKGKMCCLFINDLDAGAGRMGGTTQYTVNNQMVNATLMNIADAPTNVQLPGMYNKEENPRVPIIVTGNDFSTLYAPLIRDGRMEKFYWAPTREDRIGVCKGIFRTDNVPDEAVVRLVDTFPGQSIDFFGALRARVYDDEVRKWVGEIGVENISKRLVNSREGPPTFDQPKMTIEKLMEYGHMLVQEQENVKRVQLADKYLSEAALGQANDDAMKTGAFYGK

SEQ ID NO: 9 - DNA, 1158 bp, Triticum aestivum
atggcggccaaaaaggaacttgacgagggcaagcagaccaatgccgataggtggaagggtctcgcttacgacatctccgacgaccagcaggacatcacgagtgggaaaggtatcgtagactccctgttccaggcccccatgggcgacggcacccacgaggccatcctgagctcctacgagtacatcagccagggcctgcggaagtacgacttcgacaacaccatggacggcctctacatcgccccggcattcatggacaagctcatcgtccacctcgccaagaacttcatgacactccccaacatcaaggttcctctcatcctgggcatctggggaggcaagggacagggcaagtcgttccagtgcgagctggtgttcgccaagatgggcatcaaccccatcatgatgagcgccggtgagctggagagcggcaacgccggcgagccggcgaagctgatccggcagaggtaccgcgaggctgccgacattatcaacaagggcaagatgtgctgcctcttcatcaacgacctggacgccggcgcggggcggatgggcgggacgacgcagtacacggtgaacaaccagatggtgaacgccaccctgatgaacatcgcggacgcgcccaccaacgtgcagctccccgggatgtacaacaaggaggagaacccccgcgtgcccatcatcgtcacgggcaacgacttctcaacgctgtacgcgcccctgatccgcgatggccgcatggagaagttctactgggcgcccacccgcgaggaccgcatcggcgtgtgcaagggcatcttccgcaccgacaacgtccccgacgaggccgtggtgaggctggtggacaccttcccggggcagtccatcgacttcttcggcgcgctgcgggcgcgggtgtacgacgacgaggtgcgcaagtgggtcggcgagatcggcgtggagaacatctccaagcggctcgtcaactccagggaggggccgccgacgttcgaccagcccaagatgaccatcgagaagctcatggagtacggccacatgctggtccaggagcaggagaacgtgaagcgcgtgcagctcgccgacaagtacctcagcgaggcggcgctcggccaagccaacgacgacgccatgaagaccggcgccttctacggcaagtag

SEQ ID NO: 10 - protein, 385 aa, Triticum aestivum
MAAKKELDEGKQTNADRWKGLAYDISDDQQDITSGKGIVDSLFQAPMGDGTHEAILSSYEYISQGLRKYDFDNTMDGLYIAPAFMDKLIVHLAKNFMTLPNIKVPLILGIWGGKGQGKSFQCELVFAKMGINPIMMSAGELESGNAGEPAKLIRQRYREAADIINKGKMCCLFINDLDAGAGRMGGTTQYTVNNQMVNATLMNIADAPTNVQLPGMYNKEENPRVPIIVTGNDFSTLYAPLIRDGRMEKFYWAPTREDRIGVCKGIFRTDNVPDEAVVRLVDTFPGQSIDFFGALRARVYDDEVRKWVGEIGVENISKRLVNSREGPPTFDQPKMTIEKLMEYGHMLVQEQENVKRVQLADKYLSEAALGQANDDAMKTGAFYGK

SEQ ID NO: 11 - protein, 433 aa, Oryza sativa
MAAAFSSTVGAPASTPTNFLGKKLKKQVTSAVNYHGKSSNINRFKVMAKELDEGKQTDQDRWKGLAYDISDDQQDITRGKGFVDSLFQAPTGDGTHEAVLSSYEYLSQGLRTYDFDNTMGGFYIAPAFMDKLVVHISKNFMTLPNIKVPLILGIWGGKGQGKSFQCELVFAKMGINPIMMSAGELESGNAGEPAKLIRQRYREAADIIKKGKMCCLFINDLDAGAGRMGGTTQYTVNNQMVNATLMNIADNPTNVQLPGMYNKEDNPRVPIIVTGNDFSTLYAPLIRDGRMEKFYWAPTRDDRVGVCKGIFRTDNVPDEDIVKIVDSFPGQSIDFFGALRARVYDDEVRKWVSDTGVENIGKRLVNSREGPPEFEQPKMTIEKLMEYGYMLVKEQENVKRVQLAEQYLSEAALGDANSDAMKTGSFYGSAPSS

SEQ ID NO: 12 - protein, 431 aa, Oryza australiensis
MAAAFSSTVGAPASTPTNFLGKKLKKQVTSAVNYHGKSSNINRFKVMAKELDEDKQTDQDKWKGLAYDISDDQQDITRGKGLVDSLFQAPMGDGTHEAVLSSYEYLSQGLKMLDNTMGGFYIAPAFMDKLVVHISKNFMALPNIKVPLILGIWGGKGQGKSFQCELVFSKMGINPIMMSAGELESGNAGEPAKLIRQRYREAADIIKKGKMCCLFINDLDAGAGRMGGTTQYTVNNQMVNATLMNIADNPTNVQLPGMYNKEDNPRVPIIVTGNDFSTLYAPLIRDGRMEKFYWAPTRDDRVGVCKGIFRTDNVPDEDIVKIVDSFPGQSIDFFGALRARVYDDEVRKWVSDTGVENIGKKLVNSREGPPEFEQPKMTIDKLMEYGHMLVREQENVKRVQLADKYLSEAALGDANSDAMKTGSFYGAAPSS

SEQ ID NO: 13 - protein, 435 aa, Larrea tridentate
MAAAYSTVGAVNRAPLSLNGSGARASLVPSTAFFGSSLKKSAAKFPKASSGNFKIVAQEISEDQQTDKDKWKGLAYDISDDQQDITRGKGMVDTLFQAPMQSGTHYAVMSSYDYISQGLRQYNLDNNMDGFYIAPAFMDKLVVHITKNFLSLPNIKIPLILGIWGGKGQGKSFQCELVFAKMGINPIMMSAGELESGNAGEPAKLIRQRYREAADIIKKGKMCCLFINDLDAGAGRMGGTTQYTVNNQMVNATLMNIADNPTNVQLPGMYNKEENPRVPIIVTGNDFSTLYAPLIRDGRMEKFYWAPTREDRIGVCKGIFRTDNVPEEDIVKVVDQFPGQSIDFFGALRARVYDDEVRKWVSEVGVDTIGKKLVNSKEGPPTFEQPKMTIDKLLQYGNMLVEEQENVKRVQLADKYMSEAALGDANQDAIKRGTF

SEQ ID NO: 14 - protein, 438 aa, Musa acuminata
MAAAVSTVGAVNRVPLSLHGSSSGASVPSSAFFGSALKKANSGLSHGRVSTASFKVLAADLDESKQTSSDRWAGLASDVSDDQQDITRGKGLVDTLFQAPMGDGTHIPVMSSYEYISQGLRQYNFDNTMDGYYIAPAFMDKLVVHIAKNFMNLPNIKVPLILGIWGGKGQGKSFQCELVFAKMGINPIVMSAGELESGNAGEPAKLIRQRYREAADIIKKGKMCCLFINDLDAGAGRLGGTTQYTVNNQMVNATLMNIADNPTNVQLPGMYNKQENARVPIIVTGNDFSTLYAPLIRDGRMEKFYWAPTREDRIGVCLGIFRTDNVPMEDIVKLVDAFPGQSIDFFGALRARVYDDEVRKWVAEIGVEKVGKKLVNSLEGPPTFEQPKMTLDKLTEYGNMLVREQENVKRVQLADKYLSEAALGDANADAIKTGSFYG

SEQ ID NO: 15 - protein, 373 aa, Datisca glomerata
MASSAVSTIGAVNRSPLNLNNNGTGGLLPNTAFFGSSLKKMNSRLTNPRIAAGNIKAVADDDEEKQTSKDRWGGLAFDTSDDQQDITRGKGMVDSLFQAPMQTGTHYAVMSSYEYLSTGLRQYLDNNMDGFYIAPAFMDKLVVHITKNFMTLPNIKVPLILGIWGGKGQGKSFQCELVFAKMGINPIMMSAGELESGNAGEPAKLIRQRCREAADIIKKGKMSCLFINDLDAGAGRLGGTTQYTVNNQMVNATLMNIADNPTNVQLPGMYNKEENPRVPIIVTGNDFSTLYAPLIRDGRMEKFYWAPTREDRIGVCTGIFRSDNVAKEDIVKLVDTFPGQSIDFFGALRARVYDDEVRKWISGVGVQDVGKSL

SEQ ID NO: 16 - protein, 431 aa, Theobroma cacao
MSLSRELQWNLNGSKPAAASLPSSAFLGNTLKKVTSKISHRKVSPKNFKVVAEYDESKKTSKDRWKGLAYDESDDQQDITRGKGMVDTLFQAPMGTGTHHAIMSSYDYISQGLRMYNLDNTMDGFYIAPAFMDKVVVHITKNFLSLPNIKVPLILGIWGGKGQGKSFQCELVFAKMGINSIMMSAGELESGNAGEPAKLIRQRYREAADIIRKGKMCCLFINDLDAGAGRMGGTTQYTVNNQMVNATLMNIADNPTNVQLPGMYNKEENPRVPIIVTGNDFSTLYAPLIRDGRMEKFYWAPTREDRIGVCSGIFKTDRVPHEDSVKLVDTFPGQSIDFFSALRARVYDDEVREWVSEVGVQNVGKRLVNSKEGPPTFEQPKMTLEKLLEYGNMLVQEQENVKRVQLADKYLKEAALGEANDDAIKNESFYG

SEQ ID NO: 17 - protein, 442 aa, Nicotiana tabacum
MATSVSTIGAVNKTPLSLNNSVAGTSVPSTAFFGKTLKKVYGKGVSSPKVTNKSLRIVAEQIDVDPKKQTDSDRWKGLVQDFSDDQQDITRGKGMVDSLFQAPTGTGTHHAVLQSYEYVSQGLRQYNLDNKLDGFYIAPAFMDKLVVHITKNFLKLPNIKVPLILGIWGGKGQGKSFQCELVFRKMGINPIMMSAGELESGNAGEPAKLIRQRYREAAEIIRKGNMCCLFINDLDAGAGRMGGTTQYTVNNQMVNATLMNIADNPTNVQLPGMYNKQENARVPIIVTGNDFSTLYAPLIRDGRMEKFYWAPTREDRIGVCTGIFRTDNVPAEDVVKIVDNFPGQSIDFFGALRARVYDDEVRKWVSGTGIEKIGDKLLNSFDGPPTFEQPKMTIEKLLEYGNMLVQEQENVKRVQLADKYLKEAALGDANADAINNGSFFAS

SEQ ID NO: 18 - protein, 421 aa, Gossypium hirsutum
MAAEKEIDEETQTEKDRWKGLAYDISDDQQDITRGKGMVDSLFQAPMNDGTHYAVMSSYEYISQGLKTYNLDNNMDGFYIAPAFMDKLVVHISKNFMSLPNIKVPLILGIWGGKGQGKSFQCELVFAKMGINPIMMSAGELESGNAGEPAKLIRQRYREAADIIKKGKMCALFINDLDAGAGRMGGTTQYTVNNQMVNATLMNIADNPTNVQLPGMYNKEENPRVPIIVTGNDFSTLYAPLIRDGRMEKFYWAPTRDDRIGVCKGIFRTDGVRDEDIVKLVDTFPGQSIDFFGALRARVYDDEVRKWISEVGVASVGKKLVNSREGPPTFEQPKMTIEKLLEYGNMLVAEQENVKRVQLADKYLSEAALGEANEDSINRGTFYGKAAQQVGVPVPEGCTDPNADNFDPTARSDDGTCTYKF

SEQ ID NO: 19 - protein, 430 aa, Artificial Sequence
  amino acid consensus sequence from the “warm” species
  misc_feature (30)..(31): Xaa can be any naturally occurring amino acid
  misc_feature (35)..(36): Xaa can be any naturally occurring amino acid
  misc_feature (38)..(39): Xaa can be any naturally occurring amino acid
  misc_feature (44)..(44): Xaa can be any naturally occurring amino acid
  misc_feature (53)..(53): Xaa can be any naturally occurring amino acid
  misc_feature (362)..(362): Xaa can be any naturally occurring amino acid
  misc_feature (395)..(395): Xaa can be any naturally occurring amino acid
  misc_feature (420)..(420): Xaa can be any naturally occurring amino acid
MAASSTVGAVNRTPLSLNGSGVPSTAFFGXXLKKXXNXXKVSIXNFKVVAKEXDEDKQTDKDRWKGLAYDISDDQQDITRGKGMVDSLFQAPMGDGTHYAVMSSYEYISQGLRQYNLDNTMDGFYIAPAFMDKLVVHITKNFMSLPNIKVPLILGIWGGKGQGKSFQCELVFAKMGINPIMMSAGELESGNAGEPAKLIRQRYREAADIIKKGKMCCLFINDLDAGAGRMGGTTQYTVNNQMVNATLMNIADNPTNVQLPGMYNKEENPRVPIIVTGNDFSTLYAPLIRDGRMEKFYWAPTREDRIGVCKGIFRTDNVPDEDIVKLVDTFPGQSIDFFGALRARVYDDEVRKWVSEVGVENXGKKLVNSREGPPTFEQPKMTIEKLLEYGNMLVXEQENVKRVQLADKYLSEAALGDANXDAIKTGSFYG

SEQ ID NO: 20 - protein, 441 aa, Arabidopsis thaliana
MAAAVSTVGAINRAPLSLNGSGSGAVSAPASTFLGKKVVTVSRFAQSNKKSNGSFKVLAVKEDKQTDGDRWRGLAYDTSDDQQDITRGKGMVDSVFQAPMGTGTHHAVLSSYEYVSQGLRQYNLDNMMDGFYIAPAFMDKLVVHITKNFLTLPNIKVPLILGIWGGKGQGKSFQCELVMAKMGINPIMMSAGELESGNAGEPAKLIRQRYREAADLIKKGKMCCLFINDLDAGAGRMGGTTQYTVNNQMVNATLMNIADNPTNVQLPGMYNKEENARVPIICTGNDFSTLYAPLIRDGRMEKFYWAPTREDRIGVCKGIFRTDKIKDEDIVTLVDQFPGQSIDFFGALRARVYDDEVRKFVESLGVEKIGKRLVNSREGPPVFEQPEMTYEKLMEYGNMLVMEQENVKRVQLAETYLSQAALGDANADAIGRGTFYGKTEV

SEQ ID NO: 21 - protein, 438 aa, Brassica oleracea
MAAAVSTVGAINRAPLSLNGSGAGAASVPATTFLGKKAVTASRFTQSNNKKSNGSFKVVAVKEDKQTDGDRWKGLAYDTSDDQQDITRGKGMVDSVFQAPMGTGTHNAVLSSYEYISQGLKQYNLDNMMDGLFIAPAFMDKLVVHITKNFLTLPNIKVPLILGIWGGKGQGKSFQCELVMAKMGINPIMMSAGELESGNAGEPAKLIRQRYREAADMIKKGKMCCLFINDLDAGAGRMGGTTQYTVNNQMVNATLMNIADNPTNVQLPGMYNKEENARVPIIVTGNDFSTLYAPLIRDGRMEKFYWAPTREDRIGVCKGIFRTDNVKDEDIVTLVDQFPGQSIDFFGALRARVYDDEVRKFVEGLGVEKIGKRLVNSREGPPVFEQPAMTLEKLMEYGNMLVMEQENVKRVQLADQYLNEAALGDANADAIGRGTFYG

SEQ ID NO: 22 - protein, 440 aa, Picea sitchensis
MATAAAAVGSITVSSNLPLSSGQASILKTVPSTGFFGCGLKKTSFNASHTFTKANHFKVVAEIDEGKQTDKDKWKGLAFDESDDQMDIRRGKGKVDSLFQAPMGSGTHNVVMSTYDYISTAQRTYDFDNTMDGYYIAPSFMDKLLVHISKNFMNLPNIKVPLILGIWGGKGQGKSFQCELVFAKLGINPIMMSAGELESGDAGEPAKLLRKRYREASDIVKKGKMCVLFINDLDAGAGRMGSTTQYTVNNQMVNATLMNIADNPTNVQLPGMYNKQDNPRVPIVVTGNDFSTLYAPLIRDGRMEKFYWAPTRDDRIGVCQGIFRADNVHPDDVVRLVDTFPGQSIDFFGALRARVYDDEVRKWVAGTGIQNIGKKLINSKEGPPTFEKPAMTIEKLLEYGNMLVGEQDNVKRVQLADKYMSEAALGDANEDSIKRGTFYG

SEQ ID NO: 23 - protein, 472 aa, Spinacia oleracea
MATAVSTVGAATRAPLNLNGSSAGASVPTSGFLGSSLKKHTNVRFPSSSRTTSMTVKAAENEEKNTDKWAHLAKDFSDDQLDIRRGKGMVDSLFQAPADAGTHVPIQSSFEYESQGLRKYDIDNMLGDLYIAPAFMDKLVVHITKNFLNLPNIKIPLILGVWGGKGQGKSFQCELVFAKLGINPIMMSAGELESGNAGEPAKLIRQRYREAADLIAKGKMCALFINDLEPGAGRMGGTTQYTVNNQMVNATLLNIADNPTNVQLPGMYNKQDNARVPIIVTGNDFSTLYAPLIRDGRMEKFYWAPTREDRIGVCTGIFKTDKVPAEHVVKLVDAFPGQSIDFFGALRARVYDDEVRKWVNSVGVDNVGKKLVNSKDGPPVFEQPEMTLQKLMEYGNMLVQEQENVKRVQLADQYMSSAALGDANKDAIDRGTFFGKAAQQVSLPVAQGCTDPEAKNYDPTARSDDGSCTYNL

SEQ ID NO: 24 - protein, 428 aa, Fragaria vesca
MTLCNSFLLKTPIFPPHSHYFPPQFPTKPIFSLSCSANPSSSSDDNATNGGGEPKTKKKLSDQSSWEAEDSSGEDYLYRLGKEADNMNIAVGARAGVVDDLFVGQFLGRDSDIVFDYRQKVTRSFQYLQGDYYIAPLFMDKVVCHMTKNYLAPILNTKVPLILGIWGGKGQGKSFQTELIFQAMGVEPVIMSAGELESERAGEPGKLIRERYRTASQVVQNQGKMSCLMINDIDAGLGRFGNTQMTVNNQIVVGTLMNLCDNPTRVSIGQEWRDSDITNRVPVIVTGNDFSKIYAPLIRDGRMEKFYWQPNREDIINIVNRMYEKDGISRDEVVSIVDTFPNQALDFYGALRSRTYDRSILKWVDDIGGVESLGNQLLKRRKDDNLPVYTAPKQTVEALLESGYSLLKEQQLVMESKLSKEYMKNMDD

SEQ ID NO: 25 - protein, 442 aa, arachis duranensis
MPRGIEELLVQNEMNESPIMCKCAMAAIPSGWCAAHTSIRIEKKRLPFLRVGAKLCVRCCSDGSEKEEEKKKRLSEQSSWEAKDAQGRDYLYRLGKEADNMNIAVGQRAGVIDSLFVGDFLGKDSDIVFDYRQKATRSFEYLQGDYYIAPLFMDKVVCHIVKNYLAHLLNTKVPLILGIWGGKGQGKSFQTELIFQAMGIEPVIMSAGELESERAGEPGKLIRERYRTASQVVQNQGKMSCLMINDIDAGLGRFGNTQMTVNNQIVVGTLMNLSDNPTRVSIGQDWRESDVTNRIPIIVTGNDFSTIYAPLIRDGRMDKFYWQPNHEDIVNIVHRMYEKDGIPKDEVIRIVNTFPNQALDFYGALRSRTYDRSILKWIEDIGGIENFGKKFLKRKKDQSLPVFIPPEQTMEALVESGYSLIKEQQLIMETKLSKEYMKNIQE

SEQ ID NO: 26 - protein, 437 aa, Brachypodium distachyon
MATTSLAAASHRHILFPSRSNPNSNLRTPNRGRLCPTFPPILCSSSSSASPQPTAGGDEEEEAGRGRRLSKQSSWEATDAEGDDYLYRLGKEADNMNIAVGARSGIIDDLFVGKFLGRDSDIVFDYRQKVTRKFEYLQGDYYIAPSFLDKVACHIVKNYLANNLNIKVPLILGVWGGKGQGKTFQTELIFKAMGVEPVIMSAGELESERAGEPGRLIRDRYRTASQVIQNQGKMSCLMINDLDAGVGRFGNTQMTVNNQIVVGTLMNLADNPTRVSIGQKWRESDITHRVPIIATGNDFSTLYAPLIRDGRMEKFYWQPDREDIINIVHRMYMKDGLSFEEVSRIVDTFPNQALDFYGALRSRTYDQAILQWVNDIGGYEQLGEKLLKKKKREKLPTFIPPKPTLDALIESGDSLVKEQELIMNSKLSKEYMKNLEG

SEQ ID NO: 27 - protein, 436 aa, Arabis alpina
MALANISLRFKFPVPQLQSLSSSTFHTLISIRKPSSIACSKPSSSSSLSAGDEVSKEANNGGEKRRKLSEQSSWEVKDSEGKDYLYRLGAESDNVNIAVGARSGMIDDVFIGDFLGKDSDIVFDYRQKATRSFEHLQGDYYIAPTFLDKVAVHIVKNYLASSLNIKIPLILGIWGGKGQGKTFQTELIFKTMGVEPVIMSAGELESDRAGEPGRLIRDRYRTASQVIQNQGKMSVLMINDIDAGLGRFGETQMTVNNQIVVGTLMNLADNPTRVSVGQDWREADIVNRVPLIVTGNDFSRLYAPLIREGRMEKFYWQPTREDIVNIVSRMYEKDGISRKEVASIVDKFPNQALDFYGALRSRTYDRSILKWVDDAGGMETLGKTLLRRKKTKEVPQFIPPEQTMEALLESGYSLIREQKLIMETKLSREYMKNIDD

SEQ ID NO: 28 - protein, 456 aa, Mesembryanthemum crystallinum
MALLINPTNPTQFSNLHSLSSLTSFPHKLSLSKPRLKLSQIRQFSRRKNFSVFVELKNGAQIENGDDKPSSSVAENTPVRGTERKKRLSEQSSWEAKDALGNDYLYRLGAEADNTNITVGARPGVIDDLFTGNFLGRDSDIVFDYRQKVTRSFEYLRGDYYIAPVFLDQVVCHIVKNFMAHLLNVKVPLILGIWGGKGQGKTFQTELIFRAMGVEPVIMSAGEMESEKAGEPGRLIRERYRAASQVVQNHGKLSCLMINDLDAGIGRFGNTQVTVNNQTAAGTLMNLADNPTRVSIGQKWRENDTTHRIPVIVTGNDFSTIYAPLIRDGRMDKFYWQPTHDDIVNIVHRMYEKDGISWDEVVSIVNTFPNQALDFYGAMRSRTYDRSVLKWVDDIGGIDNLGAKLLQLRKGDELPVFVPPEQNVEALLESGYSLLREQQLINKTKLAKEYMKNMED

SEQ ID NO: 29 - protein, 429 aa, Artificial Sequence
  consensus sequence from the “cold” RCA sequences
  misc_feature (9)..(9): Xaa can be any naturally occurring amino acid
  misc_feature (17)..(17): Xaa can be any naturally occurring amino acid
  misc_feature (20)..(20): Xaa can be any naturally occurring amino acid
  misc_feature (23)..(23): Xaa can be any naturally occurring amino acid
  misc_feature (28)..(28): Xaa can be any naturally occurring amino acid
  misc_feature (30)..(30): Xaa can be any naturally occurring amino acid
  misc_feature (38)..(38): Xaa can be any naturally occurring amino acid
  misc_feature (41)..(41): Xaa can be any naturally occurring amino acid
  misc_feature (43)..(43): Xaa can be any naturally occurring amino acid
  misc_feature (46)..(46): Xaa can be any naturally occurring amino acid
  misc_feature (49)..(50): Xaa can be any naturally occurring amino acid
  misc_feature (57)..(58): Xaa can be any naturally occurring amino acid
  misc_feature (63)..(63): Xaa can be any naturally occurring amino acid
  misc_feature (97)..(97): Xaa can be any naturally occurring amino acid
  misc_feature (124)..(124): Xaa can be any naturally occurring amino acid
  misc_feature (150)..(150): Xaa can be any naturally occurring amino acid
  misc_feature (186)..(186): Xaa can be any naturally occurring amino acid
  misc_feature (202)..(202): Xaa can be any naturally occurring amino acid
  misc_feature (213)..(213): Xaa can be any naturally occurring amino acid
  misc_feature (217)..(217): Xaa can be any naturally occurring amino acid
  misc_feature (245)..(245): Xaa can be any naturally occurring amino acid
  misc_feature (248)..(248): Xaa can be any naturally occurring amino acid
  misc_feature (254)..(254): Xaa can be any naturally occurring amino acid
  misc_feature (256)..(256): Xaa can be any naturally occurring amino acid
  misc_feature (271)..(271): Xaa can be any naturally occurring amino acid
  misc_feature (277)..(277): Xaa can be any naturally occurring amino acid
  misc_feature (280)..(281): Xaa can be any naturally occurring amino acid
  misc_feature (332)..(333): Xaa can be any naturally occurring amino acid
  misc_feature (337)..(337): Xaa can be any naturally occurring amino acid
  misc_feature (360)..(361): Xaa can be any naturally occurring amino acid
  misc_feature (367)..(367): Xaa can be any naturally occurring amino acid
  misc_feature (387)..(387): Xaa can be any naturally occurring amino acid
  misc_feature (395)..(395): Xaa can be any naturally occurring amino acid
  misc_feature (405)..(405): Xaa can be any naturally occurring amino acid
  misc_feature (415)..(415): Xaa can be any naturally occurring amino acid
  misc_feature (427)..(427): Xaa can be any naturally occurring amino acid
  misc_feature (429)..(429): Xaa can be any naturally occurring amino acid
MAAAVVGAXNRAPLSLXGSXSGXSSVPXPXFLGKKLTXSCXFXQSXSSXXNGSFKVXXEVGEXKLSDQSSWEALDADGSDYLYRLGEADNMNIAVGXRGMVDSLFQGPFLGTDSDIVFDYRQKXTRSFEYLQGDYYIAPAFMDKVVVHIXKNFLANLLNIKVPLILGIWGGKGQGKSFQTELIFAXMGIEPVIMSAGELESXRAGEPGKLIRXRYRXASQVIQNQGKMSCLMINDLDAGAGRFGXTQXTVNNQXVXGTLMNLADNPTRVSXGQMWRXSDXXRVPIIVTGNDFSTLYAPLIRDGRMEKFYWQPTREDIINIVHRMYEKDGISXXEVVXIVDTFPNQALDFYGALRSRTYDXXVLKWVXDIGGVENLGKKLLNSKKEGXPVFEPPEXTLEALLESGXSLVKEQELVXRVKLAKEYMKNXDX

SEQ ID NO: 30 - protein, 427 aa, Artificial Sequence
  modified sequence of the TaRCA 2b from the subgenome B
MAAAFSSTVGAPASTPTNFLGKKLKKQVTSAVNYHGKSSKANRFTVMAAENLDEKRNTDKWKGLAYDISDDQQDITRGKGIVDSLFQAPTGDGTHEAVLSSYEYISQGLKKYDFDNTMDGFYIAPAFMDKLVVHLSKNFMTLPNIKIPLILGIWGGKGQGKSFQCELVFAKMGINPIMMSAGELESGNAGEPAKLIRQRYREAADIIKKGKMCCLFINDLDAGAGRMGGTTQYTVNNQMVNATLMNIADAPTNVQLPGMYNKEENPRVPIVVTGNDFSTLYAPLIRDGRMEKFYWAPTRDDRIGVCKGIFRTDNVPDESVVKLVDTFPGQSIDFFGALRARVYDDEVRKWVTETGIENIGKRLVNSRDGPVTFEQPKMTIEKLLEYGHMLVQEQDNVKRVQLADKYLSEAALGDANQDAMKTGSFYG

SEQ ID NO: 31 - DNA, 1143 bp, Artificial Sequence
  modified TaRCA 2b sequence from the subgenome B with 11 amino acid permutations
atggccgccgaaaatctagatgaaaaacgtaacaccgataaatggaaaggtttagcatatgacatctcagatgaccagcaggatataaccagaggtaaaggcattgtagattctcttttccaagcacctaccggggatggaacccacgaggctgttcttagttcgtatgaatatatctctcagggattaaaaaagtatgatttcgataataccatggatggtttctacatcgcgcccgcatttatggacaaactggtagttcacctctctaaaaacttcatgacgctccctaacataaaaatacctctgatattaggcatctggggaggaaaaggccaaggtaagagctttcaatgtgaactggtgttcgcgaagatgggcataaaccctatcatgatgtccgctggagagttagaaagtggtaatgcgggtgaaccagctaaattgattcgtcaaaggtatagagaggctgctgacatcatcaaaaagggaaaaatgtgttgcttgttcattaatgacctcgacgccggcgcgggccgcatgggtgggacgactcaatacacagtgaacaaccagatggtgaacgctactttaatgaacatcgctgacgcaccgactaatgtacagctacccggcatgtataataaagaggaaaacccacgtgtcccgatcgttgttacgggcaatgacttttccactctatatgcaccgttaattcgcgatggtaggatggaaaaattctattgggccccgactcgcgatgatcgaattggcgtctgtaagggtatatttagaacagataatgtgccagatgaatcagtagtgaaacttgtagatacctttccgggtcagtccattgacttctttggggcgttaagagcaagggtttatgacgatgaggtacgcaaatgggttaccgaaacgggcattgagaatattggtaaacgtcttgttaactcaagagacggaccagtaacctttgaacagccaaaaatgaccatcgaaaaacttttggaatatggtcatatgttagtccaggagcaagataacgtcaagcgtgtccaactggcagataaatacttaagtgaagctgccctgggagatgcaaaccaggatgcgatgaagacaggtagcttttatggg

SEQ ID NO: 32 - protein, 381 aa, Artificial Sequence
  modified TaRCA 2b with 11 amino acid permutations
MAAENLDEKRNTDKWKGLAYDISDDQQDITRGKGIVDSLFQAPTGDGTHEAVLSSYEYISQGLKKYDFDNTMDGFYIAPAFMDKLVVHLSKNFMTLPNIKIPLILGIWGGKGQGKSFQCELVFAKMGINPIMMSAGELESGNAGEPAKLIRQRYREAADIIKKGKMCCLFINDLDAGAGRMGGTTQYTVNNQMVNATLMNIADAPTNVQLPGMYNKEENPRVPIVVTGNDFSTLYAPLIRDGRMEKFYWAPTRDDRIGVCKGIFRTDNVPDESVVKLVDTFPGQSIDFFGALRARVYDDEVRKWVTETGIENIGKRLVNSRDGPVTFEQPKMTIEKLLEYGHMLVQEQDNVKRVQLADKYLSEAALGDANQDAMKTGSFYG

SEQ ID NO: 33 - protein, 427 aa, Artificial Sequence
  modified sequence of TaRCA 2b from subgenome B with 8 amino acid permutations
MAAAFSSTVGAPASTPTNFLGKKLKKQVTSAVNYHGKSSKANRFTVMAAENLDEKRNTDKWKGLAYDISDDQQDITRGKGIVDSLFQAPTGDGTHEAVLSSYEYISQGLKKYDFDNTMGGFYIAPAFMDKLVVHLSKNFMTLPNIKIPLILGIWGGKGQGKSFQCELVFAKMGINPIMMSAGELESGNAGEPAKLIRQRYREAADIIKKGKMCCLFINDLDAGAGRMGGTTQYTVNNQMVNATLMNIADAPTNVQLPGMYNKEENPRVPIVVTGNDFSTLYAPLIRDGRMEKFYWAPTRDDRIGVCKGIFRTDNVPDESVVKIVDTFPGQSIDFFGALRARVYDDEVRKWVTSTGIENIGKRLVNSRDGPVTFEQPKMTIEKLLEYGHMLVQEQDNVKRVQLADKYLSEAALGDANQDAMKTGSFYG

SEQ ID NO: 34 - DNA, 1143 bp, Artificial Sequence
  modified TaRCA 2b from the subgenome B with 8 amino acid permutations
atggccgcggaaaatctggacgagaaacgcaataccgataaatggaaaggtctggcctatgatatttctgatgatcagcaggatattacacgtggtaaaggcattgtggattcactgtttcaggcaccgactggtgatggaacccacgaagcagtattgtctagctacgaatatatctctcaggggcttaaaaagtatgattttgacaacaccatgggcggtttttatatcgcgccggcattcatggataagcttgttgtccacctgtcgaaaaactttatgacgctccctaatattaaaattccattgattctcggtatttggggtggtaaaggccagggtaagtctttccagtgtgagttagtttttgccaagatgggcattaatccgatcatgatgagtgccggggagctagaaagtggtaatgccggtgaacctgctaaacttattcgccaacgctaccgcgaagccgcagacatcatcaaaaaaggtaagatgtgttgcttattcatcaatgatctcgatgctggagctgggcgcatgggtgggacaacccagtatactgttaacaatcagatggtcaatgcaacgctgatgaacatcgctgatgccccaaccaatgtgcaattaccaggcatgtacaataaggaagaaaatccccgtgttcccatcgtagtcactggaaatgactttagcacactctatgcgccccttattcgtgacggtagaatggaaaaattttattgggccccaactagggacgatcgtatcggcgtctgtaagggtatattcaggacggacaatgtccccgatgagagtgttgttaagatcgttgatacatttccaggtcagtctatagatttcttcggtgcactgcgggcgcgggtctacgacgatgaggtccgcaaatgggtgacttctaccggaattgaaaatatcggaaaacgtctcgtcaatagtagggacggcccagtgacctttgagcaacccaaaatgacaatagaaaagcttttggagtatggtcatatgctcgttcaagagcaagataacgttaagcgcgttcagctggcagataaatatctgagcgaggcggccttgggggatgctaatcaggacgcaatgaaaaccgggtccttctacgga

SEQ ID NO: 35 - protein, 381 aa, Artificial Sequence
  modified TaRCA 2b from subgenome B with 8 amino acid permutations
MAAENLDEKRNTDKWKGLAYDISDDQQDITRGKGIVDSLFQAPTGDGTHEAVLSSYEYISQGLKKYDFDNTMGGFYIAPAFMDKLVVHLSKNFMTLPNIKIPLILGIWGGKGQGKSFQCELVFAKMGINPIMMSAGELESGNAGEPAKLIRQRYREAADIIKKGKMCCLFINDLDAGAGRMGGTTQYTVNNQMVNATLMNIADAPTNVQLPGMYNKEENPRVPIVVTGNDFSTLYAPLIRDGRMEKFYWAPTRDDRIGVCKGIFRTDNVPDESVVKIVDTFPGQSIDFFGALRARVYDDEVRKWVTSTGIENIGKRLVNSRDGPVTFEQPKMTIEKLLEYGHMLVQEQDNVKRVQLADKYLSEAALGDANQDAMKTGSFYG

SEQ ID NO: 36 - DNA, 4326 bp, Artificial Sequence
  T-DNA
aattacaacggtatatatcctgccagtactgggccccctcgagggcgatcgctacgtacctgcaggcccgggttaattaagcctccgattgatgcttcaccaaaaaacaatatcaacagcagtgcaaaattagaatttttgtatttttgtggtaacgcaaaccggccatcaaaggggaaaaacgtacaatgcttatgttgtatgttaagagaagtttgtgtggtgccaaatgacagtcctagcctgacggttatcgagaaagcagaatatgtgcaggtggcagagcaaaatatttgtggtagtccaactagaatacaatttgcatgccatgcctcatccaagaagccgggcaacgagaggcagcaaaaggcttttctgtggtgatgcaaaatgaagaggttatgtagtagctgagctgatgaagcaactggtcgctagctgccggccgggagacgaatgtgaggcaaggaaagaaaagaaaaaacagagagaaagagttgatcagaaatgggtgaattctgtggtgaggaaaggtcaaggaactgaagccaagagatccttcctacctacactaatacaatactcctaactcgctcacagactccgatccaggtccaagtcatgctatgctgtggatcggccggccgagattgcgccacgtgtgcagaacccaatcttcagcgtgtggcctgtgggggatctggaagctgatccacagggagggaggagtgtgtgcctctcacagcttccaacttccatggcgacgtccaatgctattgtattatttaaggcctaccgcagctcggcctctacactttgagcagcagcggctggccatcatcagtgatcctctacaatcatcgactttcagcaaattaagtccaaaaccatggcatcagcattttcgtccactgtcggagctcctgcatctactcctacaaccttcctgggcaaaaaggtgaagaaacaagctggcgcgctaaactattaccacggcggaaacaagatcaacaatcgcgtcgtacgggcaatggctgcaaagaaagaactggacgaagggaagcagacagatgcagatcgctggaaaggccttgcctatgacataagcgacgatcagcaagacataacccgaggaaaggggatagtcgatagcctgttccaagctccgatgggtgatggcacacatgaggcgatcctatctagctacgagtacatctcccaaggactccggaaatacgacttcgacaacacaatggacggcctttacatcgctcctgcattcatggacaagctgatcgtccacttggctaagaactttatgacgctgcccaacatcaaagtcccgctaatcttgggaatttggggagggaaaggccaaggaaagagctttcaatgcgagctagtgttcgcgaaaatggggatcaacccgataatgatgagcgctggagaacttgagagtggaaacgcgggagaaccagctaagctcatacgacagaggtacagagaggcagcggacatcataaaaaaggggaagatgtgctgcttattcatcaacgacttggatgctggagcaggaaggatggggggaacgactcagtacacggtcaataaccagatggtcaacgcgaccctaatgaacatcgctgatgctcccactaacgtccaattcccggggatgtacaacaaggaagagaatccgcgtgttccgatcattgtgacggggaacgacttcagcaccttatatgctcctctcatacgggatggacggatggagaaattctattgggctcctacgagggaggatagaattggcgtctgcaaagggatcttccgaactgacaacgttccggatgaggctgtcgttcaactagttgacaccttccctggtcagagcattgatttctttggcgccttaagagctcgcgtatacgacgatgaagtccgaaaatgggtcggggagataggagtcgagaacatctctaagcgcctagttaactcacgagaaggacctcccacattcgaccaacccaagatgacgatcgagaagctcatggagtacggccatatgctagtccaagagcaagagaacgtcaagcgagttcaactcgcggacaaatatctgagtgaagctgcgttgggacaagcgaacgatgacgcaatggctactggagctttctacgggaagtgattgctagcacgcgtgcaggccggcttgacgcttgctattaattatttctcctttttatgttttctctttgttctgtgtatcagatcgcgcccaagccatagctgggcatgacaagtttttgttaataataatataatataaagatatgggctatactatacatgagtatatttcatttgggtatcatttgtcaatttctgtgtccatccagtgttttcttagaacaatactgtaagacggtttactgtcagccaaaatgctaaattaagcaatggatatcattaccctgttatccctaaagcttattaatataacttcgtatagcatacattatacgaagttatgtttcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctgaaatcaccagtctctctctataaatctatctctctctctataacaatggacccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgagatcacccgttctaggatccgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaaacataacttcgtatagcatacattatacgaagttatatggatctcgaggcattacggcattacggcactcgcgagggtcccaattcgagcatggagccatttacaattgaatatatcctgccg

SEQ ID NO: 37 - DNA, 6760 bp, Artificial Sequence
  T-DNA
aattacaacggtatatatcctgccagtactgggccccctcgagggcgatcgctacgtacctgcagggcggccgcaagcttttggtggtaggaatgtagttttctgacaaagtcaattactgaatataaaaaaaatctgcacagctctgcgtcaacagttgtccaagggatgcctcaaaaatctgtgcagattatcagtcgtcacgcagaagcagaacatcatggtgtgctaggtcagcttcttgcattgggccatgaatccggttggttgttaatctctcctctcttattctcttatattaagatgcataactcttttatgtagtctaaaaaaaaatccagtggatcggatagtagtacgtcatggtgccattaggtaccgttgaacctaacagatatttatgcatgtgtatatatatagctatatagacaaaattggtgccgattatagacccaaaagcaataggtatatataatataatacagaccacaccaccaaactaagaatcgatcaaatagacaaggcatgtctccaaattgtcttaaactatttccgtaggttcagccgttcaggagtcgaatcagcctctgccggcgttttctttgcacgtacgacggacacacatgggcataccatatagctggtccatgacattaggagagagaacgtacgtgttgacctgtagctgagatataacaaggttgattataatatcaccaaacatgaaatcatccaaggatgacccataactatcactactatagtactgcatctggtaaaagaaattgtatagactctatttcgagcactaccacataacgcctgcaatgtgacaccctacctattcactaatgtgcctcttcccacacgctttccacccgtactgctcacagctttaagaaccagaacaaatgagtaatattagtgtcggttcatggctaaaaccagcactgatgtacatgaccacatatgtcaaatgctgcttctaggcatgacccgctcttactaatacctactcatcgctagaagaattttcggctgataaattttcaatttaagcaagagttatccgcgttggttcataactcaaactgatggccccaaccatattagtgcaaatttcacatatgatcataaccttttcatatgaaatcggatcgtgatgaactttatataaacattgtagctgtcgatgatacctacaattttatagttcacaacctttttatttcaagtcatttaaatgcccaaataggtgtttcaaatctcagatagaaatgttcaaaagtaaaaaaggtccctatcataacataattgatatgtaagtgagttggaaaatgataagtacgtgtgagagagatcggagatcaaattctggtgtaataatgtatgtatttcagtcataaaaattggtagcagtagttggggctctgtatatataccggtaaggatgggatggtagtagaataattctttttttgtttttagttttttctggtccaaaatttcaaatttggatcccttacttgtaccaactaatattaatgagtgttgagggcagtagaggtgcaactttaccataatccctctgtttcaggttataagacgttttgactttaaatttgactaagtttatgcgcaaatatagtaatatttataatactaaattagtttcattaaataaataattgaatatattttcataataaatttgtgttgagttcaaaatattattaattttttctacaaacttggtcaaacttaaagcagtttgactttgaccaaagtcaaaacgtcttataacttgaaacggatggattactttttttgtggggacaagtttacaatgtttaataaaagcacaatccatcttaatgttttcaagctgaatattgtaaaattcatggataaaccagcttctaaatgtttaaccgggaaaatgtcgaacgacaaattaatatttttaagtgatggggagtattaattaaggagtgacaactcaactttcaatatcgtactaaactgtgggatttattttctaaaattttataccctgccaattcacgtgttgtagatctttttttttcactaaccgacaccaggtatatcaattttattgaatatagcagcaaaaagaatgtgttgtacttgtaaacaaaaagcaaactgtacataaaaaaaaatgcactcctatataattaagctcataaagatgctttgcttcgtgagggcccaagttttgatgaccttttgcttgatctcgaaattaaaatttaagtactgttaagggagttcacaccaccatcaattttcagcctgaagaaacagttaaacaacgaccccgatgaccagtctactgctctccacatactagctgcattattgatcacaaaacaaaacaaaacgaaataaaaatcagcagcgagagtgtgcagagagagacaaaggtgatctggcgtggatatctccccatccatcctcacccgcgctgcccatcactcgccgccgcatactccatcatgtggagagaggaagacgaggaccacagccagagcccgggtcgagatgccaccacggccacaacccacgagcccggcgcgacaccaccgcgcgcgcgtgagccagccacaaacgcccgcggataggcgcgcgcacgccggccaatcctaccacatccccggcctccgcggctcgcgagcgccgctgccatccgatccgctgagttttggctatttatacgtaccgcgggagcctgtgtgcagagcagtgcatctcaagaagtactcgagcaaagaaggagagagcttggtgagctgcagagcaggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaccatggcatcagcattttcgtccactgtcggagctcctgcatctactcctacaaccttcctgggcaaaaaggtgaagaaacaagctggcgcgctaaactattaccacggcggaaacaagatcaacaatcgcgtcgtacgggcaatggctgcaaagaaagaactggacgaagggaagcagacagatgcagatcgctggaaaggccttgcctatgacataagcgacgatcagcaagacataacccgaggaaaggggatagtcgatagcctgttccaagctccgatgggtgatggcacacatgaggcgatcctatctagctacgagtacatctcccaaggactccggaaatacgacttcgacaacacaatggacggcctttacatcgctcctgcattcatggacaagctgatcgtccacttggctaagaactttatgacgctgcccaacatcaaagtcccgctaatcttgggaatttggggagggaaaggccaaggaaagagctttcaatgcgagctagtgttcgcgaaaatggggatcaacccgataatgatgagcgctggagaacttgagagtggaaacgcgggagaaccagctaagctcatacgacagaggtacagagaggcagcggacatcataaaaaaggggaagatgtgctgcttattcatcaacgacttggatgctggagcaggaaggatggggggaacgactcagtacacggtcaataaccagatggtcaacgcgaccctaatgaacatcgctgatgctcccactaacgtccaattcccggggatgtacaacaaggaagagaatccgcgtgttccgatcattgtgacggggaacgacttcagcaccttatatgctcctctcatacgggatggacggatggagaaattctattgggctcctacgagggaggatagaattggcgtctgcaaagggatcttccgaactgacaacgttccggatgaggctgtcgttcaactagttgacaccttccctggtcagagcattgatttctttggcgccttaagagctcgcgtatacgacgatgaagtccgaaaatgggtcggggagataggagtcgagaacatctctaagcgcctagttaactcacgagaaggacctcccacattcgaccaacccaagatgacgatcgagaagctcatggagtacggccatatgctagtccaagagcaagagaacgtcaagcgagttcaactcgcggacaaatatctgagtgaagctgcgttgggacaagcgaacgatgacgcaatggctactggagctttctacgggaagtgattgctagcacgcgtttaattaaatttaaatggcgcgccgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcgatatcattaccctgttatccctaaagcttattaatataacttcgtatagcatacattatacgaagttatgtttcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctgaaatcaccagtctctctctataaatctatctctctctctataacaatggacccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgagatcacccgttctaggatccgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaaacataacttcgtatagcatacattatacgaagttatatggatctcgaggcattacggcattacggcactcgcgagggtcccaattcgagcatggagccatttacaattgaatatatcctgccg

SEQ ID NO: 38 - DNA, 1284 bp, Triticum aestivum
atggctgctgccttctcctccaccgtcggtgccccggcttctacgccgaccaacttcctcgggaagaagctcaagaagcaggtgacctcggccgtgaactaccatggcaagagctccaaggccaacaggttcacagtcatggcagcggaaaacatcgacgagaagaggaacacagacaagtggaagggtcttgcctacgatatctccgacgaccagcaggacatcaccagagggaagggcatcgtggactccctcttccaggcgcccacgggcgacggcacccacgaggccgtcctcagctcctacgagtacgtcagccagggactcaagaagtacgacttcgacaacaccatgggaggcttctacatcgctcctgctttcatggacaagcttgttgtccatctctccaagaacttcatgaccctgcccaacatcaagatcccactcatcttgggtatctggggaggcaagggtcaaggaaaatccttccagtgtgagcttgtcttcgccaagatgggcatcaacccaatcatgatgagtgccggagagctggagagtggcaacgccggagagccagccaagctcatcaggcagcggtaccgtgaggctgcagacatgatcaagaagggtaagatgtgctgcctcttcatcaacgatcttgacgccggtgcgggtcggatgggcgggaccacacagtacaccgtcaacaaccagatggtgaacgccaccctcatgaacatcgccgatgcccccaccaacgtgcagctcccaggcatgtacaacaaggaggagaaccctcgtgtgcccatcgtcgtcactggtaacgatttctcaacgttgtacgcccctctcatccgtgatggtcgtatggagaagttctactgggctcccacccgcgacgaccgtatcggtgtctgcaagggtatcttccagaccgacaatgtcagcgacgagtccgtcgtcaagatcgtcgacaccttcccaggacaatccatcgactttttcggtgctctgcgtgctcgggtgtacgacgacgaggtgcgcaagtgggtgacctctaccggtatcgagaacattggcaagaagcttgtgaactcgcgggacggaccagtgacctttgagcagccaaagatgacagtcgagaagctgctagagtacgggcacatgctcgtccaggagcaggacaatgtcaagcgtgtgcagcttgctgacacctacatgagccaggcagctctgggtgatgctaaccaggatgcgatgaagactggttccttctacggttag

SEQ ID NO: 39 - protein, 427 aa, Triticum aestivum
MAAAFSSTVGAPASTPTNFLGKKLKKQVTSAVNYHGKSSKANRFTVMAAENIDEKRNTDKWKGLAYDISDDQQDITRGKGIVDSLFQAPTGDGTHEAVLSSYEYVSQGLKKYDFDNTMGGFYIAPAFMDKLVVHLSKNFMTLPNIKIPLILGIWGGKGQGKSFQCELVFAKMGINPIMMSAGELESGNAGEPAKLIRQRYREAADMIKKGKMCCLFINDLDAGAGRMGGTTQYTVNNQMVNATLMNIADAPTNVQLPGMYNKEENPRVPIVVTGNDFSTLYAPLIRDGRMEKFYWAPTRDDRIGVCKGIFQTDNVSDESVVKIVDTFPGQSIDFFGALRARVYDDEVRKWVTSTGIENIGKKLVNSRDGPVTFEQPKMTVEKLLEYGHMLVQEQDNVKRVQLADTYMSQAALGDANQDAMKTGSFYG

SEQ ID NO: 40 - DNA, 1284 bp, Triticum aestivum
atggctgctgccttctcctccaccgtcggtgccccggcttctacgccgaccaacttcctcgggaagaagctcaagaagcaggtgacctcggccgtgaactaccatggcaagagctccaaggccaacaggttcacggtcatggcagcggaaaacatcgacgagaagaggaacaccgacaagtggaagggtcttgcgtacgatatctccgacgaccagcaggacatcaccagagggaagggcatcgtggactccctcttccaggcgcccactggcgacggcacccacgaggccgtcctcagctcatacgagtacgtcagccagggactcaagaagtacgacttcgacaacaccatgggaggcttctacatcgctcctgctttcatggacaagcttgttgtccatctctccaagaacttcatgaccctgcccaacatcaagatcccactcatcttgggtatctggggaggcaagggtcaaggaaaatccttccagtgtgagcttgtcttcgccaagatgggcatcaacccaatcatgatgagtgccggagagctggagagtggcaacgccggagagccagccaagctcatcaggcagcggtaccgtgaggctgcagacatgatcaagaagggtaagatgtgctgcctcttcatcaacgatcttgacgccggtgcgggtaggatgggcgggaccacacagtacaccgtcaacaaccagatggtgaacgccaccctcatgaacatcgccgatgcccccaccaatgtgcagctcccaggcatgtacaacaaggaggagaacccacgtgtgcccatcgtcgtcactggtaacgatttctcgacgttgtacgcccctctgatccgtgatggtcgtatggagaagttctactgggctcccacccgcgacgaccgtatcggtgtctgcaagggtatcttccagaccgacaatgtcagcgacgagtccgtcgtcaagatcgtcgacaccttcccaggacaatccatcgactttttcggtgctctgcgtgctcgggtgtacgacgacgaggtgcgcaagtgggtgacctctaccggtatcgagaacattggcaagaagctggtgaactcgcgggacggaccagtgacctttgagcagccaaagatgacagtggagaagctgctagagtacgggcacatgctcgtccaggagcaggacaatgtcaagcgtgtgcagcttgctgacacctacatgagccaggcagctctgggtgatgctaaccaggatgcgatgaagactggtaccttctacggttag

SEQ ID NO: 41 - protein, 427 aa, Triticum aestivum [sequence omitted]

SEQ ID NO: 42 - DNA, 1395 bp, Triticum aestivum
atggctgctgccttctcctccaccgtcggtgccccggcttctacgccgaccaacttcctcgggaagaagctcaagaagcaggtgacctcggccgtgaactaccatggcaagagctccaaggccaacaggttcacagtcatggcagcggaaaacatcgacgagaagaggaacacagacaagtggaagggtcttgcctacgatatctccgacgaccagcaggacatcaccagagggaagggcatcgtggactccctcttccaggcgcccacgggcgacggcacccacgaggccgtcctcagctcctacgagtacgtcagccagggactcaagaagtacgacttcgacaacaccatgggaggcttctacatcgctcctgctttcatggacaagcttgttgtccatctctccaagaacttcatgaccctgcccaacatcaagatcccactcatcttgggtatctggggaggcaagggtcaaggaaaatccttccagtgtgagcttgtcttcgccaagatgggcatcaacccaatcatgatgagtgccggagagctggagagtggcaacgccggagagccagccaagctcatcaggcagcggtaccgtgaggctgcagacatgatcaagaagggtaagatgtgctgcctcttcatcaacgatcttgacgccggtgcgggtcggatgggcgggaccacacagtacaccgtcaacaaccagatggtgaacgccaccctcatgaacatcgccgatgcccccaccaacgtgcagctcccaggcatgtacaacaaggaggagaaccctcgtgtgcccatcgtcgtcactggtaacgatttctcaacgttgtacgcccctctcatccgtgatggtcgtatggagaagttctactgggctcccacccgcgacgaccgtatcggtgtctgcaagggtatcttccagaccgacaatgtcagcgacgagtccgtcgtcaagatcgtcgacaccttcccaggacaatccatcgactttttcggtgctctgcgtgctcgggtgtacgacgacgaggtgcgcaagtgggtgacctctaccggtatcgagaacattggcaagaagcttgtgaactcgcgggacggaccagtgacctttgagcagccaaagatgacagtcgagaagctgctagagtacgggcacatgctcgtccaggagcaggacaatgtcaagcgtgtgcagcttgctgacacctacatgagccaggcagctctgggtgatgctaaccaggatgcgatgaagactggttccttctacggtaaaggggcacagcaaggtactttgcctgtgccggcaggatgcaccgaccagactgccaagaacttcgacccaacggcgaggagtgacgacggcagctgcctttacaccttttaa

SEQ ID NO: 43 - protein, 464 aa, Triticum aestivum
MAAAFSSTVGAPASTPTNFLGKKLKKQVTSAVNYHGKSSKANRFTVMAAENIDEKRNTDKWKGLAYDISDDQQDITRGKGIVDSLFQAPTGDGTHEAVLSSYEYVSQGLKKYDFDNTMGGFYIAPAFMDKLVVHLSKNFMTLPNIKIPLILGIWGGKGQGKSFQCELVFAKMGINPIMMSAGELESGNAGEPAKLIRQRYREAADMIKKGKMCCLFINDLDAGAGRMGGTTQYTVNNQMVNATLMNIADAPTNVQLPGMYNKEENPRVPIVVTGNDFSTLYAPLIRDGRMEKFYWAPTRDDRIGVCKGIFQTDNVSDESVVKIVDTFPGQSIDFFGALRARVYDDEVRKWVTSTGIENIGKKLVNSRDGPVTFEQPKMTVEKLLEYGHMLVQEQDNVKRVQLADTYMSQAALGDANQDAMKTGSFYGKGAQQGTLPVPAGCTDQTAKNFDPTARSDDGSCLYTF

SEQ ID NO: 44 - DNA, 1395 bp, Triticum aestivum
atggctgctgccttctcctccaccgtcggtgccccggcttctacgccgaccaacttcctcgggaagaagctcaagaagcaggtgacctcggccgtgaactaccatggcaagagctccaaggccaacaggttcacggtcatggcagcggaaaacatcgacgagaagaggaacaccgacaagtggaagggtcttgcgtacgatatctccgacgaccagcaggacatcaccagagggaagggcatcgtggactccctcttccaggcgcccactggcgacggcacccacgaggccgtcctcagctcatacgagtacgtcagccagggactcaagaagtacgacttcgacaacaccatgggaggcttctacatcgctcctgctttcatggacaagcttgttgtccatctctccaagaacttcatgaccctgcccaacatcaagatcccactcatcttgggtatctggggaggcaagggtcaaggaaaatccttccagtgtgagcttgtcttcgccaagatgggcatcaacccaatcatgatgagtgccggagagctggagagtggcaacgccggagagccagccaagctcatcaggcagcggtaccgtgaggctgcagacatgatcaagaagggtaagatgtgctgcctcttcatcaacgatcttgacgccggtgcgggtaggatgggcgggaccacacagtacaccgtcaacaaccagatggtgaacgccaccctcatgaacatcgccgatgcccccaccaatgtgcagctcccaggcatgtacaacaaggaggagaacccacgtgtgcccatcgtcgtcactggtaacgatttctcgacgttgtacgcccctctgatccgtgatggtcgtatggagaagttctactgggctcccacccgcgacgaccgtatcggtgtctgcaagggtatcttccagaccgacaatgtcagcgacgagtccgtcgtcaagatcgtcgacaccttcccaggacaatccatcgactttttcggtgctctgcgtgctcgggtgtacgacgacgaggtgcgcaagtgggtgacctctaccggtatcgagaacattggcaagaagctggtgaactcgcgggacggaccagtgacctttgagcagccaaagatgacagtggagaagctgctagagtacgggcacatgctcgtccaggagcaggacaatgtcaagcgtgtgcagcttgctgacacctacatgagccaggcagctctgggtgatgctaaccaggatgcgatgaagactggtaccttctacggtaaaggggcacagcaaggtactttgcctgtaccggcaggatgcaccgaccagactgccaagaacttcgacccaacggcgaggagtgacgacggcagctgcctttacaccttttaa

SEQ ID NO: 45 - protein, 464 aa, Triticum aestivum
MAAAFSSTVGAPASTPTNFLGKKLKKQVTSAVNYHGKSSKANRFTVMAAENIDEKRNTDKWKGLAYDISDDQQDITRGKGIVDSLFQAPTGDGTHEAVLSSYEYVSQGLKKYDFDNTMGGFYIAPAFMDKLVVHLSKNFMTLPNIKIPLILGIWGGKGQGKSFQCELVFAKMGINPIMMSAGELESGNAGEPAKLIRQRYREAADMIKKGKMCCLFINDLDAGAGRMGGTTQYTVNNQMVNATLMNIADAPTNVQLPGMYNKEENPRVPIVVTGNDFSTLYAPLIRDGRMEKFYWAPTRDDRIGVCKGIFQTDNVSDESVVKIVDTFPGQSIDFFGALRARVYDDEVRKWVTSTGIENIGKKLVNSRDGPVTFEQPKMTVEKLLEYGHMLVQEQDNVKRVQLADTYMSQAALGDANQDAMKTGTFYGKGAQQGTLPVPAGCTDQTAKNFDPTARSDDGSCLYTF

SEQ ID NO: 46 - DNA, 1299 bp, Triticum aestivum
atggcttctgctttctcgtccaccgttggagctccggcgtcgaccccgaccaccttcctcgggaagaaggtgaagaagcaggccggtgcgttgaactactaccatggtggcaacaagatcaacaatagggtggtcagggccatggcggccaaaaaggaacttgacgagggcaagcagaccgatgccgatcggtggaagggtctcgcttacgacatctccgatgaccagcaggacatcacgagggggaaaggcatcgtggactccctgttccaggcccccatgggcgacggcacccacgaggccatcctgagctcctacgagtacatcagccagggcctgcgcaagtacgacttcgacaacaccatggacgggctgtacatcgccccggcgttcatggacaagctcatcgtccacctcgccaagaacttcatgacactccccaacatcaaggtccctctcatcctgggtatctggggaggcaagggacagggcaagtcgttccagtgcgagctggtgttcgccaagatgggcatcaaccccatcatgatgagcgccggagagctggagagcggcaacgccggcgagccggccaagctgatccggcagaggtaccgcgaggctgccgacattatcaagaagggcaagatgtgctgcctcttcatcaacgacctggacgccggcgcggggcggatgggcgggacgacgcagtacacggtgaacaaccagatggtgaacgccaccctgatgaacatcgcggacgcgcccaccaacgtgcagctcccggggatgtacaacaaggaggagaacccacgcgtgcccatcatcgtcaccggcaacgacttctcgacgctgtacgcgcccctcatccgggacggccgcatggagaagttctactgggcgcccacccgggaggaccgcatcggcgtgtgcaagggcatcttccgcaccgacaacgtccccgacgaggccgtggtgaggctggtggacaccttcccggggcagtccatcgacttcttcggcgcgctgcgggcgcgggtgtacgacgacgaggtgcgcaagtgggtcggcgagatcggcgtcgagaacatctccaagcggctcgtcaactccagggaggggccgccgacgttcgaccagcccaagatgaccatcgagaagctcatggagtacggccacatgctggtccaggagcaggagaacgtgaagcgcgtgcagctcgccgacaagtacctcagcgaggcggcgctcggccaagccaacgacgacgccatggcgaccggcgccttctacggcaagtag

SEQ ID NO: 47 - protein, 432 aa, Triticum aestivum
MASAFSSTVGAPASTPTTFLGKKVKKQAGALNYYHGGNKINNRVVRAMAAKKELDEGKQTDADRWKGLAYDISDDQQDITRGKGIVDSLFQAPMGDGTHEAILSSYEYISQGLRKYDFDNTMDGLYIAPAFMDKLIVHLAKNFMTLPNIKVPLILGIWGGKGQGKSFQCELVFAKMGINPIMMSAGELESGNAGEPAKLIRQRYREAADIIKKGKMCCLFINDLDAGAGRMGGTTQYTVNNQMVNATLMNIADAPTNVQLPGMYNKEENPRVPIIVTGNDFSTLYAPLIRDGRMEKFYWAPTREDRIGVCKGIFRTDNVPDEAVVRLVDTFPGQSIDFFGALRARVYDDEVRKWVGEIGVENISKRLVNSREGPPTFDQPKMTIEKLMEYGHMLVQEQENVKRVQLADKYLSEAALGQANDDAMATGAFYGK

SEQ ID NO: 48 - DNA, 1299 bp, Triticum aestivum
atggcttctgctttctcatccaccgttggagctccggcgtcgaccccgaccaccttcctcgggaagaaggtgaagaagcaggccggtacgttgaactactaccatggtggcaacaagatcaacagtagggtggtcagggccatggcggccaaaaaggaacttgacgagggcaagcagaccgatgccgatcggtggaagggtctcgcttacgacatctccgacgaccagcaggacatcaccagggggaaaggtatcgtggactccctgttccaggcccccatgggcgacggcacccacgaggccatcctgagctcctacgagtacatcagccagggcctgcggaagtacgacttcgacaacaccatggacgggctctacatcgccccggcattcatggacaagctcatcgtccacctcgccaagaacttcatgacgctccccaacatcaaggtccctctcatcctgggcatctggggaggcaagggacagggcaagtcgttccagtgcgagctggtgttcgccaagatgggcatcaaccccatcatgatgagcgccggagagctggagagcggcaacgccggcgagccggccaagctgatccggcagaggtaccgcgaggcggcggacattatcaagaagggcaagatgtgctgcctcttcatcaacgacctggacgccggcgcggggcggatgggcgggacgacgcagtacacggtgaacaaccagatggtgaacgccaccttgatgaacatcgcggacgcgcccaccaacgtgcagctcccggggatgtacaacaaggaggagaacccacgcgtgcccatcatcgtcaccggcaacgacttctcgacgctgtacgcgcccctcatccgcgacggccgcatggagaagttctactgggcgcccacccgcgaggaccgcatcggggtgtgcaagggcatcttccgcaccgacaacgtcccggacgaggctgtggtgaggctggtggacaccttcccggggcaatccatcgacttcttcggcgcgctgcgggcgcgggtgtacgacgacgaggtgcgcaagtgggtcggcgagatcggcgtcgagaacatctccaagcgcctcgtcaactccagggaggggccgccgacgttcgaccagcccaagatgaccatcgagaagctcatggagtacggccacatgctggtccaggagcaggagaacgtgaagcgtgtgcagctcgccgacaagtacctcagcgaggcggcgctcggccaagccaacgacgacgccatggcgaccggcgccttctacggcaagtag

SEQ ID NO: 49 - protein, 432 aa, Triticum aestivum
MASAFSSTVGAPASTPTTFLGKKVKKQAGTLNYYHGGNKINSRVVRAMAAKKELDEGKQTDADRWKGLAYDISDDQQDITRGKGIVDSLFQAPMGDGTHEAILSSYEYISQGLRKYDFDNTMDGLYIAPAFMDKLIVHLAKNFMTLPNIKVPLILGIWGGKGQGKSFQCELVFAKMGINPIMMSAGELESGNAGEPAKLIRQRYREAADIIKKGKMCCLFINDLDAGAGRMGGTTQYTVNNQMVNATLMNIADAPTNVQLPGMYNKEENPRVPIIVTGNDFSTLYAPLIRDGRMEKFYWAPTREDRIGVCKGIFRTDNVPDEAVVRLVDTFPGQSIDFFGALRARVYDDEVRKWVGEIGVENISKRLVNSREGPPTFDQPKMTIEKLMEYGHMLVQEQENVKRVQLADKYLSEAALGQANDDAMATGAFYGK

SEQ ID NO: 50 - DNA, 19 bp, Artificial Sequence
  primer
gggtcggcgagatcggcgt

SEQ ID NO: 51 - DNA, 23 bp, Artificial Sequence
  primer
ccagcatgtggccgtactccatg

SEQ ID NO: 52 - DNA, 23 bp, Artificial Sequence
  primer
ccttctacggtaaaggggcacag

SEQ ID NO: 53 - DNA, 21 bp, Artificial Sequence
  primer
tgtaaaggcagctcccgtcgt

SEQ ID NO: 54 - DNA, 24 bp, Artificial Sequence
  primer
ccatacacacccaccatctcttgc

SEQ ID NO: 55 - DNA, 21 bp, Artificial Sequence
  primer
tgtaaaggcagctcccgtcgt

SEQ ID NO: 56 - DNA, 25 bp, Artificial Sequence
  primer
caaatacgccatcagggagaacatc

SEQ ID NO: 57 - DNA, 20 bp, Artificial Sequence
  primer
cgctgccgaaaccacgagac

SEQ ID NO: 58 - DNA, 23 bp, Artificial Sequence
  primer
ttcttttctcacaacccaacgac

SEQ ID NO: 59 - DNA, 21 bp, Artificial Sequence
  primer
gcctcccgacattgccatctg

SEQ ID NO: 60 - DNA, 5739 bp, artificial
  T-DNA
aattacaacggtatatatcctgccagtactcggccgtcgaccgcggtaccccggaattttgtggcgctctatcatagctataaacctattcagcacaatattagttagctaggggccccctcgagggcgatcgctacgtacctgcagggcggccgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgagctcgttgggtcgaagttcttggcagtctggtcggtgcatcctgccggtacaggcaaagtaccttgctgtgcccctttaccgtagaaggtaccagtcttcatcgcatcctggttagcatcacccagagctgcctggctcatgtaggtgtcagcaagctgcacacgcttgacattgtcctgctcctggacgagcatgtgcccgtactctagcagcttctccactgtcatctttggctgctcaaaggtcactggtccgtcccgcgagttcaccagcttcttgccaatgttctcgataccggtagaggtcacccacttgcgcacctcgtcgtcgtacacccgagcacgcagagcaccgaaaaagtcgatggattgtcctgggaaggtgtcgacaatcttgacgacggactcgtcgctgacattgtcgggtccgccttgtttctcctctgtctcttgatctgactaatcttggtttatgattcgttgagtaattttggggaaagctagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagccgacaatgtcagcgacgagtccgtcgtcaagattgtcgacaccttcccaggacaatccatcgactttttcggtgctctgcgtgctcgggtgtacgacgacgaggtgcgcaagtgggtgacctctaccggtatcgagaacattggcaagaagctggtgaactcgcgggacggaccagtgacctttgagcagccaaagatgacagtggagaagctgctagagtacgggcacatgctcgtccaggagcaggacaatgtcaagcgtgtgcagcttgctgacacctacatgagccaggcagctctgggtgatgctaaccaggatgcgatgaagactggtaccttctacggtaaaggggcacagcaaggtactttgcctgtaccggcaggatgcaccgaccagactgccaagaacttcgacccaacgcgttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcactagaattcgatatcattaccctgttatccctaaagcttattaatataacttcgtatagcatacattatacgaagttatgtttcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctgaaatcaccagtctctctctataaatctatctctctctctataacaatggacccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgagatcacccgttctaggatccgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaaacataacttcgtatagcatacattatacgaagttatcaaaacgtcgtgagacagtttggttaactataacggtcctaaggtagcgattagttagctagcgagcggcgaactaataactccgctctaccgaaagttactcgaggcattacggcattacggcactcgcgagggtcccaattcgagcatggagccatttacaattgaatatatcctgccg

SEQ ID NO: 61 - DNA, 23 bp, Artificial Sequence
  guide RNA
acctcggccgtgaactaccatgg

SEQ ID NO: 62 - DNA, 23 bp, Artificial Sequence
  guide RNA
tccctcttccaggcgcccacggg

SEQ ID NO: 63 - DNA, 23 bp, Artificial Sequence
  guide RNA
tgacgacgatgggcacacgaggg

SEQ ID NO: 64 - DNA, 23 bp, Artificial Sequence
  guide RNA
tgacgtactcgtatgagctgagg

SEQ ID NO: 65 - DNA, 23 bp, Artificial Sequence
  guide RNA
ctacatgagccaggcagctctgg

SEQ ID NO: 66 - DNA, 23 bp, Artificial Sequence
  guide RNA
aaccaggatgcgatgaagactgg

SEQ ID NO: 67 - DNA, 23 bp, Artificial Sequence
  guide RNA
cccaccatctcttgctgcatagg

SEQ ID NO: 68 - DNA, 23 bp, Artificial Sequence
  guide RNA
accatctcttgctgcataggagg

SEQ ID NO: 69 - DNA, 23 bp, Artificial Sequence
  guide RNA
attcttcttacctgttcttctgg